SEQUENCE LISTING (13 sequences)

SEQ ID NO: 1 - protein, 4 aa, Artificial Sequence
  Synthetic
GFLG

SEQ ID NO: 2 - protein, 4 aa, Artificial Sequence
  Synthetic
KPLR

SEQ ID NO: 3 - protein, 23 aa, Artificial Sequence
  Synthetic
YNKRKRIHIGPGRAFYTTKNIIG

SEQ ID NO: 4 - protein, 13 aa, Artificial Sequence
  Synthetic
AKFVAAWTLKAAA

SEQ ID NO: 5 - protein, 4 aa, Artificial Sequence
  Synthetic
YLLL

SEQ ID NO: 6 - protein, 17 aa, influenza
LNDKHSNGTIKDRSPYR

SEQ ID NO: 7 - protein, 13 aa, influenza
DPNGWTGTDNNFS

SEQ ID NO: 8 - protein, 11 aa, influenza
RNNILRTQESE

SEQ ID NO: 9 - protein, 9 aa, hepatitis B
PRVRGLYFL

SEQ ID NO: 10 - protein, 20 aa, HPV
QLYQTCKAAGTCPSDVIPKI

SEQ ID NO: 11 - protein, 35 aa, Malaria
EDNEKLRKPKHKKLKQPADGNPDPNANPNVDPNAN

SEQ ID NO: 12 - protein, 52 aa, Malaria
ILRNQYNNIIELEKTKHIIHNKKDTYKYDIKLKESDILMFYMKEETIVESGN

SEQ ID NO: 13 - protein, 56 aa, Malaria
VLNKKEKKPRGIDFTETDELEQTDIVQNGNDKLVKVKENETIHFKFNSNQKLEIKE

(19) United States
(12) Patent Application Publication
(10) Pub. No.: US 2023/0026627 A1
(43) Pub. Date: Jan. 26, 2023

(54) Compositions and Methods of Manufacturing Star Polymers for Ligand Display and/or Drug Delivery

(71) Applicants: Vaccitech North America, Inc., Baltimore, MD (US); Institute of Macromolecular Chemistry, Prague 6 (CZ); The United States of America, as represented by the Secretary, Department of Health and Human Servic, Bethesda, MD (US)

(72) Inventors: Geoffrey Lynn, Baltimore, MD (US); Yaling Zhu, Gaithersburg, MD (US); Jacob Holechek, Union Bridge, MD (US); David Wilson, Baltimore, MD (US); Joe Francica, Washington, DC (US); Richard Laga, Prague (CZ); Gabriela Muzíková, Prague (CZ)

(73) Assignees: Vaccitech North America, Inc., Baltimore, MD (US); Institute of Macromolecular Chemistry, Prague 6 (CZ); The United States of America, as represented by the Secretary, Department of Health and Human Servic, Bethesda, MD (US)

(21) Appl. No.: 17/604,227

(22) PCT Filed: Apr. 16, 2020
(86) PCT No.: PCT/US2020/028586

§ 371 Date: Oct. 15, 2021

Related U.S. Application Data
(60) Provisional application No. 62/835,268, filed on Apr. 17, 2019.

(51) Int. Cl. C08G 83/00; A61K 47/64; A61K 39/39

(52) U.S. Cl. CPC C08G 83/003; A61K 47/641; A61K 39/39

(57) ABSTRACT

A star polymer of formula O[P1]-([X]-A[P2]-[Z]-[P3])n where O is a core; A is a polymer arm attached to the core; X is a linker molecule between the core and the polymer arm; Z is a linker molecule between the end of the polymer arm and P3; P1, P2 and P3 are each independently one or more pharmaceutically active compounds that act extracellularly or intracellularly, n is an integer number; [ ] denotes that the group is optional; and at least one of P1, P2 or P3 is present.

### PRIORITY DOCUMENT

The present application claims priority from U.S. Provisional Patent Application No. 62/835,268 titled “COMPOSITIONS AND METHODS OF MANUFACTURING STAR POLYMERS FOR LIGAND DISPLAY AND/OR DRUG DELIVERY” and filed on 17 Apr. 2019, the content of which is hereby incorporated by reference in its entirety.

This invention was created in the performance of a Cooperative Research and Development Agreement with the National Institutes of Health, an Agency of the Department of Health and Human Services. The Government of the United States has certain rights in this invention.

### TECHNICAL FIELD

The present disclosure relates to systems for displaying and/or delivering pharmaceutically active compounds.

### BACKGROUND

Particle delivery systems can be used to modulate the pharmacokinetics of pharmaceutically active compounds used for a variety of applications. For example, particle delivery systems based on liposomes, micelles and linear polymers have been used to package small molecule cytotoxic drugs (‘chemotherapeutics’) used for cancer treatment. Particle delivery systems for packaging small molecule drugs have been used to perform any one or all of the following functions: (i) improve drug solubility; (ii) limit distribution and passively or actively target drug molecules to specific tissues; (iii) control the release of drug into specific tissues; and (iv) protect drug molecules from degradation.

In addition to the aforementioned functions, particle delivery systems used with ligands that bind to extracellular receptors may also perform the function of providing a scaffold for arraying the ligand to optimally engage its cognate extracellular receptor (i.e. ligand display). Applications of particle delivery systems for arraying ligands for binding extracellular receptors include the use of delivery systems to array B cell immunogens to optimally engage B cell receptors as a means for inducing antibody responses for the treatment or prevention of infectious diseases as well as cancer. Other applications include the array of peptide-MHC complexes on particles to engage T cells as a means to induce tolerance. Another application includes the use of particle delivery systems to array therapeutic monoclonal antibodies or antibody fragments that can be used for the treatment of variety of diseases that rely on recombinant antibody technologies.

There are a variety of challenges that presently limit the utility of particle delivery systems. Many particle delivery systems are often limited by relatively low loading of pharmaceutically active compounds, i.e. low mass ratio of compound to polymer mass, which limits the concentration of active compound that can reach tissues where it is needed. Therefore, next generation delivery systems should be developed to maximize loading of pharmaceutically active compounds.

Another challenge is that many particle delivery systems, such as liposomes and PLGA particles, are often larger than >100 nm or may form aggregates that may be too large for the intended application and/or may induce immune activation due to interaction of the aggregates with monocyte populations. In this regard, particles between 10-100 nm in size have been proposed to be an optimal size range for use in a variety of applications, including for array of B cell immunogens for use as vaccines, as well as for the intravenous delivery of chemotherapeutics and/or immunostimulants to cancers.

A further challenge is that particle delivery systems based on amphiphilic materials often require high net charge (i.e. positive or negative zeta potential) to keep the particles from aggregating. This high net charge can lead to unwanted interactions of the materials with certain tissues, such as non-specific interactions of positively charged particles with cell surfaces. Therefore, novel delivery systems that do not carry high net charge are needed as a means to improve delivery of pharmaceutically active compounds to target tissues by avoiding non-specific interactions with other tissues.

An especially pronounced challenge that has not been adequately addressed by contemporary technologies is the induction of unwanted antibodies against the delivery system or cargo that can lead to rapid clearance of the delivery system from the blood following two or more injections, referred to as “accelerated blood clearance.” The utility of any delivery system of pharmaceutically active compounds may be limited by the induction of unwanted antibody responses. Therefore, approaches for limiting the induction of antibodies that lead to accelerated blood clearance are needed.

Finally, manufacturability remains a major challenge to the translation of particle delivery systems. Particle delivery systems based on emulsions often have high and variable loading as well as broad ranges of particle sizes. Therefore, chemically defined approaches to achieving precise and reproducible loading on narrow range sizes of particles are needed.

There is thus a need to provide particle delivery systems for displaying and/or delivering pharmaceutically active compounds that address one or more of the aforementioned challenges.

### SUMMARY

In a first aspect, provided herein is a star polymer of formula O[P1]-([X]-A[P2]-[Z]-[P3])n where O is a core; A is a polymer arm attached to the core; X is a linker molecule between the core and the polymer arm; Z is a linker molecule between the end of the polymer arm and P3; P1, P2 and P3 are each independently one or more compounds that act extracellularly or intracellularly, n is an integer number; [ ] denotes that the group is optional; and at least one of P1, P2 or P3 is present.

In certain embodiments of the star polymer of the first aspect, any one or more of P1, P2 or P3 is a ligand (L) comprising a pharmaceutically active compound that acts extracellularly.

In certain embodiments of the star polymer of the first aspect, any one or more of P2 and P3 is a ligand L.

In certain embodiments of the star polymer of the first aspect, any one or more of P1, P2 or P3 is a drug (D) comprising a pharmaceutically active compound that acts intracellularly.

In a second aspect, disclosed herein is a star polymer of formula O-([X]-A[(D)]-[Z]-L)n, where O is a core; A is a polymer arm attached to the core; X is a linker molecule between the core and the polymer arm; Z is a linker molecule between the end of the polymer arm a ligand, L; D is a drug comprising a pharmaceutically active compound that acts intracellularly; L is a ligand comprising a pharmaceutically active compound that acts extracellularly; n is an integer number greater than or equal to 2; and [ ] denotes that the group is optional.

In certain embodiments of the star polymer of the second aspect, n is greater than or equal to 5.

In certain embodiments of the star polymer of the second aspect, the majority monomer units comprising the polymer arm (A) are selected from hydrophilic monomers.

In certain embodiments of the star polymer of the second aspect, the polymer arms (A) comprise negatively charged functional groups.

In certain embodiments of the star polymer of the second aspect, the polymer arm (A) comprises 1 to 20 mol % co-monomers comprising negatively charged functional groups.

In certain embodiments of the star polymer of the second aspect, the co-monomers comprising negatively charged functional groups comprise poly(anionic) oligomers or polymers.

In certain embodiments of the star polymer of the second aspect, the polymer arms (A) comprise a di-block copolymer architecture.

In certain embodiments of the star polymer of the second aspect, any co-monomers comprising negatively charged functional groups are on a first block of the di-block copolymer that is proximal to the ligand (L)

In certain embodiments of the star polymer of the second aspect, one or more drugs (D), if present, are attached to co-monomers on a second block of the di-block copolymer that is proximal to the core (O), and the first block is solvent exposed and is not attached to any drugs (D).

In certain embodiments of the star polymer of the second aspect, the polymer arm length is selected to increase the size of the star polymer as a means to increase the persistence of activity of the star polymer in selected tissues.

In certain embodiments of the star polymer of the second aspect, the polymer arm length is selected to control the hydrodynamic radius of the star polymer.

In certain embodiments of the star polymer of the second aspect, the polymer arm molecular weight is greater than about 10,000 Daltons.

In certain embodiments of the star polymer of the second aspect, the hydrodynamic radius of the star polymer is greater than about 10 nm.

In certain embodiments of the star polymer of the second aspect, comprising two or more ligands (L), which may be the same or different, the ligands are selected from compounds that bind to extracellular receptors selected from protein or peptide antigens, therapeutic antibodies or antibody fragments, peptide-MHC complexes, agonists of TLRs 1, 2, 4, 5, 6, CLRs or NLRs, or combinations thereof.

In certain embodiments of the star polymer of the second aspect, the star polymer further comprises one or more amplifying linkers that enable attachment of two or more ligands (L), which may be the same or different, on the ends of at least some of the polymer arms (A).

In certain embodiments of the star polymer of the second aspect, the density of ligands (L) attached to the star polymer is greater than 5.

In certain embodiments of the star polymer of the second aspect, saccharides that bind to the lectin receptor, CD22L, are placed at or near the ends of the polymer arms (A) proximal to the ligand (L).

In certain embodiments of the star polymer of the second aspect, drug(s), if present, are arrayed along the polymer arms (A) at a density greater than about 3 mol %.

In certain embodiments of the star polymer of the second aspect, the drug (D), if present, has a molecular weight of between about 200-1,000 Da and the drug (D) is arrayed along the polymer arms (A) at a density of between about 4.0 to about 50 mol % to achieve a mass percent of about 10 to about 80 mass %.

In certain embodiments of the star polymer of the second aspect, the polymer arm (A) comprises hydrophilic monomers selected from acrylates, (meth)acrylates, acrylamides, (meth)acrylamides, allyl ethers, vinyl acetates, vinyl amides, substituted styrenes, amino acids, acrylonitrile, heterocyclic monomers (i.e. ethylene oxide), saccharides, phosphoesters, phosphonamides, sulfonate esters, sulfonamides, or combinations thereof.

In certain embodiments of the star polymer of the second aspect, the core (O) has greater than 5 points of attachment for polymer arms (A).

In certain embodiments of the star polymer of the second aspect, the core (O) comprises a branched polymer or dendrimer.

In certain embodiments of the star polymer of the second aspect, the dendrimer or branched polymer that is used to form the core (O) has surface amine groups used for the attachment of polymer arms (A).

In certain embodiments of the star polymer of the second aspect, the core (O) is a dendrimer selected from PAMAM, bis(MPA) or lysine.

In certain embodiments of the star polymer of the second aspect, the core (O) is a branched polymer that comprises monomers selected from poly(amino acids) or saccharides.

In a third aspect, disclosed herein is a star polymer of formula O-([X]-A(D)-[Z]-[L])n, where O is a core; A is a polymer arm attached to the core; X is a linker molecule between the core and the polymer arm; Z is a linker molecule between the end of the polymer arm and a ligand, L; D is a drug comprising a pharmaceutically active compound that acts intracellularly; L is a ligand comprising a pharmaceutically active compound that acts extracellularly; n is an integer number; and [ ] denotes that the group is optional.

In certain embodiments of the star polymer of the third aspect, n is greater than or equal to 5.

In certain embodiments of the star polymer of the third aspect, the majority monomer units comprising the polymer arm (A) are selected from hydrophilic monomers.

In certain embodiments of the star polymer of the third aspect, the polymer arms (A) comprise negatively charged functional groups.

In certain embodiments of the star polymer of the third aspect, the polymer arm (A) comprises 1 to 20 mol % co-monomers comprising negatively charged functional groups.

In certain embodiments of the star polymer of the third aspect, the co-monomers comprising negatively charged functional groups comprise poly(anionic) oligomers or polymers.

In certain embodiments of the star polymer of the third aspect drug(s), (D) are arrayed along the polymer arms (A) at a density greater than about 3 mol %.

In certain embodiments of the star polymer of the third aspect, the drug (D), if present, has a molecular weight of between about 200-1,000 Da and the drug (D) is arrayed along the polymer arms (A) at a density of between about 4.0 to about 50 mol % to achieve a mass percent of about 10 to about 80 mass %.

In certain embodiments of the star polymer of the third aspect, the polymer arms (A) comprises a di-block copolymer architecture.

In certain embodiments of the star polymer of the third aspect, any co-monomers comprising negatively charged functional groups are on a first block of the di-block copolymer that is distal to the core (O) and solvent exposed.

In certain embodiments of the star polymer of the third aspect, the one or more drugs (D) are attached to co-monomers on a second block of the di-block copolymer that is proximal to the core (O), and the first block is solvent exposed and is not attached to any pharmaceutically active compounds.

In certain embodiments of the star polymer of the third aspect, the polymer arm length is selected to increase the size of the star polymer as a means to increase the persistence of activity of the star polymer in selected tissues.

In certain embodiments of the star polymer of the third aspect, the polymer arm length is selected to control the hydrodynamic radius of the star polymer.

In certain embodiments of the star polymer of the third aspect, the polymer arm molecular weight is between about than about 5,000 to about 50,000 Daltons.

In certain embodiments of the star polymer of the third aspect, the hydrodynamic radius of the star polymer is between about 5 nm and about 15 nm.

In certain embodiments of the star polymer of the third aspect, the ligand (L), if present, is selected from compounds that bind to extracellular receptors selected from protein or peptide antigens, therapeutic antibodies or antibody fragments, peptide-MHC complexes, agonists of TLRs 1, 2, 4, 5, 6, CLRs or NLRs; or combinations thereof.

In certain embodiments of the star polymer of the third aspect, further comprises one or more amplifying linkers that enable attachment of two or ligands (L), which may be the same or different, on the ends of at least some of the polymer arms (A).

In certain embodiments of the star polymer of the third aspect, the density of ligands (L) attached to the star polymer is greater than 5.

In certain embodiments of the star polymer of the third aspect, saccharides that bind to the lectin receptor, CD22L, are placed at or near the ends of the polymer arms (A) proximal to the ligand (L).

In certain embodiments of the star polymer of the third aspect, the polymer arm (A) comprises hydrophilic monomers selected from acrylates, (meth)acrylates, acrylamides, (meth)acrylamides, allyl ethers, vinyl acetates, vinyl amides, substituted styrenes, amino acids, acrylonitrile, heterocyclic monomers (i.e. ethylene oxide), saccharides, phosphoesters, phosphonamides, sulfonate esters, sulfonamides, or combinations thereof.

In certain embodiments of the star polymer of the third aspect, the core (O) has greater than 5 points of attachment for polymer arms (A).

In certain embodiments of the star polymer of the third aspect, the core (O) comprises a branched polymer or dendrimer.

In certain embodiments of the star polymer of the third aspect, the dendrimer or branched polymer that is used to form the core (O) has surface amine groups used for the attachment of polymer arms (A).

In certain embodiments of the star polymer of the third aspect, the core (O) is a dendrimer selected from PAMAM, bis(MPA) or lysine.

In certain embodiments of the star polymer of the third aspect, the core (O) is a branched polymer that comprises monomers selected from poly(amino acids) or saccharides.

In a fourth aspect, disclosed herein is a composition for sustaining activity of a pharmaceutically active compound that acts extracellularly comprising the star polymer of any of the first to third aspects, wherein L is present and the star polymer has a hydrodynamic radius greater than 20 nm Rh.

In a fifth aspect, disclosed herein is an antitumor composition comprising the star polymer of any of the first to third aspects, wherein D is present and selected from small molecule chemotherapeutic and/or immunostimulant drugs (D) and the star polymer has a hydrodynamic radius of from about 5 to about 15 nm Rh.

In a sixth aspect, disclosed herein is an antiviral composition comprising the star polymer of any of the first to third aspects, wherein L is present in the star polymer.

In a seventh aspect, disclosed herein is a vaccine composition for inducing antibody responses comprising the star polymer of any of the first to third aspects, wherein the polymer arm molecular weights are an average of about 10 kDa to about 60 kDa.

In an eight aspect, disclosed herein is a process for preparing a star polymer, the process comprising: reacting a heterotelechelic polymer arm (A) comprising a linker precursor Z1 with a ligand (L) comprising a linker precursor Z2 under conditions to form a linker molecule (Z) between the polymer arm (A) and the ligand (L):

[X2]-A[P2]-Z1+Z2-L→[X2]-A[P2]-Z-L,

and reacting the polymer arm-linker-ligand molecule comprising a linker precursor X2 with a core comprising a plurality of linker precursors X1 to form the star polymer:

O-X1+X2-A[P2]-Z-L→O(X-A[P2]-Z-L)n.

In a ninth aspect, disclosed herein is a process for preparing a star polymer, the process comprising: reacting a heterotelechelic polymer arm (A) comprising a linker precursor X2 with a core comprising a plurality of linker precursors X1 under conditions to form a core (O) attached to a plurality of polymer arms (A) via a linker molecule (X):

O-X1+X2-A[P2]-Z1→O(X-A[P2]-Z1)n where n is an integer number, and

reacting the core-linker-polymer arm molecule comprising a linker precursor Z1 with a ligand (L) comprising a linker precursor Z2 under conditions to form a linker molecule (Z) between the polymer arm (A) and the ligand (L) to form the star polymer O(X-A[P2]-Z1)n+Z2-L→O(X-A[P2]-Z-L)n.

In a tenth aspect, disclosed herein is a process for preparing star polymers, the process comprising:
(a) reacting monomers in the presence of a chain transfer agent, optionally linked to Z1 or P3 directly or indirectly through Z, and an initiator under conditions to form a polymer arm of formula A-[-Z1, -P3 or -Z-P3] wherein A is a polymer arm, Z1 is a linker precursor, Z is a linker molecule, P3 is one or more compounds that act extracellularly or intracellularly and [ ] denotes that any of Z1, P3 or Z-P3 may or may not be present,
(b) reacting the polymer arm of formula A-[-Z1, -P3 or -Z-P3] with excess initiator functionalized with linker precursor X2 under conditions to form a polymer arm of formula A-[-Z1, -P3 or -Z-P3]→X2-A-[-Z1, -P3 or -Z-P3], and
(c) reacting the polymer arm of formula X2-A-[-Z1, -P3 or -Z-P3] with a core (O) comprising a plurality of linker precursors X1 under conditions to form the star polymer O-X1+X2-A-[Z1, -P3 or -Z-P3]→O(X-A-[Z1, -P3, -Z-P3])n where X is a linker molecule and n is an integer number.

In an eleventh aspect, disclosed herein is a process for preparing a star polymer, the process comprising:
(a) reacting a core (O) with a linker precursor X1 comprised of 4 or more ethylene oxide units to produce a core comprising a plurality of linker precursors X1 with 4 or more ethylene oxide units, and
(b) reacting a heterotelechelic polymer arm (A) comprising a linker precursor X2 with the core comprising the plurality of linker precursors X1 with 4 or more ethylene oxide units under conditions to form a core (O) attached to a plurality of polymer arms (A) via a linker molecule (X) comprising 4 or more ethylene oxide units.

### BRIEF DESCRIPTION OF DRAWINGS

Embodiments of the present invention will be discussed with reference to the accompanying drawings wherein:

FIG. 1 shows a schematic depiction of star polymers composed of a PAMAM dendrimer core and HPMA-based polymers arms. Multiple peptide-based antigens (yellow and purple) are linked to the ends of the polymer arms. Small molecule immunostimulant drugs (D) may also be attached, shown as blue polygons in the lower row scheme;

FIG. 2 is a generic structure of a star polymer of the present disclosure used for ligand array, wherein a dendrimer core (O) is linked through a linker X to an integer number (n) of polymer arms (A) that are linked to a ligand (L) through a linker Z;

FIG. 3 shows a synthetic route for the synthesis of star polymer carriers of a peptide-based antigen comprising an HIV minimal immunogen as the ligand (L). HPMA monomers (1) are polymerized to yield 10 kDa polymer arms (2); the polymer arms are conjugated to G5 PAMAM dendrimers (3) by acylation to yield star polymers (4); then, a peptide immunogen (5) is conjugated to the HPMA grafts by Cu¹ catalyzed cycloaddition to yield a star polymer arraying multiple peptide-based antigens comprised of an HIV minimal immunogen (6);

FIG. 4 shows dynamic light scattering analysis of star polymers of the present disclosure based on peptide-based antigens comprising an HIV minimal immunogen linked to 10 kDa HPMA polymer arms linked to G5 PAMAM dendrimers;

FIG. 5 shows that star polymers of the present disclosure restrict the biodistribution and increase retention of arrayed ligands (L), which in this case is a peptide-based antigen comprising an HIV minimal immunogen. Mice were immunized subcutaneously in the left footpad with star polymers of the present disclosure bearing AlexaFluor647-labeled V3 peptide ligands (L); control mice were immunized with soluble AlexaFluor647-labeled V3 peptides. Mice were imaged at the indicated time points following vaccination. Composite overlays of x-ray and fluorescent images are shown;

FIG. 6 shows the injection site kinetics of star polymer carriers of peptide-based antigen as compared with peptide-based antigen alone following subcutaneous administration, which was measured by quantifying fluorescence in the left footpad at the time points indicated. Data points indicate group geometric means and 95% confidence intervals; vertical line indicates immunization; *, statistical difference by ANOVA, comparing between groups at each time point;

FIG. 7 shows optimization of immunogenic compositions of star polymers displaying peptide-based antigens as ligands (L). Mice were immunized subcutaneously with star polymers bearing 5, 15, or 30 peptide-based antigens comprising an HIV minimal immunogen (“V3”) per star polymer, either or alone or co-delivering the peptide-based antigen PADRE for T cell help. The V3 dose (5 μg) was constant across all groups; all vaccines were adjuvanted by admixing with a soluble TLR7/8 agonist;

FIG. 8 shows that immunogenic compositions of star polymers comprising two types of ligands both V3, a B cell immunogen, and PADRE, a helper T cell epitope, lead to optimal antibody responses. Mice were immunized with either soluble V3 alone; soluble V3 plus star polymers linked to PADRE; star polymers bearing V3 plus star polymers linked to PADRE; or, star polymers linked to both V3 and PADRE. The density (15 per star polymer) and dose (5 μg) of V3 was constant across all star polymer groups; all vaccines were adjuvanted by admixing with a soluble TLR7/8 agonist;

FIG. 9 shows a comparison of different adjuvants for use with star polymers of the present disclosure displaying a peptide-based antigen comprising an HIV minimal immunogen as the ligand (L). Star polymers bearing V3 and PADRE were left unadjuvanted, or were either admixed with a TLR7/8 agonist, the emulsion adjuvant AddaVax, Alhydrogel or Adju-Phos;

FIG. 10 shows a comparison of different vaccination routes of immunogenic compositions of star polymers of the present disclosure. Star polymers bearing TLR7/8 agonist immunostimulant drugs (D) (linked to the core, i.e. at P1), as well as V3 and PADRE (linked to the polymer arms) were administered intramuscularly (IM), subcutaneously (SC) or intravenously (IV). In all studies, serum antibody responses were measured by ELISA after 2 homologous immunizations;

FIG. 11 shows antibody responses induced by different compositions of a peptide-based antigen comprising an HIV minimal immunogen (V3). The peptide-based antigen, V3, was administered to mice as either soluble V3 admixed with adjuvant, V3 at either 3 or 5 mol % density on a statistical copolymer admixed with adjuvant, or V3 arrayed on the surface of a star polymer co-delivering TLR-7/8 agonist immunostimulant drugs (D) (linked to the core, i.e. at P1) as adjuvant; and

FIG. 12 shows the impact that polymer arm density, polymer arm molecular weight and dendrimer core generation have on the size (Rg) of star polymers based on HPMA-based polymer arms linked to PAMAM-based dendrimer cores. These results demonstrate that star polymer hydrodynamic size can be precisely tuned principally by varying the molecular weight of the polymer arms.

FIG. 13 shows the impact that polymer arm length (expressed as molecular weight; see Table 1) and ligand (L) density have on star polymer hydrodynamic radius (Rh).

FIG. 14 shows that the synthetic route used to synthesize polymer arms (A) can impact the propensity of star polymers to cross-link, which results in increased molecular weight and polydispersity index (PDI) determined by gel permeation chromatography (GPC) in tandem with multi-angle light scattering (MALS) and refractive index (RI) detectors, which provided Mw and Mn, respectively. The figure shows polydispersity index (PDI: Mw/Mn) change over time for star polymers produced using polymer arms with the linker precursor X2 added to the polymer arm either (i) during polymerization or (ii) during the capping step.

FIGS. 15 and 16 show turbidity for different polymer arms in aqueous buffer (i.e. PBS) over a pH range of 5.5 to 7.5. Note: turbidity (OD at 490 nm)>0.05 indicates that the polymers are aggregating.

FIG. 17 shows survival curves for C57BL/6 mice that were implanted subcutaneously with MC38 tumors, randomized to groups and then provided the indicated treatment (normalized to 50 nmol of TLR-7/8a, 2BXy) by direct intratumoral injection between days 7-10 after tumor implantation.

### DESCRIPTION OF EMBODIMENTS

Details of terms and methods are given below to provide greater clarity concerning compounds, compositions, methods and the use(s) thereof for the purpose of guiding those of ordinary skill in the art in the practice of the present disclosure. The terminology in this disclosure is understood to be useful for the purpose of providing a better description of particular embodiments and should not be considered limiting.

About: In the context of the present disclosure, “about” when referring to a measurable value such as an amount, a temporal duration, and the like, is meant to encompass variations of ±20%, 10%, +5%, 1%, or +0.1% from the specified value, as such variations are appropriate to perform the disclosed methods.

Adjuvant: Any material added to vaccines to enhance or modify the immunogenicity of an antigen. Adjuvants can be delivery systems, such as particles based on inorganic salts (e.g., aluminum hydroxide or phosphate salts referred to as alum), water-in-oil or oil-in-water emulsions or polymer particles (e.g., PLGA) in which antigen is simply admixed with or adsorbed, incorporated within or linked indirectly or directly through covalent interactions. Alternatively, adjuvants can be chemically defined molecules that bind to specific receptors and induce downstream signalling, including pattern recognition receptor (PRR) agonists, such as synthetic or naturally occurring agonists of Toll-like receptors (TLRs), stimulator of interferon genes (STING), nucleotide-binding oligomerization domain-like receptors (NLRs), retinoic acid-inducible gene-I-like receptors (RLRs) or C-type lectin receptors (CLRs), as wells as biological molecules (a “biological adjuvant”), such as IL-2, RANTES, GM-CSF, TNF-α, IFN-γ, G-CSF, LFA-3, CD72, B7-1, B7-2, OX-40L, 4-1BBL. Small molecule analogs of nucleotide bases, such as hydroxyadenine and imidazoquinolines, that bind to Toll-like receptors-7 (TLR-7) and TLR-7/8a are used as exemplary PRR agonists in the present disclosure. The person of ordinary skill in the art is familiar with adjuvants (see: Perrie et al., Int J Pharm 364:272-280, 2008 and Brito et al., Journal of controlled release, 190C:563-579, 2014). For clarity, certain pharmaceutically active compounds that act intracellularly (D), such as small molecule drugs that bind intracellular receptors, or pharmaceutically active compounds that that act extracellularly, referred to herein as ligands (L), and have immunostimulatory properties can act as adjuvants when used in vaccines but may also be used for other applications.

Administration: To provide or give to a subject an agent, for example, an immunogenic composition comprising a star polymer as described herein, by any effective route. Exemplary routes of administration include, but are not limited to, oral, injection (such as subcutaneous, intramuscular, intradermal, intraperitoneal, and intravenous), transdermal (for example, topical), intranasal, vaginal, and inhalation routes.

“Administration of” and “administering a” compound should be understood to mean providing a compound, a prodrug of a compound, a star polymer composition or a pharmaceutical composition as described herein. The compound or composition can be administered by another person to the subject or it can be self-administered by the subject.

Antigen-presenting cell (APC): Any cell that presents antigen bound to MHC class I or class II molecules to T cells, including but not limited to monocytes, macrophages, dendritic cells, B cells, T cells and Langerhans cells.

Antigen: Any molecule that contains an epitope that binds to a T cell or B cell receptor and can stimulate an immune response, in particular, a B cell response and/or a T cell response in a subject. The epitopes may be comprised of peptides, glycopeptides, lipids or any suitable molecules that contain an epitope that can interact with components of specific B cell or T cell proteins. Such interactions may generate a response by the immune cell. “Epitope” refers to the region of a peptide antigen to which B and/or T cell proteins, i.e., B-cell receptors and T-cell receptors, interact.

Amphiphilic: The term “amphiphilic” is used herein to mean a substance containing both hydrophilic or polar (water-soluble) and hydrophobic (water-insoluble) groups.

CD4: Cluster of differentiation 4, a surface glycoprotein that interacts with MHC Class II molecules present on the surface of other cells. A subset of T cells express CD4 and these cells are commonly referred to as helper T cells.

CD8: Cluster of differentiation 8, a surface glycoprotein that interacts with MHC Class I molecules present on the surface of other cells. A subset of T cells express CD8 and these cells are commonly referred to as cytotoxic T cells or killer T cells.

Charge: A physical property of matter that affects its interactions with other atoms and molecules, including solutes and solvents. Charged matter experiences electrostatic force from other types of charged matter as well as molecules that do not hold a full integer value of charge, such as polar molecules. Two charged molecules of like charge repel each other, whereas two charged molecules of different charge attract each other. Charge is often described in positive or negative integer units.

Charged monomers (C): refers to monomers that have one or more functional groups that are positively or negatively charged. The functional groups comprising the charged monomers may be partial or full integer values of charge. A charged monomer may have a single charged functional group or multiple charged functional groups, which may be the same or different. Functional groups may be permanently charged or the functional groups comprising the charged molecule may have charge depending on the pH. The charged monomer may be comprised of positively charged functional groups, negatively charged functional groups or both positive and negatively charged functional groups. The net charge of the charged monomer may be positive, negative or neutral. The charge of a molecule, such as a charged monomer, can be readily estimated based on a molecule's Lewis structure and accepted methods known to those skilled in the art. Charge may result from inductive effects, e.g., atoms bonded together with differences in electron affinity may result in a polar covalent bond resulting in a partially negatively charged atom and a partially positively charged atom. For example, nitrogen bonded to hydrogen results in partial negative charge on nitrogen and a partial positive charge on the hydrogen atom. Alternatively, an atom may be considered to have a full integer value of charge when the number of electrons assigned to that atom is less than or equal to the atomic number of the atom. The charge of a functional group is determined by summing the charge of each atom comprising the functional group. The net charge of the charged monomer is determined by summing the charge of each atom comprising the molecule. Those skilled in the art are familiar with the process of estimating charge of a molecule, or individual functional groups, by summing the formal charge of each atom in a molecule or functional group, respectively.

Charged monomers may comprise negatively charged functional groups such as those that occur as the conjugate base of an acid at physiologic pH (e.g., functional groups with a pKa less than about 6.5), e.g., at a pH of about 7.4. These include but are not limited to molecules bearing carboxylates, sulfates, sulfonates, phosphates, phosphoramidates, and phosphonates. Charged monomers may comprise positively charged functional groups such as those that occur as the conjugate acid of a base at physiologic pH (e.g., functional groups wherein the pKa of the conjugate acid of a base is greater than about 8.5). These include but are not limited to molecules bearing primary, secondary and tertiary amines, as well as ammonium and guanidinium. Charged monomers may comprise functional groups with charge that is pH independent, including quaternary ammonium, phosphonium and sulfonium functional groups. Charged monomers useful for the practice of the invention of the present disclosure are disclosed herein. Charged monomers on a copolymer are sometimes referred to as charged comonomers.

Chemotherapeutic: Chemotherapeutic agents are chemical compounds useful in the treatment of cancer and include growth inhibitory agents or other cytotoxic agents and include alkylating agents, anti-metabolites, anti-microtubule inhibitors, topoisomerase inhibitors, receptor tyrosine kinase inhibitors, angiogenesis inhibitors and the like. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; members of taxoid or taxane family, such as paclitaxel (TAXOL®docetaxel (TAXOTERE®) and analogues thereof; chlorambucil; gemcitabine; 6-thioguanine; mercaptopurine; methotrexate; platinum analogues such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; inhibitors of receptor tyrosine kinases and/or angiogenesis, including sorafenib (NEXAVAR®), sunitinib (SUTENT®), pazopanib (VOTRIENT™), toceranib (PALLADIA™), vandetanib (ZACTIMA™), cediranib (RECENTIN®), regorafenib (BAY 73-4506), axitinib (AG013736), lestaurtinib (CEP-701), erlotinib (TARCEVA®), gefitinib (IRESSA™), BIBW 2992 (TOVOK™), lapatinib (TYKERB®), neratinib (HKI-272), and the like, and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (FARESTON®); and anti-androgens such as flutamide, nilutamide, bicalutamide, leuprolide, and goserelin; and pharmaceutically acceptable salts, acids or derivatives of any of the above. Other conventional cytotoxic chemical compounds as those disclosed in Wiemann et al., 1985, in Medical Oncology (Calabresi et al, eds.), Chapter 10, McMillan Publishing, are also suitable chemotherapeutic agents. Chemotherapeutics are a type of pharmaceutically active compound and chemotherapeutics that act intracellularly are referred to herein as drugs (D). Chemotherapeutics that act intracellularly and are of relatively low molecular weight are referred to herein as small molecule drugs.

Click chemistry reaction: A bio-orthogonal reaction that joins two compounds together under mild conditions in a high yield reaction that generates minimal, biocompatible and/or inoffensive byproducts. An exemplary click chemistry reaction used in the present disclosure is the reaction of an azide group provided on a linker precursor Z1 with an alkyne provided on a linker precursor Z2 that forms a triazole linker (Z) through strain-promoted [3+2] azide-alkyne cyclo-addition.

Copolymer: A polymer derived from two (or more) monomeric species of polymer, as opposed to a homopolymer where only one monomer is used. Since a copolymer includes at least two types of constituent units (also structural units), copolymers may be classified based on how these units are arranged along the chain. A copolymer may be a statistical copolymer wherein the two or monomer units are distributed randomly; or, the copolymer may be an alternating copolymer wherein the two or more monomer units are distributed in an alternating sequence. The term “block copolymer” may be used herein to refer to a copolymer that comprises two or more homopolymer subunits linked by covalent bonds in which the union of the homopolymer subunits may require an intermediate non-repeating subunit, such as a junction block or linker. The term “block copolymer” may also be used herein to refer to a copolymer that comprises two or more copolymer subunits linked by covalent bonds in which the union of the copolymer subunits may require an intermediate non-repeating subunit, such as a junction block or linker. Block copolymers with two or three distinct blocks are referred to herein as “di-block copolymers” and “tri-block copolymers,” respectively. Copolymers may be referred to generically as polymers, e.g., a statistical copolymer may be referred to as a polymer or copolymer. Similarly a block copolymer may be referred to generically as a polymer.

Drug(s): in the broadest use of the term may be used to describe any pharmaceutically active compound; however, drug(s) and drug molecule(s) are used herein to describe pharmaceutically active compounds that act intracellularly and are indicated by a capital “D,” such as that used in the formulae of certain embodiments of star polymers. Pharmaceutically active compounds that act intracellularly, i.e. drugs (D), that are of relatively low molecular weight, no more than 10,000 Daltons, typically no more than 2,000 Daltons, often between about 200 to 1,000 Daltons, are referred to as small molecule drugs (D). Drug(s) (D) may act intracellularly by binding or associating with molecules inside of a cell to exert an effect at the cellular or organismal level.

Graft polymer: May be described as a polymer that results from the linkage of a polymer of one composition to the side chains of a second polymer of a different composition. A first polymer linked through co-monomers to a second polymer is a graft co-polymer. A first polymer linked through an end group to a second polymer may be described as a block polymer (e.g., A-B type di-block) or an end-grafted polymer. Polymer arms linked (or ‘grafted’) to cores (O) based on branched polymers or dendrimers may be referred to as graft polymers.

Hydrophilic: Refers to the tendency of a material to disperse freely in aqueous media. A material is considered hydrophilic if it has a preference for interacting with other hydrophilic material and avoids interacting with hydrophobic material. In some cases, hydrophilicity may be used as a relative term, e.g., the same molecule could be described as hydrophilic or not depending on what it is being compared to. Hydrophilic molecules are often polar and/or charged and have good water solubility, e.g., are soluble up to 0.1 mg/mL or more.

Hydrophobic: Refers to the tendency of a material to avoid contact with water. A material is considered hydrophobic if it has a preference for interacting with other hydrophobic material and avoids interacting with hydrophilic material. Hydrophobicity is a relative term; the same molecule could be described as hydrophobic or not depending on what it is being compared to. Hydrophobic molecules are often non-polar and non-charged and have poor water solubility, e.g., are insoluble down to 0.1 mg/mL or less.

Immune response: A change in the activity of a cell of the immune system, such as a B cell, T cell, or monocyte, as a result of a stimulus, either directly or indirectly, such as through a cellular or cytokine intermediary. In one embodiment, the response is specific for a particular antigen (an “antigen-specific response”). In one embodiment, an immune response is a T cell response, such as a CD4 T cell response or a CD8 T cell response. In one embodiment, an immune response results in the production of additional T cell progeny. In one embodiment, an immune response results in the movement of T cells. In another embodiment, the response is a B cell response, and results in the production of specific antibodies or the production of additional B cell progeny. In other embodiments, the response is an antigen-presenting cell response. “Enhancing an immune response” refers to co-administration of an adjuvant and an immunogenic agent, such as a peptide antigen, as part of a peptide antigen conjugate, wherein the adjuvant increases the desired immune response to the immunogenic agent compared to administration of the immunogenic agent to the subject in the absence of the adjuvant. In some embodiments, an antigen is used to stimulate an immune response leading to the activation of cytotoxic T cells that kills virally infected cells or cancerous cells. In some embodiments, an antigen is used to induce tolerance or immune suppression. A tolerogenic response may result from the unresponsiveness of a T cell or B cell to an antigen. A suppressive immune response may result from the activation of regulatory cells, such as regulatory T cells that downregulate the immune response, i.e. dampen then immune, response. Antigens administered to a patient in the absence of an adjuvant are generally tolerogenic or suppressive and antigens administered with an adjuvant are generally stimulatory and lead to the recruitment, expansion and activation of immune cells.

Immunogenic composition: A formulation of materials comprising an antigen and optionally an adjuvant that induces a measurable immune response against the antigen.

Immunostimulants: refers to a type of pharmaceutically active substance that activates cells of the immune system. Immunostimulants include ligands (L) that bind to certain extracellular receptors, such as agonists that bind to extracellular PRRs, interleukins, chemokines or certain antibodies, antibody fragments or synthetic peptides that activate immune cells, e.g., through binding to stimulatory receptors, e.g., anti-CD40, or, e.g., by blocking inhibitory receptors, e.g., anti-CTLA4 anti-PD1, as well as drugs (D), particularly small molecule drugs, that bind to certain intracellular receptors, such as agonists of intracellular PRRs.

Ligand(s): in the broadest use of the term may be used to describe any molecule that forms a complex with a biomolecule; however, ligand(s) and ligand molecule(s) are used herein to describe pharmaceutically active compounds that act extracellularly and are indicated by a capital “L,” such as that used in the formulae of a star polymer. Ligands (L) may act extracellularly by binding or associating with soluble molecules and/or cell surface bound molecules to exert a physiological effect.

Net charge: The sum of electrostatic charges carried by a molecule or, if specified, a section of a molecule.

Pattern recognition receptors (PRRs): Receptors expressed by various cell populations, particularly innate immune cells that bind to a diverse group of synthetic and naturally occurring molecules referred to as pathogen-associated molecular patterns (PAMPS) as well as damage associated molecular patterns (DAMPs). PAMPs are conserved molecular motifs present on certain microbial organisms and viruses. DAMPs are cellular components that are released or expressed during cell death or damage.

PAMP or DAMP activation of pattern recognition receptors induces an intracellular signaling cascade resulting in the alteration of the host cell's physiology. Such physiological changes can include changes in the transcriptional profile of the cell to induce expression of a range of pro-inflammatory and pro-survival genes. The coordinated expression of these genes may enhance adaptive immunity.

There are several classes of PRRs. Non-limiting examples of PRRs include Toll-like receptors (TLRs), RIG-I-like receptors (RLRs), NOD-like receptors (NLRs), Stimulator of Interferon Genes receptor (STING), and C-type lectin receptors (CLRs). Agonists of such PRRs can be used to enhance an immune response to a target antigen.

Agonists of PRRs are adjuvants and are referred herein as ligands (L) or drugs (D) depending on whether they act extracellularly or intracellularly, respectively. In some embodiments of the present disclosure, PRR agonists are used as adjuvants to enhance the immune response to a peptide antigen.

Toll-like receptors (TLRs) 1-13 are transmembrane PRRs that recognize a diverse range of PAMPs. There are two broad categories of TLRs: those that are localized to the cell surface and those that are localized to the endosomal lumen. TLRs that are present on the cell surface are typically important in recognition of bacteria. TLRs that are localized to the lumen of endosomes, such as TLRs 3, 7, 8, and 9, serve to recognize nucleic acids and are thus typically important in recognition of viruses and therefore in the promotion of antiviral immune responses. Polyinosinic-polycytidylic acid is a ligand for TLR-3. TLR-7 and TLR-8 recognize single stranded RNA as well as nucleotide base analogs and imidazoquinolines. TLR-9 recognizes unmethylated deoxycytidylate-phosphate-deoxyguanylate (CpG) DNA, found primarily in bacteria.

The NOD-like receptors (NLRs) and the RIG-I-like receptors (RLRs) are localized to the cytoplasm. Non-limiting examples of RLRs include RIG-I, MDA5, and LGP2. There are 22 human NLRs that can be subdivided into the five structurally related NLR families A, B, C, P, and X. All NLRs have three domains: an N-terminal domain involved in signaling, a nucleotide-binding NOD domain, and a C-terminal leucine rich region (LRR) important for ligand recognition. Non-limiting examples of NLRs include NALP3 and NOD2.

For more information on pattern recognition receptors, see Wales et al., Biochem Soc Trans., 35:1501-1503, 2007.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compositions, such as one or more therapeutic cancer vaccines, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutically active compound: Any protein, peptide, sugar, saccharide, nucleoside, inorganic compound, lipid, nucleic acid, small synthetic chemical compound, such as a small molecule drug or organic compound, or any combinations thereof, that has a physiological effect when ingested or otherwise introduced or administered into the body. Pharmaceutically active compounds can be selected from a variety of known classes of compounds, including, for example, analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, such as therapeutic antibodies and antibody fragments, MHC-peptide complexes, cytokines and growth factors, glycoproteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines. For clarity, drugs (D) that act intracellularly and ligands (L) that act extracellularly are types of pharmaceutically active compounds. Pharmaceutically active compounds may also be referred to as pharmaceutically active agents, pharmaceutically active substances or biologically active compounds or bioactive molecules.

Plurality: The word “plurality” is used herein to mean more than one.

Polar: A description of the properties of matter. Polar is a relative term, and may describe a molecule or a portion of a molecule that has partial charge that arises from differences in electronegativity between atoms bonded together in a molecule, such as the bond between nitrogen and hydrogen. Polar molecules have a preference for interacting with other polar molecules and typically do not associate with non-polar molecules. In specific, non-limiting cases, a polar group may contain a hydroxyl group, or an amino group, or a carboxyl group, or a charged group. In specific, non-limiting cases, a polar group may have a preference for interacting with a polar solvent such as water. In specific, non-limiting cases, introduction of additional polar groups may increase the solubility of a portion of a molecule.

Polymer: A molecule containing repeating structural units (monomers). Polymers linked to cores (O) are referred to as polymer arms (A).

Purified: Having a composition that is relatively free of impurities or substances that adulterate or contaminate a substance. The term purified is a relative term and does not require absolute purity. Thus, for example, a purified peptide preparation is one in which the peptide or protein is more enriched than the peptide or protein is in its natural environment, for example, within a cell. In one embodiment, a preparation is purified such that the peptide antigen conjugate represents at least 50% of the total content of the preparation. Substantial purification denotes purification from other proteins or cellular components. A substantially purified protein is at least 60%, 70%, 80%, 90%, 95%, 98%, or 99% pure. Thus, in one specific, non-limiting example, a substantially purified protein is 90% free of other proteins or cellular components or contaminating peptides.

Soluble: Capable of becoming molecularly or ionically dispersed in a solvent to form a homogeneous solution. A soluble molecule is understood to be freely dispersed as single molecules in solution and does not assemble into multimers or other supramolecular structures through interactions. Solubility can be determined by visual inspection, by turbidity measurements or by dynamic light scattering.

Subject and patient: These terms may be used interchangeably herein to refer to both human and non-human animals, including birds and non-human mammals, such as rodents (for example, mice and rats), non-human primates (for example, rhesus macaques), companion animals (for example domesticated dogs and cats), livestock (for example pigs, sheep, cows, llamas, and camels), as well as non-domesticated animals (for example big cats).

T Cell: A type of white blood cell that is part of the immune system and may participate in an immune response. T cells include, but are not limited to, CD4 T cells and CD8 T cells. A CD4 T cell displays the CD4 glycoprotein on its surface and these cells are often referred to as helper T cells. These cells often coordinate immune responses, including antibody responses and cytotoxic T cell responses, however, CD4 T cells can also suppress immune responses or CD4 T cells may act as cytotoxic T cells. A CD8 T cell displays the CD8 glycoprotein on its surface and these cells are often referred to as cytotoxic or killer T cells, however, CD8 T cells can also suppress immune responses.

Telechelic: Is used to describe a polymer that has one or two reactive ends that may be the same or different. The word is derived from telos and chele, the Greek words for end and claw, respectively. A semi-telechelic polymer describes a polymer with only a single end group, such as a reactive functional group that may undergo additional reactions, such as polymerization. A heterotelechelic polymer describes a polymer with two end groups, such as reactive functional groups, that have different reactive properties. Herein, polymer arms (A) with different linkers precursors at each end, i.e., X2 and Z1, are heterotelechelic polymers.

Treating, preventing, or ameliorating a disease: “Treating” refers to an intervention that reduces a sign or symptom or marker of a disease or pathological condition after it has begun to develop. For example, treating a disease may result in a reduction in tumor burden, meaning a decrease in the number or size of tumors and/or metastases, or treating a disease may result in immune tolerance that reduces systems associated with autoimmunity. “Preventing” a disease refers to inhibiting the full development of a disease. A disease may be prevented from developing at all. A disease may be prevented from developing in severity or extent or kind. “Ameliorating” refers to the reduction in the number or severity of signs or symptoms or marker of a disease, such as cancer.

Reducing a sign or symptom or marker of a disease or pathological condition related to a disease, refers to any observable beneficial effect of the treatment and/or any observable effect on a proximal, surrogate endpoint, for example, tumor volume, whether symptomatic or not. Reducing a sign or symptom associated with a tumor or viral infection can be evidenced, for example, by a delayed onset of clinical symptoms of the disease in a susceptible subject (such as a subject having a tumor which has not yet metastasized, or a subject that may be exposed to a viral infection), a reduction in severity of some or all clinical symptoms of the disease, a slower progression of the disease (for example by prolonging the life of a subject having a tumor or viral infection), a reduction in the number of relapses of the disease, an improvement in the overall health or well-being of the subject, or by other parameters well known in the art (e.g., that are specific to a particular tumor or viral infection). A “prophylactic” treatment is a treatment administered to a subject who does not exhibit signs of a disease or exhibits only early signs for the purpose of decreasing the risk or severity of developing pathology.

Tumor or cancer or neoplastic: An abnormal growth of cells, which can be benign or malignant, often but not always causing clinical symptoms. “Neoplastic” cell growth refers to cell growth that is not responsive to physiologic cues, such as growth and inhibitory factors.

A “tumor” is a collection of neoplastic cells. In most cases, tumor refers to a collection of neoplastic cells that forms a solid mass. Such tumors may be referred to as solid tumors. In some cases, neoplastic cells may not form a solid mass, such as the case with some leukemias. In such cases, the collection of neoplastic cells may be referred to as a liquid cancer.

Cancer refers to a malignant growth of neoplastic cells, being either solid or liquid. Features of a cancer that define it as malignant include metastasis, interference with the normal functioning of neighboring cells, release of cytokines or other secretory products at abnormal levels and suppression or aggravation of inflammatory or immunological response(s), invasion of surrounding or distant tissues or organs, such as lymph nodes, etc.

A tumor that does not present substantial adverse clinical symptoms and/or is slow growing is referred to as “benign.”

“Malignant” means causing, or likely to cause in the future, significant clinical symptoms. A tumor that invades the surrounding tissue and/or metastasizes and/or produces substantial clinical symptoms through production and secretion of chemical mediators having an effect on nearby or distant body systems is referred to as “malignant.”

“Metastatic disease” refers to cancer cells that have left the original tumor site and migrated to other parts of the body, e.g., via the bloodstream, via the lymphatic system, or via body cavities, such as the peritoneal cavity or thoracic cavity.

The amount of a tumor in an individual is the “tumor burden”. The tumor burden can be measured as the number, volume, or mass of the tumor, and is often assessed by physical examination, radiological imaging, or pathological examination.

An “established” or “existing” tumor is a tumor that exists at the time a therapy is initiated. Often, an established tumor can be discerned by diagnostic tests. In some embodiments, an established tumor can be palpated. In some embodiments, an established tumor is at least 500 mm³, such as at least 600 mm³, at least 700 mm³, or at least 800 mm³ in size. In other embodiments, the tumor is at least 1 cm long. With regard to a solid tumor, an established tumor generally has a newly established and robust blood supply, and may have induced the regulatory T cells (Tregs) and myeloid derived suppressor cells (MDSC).

A person of ordinary skill in the art would recognize that the definitions provided above are not intended to include impermissible substitution patterns (e.g., methyl substituted with 5 different groups, and the like). Such impermissible substitution patterns are easily recognized by a person of ordinary skill in the art. Any functional group disclosed herein and/or defined above can be substituted or unsubstituted, unless otherwise indicated herein. Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. The term “comprises” means “includes.” Therefore, comprising “A” or “B” refers to including A, including B, or including both A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described herein. In case of conflict, the present specification, including explanations of terms, will control. In addition, the materials, methods, and examples are illustrative only and not intended to be limiting.

The present disclosure arises from the inventors' development of novel compositions of matter and methods of manufacturing star polymers having linear polymer arms radiating from branched core structures. The branched core serves as a scaffold for arraying two or more polymer arms to create a star polymer. The star polymer serves as a scaffold for arraying various types of pharmaceutically active compounds, including ligands that act extracellularly, such as by binding to extracellular receptors, as well as compounds that act intracellularly, such as small molecule immunostimulatory and/or chemotherapeutic drugs.

When the star polymers of the present disclosure are used for array of ligands that act extracellularly, the present inventors have found: (i) a range of hydrodynamic sizes of star polymers that are suitable for applications for delivery of extracellular receptor binding partners, such as B cell immunogens, as well as for delivering therapeutic biologics molecules, including antibodies, to specific tissues; (ii) a range of polymer arms and ligand densities needed to optimally engage cognate receptors; (iii) the compositions and synthetic routes that lead to the optimal ranges of star polymer hydrodynamic size and ligand density; and (iv) compositions of star polymers that prevent unwanted antibody responses that can lead to accelerated blood clearance.

When the star polymers of the present disclosure are used for delivery of pharmaceutically active compounds that act intracellularly, referred to herein as drug molecule(s) or drug(s), selected from chemotherapeutic and/or immunostimulant drugs for cancer treatment, the present inventors have found: (i) a range of hydrodynamic sizes of star polymers that lead to optimal tumor uptake following intravenous administration; (ii) the location and density of drug attachment on polymer arms needed to maximize drug loading; (iii) compositions and architecture of polymer arms that allows for high drug loading; (iv) compositions and synthetic routes that lead to the optimal ranges of star polymer hydrodynamic size and drug density required for intravenous delivery; (iv) compositions of star polymers that prevent unwanted antibody responses that lead to accelerated blood clearance; and (v) compositions of stimuli-responsive star polymers that lead to increased accumulation in tumors.

Disclosed herein is a star polymer of formula O[P1]-([X]-A[P2]-[Z]-[P3])n where O is a core; A is a polymer arm attached to the core; X is a linker molecule between the core and the polymer arm; Z is a linker molecule between the end of the polymer arm and P3; P1, P2 and P3 are each independently one or more compounds that act extracellularly or intracellularly, n is an integer number; [ ] denotes that the group is optional; and at least one of P1, P2 or P3 is present.

In certain embodiments, any one or more of P1, P2 or P3 is a ligand (L) comprising a compound that acts extracellularly, preferably any one or more of P2 and P3 is a ligand L. In these embodiments, the star polymer is suitable for use as a star polymer ligand display system. These embodiments therefore provide a star polymer of formula O[L1]-([X]-A[L2]-[Z]-[L3])n. In certain preferred embodiments, the star polymer has any one of the following formulae: O-([X]-A-[Z]-L3)n, 0-([X]-A(L2)-[Z])n, and O-([X]-A(L2)-[Z]-L3)n. In certain particularly preferred embodiments, the star polymer has the formula O-([X]-A-[Z]-L3)n.

In certain embodiments, any one or more of P1, P2 or P3 is a drug (D) comprising a pharmaceutically active compound that acts intracellularly. In these embodiments, the star polymer is suitable for use as a drug delivery system, for example, to deliver small molecule drugs to tumors. These embodiments therefore provide a star polymer of formula O[D1]-([X]-A[D2]-[Z]-[D3])n. In certain preferred embodiments, the star polymer has any one of the following formulae: O(D1)-([X]-A-[Z])n, 0-([X]-A(D2)-[Z])n, O-([X]-A-[Z]-D3)n, O(D1)-([X]-A(D2)-[Z])n, O-([X]-A(D2)-[Z]-D3)n, and O(D1)-([X]-A(D2)-[Z]-D3)n.

The star polymer may comprise a ligand (L) and a pharmaceutically active compound (D). Thus, in certain embodiments the star polymer has any one of the following formulae: O(D1)-([X]-A(L2)-[Z])n, O(D1)-([X]-A-[Z]-L3)n, O(D1)-([X]-A(L2)-[Z]-L3)n, O-([X]-A(D2)-[Z]-L3)n, O(D1)-([X]-A(D2)-[Z]-L3)n, O-([X]-A(L2)-[Z]-D3)n, and O(D1)-([X]-A(L2)-[Z]-D3)n. In certain particularly preferred embodiments, the star polymer has the formula O(D1)-([X]-A-[Z]-L3)n. In certain other particularly preferred embodiments, the star polymer has the formula O-([X]-A(D2)-[Z]-L3)n. In certain further particularly preferred embodiments, the star polymer has the formula O(D1)-([X]-A(D2)-[Z]-L3)n.

In the foregoing discussion and elsewhere in this specification, the designations -A(P2)-, -A(L2)-, and -A(D2)- are intended to mean that the compound that acts extracellularly or intracellularly (P), the ligand (L) and the drug (D) are linked to monomer units distributed along the polymer arms (A). Similarly, the designations -O(P1)-, -O(L1)-, and -O(D1)- are intended to mean that the compound that acts extracellularly or intracellularly (P), the ligand (L) and drug (D) are linked to functional groups attached to the core (O).

It will be appreciated from the foregoing discussion that certain embodiments of the star polymer have the formula O-([X]-A(D)-[Z]-[L])n, where O is a core; A is a polymer arm attached to the core; X is a linker molecule between the core and the polymer arm; Z is a linker molecule between the polymer arm and a ligand; L is a ligand comprising a pharmaceutically active compound that acts extracellularly; D is drug comprising a pharmaceutically active compound that acts intracellularly; n is an integer number; [ ] denotes that the group is optional; D may or may not be present; and at least one of D or L is present.

Also disclosed herein is a star polymer ligand display system comprising a star polymer having the formula O-([X]-A(D)-[Z]-L)n, where n is greater than or equal to 2.

Also disclosed herein is a drug delivery system comprising a star polymer having the formula O-([X]-A(D)-[Z]-[L])n, where D is present.

Core (O)

Any suitable material can be used for the core (O) with the proviso that the core should be selected to ensure that a sufficient number of polymer arms (A) can be attached for the intended application. In some embodiments of star polymers used as a vaccine for displaying immunogens, such as B cell immunogens, a core (O) is selected to allow for attachment of five or more polymer arms (A) to enable display of five or more antigens. In certain embodiments, the core (O) is selected so that fifteen or more polymer arms (A) can be attached to enable display of fifteen or more ligands (L). In other embodiments, the number of polymer arm (A) attachment points on the core (O) is increased through the use of an amplifying linker, such that a core (O) with an integer number of attachment points is increased by an integer multiple, e.g., 2, 3, 4, 5, 6, 7, 8, 9 or 10, through the use of a heterofunctional linker. Suitable amplifying linkers are described elsewhere.

Herein, we describe methods of designing and manufacturing star polymers to maximize loading of polymer arms (A) on cores (O). For some compositions of cores (O) and polymer arms (A), the loading of polymer arms (A) on the core (O) may be complete, i.e., all reactive groups on the core (O) are linked to a polymer arm (A). For certain other compositions of cores (O) and polymer arms (A), polymer arm (A) loading on the core may be incomplete. Thus, for the assembly of certain compositions of star polymers, cores may be selected to include twice as many arm attachment points as needed. In a non-limiting example of a vaccine based on star polymers of the present disclosure with 15 or more ligands, a core with 30 or more attachment points is used, such as between 30 and 512 attachment points. In certain embodiments, the core (O) has between 32 and 128 attachment points.

In some embodiments, the core (O) is based on a dendron or dendrimer. Dendrons and dendrimers are a class of highly branched, chemically defined (precise architecture) and monodisperse macromolecules. Dendrimers are typically core-shell structures that are symmetric around the core. In dendrons, the core is usually a chemically addressable group called the focal point. The core of a dendrimer affects its three-dimensional shape, i.e., spheric, ellipsoidic, or cylindric. The surface of a dendrimer is densely packed with functional groups, with the number of functional groups dictated by the generation of the dendrimer. The surface functional groups can be directly used or further modified for the attachment of other components, such as polymer arms (A), ligands (L) or drugs (D). Dendrimers include but are not limited to polyamidoamine (PAMAM), poly(L-lysine) (PLL), polyamide, polyester, polypropylenimine (PPI), and poly(2,2-bis(hydroxylmethyl)propionic acid) (bis-MPA).

In certain embodiments, the core (O) comprises a polyamidoamine (PAMAM) dendrimer with amine functional groups. In these embodiments, the polyamidoamine dendrimer has surface amine groups, referred to as X1, that react with the linker precursors X2 attached to the polymer arm (A) to link the polymer arm (A) to the core (O) via the linker (X). In certain embodiments, the polyamidoamine dendrimer is a fifth-generation dendrimer with 128 functional groups on the surface. In preferred embodiments, the functional groups on the polyamidoamine dendrimer are amines.

The present inventors have found that star polymers comprising polymer arms (A) linked to dendrimer-based cores (O) lead to macromolecules that have lower viscosity than linear polymers of equivalent molecular weight. A non-binding explanation is that the highly-branched polymer structure eliminates chain entanglements in contrast to its linear analogue and the branching also results in high solubility and low melt- and solution viscosity.

Cores (O) may also be selected from hyperbranched polymers, which can have similar properties to dendrimers and dendrons. Unlike chemically defined dendrimers or dendrons, however, hyperbranched polymers are often constructed based on one-pot reactions of AB₂ or AB₃ monomers, requiring essentially no work-up.

A challenge with hyperbranched polymers is that they can have wide molecular weight distributions (and high polydispersity) and are challenging to characterize. Thus, with the exception of hyperbranched polymers produced by solid-phase synthesis, such as hyperbranched poly(amino acids) produced by solid-phase peptide synthesis, cores (O) based on dendrons and dendrimers are preferred.

Polymer Arm (A)

The polymer arm (A) is linked to the core (O) either directly (i.e. X is not present) or indirectly (i.e. via linker molecule (X)). The number of polymer arms is an integer value, n.

The polymer arms (A) radiating from the core (O) may be water-soluble under physiologic pH and salt concentrations and principally serve to increase the hydrodynamic radius of the star polymer.

Star polymers comprising polymer arms (A) for ligand display serve the additional function of providing distance between ligands (L), which may be linked to the polymer ends, and may either be flexible or rigid, depending on the application.

Star polymers comprising polymer arms (A) used for the delivery of small molecule chemotherapeutic and/or immunostimulant drugs (D) for cancer treatment should be selected to increase drug solubility, reduce/prevent drug degradation and provide a stealth coating to prevent the uptake of the star polymer by cells of the reticuloendothelial system (RES). Polymer arms (A) comprising star polymers used for chemotherapeutic and/or immunostimulant delivery principally function to prevent star polymer uptake by phagocytic cells and therefore should be flexible, non-rigid and non-reactive for serum proteins. Unexpectedly, the present inventors found that hydrophilic arms comprised of anionic monomers can function to improve solubility of star polymers carrying high densities of hydrophobic or amphiphilic small molecule drugs; extend the polymer arm (A) to increase the star polymer hydrodynamic size; and prevent antibody responses, which was found to reduce accelerated blood clearance upon repeat dosing.

Polymer arms (A) used for star polymers can be derived from either natural or synthetic sources and may be prepared by any suitable means. Polymer arms (A) are typically prepared by polymerization, which may be described as a chemical reaction, usually carried out with a catalyst, heat or light, in which monomers combine to form a chainlike, or cross-linked, macromolecule (a polymer).

There are mainly two types of mechanisms to prepare synthetic polymers: step-growth (i.e. condensation) polymerization and chain-growth (i.e. free radical, anionic, or cationic) polymerization. In terms of polymerization process, solution polymerization, bulky polymerization, dispersion polymerization, and emulsion polymerization are available.

In certain embodiments, polymer arms (A) are prepared by controlled “living” radical polymerization methods to minimize premature termination and enable more precise control over the polymer composition, molecular weight, polydispersity, and functionality. In the context of controlled radical polymerization, highly reactive free radicals generated from the decomposition of an initiator (radical source) are capable of initiating the polymerization of monomers. Chain propagation proceeds as the radical center continues to add monomers; however, for controlled, living radical polymerization, the reversible deactivation of radicals occurs, either by metal complex via atom transfer radical polymerization (ATRP) mechanism, dithioester or trithioester chain transfer agent (CTA) via reversible addition-fragmentation chain-transfer (RAFT) polymerization mechanism, or nitroxide radical via nitroxide-mediated polymerization (NMP) mechanism. These mechanisms lower the effective concentration of active radicals at any moment during the polymerization process, which prevents potential premature chain termination. The fast and reversible radical activation-deactivation process allows all propagating chains equal opportunity to grow when monomers are presented, resulting in polymers with very narrow molecular weight distribution and low polydispersity.

Controlled radical polymerization allows polymer arms (A) with a wide range of different polymer functionalities, either introduced through monomer selection, the initiation or quenching of the propagating polymer chain, or post-polymerization modification, sometimes referred to as polymer analogous reaction. While functional groups distributed along the backbones of polymers arms (A) can be modulated through choice of monomer, both end groups of polymer arms (A) can be modulated by selecting suitable initiators and CTAs used for RAFT polymerization. Accordingly, an initiator comprising a functional group (FG), ligand (L) or drug (D) used to initiate polymerization of monomers in the presence of CTA will lead to polymer arms (A) with one end functionalized with the FG, ligand (L) or drug (D) and the other end will comprise a dithioester or trithioester that is introduced by the CTA. The dithioester or trithioester enables the use of such polymers as a macro-CTA to induce the RAFT polymerization of other monomers, thus providing a simple route for the preparation of block copolymers, such as A-B type di-block copolymers. Alternatively, the dithioester or trithioester may be reduced (to a thiol) and capped with a thiol-reactive moiety or may be capped using an initiator comprising a functional group (FG), ligand (L) or drug (D).

In certain embodiments, the X2 and Z1 linker precursors are introduced by reacting an initiator functionalized with an X2 or Z1 linker precursor, ligand (L) or drug (D) with monomers in the presence of CTA to produce a polymer arm intermediate, X2-polymer-CTA, Z1-polymer-CTA, L-polymer-CTA, or D-polymer-CTA, which is capped using an initiator or thiol-reactive compounds functionalized with an X2 or Z1 linker precursor, ligand (L) or drug (D) to obtain a heterotelechelic polymer arm, X2-polymer-Z1, L-polymer-X2 or D-polymer-X2. Specific examples of polymer arms (A) produced in this manner are described later.

In some embodiments, (meth)acrylamide- and (meth)acrylate-based polymers are synthesized by reversible addition-fragmentation chain-transfer (RAFT) polymerization. In additional embodiments, poly(amino acids) and poly(phosphoesters) are synthesized by ring opening polymerization. For polymers produced by ring opening polymerization, the compounds used for initiating polymerization can be used to introduce functionalities at one end and the other end of the resulting polymer can be capped by any suitable means to introduce the desired functionality. In still other embodiments, peptide-based biopolymers are synthesized by solid-phase peptide synthesis.

The architecture of the polymer arm (A) is selected to address the specific demands of the application. In some embodiments, linear polymer arms (A) are used to link ligands (L) indirectly via the polymer arm (A) to the core (O) of the star polymer. In other embodiments, the polymer arm (A) is a brush polymer that is used as an amplifying linker and/or to provide additional surface area coverage of the star polymer. In some embodiments, polymer arms (A) with brush polymer architecture are used on star polymer carriers of drugs (D), such as small molecule immunostimulant and/or chemotherapeutic drugs. Coating star polymers with polymer arms with brush architecture led to increased tumor uptake as compared with star polymers comprising linear polymer arms (A). A non-binding explanation is that increased surface area coverage by the hydrophilic polymer arm (A) reduced blood protein binding and/or reduced uptake by phagocytic cells, thereby increasing circulation time and star polymer uptake into tumors.

In other embodiments, polymer arms with di-block architecture are used to segregate different components comprising the star polymer. In some embodiments, di-block copolymers are used to segregate drugs (D), such as small molecule chemotherapeutics and/or immunostimulant drugs, to one block of the di-block polymer. In other embodiments, di-block polymers are used to segregate charged monomers, i.e., charged monomers are only placed on one block of the di-block polymer. In still other embodiments, di-block polymers are used to segregate the two or more different components, such as drugs (D) and charged monomers.

Unexpectedly, it was found that charged monomers placed on the polymer arms (A) led to increased tissue retention in vivo and reduction in antibodies induced to any ligand (L) (or drug (D)) arrayed on the star polymer. Non-binding explanations for these findings are that (i) the charged monomers promote an extended confirmation of the polymer arms, thereby increasing Rh and improving duration of activity through increased tissue retention; and, (ii) the charge, specifically, negatively charged monomers, proximal to the ligand (or drug), reduce interactions with B cells, thereby reducing the tendency of the multivalent ligand to induce IgM antibodies. Thus, in preferred embodiments, star polymers used for display of ligands (L), other than B cell immunogens, and/or for delivery of drugs (D) to specific tissues include charged monomers, particularly negatively charged monomers, as a means to improve tissue retention and prevent the induction of antibody responses.

Each of the monomer units comprising the polymer arm (A) is selected to meet the demands of the application. Suitable polymer arms minimally comprise a hydrophilic monomer (B) with an integer number, b, of hydrophilic monomer units. The polymer arms (A) may additionally comprise an integer number, c, of charged monomer units (C) and/or may additionally include an integer number, e, of reactive co-monomers, E, that comprise a functional group enabling attachment of drugs (D) or optionally ligands (L).

Polymer arms (A) of Formula I are polymers arms (A) that include neutral hydrophilic monomers (B), and optionally either or both a charged monomer (C) and/or a reactive monomer (E), which may be represented as (B)b-[(C)c]-[(E)e], wherein b is equal to an integer number of repeating units of a neutral, hydrophilic co-monomer, B; c is an integer number of a repeating units of a charged co-monomer, C; e is equal to an integer number of repeating units of a reactive co-monomer, E, used for drug (D) (or optionally ligand (L)) attachment; and, [ ] denotes that the monomer unit is optional.

In some embodiments, the polymer arm (A) is a terpolymer comprising neutral hydrophilic monomers, charged monomers and reactive monomers linked to drug (D), which may be represented schematically:

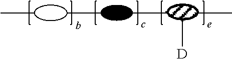

In some embodiments, the polymer arm (A) is a copolymer comprising hydrophilic monomers and charged monomers, which may be represented schematically:

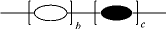

In some embodiments, the polymer arm (A) is a copolymer comprising hydrophilic monomers and reactive monomers linked to drug (D), which may be represented schematically:

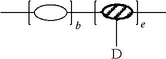

In some embodiments, the polymer arm (A) is a homopolymer comprising only hydrophilic monomers, which may be represented schematically

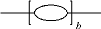

In some embodiments, the polymer arm (A) is a di-block co-polymer that comprises monomers linked to drug and hydrophilic monomers on one block and only hydrophilic monomers on the other block, which may be represented schematically:

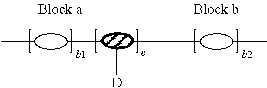

For star polymers comprising di-block polymer arms (A) with monomers (E) linked to amphiphilic or hydrophobic small molecule drugs and hydrophilic monomers (B) on one block and only hydrophilic monomers (B) on the other block, it was found that placing the monomers linked to the amphiphilic or hydrophobic small molecule drugs (D) on the block of the di-block polymer arms (A) proximal to the core of the star polymers resulted in improved stability, i.e. reduced propensity of the star polymers to aggregate.

In some embodiments, the polymer arm (A) is a di-block polymer, and includes monomers linked to drug (D) and charged monomers on opposite blocks, which may be represented schematically:

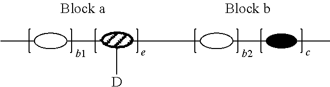

For star polymers comprising di-block polymer arms (A) with monomers linked to amphiphilic or hydrophobic small molecule drugs and hydrophilic monomers on one block and hydrophilic monomers and charged monomers on the other block, it was found that placement of the monomers linked to the amphiphilic or hydrophobic small molecule drugs (D) proximal to the core and the charged monomers on the opposite block of polymer arms (A) distal to the core led to improved stability of the resulting star polymers. A non-binding explanation for this finding is that the charged block, i.e. the polymer block comprising charged monomers, allows improved solubility and shields the block bearing the amphiphilic or hydrophobic small molecule drug (D).

In some embodiments, the polymer arm is a di-block polymer, and includes drugs (D) on one of the blocks, which may be represented schematically:

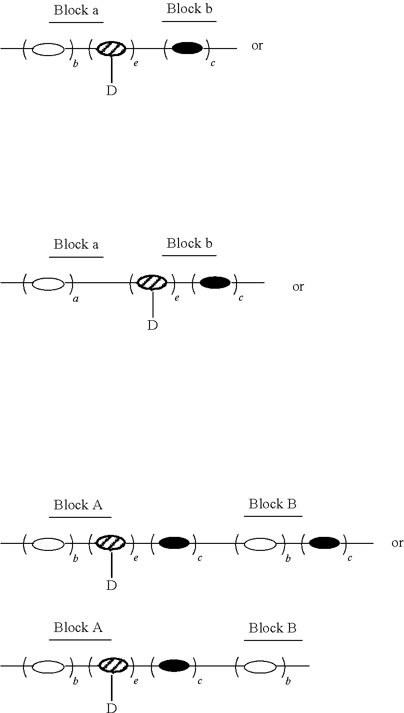

For star polymers bearing Ligands at the ends of the polymer arms, it was found that charged monomers placed on the block of di-block polymer arms (A) most proximal to the ligand (L) (distal to the core (O)) led to improved pharmacokinetics and reduced magnitude of antibodies induced to the ligand (L) and other components of the star polymer. Non-binding explanations for these findings are that (i) the charge promotes an extended confirmation of the polymer arms, thereby increasing Rh and improving duration of activity through increased tissue retention; and, (ii) the charge, specifically, negatively charged monomers, proximal to the ligand (L), reduces interactions with B cells and the tendency of the multivalent ligand (L) (or other components of the star polymer) to cross-link B cell receptors to induce antibodies.

In some embodiments, the polymer arm (A) includes neutral, hydrophilic monomers selected from acrylates, (meth)acrylates, acrylamides, (meth)acrylamides, allyl ethers, vinyl acetates, vinyl amides, substituted styrenes, amino acids, acrylonitrile, heterocyclic monomers (i.e. ethylene oxide), saccharides, phosphoesters, phosphonamides, sulfonate esters, sulfonamides, or combinations thereof.

In some embodiments, the polymer arm (A) comprises neutral hydrophilic monomers, monomer B of Formula I, selected from (meth)acrylates and (meth)acrylamides with chemical structure CH₂═CR₂—C(O)—R₁, wherein the acryl side group R₁ may be selected from one or more of the groups consisting of —OR₃, —NHR₃ or —N(CH₃)R₃, where R₂ can be H or CH₃, and R₃ is independently selected from any hydrophilic substituent. Non-limiting examples of R₃ include but are not limited to H (except for OR₃), CH₃, CH₂CH₃, CH(CH₃)₂, CH₂CH₂N(CH₃)₂, CH₂CH₂N(CH₂CH₃)₂, CH₂CH₂OH, CH₂(CH₂)₂₀H, CH₂CH(OH)CH₃, CHCH₃CH₂OH, (CH₂CH₂O)ᵢH, (CH₂CH₂O)ᵢCH₃, (CH₂CH₂O)ᵢCH₂CH₃, where i is an integer number of repeating units. Note: hydrophilic monomers and neutral hydrophilic monomers are used interchangeably throughout and are meant to describe hydrophilic monomers that are neutral, i.e. they lack charge at physiologic pH, pH 7.4.

A non-limiting example of a neutral hydrophilic monomer, wherein R₁═NHR₃, R₂═CH₃, and R₃═CH₂CH(OH)CH₃ is:

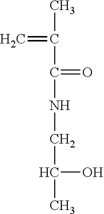

The above example, N-(2-hydroxpropyl(methacrylamide)) (HPMA), is an example of a neutral hydrophilic monomer, e.g., monomer, B, of Formula I.

In some embodiments, the polymer arm (A) may comprise monomers, C, that contain a charged functional group. Non-limiting examples of such monomers include amino acid N-carboxyanhydrides (NCA), (meth)acrylamides and (meth)acrylates that contain (latent), amine, quaternary ammonium, sulfonic acid, sulfuric acid, phosphoric acid, phosphonic acid, carboxylic acid and/or boronic acid functional groups.

In some embodiments, the polymer arm (A) comprises charged hydrophilic monomers (C) selected from (meth)acrylates and (meth)acrylamides with chemical structure CH₂═CR₅—C(O)—R₄. The acryl side group R₄ may be selected from one or more of the groups consisting of —OR₆, —NHR₆ or —N(CH₃)R₆, where R₅ can be H or CH₃ and R₆ can be selected from, but is not limited to H (except for NHR₆ or N(CH₃)R₆), linear alkyl structures such as (CH₂)ⱼNH₂, (CH₂)ⱼCH(NH₂)COOH, (CH₂)ⱼCOOH, (CH₂)ⱼPO₃H₂, (CH₂)ⱼOPO₃H₂, (CH₂)ⱼSO₃H, (CH₂)ⱼOSO₃H, (CH₂)ⱼB(OH)₂, where j is an integer number of a repeating units, typically between 1 to 6, and more versatile structures such as CH₂CH₂N(CH₃)₂, CH[CH₂N(CH₃)₂]₂, CH(COOH)CHCH₂COOH, [CH₂CH(CH₃)O]₅PO₃H₂, (CH₂)₃CH(OPO₃H₂)(CH₂)₂CH(OPO₃H₂)(CH₂)₃CH₃, C(CH₃)₂CH₂SO₃H, and C₆H₄B(OH)₂.

A non-limiting example of a charged monomer wherein R₄═—OR₆, R₅═CH₃ and R₆═H is:

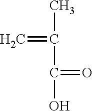

wherein in this example, i.e. methacrylic acid, the monomer would be expected to be deprotonated at physiologic pH (i.e. pH 7.4) and carry a negative charge. The above structure is an example of a charged monomer, monomer C of Formula I. Note: charged monomers are meant to describe monomers have charge at physiologic pH, pH 7.4.

In some embodiments, polymer arms (A) comprise a monomer, E, that is reactive towards drugs (D) (or optionally ligands (L)). Suitable reactive monomers include but are not limited to any monomer unit bearing a functional group suitable for attachment of drugs (D) (or optionally ligands (L)), including monomers with azide, alkyne, protected hydrazine (which is deprotected after polymerization), heterocyclic rings, isocyanate, isothiocyanate, aldehyde, ketone, activated carboxylic acid, protected maleimide, and latent amine. Suitable linker chemistries used to link drug molecules (D) to the polymer backbone are discussed throughout the present specification. Note, ligands (L) that act extracellularly may optionally be linked to reactive co-monomers distributed along the backbone of the polymer arm (A), though, in preferred embodiments any ligands (L) present are linked to the ends of the polymer arms (A) to maximize solvent exposure.

In some embodiments, the polymer arm (A) comprises reactive monomers (E) selected from (meth)acrylates and (meth)acrylamides with chemical structure CH₂═CR₈—C(O)—R₇. The acryl side group R₇ may be selected from one or more of the groups consisting of —OR₉, —NHR₉ or —N(CH₃)R₉, where R₈ can be H or CH₃ and R₉ can be independently selected, but is not limited to, linear alkyl structures such as (CH₂)ₖR₁₀, (CH₂)ₖC(O)NHR₁₀ or (CH₂CH₂O)ₖCH₂CH₂C(O)NHR₁₀, where k is an integer number of repeating units, typically between 0 to 6, and R₁₀ is independently selected from (CH₂)ₕ—FG, (CH₂CH₂O)hCH₂CH₂—FG or (CH₂CH₂O)ₕCH₂CH₂—FG, where h is an integer number of repeating units, typically between 0 and 6, and FG is any functional group, which may be selected from, but not limited to, carboxylic acid and activated carboxylic acids (e.g., carbonylthiazolidine-2-thione, tert-butyl and/or nitrobenzyl protected carboxylic acid), anhydride, aldehyde, ketone, amine and protected amines (e.g. tert-butyloxycarbonyl protected amine), hydrazine and protected hydrazine (e.g., tert-butyloxycarbonyl protected hydrazine), OSi(CH₃), CCH, N₃, propargyl, halogen (e.g. fluoride, chloride), olefins and endo cyclic olefins (e.g. allyl), CN, OH, and epoxy.

A non-limiting example of a reactive methacrylamide monomer wherein R₇ is NHR₉, R₈ is CH₃, R₉ is (CH₂)ₖC(O)NHR₁₀, k is equal to 2 and R₁₀ is propargyl is:

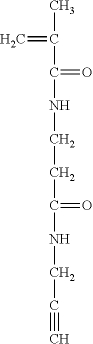

In some embodiments, the polymer arm (A) comprises a hydrophilic meth(acrylamide)-based homopolymer. A non-limiting example of a homopolymer arm (A) comprising meth(acrylamide)-based monomers is:

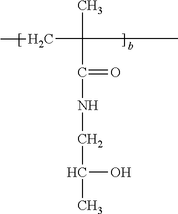

wherein the hydrophilic monomer B is N-(2-hydroxpropyl(methacrylamide)) (HPMA), b is an integer number of monomer units, typically between about 50 to about 450, such as between about 70 to 420 for a target molecular weight between about 10 kDa to about 60 kDa, and wherein the ends of the polymer may be linked to any suitable heterogeneous molecules, such as X1 and Z2 linker precursors, a core (O) and a ligand (L), a core (O) and a drug (D) or a core (O) and a capping group.

In some embodiments, the polymer arm (A) comprises a meth(acrylamide)-based co-polymer comprising both hydrophilic and charged co-monomers. A non-limiting example of a polymer arm (A) comprising a meth(acrylamide)-based co-polymer comprising hydrophilic and charged co-monomers is:

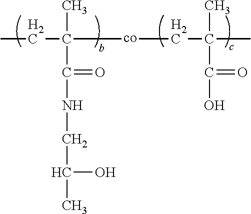

In some embodiments, the polymer arm (A) comprises a meth(acrylamide)-based co-polymer comprising both hydrophilic and reactive co-monomers. A non-limiting example of a polymer arm (A) comprising a meth(acrylamide)-based co-polymer comprising hydrophilic and reactive co-monomers is:

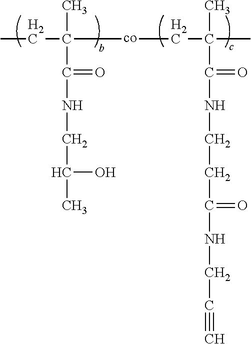

In some embodiments, the polymer arm (A) comprises a meth(acrylamide)-based ter-polymer comprising hydrophilic, reactive and charged monomers. A non-limiting example of a polymer arm (A) comprising a meth(acrylamide)-based ter-polymer comprising hydrophilic, charged and reactive co-monomers is:

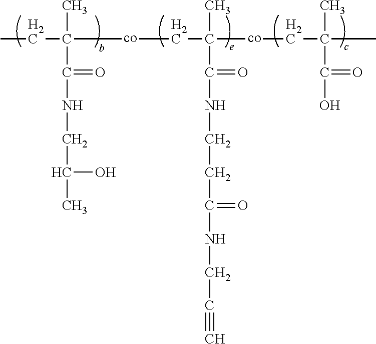

In some embodiments, the polymer arm (A) comprises a meth(acrylamide)-based di-block copolymer. A non-limiting example of a polymer arm (A) comprising a meth(acrylamide)-based di-block copolymer comprising a hydrophilic block with reactive co-monomers that is linked to only one block of the di-block copolymer is:

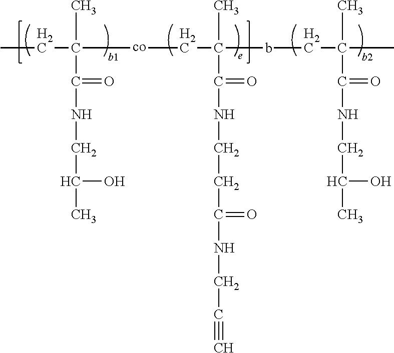

wherein one block comprises an integer number of repeating units of hydrophilic and reactive monomers denoted by b1 and e; and the other block comprises an integer number of repeating units of a hydrophilic monomer denoted by b2; note that the two blocks in the schematic are separated by brackets [ ], and that, b, delineates the two blocks.

In some embodiments, the polymer arm (A) comprises a meth(acrylamide)-based di-block copolymer, wherein one block comprises reactive co-monomers and the other block comprises charged co-monomers. A non-limiting example of a polymer arm (A) comprising a meth(acrylamide)-based di-block comprising a hydrophilic block with reactive co-monomers that is linked to another block comprising charged co-monomers is:

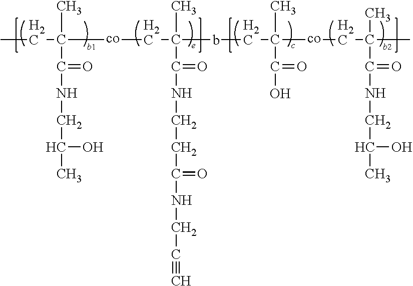

wherein one block comprises an integer number of repeating units of hydrophilic and reactive monomers denoted by b1 and e; and the other block comprises an integer number of repeating units of charged and hydrophilic co-monomers denoted by c and b2; note that the two blocks in the schematic are separated by brackets [ ], and that, b, delineates the two blocks.

In the above examples, the reactive co-monomers may be used to link drug molecules (D) or optionally ligands (L). Other examples of reactive co-monomers are described elsewhere.

In some embodiments, the polymer arm (A) comprises a meth(acrylamide)-based di-block copolymer, wherein one block comprises a terpolymer consisting of reactive monomers, charged monomers and hydrophilic monomers and the other block comprises charged co-monomers and hydrophilic monomers. A non-limiting example of a polymer arm (A) comprising a meth(acrylamide)-based di-block comprising a hydrophilic terpolymer block with reactive co-monomers and charged monomers that is linked to one block comprising charged monomers is:

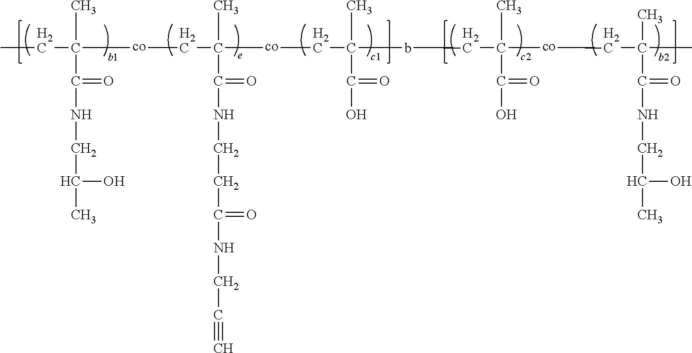

wherein one block comprises an integer number of repeating units of hydrophilic, reactive and charged monomers denoted by b1, e and c1; and the other block comprises an integer number of repeating units of charged and hydrophilic co-monomers denoted by c2 and b2; note that the two blocks in the schematic are separated by brackets [ ], and that, b, delineates the two blocks.

Effect of Polymer Arm (A) Length

The present inventors have identified the optimal polymer arm (A) length, expressed as molecular weight (MW), i.e. weight average (Mw) or number average (Mn), to achieve the hydrodynamic radius (Rh) and high ligand (L) density required for certain applications. The interplay between polymer arm molecular weight, star polymer Rh, ligand (L) density and biological activity were previously unexplored. Note: sometimes radius of gyration (Rg) is used in place of Rh.

Unexpectedly, it was observed that there is a direct, linear correlation between polymer arm (A) molecular weight and star polymer radius, and that increasing star polymer radius results in improved biological activity in certain applications. For example, increasing the radius of a star polymer from about 7.5 nm to about 15 nm, by increasing the molecular weight of an HPMA-based polymer arm (A) from about 15 kDa to about 50 kDa, resulted in a marked increase in the magnitude of antibodies generated against a peptide-based B cell immunogen as the ligand (L) arrayed on the star polymer, following local, subcutaneous administration of the star polymer arraying the B cell immunogen. A non-binding explanation is that the increased size of the star polymer from 7.5 to 15 nm Rh—that results in increased retention in subcutaneous tissue (reduced rate of clearance) at which the star polymer was administered—leads to prolonged activity in draining lymph nodes, i.e. sustained engagement of the ligands (L) with cognate receptors. Therefore, in certain embodiments of star polymers requiring sustained activity, we disclose the unexpected finding that the polymer arm length can be tuned to increase the size of the star polymers as a means to increase the persistence of activity of the star polymer in certain tissues.

An additional finding was that as the polymer arm (A) molecular weight is increased, the maximal loading of polymer arms (A) that can be loaded onto each core (O) decreases. Therefore, the polymer arm molecular weight must be selected to achieve the optimal balance of polymer arm (A) density (and therefore bioactive ligand molecule density) and hydrodynamic size (i.e. Rh) for the desired application.

As some applications may require high ligand density and high molecular weight polymer arms (A) to achieve sufficient densities of ligands (L) on star polymers of sufficient hydrodynamic size, the present inventors developed novel compositions of star polymers with amplifying linkers that enable the attachment of two or more ligands (L), which may be the same or different, on the ends of each of the polymer arms (A) radiating from the core (O), thereby allowing for an increase in ligand density even when using relatively high molecular weight polymer arms that can otherwise limit the density of polymer arms arrayed on the star polymer surface.

Suitable amplifying linkers include any bi-functional linker molecule that can join two or more ligands (L) to a single polymer arm (A). Amplifying linkers may be expressed by the formula, (FG1)-T-(FG2)m, wherein FG1 and FG2 are any functional group, T is any suitable linker and m represents the number of FG2 linked to the amplifying linkers and is any integer greater than 1, typically between 2 to 16.

In some embodiments, the amplifying linker comprises a polymer of formula FG1-linker (M(FG2))m, wherein FG1 is linked to an oligomer comprised of an integer number of repeating units, m, of monomers linked to FG2. In other embodiments, the amplifying linker, T, is a dendritic amplifying linker, wherein each monomer of the dendron has an integer number of branches, 3, and the dendron can be any generation represented by an integer number, γ. Thus, the multiple by which dendritic amplifying linkers increase functionality (FG1→FG2) can be expressed as g=β⁷. In a non-limiting example, for a 4^{th} generation dendron comprised of monomers with 2 branch points, g is equal to 16.

A non-limiting example of a second-generation lysine-based dendron, wherein g=4, is:

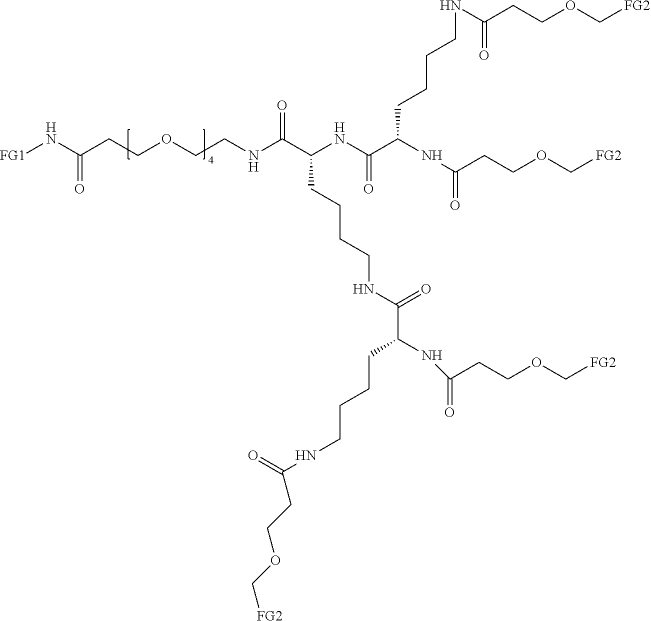

In some embodiments, the amplifying linker has the formula (sulfo-DBCO)-T-(Maleimide)m and is used to install multiple maleimide functional groups onto a polymer arm (A) terminated with an azide functional group. A non-limiting example of a (sulfo-DBCO)-T-(Maleimide)m amplifying linker is:

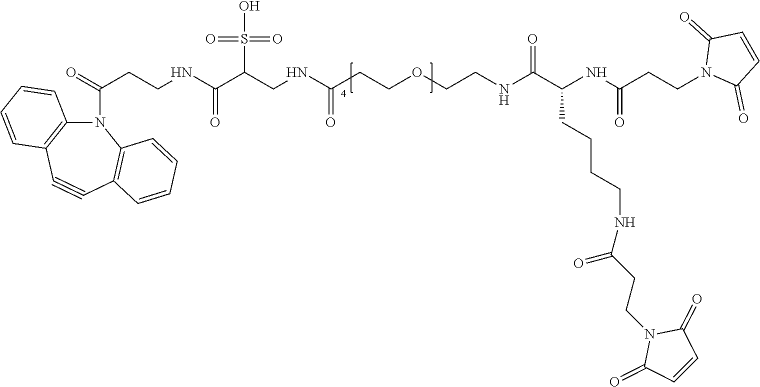

In other embodiments, the amplifying linker has the formula (sulfo-DBCO)-T-(alkyne)m and is used to install multiple alkyne functional groups onto the end of a polymer arm (A) terminated with an azide functional group. A non-limiting example of a (sulfo-DBCO)-T-(alkyne)m amplifying linker is:

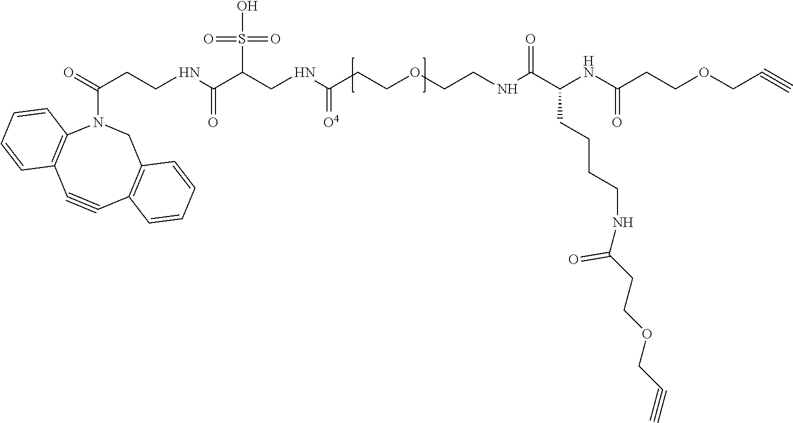

While increasing hydrodynamic radius of the star polymers of the present disclosure may be beneficial for certain applications, it was found, unexpectedly, that narrow ranges of star polymer hydrodynamic radii were optimal for certain other applications, including targeting tumors following intravenous administration of the star polymers of the present disclosure. As a non-limiting example, it was found unexpectedly that star polymers with hydrodynamic radii between about 5-15 nm Rh, corresponding to star polymers with polymers arms between about 5 to 50 kDa molecular weight, were optimal for uptake into tumors, whereas those with lower radii (<5 nm) were found to be more rapidly cleared from the blood; and those with larger size showed overall lower accumulation into the tumor.

The unexpected findings related to the ability to tightly control the hydrodynamic radius of the star polymer by modulating polymer arm (A) length, combined with the identification of the optimal star polymer size and ligand density needed for certain applications, has led to the present inventors determination of the ranges of polymer arm (A) lengths and compositions that enable the appropriate size (i.e. Rh) and ligand density of novel star polymer compositions needed to achieve improvements in biological activity.

For example, the present inventors found that both ligand density and hydrodynamic radius impact the ability of vaccines based on star polymers of the present disclosure to induce antibody responses. Importantly, polymer arm molecular weight is directly proportional to hydrodynamic size but inversely related to arm loading (i.e. density on the surface of the star polymer). Therefore, polymer arm (A) molecular weight should be selected to achieve sufficient size while not sacrificing arm loading. Thus, in certain embodiments of star polymers of the present disclosure used as vaccines for inducing antibody responses, the polymer arm molecular weights are typically an average size of about 10 kDa to about 60 kDa, which ensures an appropriate balance between hydrodynamic size and arm loading.

As disclosed herein, polymer arm (A) molecular weight is a key parameter that impacts hydrodynamic radius. Unexpectedly, increasing the molecular weight of the polymer arms comprising star polymers of the present disclosure led to increased hydrodynamic size, which was associated with increased retention at the site of injection following administration. Thus, polymer arm molecular weight can be used as a means to modulate the distribution and kinetics of star polymers of the present disclosure following administration to a subject. In some embodiments, wherein the star polymers of the present disclosure are used for an application where they must freely cross capillary beds, or extravasate and penetrate tumors, the polymer arm molecular weight is selected to have a molecular weight less than about 50 kDa to achieve a radius less than about 15 nm. In other embodiments, wherein the star polymers of the present disclosure are used for an application that requires persistence at the site of administration, the polymer arm molecular weight is selected to have a molecular weight greater than about 10 kDa to achieve a radius greater than about 5 nm.

Optimisation of Architecture and Compositions of Polymer Arms for Ligand Array

Based on the finding that increasing size (Rh) of star polymers arraying ligands (L) comprising extracellular receptor binding ligands (L) led to increased biological activity in certain contexts, such as the delivery of ligands (L) to local tissues sites, e.g., B cell immunogens to draining lymph nodes following subcutaneous injection, the impact that polymer arm composition has on Rh and biological activity was evaluated.

Unexpectedly, for applications other than use as vaccines, star polymers comprising polymers arms (A) with between about 0.5 to about 20 mol % co-monomers comprising negatively charged functional groups had higher Rh and improved biological activity as compared with star polymers comprising polymers arms with the same molecular weight but with either positively charged functional groups or non-charged (i.e. only neutral) functional groups.

Optimisation of Composition and Architecture of Polymer Arms (A) for Passive Tumor Targeting

Polymer arm composition and architecture were also found to impact the size (Rh) and activity of star polymer carriers of small molecule chemotherapeutic and/or immunostimulant drugs (D) used for cancer treatment. For instance, attachment of drugs (D) with relatively low molecular weight and amphiphilic or hydrophobic properties, e.g., aromatic heterocycles such as imidazoquinolines or amidobenzimidazoles or aromatic chemotherapeutic drugs, such as anthracyclines, to polymer arms (A) based on statistical co-polymers at densities greater than about 5 mol % increased the propensity of such star polymers to aggregate, whereas attachment of greater than 5 mol %, up to 40 mol %, of drugs to polymer arms was achieved on star polymers comprising di-block copolymer arms without aggregation occurring, provided, however, that the drugs were attached to the block of the di-block copolymer that was proximal to the core (O). Thus, in certain embodiments of star polymers used as carriers of small molecule drugs (D), the polymer arm (A) comprises a di-block copolymer and the drugs (D) are only attached to the block of the di-block copolymer that is proximal to the core (O). An additional finding was that charged co-monomers included on terpolymers or di-block copolymers with high densities of amphiphilic or hydrophobic drugs improved the solubility of the star polymer carriers of the amphiphilic or hydrophobic drugs (D) and thereby prevented aggregation. Thus, di-block copolymers with between about 0.5 to about 20 mol % charged co-monomers are used in certain embodiments of star polymer carriers of drugs (D), particularly amphiphilic small molecule drugs.

The molecular weight of polymer arms (A) of star polymers used for cancer treatment are chosen to ensure that the hydrodynamic size of the star polymer is of sufficient size to prevent renal elimination following intravenous administration but not too large so as to prevent extravasation and entry into the tumor. The optimal polymer arm (A) molecular weight is between about 5 kDa and 50 kDa, such as 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, 33 kDa, 34 kDa, 35 kDa, 36 kDa, 37 kDa, 38 kDa, 39 kDa, 40 kDa, 41 kDa, 42 kDa, 43 kDa, 44 kDa, 45 kDa, 46 kDa, 47 kDa, 48 kDa, 49 kDa, or 50 kDa. In certain embodiments, the polymer arm (A) molecular weight is between about 10 kDa to about 25 kDa or about 20 kDa to about 40 kDa. In certain embodiments, wherein the polymer arm is a di-block copolymer, the polymer arm molecular weight is between about 20 kDa to about 40 kDa, or 10 kDa to about 25 kDa; the mass ratio of the blocks is about 1:1, i.e. the mass of one block is about 12.5 kDa and the mass of the other block is about 12.5 kDa for a di-block copolymer with a molecular weight of 25 kDa; and drugs (D), such as small molecule chemotherapeutics and/or immunostimulant drugs are distributed along one block of the di-block copolymer, i.e., the block that is proximal to the core.

In addition to molecular weight, the number of polymer arms (A) attached should also be chosen to meet the demands of the application. For star polymers arraying ligands (L) for cancer treatment, the optimal arm number is greater than 5, such as between 5 and 45, preferably between 10 and 30 arms. In general, star polymers arraying ligands (L) for cancer treatment should have 5, preferably greater than 15, or more arms to ensure adequate receptor clustering. For star polymers delivering small molecule drugs, there is less dependence on arm number for activity but a greater dependence on mol % of attached drug (as described elsewhere); nonetheless, arm numbers are typically between about 5 to 30, such as 5, 6, 7, 8, 9, 10, 11, 12, 13, 14, 15, 16, 17, 18, 19, 20, 21, 22, 23, 24, 25, 26, 27, 28, 29, 30, with preferred embodiments having between 10-30 arms, preferably about 15-25 arms per star polymer carrier of small molecule drugs (D).

Compositions that Prevent the Induction of Antibodies

The induction of antibodies directed against pharmaceutically active compounds is a major challenge facing any delivery technology that presents structures in a multivalent array that can engage B cell receptors, even at low affinity, and lead to induction of antibodies. The induction of antibodies against ligands (L) displayed on star polymers, for uses other than as vaccines, would necessarily block activity of the ligand (L) and/or result in what has been referred to as accelerated blood clearance upon repeat dosing. Similarly, the induction of antibodies against drugs (D) displayed on star polymers would necessarily block activity of the drugs (D) and reduce activity upon repeat administration.

Therefore, a major advance disclosed herein is the identification of star polymer compositions that reduce or mitigate the induction of antibodies against ligands (L) and/or drugs (D) following repeat administration of star polymers displaying such ligands (L) and/or carrying such drugs (D).

Specifically, the present inventors unexpectedly found that high densities of negatively charged functional groups at or near the ligand (L) abrogated antibody responses directed to the ligand (L); and that high densities of negatively charged functional groups distributed along the backbone of polymer arms (A) linked to drugs (D) abrogated antibody responses directed against the drugs (D).

Additionally, it was observed, unexpectedly, that when certain saccharides that bind to the lectin receptor, CD22L, were placed at or near the ligand (L) at high densities in the star polymers of the present disclosure, antibody responses induced against the ligand (L) were reduced and in some cases abrogated. Similarly, saccharides that bind CD22L placed at or near the surface of star polymer carriers of drugs (D) were found to mitigate the induction of antibody responses against the drugs (D).

Linkers

Linkers generally refer to any molecules that join together any two or more different molecules of star polymers, which may additionally perform any one or more of the following functions: I) increase or decrease water solubility; II) increase distance between any two components, i.e. different molecules, of the star polymer; III) impart rigidity or flexibility; or IV) control/modulate the rate of degradation/hydrolysis of the link between any two or more different molecules.

Linkers may be used to join any two components of the star polymer, for example, a polymer arm (A) to the core (O) by any suitable means. The linker may use covalent or non-covalent means to join any two or more components, i.e. different molecules, for example a polymer arm (A) to the core (O), or a ligand (L) to the polymer arm (A).

In certain embodiments, a linker may join, i.e. link, any two components of the star polymer through a covalent bond. Covalent bonds are the preferred linkages used to join any two components of the star polymer and ensure that no component is able to immediately disperse from the other components, e.g., the ligand (L) from the star polymer, following administration to a subject. Moreover, covalent linkages typically provide greater stability over non-covalent linkages and help to ensure that each component of the star polymer is co-delivered to specific tissues and/or cells at or near the proportions of each component that was administered.

In a non-limiting example of a covalent linkage, a click chemistry reaction may result in a triazole that links, i.e. joins together, any two components of the star polymer. In certain embodiments, the click chemistry reaction is a strain-promoted[3+2] azide-alkyne cyclo-addition reaction. An alkyne group and an azide group may be provided on respective molecules comprising the star polymer to be linked by “click chemistry”. In some embodiments, a ligand (L), such as a B cell epitope contains a Z2 linker precursor bearing an azide functional group that is reactive towards a Z1 functional group on the polymer arms (A), wherein the Z1 functional group comprises an alkyne, for example, an acetylene or a dibenzylcyclooctyne (DBCO).

In some embodiments, a Z2 linker precursor bearing a thiol functional group is linked to the polymer arms (A) through an appropriate reactive group such as an alkyne, alkene, maleimide, resulting in a thioether bond, or the thiol may be reacted with a pyridyl disulfide, e.g., resulting in a disulfide linkage.

In some embodiments, an amine is provided on one molecule and may be linked to another molecule by reacting the amine with any suitable electrophilic group such as carboxylic acids, acid chlorides or activated esters (for example, NHS ester), which results in an amide bond, or the amine may be reacted with alkenes (via Michael addition), aldehydes, and ketones (via Schiff base).

There are many suitable linkers that are well known to those of skill in the art and include, but are not limited to, straight or branched-chain carbon linkers, heterocyclic carbon linkers, rigid aromatic linkers, flexible ethylene oxide linkers, peptide linkers, or a combination thereof. In some embodiments, the carbon linker can include a C1-C18 alkane linker, such as a lower alkyl C4; the alkane linkers can serve to increase the space between two or more molecules, i.e. different components, comprising the star polymer, while longer chain alkane linkers can be used to impart hydrophobic characteristics. Alternatively, hydrophilic linkers, such as ethylene oxide linkers, may be used in place of alkane linkers to increase the space between any two or more molecules and increase water solubility. In other embodiments, the linker can be an aromatic compound, or poly(aromatic) compound that imparts rigidity. The linker molecule may comprise a hydrophilic or hydrophobic linker. In several embodiments, the linker includes a degradable peptide sequence that is cleavable by an intracellular enzyme (such as a cathepsin or the immuno-proteasome).

In some embodiments, the linker may comprise poly(ethylene oxide) (PEG). The length of the linker depends on the purpose of the linker. For example, the length of the linker, such as a PEG linker, can be increased to separate components of an immunogenic composition, for example, to reduce steric hindrance, or in the case of a hydrophilic PEG linker can be used to improve water solubility. The linker, such as PEG, may be a short linker that may be at least 2 monomers in length. The linker, such as PEG, may be between about 4 and about 24 monomers in length, such as 4, 5, 6, 7, 8, 9, 10, 11, 12, 13, 14, 15, 16, 17, 18, 19, 20, 21, 22, 23, 24 monomers in length or more. In some embodiments, a ligand (L) is linked to polymer arms (A) though a PEG linker.

In other embodiments, polymer arms (A) are linked to the core (O) through a linker X comprising 4 or more ethylene oxide units. Unexpectedly, it was found that X1 linker precursors comprising PEG grafted to the core (O) improved the efficiency of polymer arm (A) coupling to the core (O) to generate star polymers of the formula O-(X-A(D))n, O-(X-A[(P2)]-[Z]-L)n or O-(X-A(L)-[Z]-[P3])n. Specifically, it was observed that the coupling of polymer arms (A) with high densities of drugs (D) or relatively high molecular weight (e.g., >10 kDa) ligands (L) linked to the polymers arms could be improved be using an ethylene oxide linker between the core surface and the functional group (FG) on X1 that reacts with the FG on X2 on the polymer arm to form the linker X. Non-limiting explanations for these findings are that extending the FG present on X1 away from the core into the solvent by using 4 or more ethylene oxide units enables improved coupling by reducing the steric hindrance close to the core. Thus, in preferred embodiments of star polymers linked to arms with high densities of drug molecules (e.g., >5 mol % or >10 mol %) and/or relatively high molecular weight ligands (L), the X1 linker precursor is linked to the core through 4 or more ethylene oxide units, preferably between 4 and 36 ethylene oxide units, such as 4, 5, 6, 7, 8, 9, 10, 11, 12, 13, 14, 15, 16, 17, 18, 19, 20, 21, 22, 23, 24, 25, 26, 27, 28, 29, 30, 21, 31, 33, 34, 35, or 36 ethylene oxide units.

In some embodiments, where the linker comprises a carbon chain, the linker may comprise a chain of between about 1 or 2 and about 18 carbons, such as 1, 2, 3, 4, 5, 6, 7, 8, 9, 10, 11, 12, 13, 14, 15, 16, 17, 18 carbons in length or more. In some embodiments, where the linker comprises a carbon chain, the linker may comprise a chain of between about 12 and about 20 carbons. In preferred embodiments, drugs (D) are linked to polymer arms through short alkane linkers typically, no more than 6 carbon atoms in length.

In some embodiments, the linker is cleavable under intracellular conditions, such that cleavage of the linker results in the release of any component linked to the linker, for example, a small molecule immunostimulant or chemotherapeutic drug (D).

For example, the linker can be cleavable by enzymes localized in intracellular vesicles (for example, within a lysosome or endosome or caveolea) or by enzymes, in the cytosol, such as the proteasome, or immuno-proteasome. The linker can be, for example, a peptide linker that is cleaved by protease enzymes, including, but not limited to proteases that are localized in intracellular vesicles, such as cathepsins in the lysosomal or endosomal compartment. The peptide linker is typically between 1-6 amino acids, such as 1, 2, 3, 4, 5, 6.

Certain dipeptides are known to be hydrolyzed by proteases that include cathepsins, such as cathepsins B and D and plasmin, (see, for example, Dubowchik and Walker, 1999, Pharm. Therapeutics 83:67-123). For example, a peptide linker that is cleavable by the thiol-dependent protease cathepsin-B, can be used (for example, a Phe-Leu or a Gly-Phe-Leu-Gly (SEQ ID NO: 1) linker). Other examples of such linkers are described, for example, in U.S. Pat. No. 6,214,345, incorporated herein by reference. In a specific embodiment, the peptide linker cleavable by an intracellular protease is a Val-Cit linker or a Phe-Lys linker (see, for example, U.S. Pat. No. 6,214,345, which describes the synthesis of doxorubicin with the Val-Cit linker).

Particular sequences for the cleavable peptide in the linker can be used to promote processing by immune cells following intracellular uptake. For example, embodiments of star polymers used as vaccines are internalized by immune cells, such as antigen-presenting cells (e.g., dendritic cells). The cleavable peptide linker can be selected to promote processing (i.e. hydrolysis) of the peptide linker following intracellular uptake by the immune cells. The sequence of the cleavable peptide linker can be selected to promote processing by intracellular proteases, such as cathepsins in intracellular vesicles or the proteasome or immuno-proteasome in the cytosolic space.

In several embodiments, linkers comprised of peptide sequences of the formula Pn . . . P4-P3-P2-P1 are used to promote recognition by cathepsins, wherein P1 is selected from arginine, lysine, citrulline, glutamine, threonine, leucine, norleucine, or methionine; P2 is selected from glycine, leucine, valine or isoleucine; P3 is selected rom glycine, serine, alanine, proline or leucine; and P4 is selected from glycine, serine, arginine, lysine aspartic acid or glutamic acid. In a non-limiting example, a tetrapeptide linker of the formula P4-P3-P2-P1 linked through an amide bond to another molecule and has the sequence Lys-Pro-Leu-Arg (SEQ ID NO: 2). For clarity, the amino acid residues (Pn) are numbered from proximal to distal from the site of cleavage, which is C-terminal to the P1 residue, for example, the amide bond between P1-P1′ is hydrolyzed. Suitable peptide sequences that promote cleavage by endosomal and lysosomal proteases, such as cathepsin, are well described in the literature (see: Choe, et al., J. Biol. Chem., 281:12824-12832, 2006).

In several embodiments, linkers comprised of peptide sequences are selected to promote recognition by the proteasome or immuno-proteasome. Peptide sequences of the formula Pn . . . P4-P3-P2-P1 are selected to promote recognition by proteasome or immuno-proteasome, wherein P1 is selected from basic residues and hydrophobic, branched residues, such as arginine, lysine, leucine, isoleucine and valine; P2, P3 and P4 are optionally selected from leucine, isoleucine, valine, lysine and tyrosine. In a non-limiting example, a cleavable linker of the formula P4-P3-P2-P1 that is recognized by the proteasome is linked through an amide bond at P1 to another molecule and has the sequence Tyr-Leu-Leu-Leu (SEQ ID NO:5). Sequences that promote degradation by the proteasome or immuno-proteasome may be used alone or in combination with cathepsin cleavable linkers. In some embodiments, amino acids that promote immuno-proteasome processing are linked to linkers that promote processing by endosomal proteases. A number of suitable sequences to promote cleavage by the immuno-proteasome are well described in the literature (see: Kloetzel, et al., Nat. Rev. Mol. Cell Biol., 2:179-187), 2001, Huber, et al., Cell, 148:727-738, 2012, and Harris et al., Chem. Biol., 8:1131-1141, 2001).

In some embodiments, the linkers, X and/or Z joining together the polymer arm (A) and core (C), and the polymer arm and ligand (L) (or optionally drug (D)) comprise a degradable peptide that is recognized by proteases.

In other embodiments, any two or more components of the star polymer may be joined together through a pH-sensitive linker that is sensitive to hydrolysis under acidic conditions. A number of pH-sensitive linkages are familiar to those skilled in the art and include for example, a hydrazone, semicarbazone, thiosemicarbazone, cis-aconitic amide, orthoester, acetal, ketal, or the like (see, for example, U.S. Pat. Nos. 5,122,368; 5,824,805; 5,622,929; Dubowchik and Walker, 1999, Pharm.

Therapeutics 83:67-123; Neville et al., 1989, Biol. Chem. 264:14653-14661). In certain embodiments, the linkage is stable at physiologic pH, e.g., at a pH of about 7.4, but undergoes hydrolysis at lysosomal pH, pH 5-6.5. In some embodiments, chemotherapeutic and/or immunostimulant small molecule drugs (D), such as TLR-7/8 agonists, are linked to polymer arms (A) through a functional group that forms a pH-sensitive bond, such as the reaction between a ketone and a hydrazine to form a pH labile hydrazone bond. A pH-sensitive linkage, such as a hydrazone, provides the advantage that the bond is stable at physiologic pH, at about pH 7.4, but is hydrolyzed at lower pH values, such as the pH of intracellular vesicles.

In other embodiments, the linker comprises a linkage that is cleavable under reducing conditions, such as a reducible disulfide bond. Many different linkers used to introduce disulfide linkages are known in the art (see, for example, Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987); Phillips et al., Cancer Res. 68:92809290, 2008). See also U.S. Pat. No. 4,880,935.).

In yet additional embodiments the linkage between any two components of the star polymer can be formed by an enzymatic reaction, such as expressed protein ligation or by sortase (see: Fierer, et al., Proc. Natl. Acad. Sci., 111:W1176-1181, 2014 and Theile et al., Nat. Protoc., 8:1800-1807, 2013.) chemo-enzymatic reactions (Smith, et al., Bioconjug. Chem., 25:788-795, 2014) or non-covalent high affinity interactions, such as, for example, biotin-avidin and coiled-coil interactions (Pechar, et al., Biotechnol. Adv., 31:90-96, 2013) or any suitable means that are known to those skilled in the art (see: Chalker, et al., Acc. Chem. Res., 44:730-741, 2011, Dumas, et al., Agnew Chem. Int. Ed. Engl., 52:3916-3921, 2013).

Linkers X and Z

A subset of linkers that perform the specific function of site-selectively coupling, i.e. joining or linking together the core (O) with the polymer arm (A), or polymer arm (A) with the ligand (L) (or optionally drug (D)) are referred to as linkers, X and Z, respectively. The linker X forms as a result of the reaction between a linker precursor X1 and a linker precursor X2. For instance, a linker precursor X1 that is linked to the core (O) may react with a linker precursor X2 attached to the polymer arm (A) to form the linker X that joins the polymer arm (A) to the core (O). The linker Z forms as a result of the reaction between a linker precursor Z1 and a linker precursor Z2. For instance, a linker precursor Z1 that is linked to the polymer arm (A) may react with a linker precursor Z2 attached to a ligand (L) to form the linker Z that joins the polymer arm (A) to the ligand (L). The linkers X and Z may be formed by any suitable means. In preferred embodiments, the linker precursors used to form X and Z are selected for site-selectivity, i.e., a reaction only takes place between X1 and X2 and/or Z1 and Z2, and between no other groups.

In some embodiments, the linkers X and/or Z are formed as a result of a bio-orthogonal “click chemistry” reaction between the linker precursors, X1/X2 and Z1/Z2, respectively. In some embodiments, the click chemistry reaction is a catalyst free click chemistry reaction, such as a strain-promoted azide-alkyne cycloaddition reaction that does not require the use of copper or any catalyst. Non-limiting examples of linker precursors that permit bio-orthogonal reactions include molecules comprising functional groups selected from azides, alkynes, tetrazines and transcyclooctenes. In some embodiments, a linker precursor Z1 comprising an azide reacts with a linker precursor Z2 to form a triazole linker Z. In other embodiments, a linker precursor X2 comprising a tetrazine reacts with a linker precursor X1 comprising a transyclooctene (TCO) to form a linker X comprising the inverse demand Diels-Alder ligation product. In some embodiments, a linker precursor X2 comprising an azide reacts with a linker precursor X1 to form a triazole linker X.

In other embodiments, linker precursors that may permit site-selective reactivity depending on the composition of the different components comprising the star polymer may include thiols, hydrazines, ketones and aldehydes. In some embodiments, a linker precursor Z2 comprising a thiol reacts with a linker precursor Z1 comprising a pyridyl-disulfide or maleimide to form a disulfide or thioether linker Z, respectively. In other embodiments, a linker precursor X1 comprising a hydrazine reacts with a linker precursor X2 comprising a ketone or aldehyde to form a hydrazone linker X. In some embodiments, the linker precursor X1 is a natural or non-natural amino acid residue with a thiol functional group, such as a cysteine, that reacts with a linker precursor X2 comprising a thiol reactive functional group such as maleimide or pyridyl disulfide.

In some embodiments, the linker precursor Z1 is a peptide sequence that is ligated to another peptide sequence comprising the linker precursor Z2. In other embodiments, the linker precursor Z1 binds to a complementary molecule comprising the linker precursor Z2 through high affinity, non-covalent, interactions, for example, through coiled-coil interactions or electrostatic interactions. In other embodiments, the linker precursor Z1 binds to a protein, for example, biotin, which forms high affinity interactions with a protein, Z2, for example, streptavidin.

Linker Molecule (Z) Between the Ligand and the Polymer Arm

Linker molecule (Z) (if present) between the polymer arm and pharmaceutically active compound (P3) at the ends of the polymer arms (A) are formed by the reaction of linker precursors Z1 and Z2 where Z1 is a linker precursor comprising a first reactive functional group and Z2 is a linker precursor comprising a second reactive functional group. A non-limiting example is as follows:

O-[X]-A[(D)]-Z1+Z2-P3→O-([X]-A[(D)]-Z-P3)n

or

[X2]-A[(D)]-Z1+Z2-P3→[X2]-A[(D)]-Z-P3,
wherein P3 is a compound that acts extracellularly or intracellularly.

Linker Molecule (X) Between the Core and the Polymer Arm

Linker molecule (X) is formed by the reaction of linker precursors X1 and X2 where X1 is a linker precursor comprising a first reactive functional group and X2 is a linker precursor comprising a second reactive functional group. A non-limiting example is as follows:

O-X1+X2-A[(D)]-[Z]-P3→O-(X-A(D)-[Z]-P3)n,
wherein P3 is a compound that acts extracellularly or intracellularly.

Linker precursors X1 and X2 allow for coupling of the polymer arm (A) with the core (O). The linker molecule (X) is attached to the core (O) as a result of the reaction between linker precursor X1 and linker precursor X2. For example, a linker precursor X1 that is linked directly or indirectly (e.g. via an extension) to the core (O) may react with a linker precursor X2 that is linked directly or indirectly to the polymer arm (A) to form the linker molecule (X) between the core (O) and the polymer arm (A).

Suitable linker precursors X1 are those that react selectively with linker precursors X2 attached to the polymer arm (A) without linkages occurring at any other site of the polymer arm (A), the linker (Z) (if present) and/or the ligand (L) (if present). This selectivity is important for ensuring a linkage can be formed between the polymer arm (A) and the core (O) without modification to the polymer arm (A) or any ligands (L) or drugs (D), which may otherwise be coupled to the polymer (A).

In certain embodiments, X1 is a nucleophilic species present on the surface of the core (O). The nucleophilic species may be selected from one or more of the group consisting of —OR₁, —NR₁R₂ and —SR₁ where R₁ is selected from H and R₂ is selected from H, NHR₃ or C₁-C₆-alkyl and R₃ is selected from H or C₁-C₆-alkyl. In these embodiments, the linker molecule (X) can be attached to the core (O) by amidation, hydroxylation or sulfation of a carboxyl moiety present in linker precursor X2. In certain embodiments, X1 is NR₁R₂. R₁ and R₂ are each independently selected from the group consisting of H and C₁-C₆-alkyl. In certain specific embodiments, R₁ and R₂ are both H, i.e. X1 on the core is an amine and can be linked to X2 comprising a carboxyl moiety to form an amide bond.

In certain embodiments, the aforementioned acylation can be carried out using a suitable coupling agent. Suitable coupling agents include but are not limited to BOP reagent, DEPBT, N,N′-dicyclohexylcarbodiimide, N,N′-diisopropylcarbodiimide, DMTMM, HATU, HBTU, HCTU, 1-hydroxy-7-azabenzotriazole, hydroxybenzotriazole, PyAOP reagent, PyBOP, thiocarbonyldiimidazole and the like.

In certain other embodiments, the acylation can be carried out by reacting the nucleophilic X1 group with an activated carbonyl moiety. In these embodiments, X2 is an activated carbonyl group of formula —C(O)W where W is a leaving group. Suitable leaving groups include halogen, thiazolidine-2-thione (TT), etc. In certain specific embodiments, W is a thiazolidine-2-thione moiety, e.g., X2 comprises thiazolidine-2-thione (TT) and is reacted with X1 comprising an amine to form an amide bond.

In certain embodiments, the linker molecule (X) comprises an optionally substituted alkyl or optionally substituted heteroalkyl group. In certain embodiments, the linker molecule (X) comprises the core structure of a CTA used in a RAFT polymerization to form the polymer arm (A). For example, when the chain transfer agent is 4, 4′-azobis (4-cyanovaleric acid) initiator (ACVA) the linker molecule (X) will be a 4-cyanovaleric acid derivative (or 4-cyanopentanoic acid derivative) having the formula —C(O)(CH₂)₂C(CN)(CH₃)—.

In some embodiments, the linker precursor X1 and linker precursor X2 are each covalently attached to both the moieties being coupled. In some embodiments, linker precursor X1 and linker precursor X2 are bifunctional, meaning the linkers include a functional group at two sites, wherein the functional groups are used to couple the linker to the two moieties. The two functional groups may be the same (which would be considered a homobifunctional linker) or different (which would be considered a heterobifunctional linker).

Selection of X and Z to Meet the Specific Demands of the Application

The linkers, X and Z, may be selected to meet the specific demands of the application.

For example, the composition of the linkers X and Z, are selected to achieve high polymer arm (A) and ligand (L) loading and to ensure that coupling of the polymer arm (A) and ligand (L) is regioselective.

A non-limiting example of a route for producing star polymers of the present disclosure, referred to as Route 1, is to link one or more ligands (L) (or alternatively drugs (D)) to a heterotelechelic polymer arm (A), and then attach the ligand (L) functionalized polymer arms to the core (O), as follows:

[X2]-A[P2]-Z1+Z2-L→[X2]-A[P2]-Z-L

O-[X1]+[X2]-A[P2]-[Z]-L→O([X]-A[P2]-[Z]-L)n

where O, A, X1, X2, X, Z1, Z2, P2, L, n and [ ] are as previously defined herein; alternatively wherein L may be replaced with D.

Another example of Route 1 is to link one or more drugs to a polymer arm (A) functionalized with a linker precursor X2, and then attach the polymer arm linked to drugs (X2-A(D)-)) to a core (O) with linker precursor X1, to generate a star polymer of formula, O-(X-A(D))n.

Another non-limiting example, referred to as Route 2, is to link heterotelechelic polymer arms (A) to the core (O) and then attach multiple ligands (L) (or alternatively drugs (D)) to the polymer arms (A) radiating therefrom, as follows:

O-[X1]+[X2]-A[P2]-[Z1]→O([X]-A[P2]-[Z1])n

O([X]-A[P2]-[Z1])n+Z2-L→O([X]-A(D)-[Z]-L)n.

where, O, A, X1, X2, X, Z1, Z2, P2, L, n and [ ] are as previously defined herein; alternatively wherein L may be replaced with D.

In certain methods of preparing a star polymer, such as a star polymer ligand display system (or alternatively star polymer drug carrier), using the Route 1 synthetic scheme, the linker precursors Z1 and Z2 are selected to achieve regioselectivity for attachment of the polymer arm (A) to the ligand (L) (or alternatively drug (D)). In certain embodiments, the Z2 linker precursor comprises a clickable functional group, e.g., azides, alkynes, tetrazines, transcyclooctynes or other any such suitable molecule, and the Z1 linker precursor is selected to specifically react with the Z2 linker, such as azide/alkyne or tetrazine/transcyclooctyne. In other embodiments, the linker precursor Z2 comprises a thiol or amine, such as a cysteine or lysine that permits regioselective linkage, e.g., to a linker precursor Z2 that comprises a maleimide or activated carbonyl. In certain other embodiments, an amino acid on the ligand (L) (or alternatively drug (D)), e.g., a cysteine, lysine or alpha-amine of the N-terminal amino acid, is converted to a clickable functional group using a hetero-bifunctional cross-linker. Non-limiting examples include a hetero-bifunctional cross-linker comprising a maleimide linked to an azide; a maleimide linked to an alkyne; a maleimide linked to a tetrazine; a maleimide linked to a transcyclooctyne; an activated carbonyl, e.g., reactive ester linked to an azide; a reactive ester linked to an alkyne; a reactive ester linked to a tetrazine; or a reactive ester linked to a transcyclooctyne, wherein the functional groups of the heterofunctional linker may be linked through any suitable means.

In some embodiments, a star polymer, such as a star polymer ligand display system (or alternatively star polymer drug carrier), is prepared in either aqueous or organic solvents using the Route 1 synthetic scheme. In certain preparations of a star polymer using the Route 1 synthetic scheme in organic or aqueous solvents, a polymer arm (A) bearing a thiol-reactive functional group, e.g., maleimide, is reacted with a linker precursor Z2 bearing a thiol to form a linker, Z, comprising a thioether bond; then a linker precursor X1 bearing an azide or transcyclooctyne is reacted with a linker precursor X2 bearing an alkyne or tetrazine to form a Linker, X, thereby resulting in a fully assembled star polymer ligand display system. In other preparations of a star polymer using the Route 1 synthetic scheme in organic or aqueous solvents, a thiol group present on an ligand (L) (or alternatively a drug (D)) is converted to a clickable group, such as an azide or tetrazine, and the azide or tetrazine Z2 group is reacted with a polymer arm (A) bearing either an alkyne or transcyclooctyne linker precursor Z1 to form a linker, Z; then, the resulting polymer arm (A) and ligand (L) (or alternatively drug (D)) conjugate is reacted to a core, (O), using X1/X2 linker precursor pairs selected from either tetrazine/transcyclooctyne or alkyne/azide, respectively.

In other preparations of a star polymer, such as a star polymer ligand display system (or alternatively star polymer drug carrier), using the Route 1 synthetic scheme in organic or aqueous solvents, a polymer arm (A) bearing an amine-reactive functional group, e.g., activated-ester, is reacted with a linker precursor Z2 bearing an amine to form a linker, Z, comprising an amide bond; then a linker precursor X1 bearing an azide or transcyclooctyne is reacted with a linker precursor X2 bearing an alkyne or tetrazine to form a linker, Z, thereby resulting in a fully assembled star polymer. In other preparations of a star polymer using the Route 1 synthetic scheme in organic or aqueous solvents, an amine group present on an ligand (L) (or alternatively drug (D)) is converted to a clickable group, such as an azide or tetrazine, and the azide or tetrazine Z2 group is reactive with a polymer arm (A) bearing either an alkyne or transcyclooctyne linker precursor Z1 to form a linker, Z; then, the resulting polymer arm (A) and ligand (L) (or alternatively drug (D)) conjugate is reacted to a core, (O), using X1/X2 linker precursor pairs selected from either tetrazine/transcyclooctyne or alkyne/azide, respectively.

In still other preparations of a star polymer, such as a star polymer ligand display system (or alternatively a star polymer drug carrier), using the Route 1 synthetic scheme in organic or aqueous solvents, Z2 comprising a clickable reactive group, such as an azide or tetrazine, is introduced to the ligand (L) (or alternatively drug (D)) during production of the ligand (or drug (D)), and the azide or tetrazine Z2 group is reacted with a polymer arm (A) bearing either an alkyne or transcyclooctyne linker precursor Z1 to form a linker, Z; then, the resulting polymer arm (A) and ligand (L) (or alternatively drug (D)) conjugate is reacted to a core, (O), using X1/X2 linker precursor pairs selected from either tetrazine/transcyclooctyne or alkyne/azide, respectively. In some embodiments, the Z1 linker precursor comprises 1 or more amino acids that are recognized by an enzyme that catalyzes the linkage of Z1 to Z2 to form the linker Z.

In some embodiments, a star polymer, such as a star polymer ligand display system (or alternatively star polymer drug carrier), is prepared in organic solvents using the Route 2 synthetic scheme. In certain preparations of a star polymer using the Route 2 synthetic scheme and an organic solvent, a linker precursor X1 bearing an amine functional group is reacted with a linker precursor X2 bearing an activated ester to form a linker, X, comprising an amide bond, and then a linker precursor Z1 bearing an azide is reacted with a linker precursor Z2 bearing an alkyne to form a Linker, Z, comprising a triazole. In other preparations of a star polymer using the Route 2 synthetic scheme and an organic solvent, a linker precursor X1 bearing an amine functional group is reacted with a linker precursor X2 bearing an activated ester to form a linker, X, comprising an amide bond, and then a linker precursor Z1 bearing a tetrazine is reacted with a linker precursor Z2 bearing an TCO to form a Linker, Z. In additional preparations of a star polymer using the Route 2 synthetic scheme and an organic solvent, a linker precursor X1 bearing an amine functional group is reacted with a linker precursor X2 bearing an activated ester to form a linker, X, comprising an amide bond and any unreacted amines are reacted (“capped”), e.g., with acetyl groups by reaction with acetyl chloride or acetic anhydride; then a thiol-reactive Z1 group, e.g., maleimide, is installed on the polymer arms (A), which are reacted with a linker precursor Z2 bearing a thiol group to form a Linker, Z, comprising a thioether linkage. In still other preparations of a star polymer using the Route 2 synthetic scheme and an organic solvent, a linker precursor X1 bearing a TCO group is reacted with a linker precursor X2 bearing a Tetrazine to form a linker, X, and then a linker precursor Z1 bearing an activated ester is reacted with a linker precursor Z2 bearing an amine to form a Linker, Z, comprising an amide bond.

In some embodiments, a star polymer, such as a star polymer ligand display system (or alternatively star polymer drug carrier), is prepared using the Route 2 synthetic scheme, wherein in the first step either an organic solvent or aqueous solution is used but in the second step an aqueous solution is used, such as may be required due to incompatibility of the ligand (L) (or drug (D)) with organic solvents. A non-limiting example includes the preparation of a star polymer, wherein in the first step in either an organic solvent or aqueous solution, a linker precursor X1 bearing an amine functional group is reacted with a linker precursor X2 bearing an activated ester to form a linker, X, comprising an amide bond, and then in the second step in an aqueous solution a linker precursor Z1 bearing an azide is reacted with a linker precursor Z2 bearing an alkyne to form a linker, Z, comprising a triazole. An additional non-limiting example includes the preparation of a star polymer using the Route 2 synthetic scheme, wherein in the first step in either an organic solvent or aqueous solution, a linker precursor X1 bearing an amine functional group is reacted with a linker precursor X2 bearing an activated ester to form a linker, X, comprising an amide bond and any unreacted amines are reacted (“capped”) prior to installing a thiol-reactive Z1 group, e.g., maleimide, on the polymer arms (A); then in the second step in an aqueous solution, Z1 is reacted with a linker precursor Z2 bearing a thiol group to form a Linker, Z, comprising a thioether linkage. Another non-limiting example includes the preparation of a star polymer using the Route 2 synthetic scheme, wherein in the first step in an organic solvent or aqueous solution, a linker precursor X1 bearing a TCO group is reacted with a linker precursor X2 bearing a Tetrazine to form a linker, X, and then in the second step in an aqueous solution a linker precursor Z1 bearing an activated ester is reacted with a linker precursor Z2 bearing an amine to form a Linker, Z, comprising an amide bond.

The synthetic route as well as the choice of linkers used to prepare star polymer ligand display system depends, in part, on the composition of the ligand (L).

For instance, it was observed unexpectedly that the density of relatively high molecular weight, e.g., greater than 10,000 Da, ligand (L) depends on the synthetic route used for attachment of the ligand (L) to the star polymer. Accordingly, the loading of certain ligands (L) with relatively high molecular weight, e.g., greater than 10,000 Da, was higher when the route 1 synthetic scheme was used as compared with the route 1 scheme. Therefore, in preferred methods of manufacturing star polymer ligand display systems comprising relatively high molecular weight, e.g., greater than 10,000 Da, ligand (L), the route 1 synthetic scheme is used wherein the ligand (L) is first linked to the polymer arm (A), and then the resulting polymer arm ligand conjugate (A-L) is linked to the core (O) to form the fully assembled star polymer ligand display system. While Route 1 may be used to assemble a star polymer ligand display system bearing any composition of ligand (L), in preferred methods of assembling star polymer ligand display systems bearing ligands (L) with molecular weights greater than about 10,000 Da, the Route 1 scheme is used.

In some embodiments, the star polymer ligand display system comprises a ligand (L) based on a recombinant protein or glycoprotein that is not suitable for use in organic solvents. Wherein the recombinant protein or glycoprotein is greater than 10,000 Da in molecular weight and not suitable for use in organic solvent, the route 1 synthetic scheme using aqueous solutions is preferred.

Ligands (L) that are relatively low molecular weight, e.g., less than 10,000 Da, produced by synthetic means and are suitable for use in organic solvents are least restrictive in terms of options for linker chemistries available for forming the Linkers, X and Z and may be produced by either route 1 or 2 in organic or aqueous conditions. Unexpectedly, it was observed that the highest densities of ligands on star polymer ligand display systems could be achieved using synthetic route 2 and organic solvents for the assembly of star polymers displaying ligands (L) that are low molecular weight (<10,000 Da) and suitable for use in organic solvents.

Particular linker precursors (X1 and X2, and Z1 and Z2) and resulting linkers (X and Z) presented in this disclosure provide unexpected improvements in manufacturability and improvements in biological activity. Many such linker precursors (X1 and X2, and Z1 and Z2) and linkers (X and Z) may be suitable for the practice of the invention and are described in greater detail throughout.

Transposition

Those skilled in the art recognize that suitable pairs of functional groups, or complementary molecules, selected to join any two components may be transposable; e.g., functional groups used to join a drug (D) to a monomer may be transposable between the drug and the monomer; linker precursors X1 and X2 may be transposable between X1 and X2; linker precursors for Z1 and Z2 may be transposable between Z1 and Z2; and, linker precursors for X1 and X2 may be transposable between Z2 and Z2. For example, a linker (X) comprised of a triazole may be formed from linker precursors X1 and X2 comprising an azide and alkyne, respectively, or from linker precursors X1 and X2 comprising an alkyne and azide, respectively. Thus, unless stated otherwise herein, any suitable functional group pair resulting in a linker (X or Z, or, e.g., a linker between a pharmaceutically active compound, such as a drug (D) and a monomer) may be placed on either X1 or X2 and Z1 or Z2 or the drug and the reactive monomer.

As disclosed herein, certain linker precursor combinations were found to lead to improved manufacturability. For instance, in the preparation of star polymer ligand display systems (i.e. star polymers displaying ligands (L) on the surface) using the route 1 synthetic scheme in aqueous conditions, wherein the linker X comprises a triazole bond, the combination of a linker precursor X1 comprising an azide and the linker precursor X2 comprising an alkyne was found to lead to improved arm loading (density) as compared with the linker precursor X1 comprising an alkyne and the linker precursor X2 comprising an azide. A non-binding explanation is that the azide is more accessible than the alkyne for coupling the core (O) to the polymer arm (A) in aqueous conditions.

In other embodiments, wherein the linker X is formed as a result of a reaction between a tetrazine and transcyclooctyne, the combination of a linker precursor X1 comprising a TCO and the linker precursor X2 comprising a tetrazine was found to lead to improved arm loading (density) as compared with the linker precursor X1 comprising a tetrazine and the linker precursor X2 comprising a TCO. A non-binding explanation is that tetrazine functional group was unexpectedly found to be unstable on certain cores (O) comprising multiple amine functional groups. Therefore, in preferred embodiments, wherein the dendrimer core comprises primary amines, the Z2 comprising TCO is used.

Incorporation of X2 and Z1 onto the Polymer Arms (A)

The linker precursors X2 and Z1 may be introduced onto the polymer through any suitable means.

For polymer arms (A) produced by RAFT polymerization, the linker precursors X2 and Z1 may be selectively introduced at the ends of the polymer arms during polymerization and capping steps.

Introduction of X2 and Z1 onto the polymer arms (A) using RAFT polymerization can be achieved using specialized CTAs and initiators. In a non-limiting example, the CTA is selected from dithiobenzoates and has the generic structure,

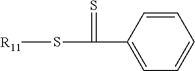

wherein Ru is X2 (or Z1); and, the initiator is selected from the azo class of initiators and has the generic structure, R₁₂—N═N—R₁₂, wherein, R₁₂ in this example is equivalent to R₁₁ and is X2 (or Z1).

In a non-limiting example, X2 (or Z1) is introduced to the polymer arm during polymerization using a functionalized azo-initiator and a functionalized dithiobenzoate-based CTA:

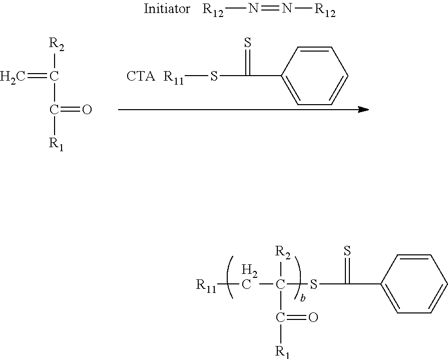

wherein R₁ is —OR₃, —NHR₃ or —N(CH₃)R₃, where R₂ can be H or CH₃, and R₃ is independently selected from any hydrophilic substituent; Rn dithiobenzoate-based CTA and R₁₂ on the initiator are the same and are both X2 (or Z1); and, the resulting polymer comprises an integer number, b, of repeating units of hydrophilic monomers. In this example, in the second step, the dithiobenzoate group on the end of the polymer chain is removed and capped with Z1 (or X2) using a functionalized azo-initiator as shown here:

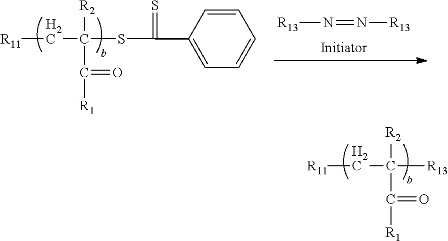

wherein R₁ is —OR₃, —NHR₃ or —N(CH₃)R₃, where R₂ can be H or CH₃, and R₃ is independently selected from any hydrophilic substituent; Rn is X2 (or Z1); b is an integer number of repeating units of hydrophilic monomers and R₁₃ is Z1 (or X2).

In some embodiments, the CTA is based on dithiobenzoate and comprises an activated carbonyl, such as an activated ester, and has the structure

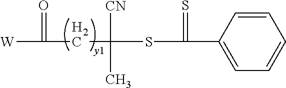

wherein y1 denotes an integer number of methylene units, typically between 1 to 6, and W is a leaving group. A non-limiting example of a dithiobenzoate-based CTA comprising an activated carbonyl is:

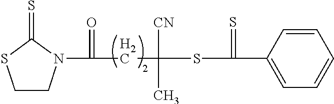

In some embodiments, the CTA is based on dithiobenzoate and comprises a functional group (FG) linked to the CTA through an amide bond and has the structure:

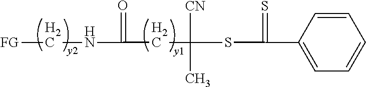

wherein y1 and y2 denote an integer number of repeating methylene units, typically between 1 to 6, and FG is any functional group, such as an azide, alkyne, tert-butyloxycarbonyl protected amine (NH₂-Boc), tert-butyloxycarbonyl protected hydrazide (NHNH-Boc). In a non-limiting example of a dithiobenzoate-based CTA linked to a functional group through an amide bond, the FG is an alkyne, y1=2 and y2=1 and the structure is:

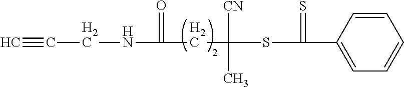

In some embodiments, the azo-initiator comprises an activated carbonyl and has the structure

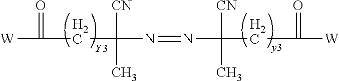

wherein y3 denotes an integer number of methylene units, typically between 1 to 6, and W is a leaving group. A non-limiting example of an azo-initiator comprising an activated carbonyl wherein y3=2 and W is thiazoline-2-thione is:

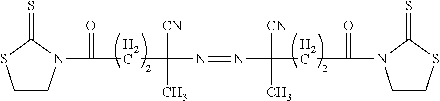

In some embodiments, the azo-initiator comprises a functional group (FG) linked to the initiator through an amide bond, and has the structure:

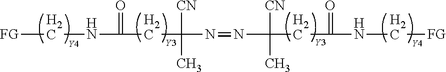

wherein y3 and y4 denote an integer number of methylene units, typically between 1 to 6, and the FG is any functional group, e.g. azide, alkyne, tert-butyloxycarbonyl protected amine (NH₂-Boc), tert-butyloxycarbonyl protected hydrazide (NHNH-Boc), dibenzocyclooctyne (DBCO), bicyclononyne (BCN), methyltetrazine (mTz). In some embodiments, the linker joining the FG to the amide bond may include an ethylene oxide spacer alone or in combination with an aliphatic linker. A non-limiting example of an azo-initiator, wherein in FG is an alkyne, y3=2 and y4=1 is:

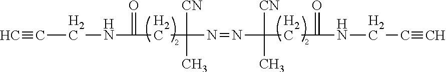

Functionalized initiators and CTAs can be used to incorporate the suitable X2 and Z1 linker precursors onto the polymer during polymerization. In certain embodiments, polymer arms with X2 comprising an activated carbonyl and Z1 comprising an azide are produced in a two-step reaction. In a non-limiting example for the preparation of a polymer arm (A) comprising an activated carbonyl for X2 and an azide for Z1, acrylamide-based monomers are polymerized in the presence of CTA and initiator containing an activated carbonyl as shown here:

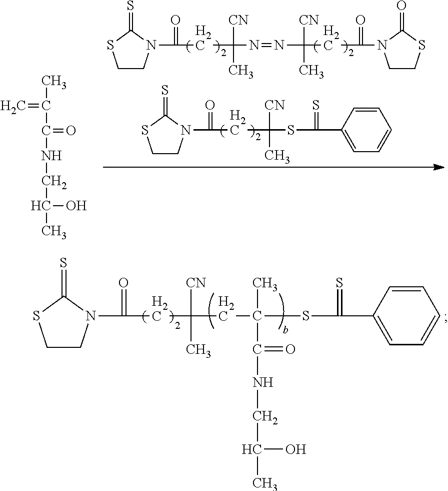

in the second step, the dithiobenzoate group of the polymer arm is replaced with Z1 by reacting (“capping”) the polymer with an initiator containing an azide functional group, as shown here:

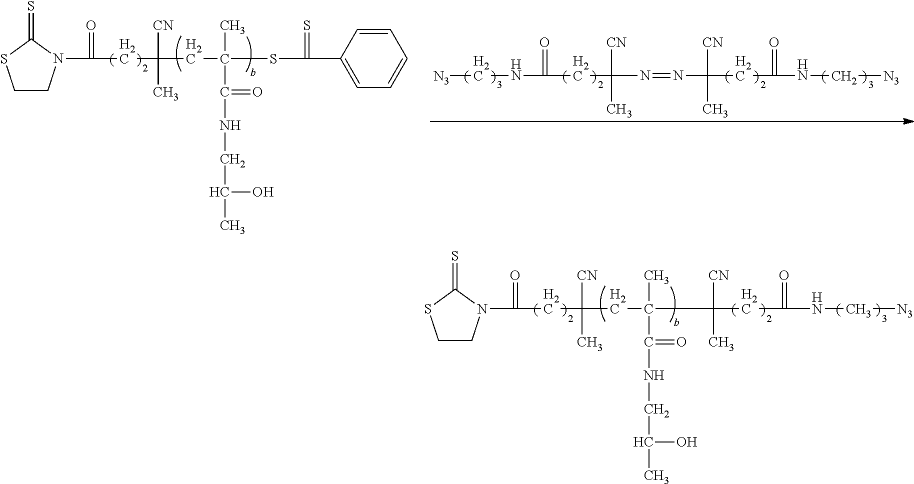

In an alternative non-limiting example for the preparation of a polymer arm (A) comprising an activated carbonyl for X2 and an azide for Z1, acrylamide-based monomers are polymerized in the presence of CTA and initiator containing an azide as shown here:

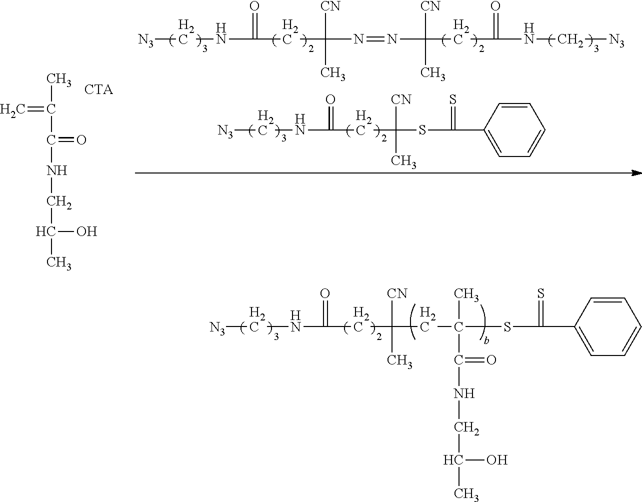

in the second step, the dithiobenzoate group of the polymer arm is replaced with X1 by reacting (“capping”) the polymer with an initiator containing an activated carbonyl group, as shown here:

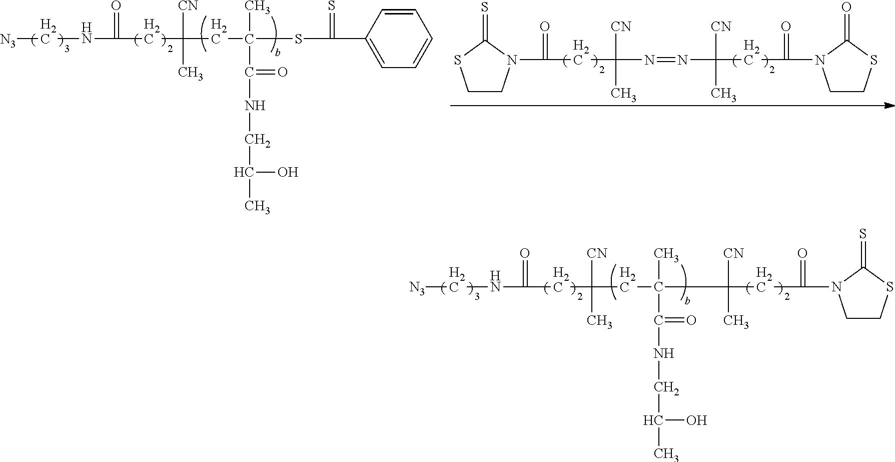

Unexpectedly, it was found that the addition of the Z1 precursor to the polymer arm (A) in the first step, i.e. polymerization of monomers in the presence of Z1-functionalized CTA and Z1-functionalized initiator, followed by the addition of the X2 precursor to the polymer (A) in the second step (i.e. by capping the polymer arm with excess X2 functionalized initiator) led to polymer arms (A) that were less prone to cross-linking cores than polymers arms (A) wherein the X2 is added in the first step. A non-limiting explanation is that the linker precursor X2 or Z1 introduced onto the polymer arm in the first step (polymerization) has the propensity to form a homo-bifunctional polymer arm, X2-A-X2 or Z1-A-Z1, respectively, in the second step (capping). Since X2-A-X2 can cross-link cores, e.g., O-X1+X2-A-X2+X1-O to form O-X-A-X-O, but Z1-A-Z1 cannot, it was determined herein that the route that does not lead to cross-linking, i.e. adding X2 during or after capping is preferred. Therefore, in preferred embodiments of star polymers, the Z1 linker precursor is optionally added to the polymer arm (A) during polymerization in a first step, and the linker precursor X2 is added to the polymer arm (A) in a second step (capping) by reacting the polymer arm with excess initiator functionalized with X2. This process led to unexpected improvements in manufacturing of star polymers.

Methods for preparing polymer arms with different X2 and Z1 linker precursors groups are described throughout the specification.

Ligand (L)

In certain embodiments, the star polymer comprises one or more ligands (L). The ligand (L) can be any molecule that acts extracellularly, such as by binding to or associating with soluble or cell surface bound receptors, such as extracellular receptors. The extracellular receptors to which the ligand (L) binds may be free, or membrane or cell associated. Non-limiting examples of ligands (L) include synthetic or naturally occurring compounds. Non-limiting examples include protein, peptide, polysaccharide, glycopeptide, glycoprotein, lipid, or lipopeptide-based ligands (L). Examples of proteins include naturally occurring protein ligands, as well as antibodies or antibody fragments that are agonists or antagonists of extracellular receptors. The antibody may be engineered or naturally occurring, i.e., derived from an organism, or a combination thereof, e.g., a partially engineered antibody or antibody fragment. Other examples include synthetic, low-molecular-weight molecules, such as non-naturally occurring heterocycles that bind to extracellular receptors.

The present inventors have unexpectedly found that arrays of ligands (L) on star polymers of formula O-([X]-A[(D)]-[Z]-L)n show improved receptor binding as well as enhanced biological activity as compared with that observed with ligands arrayed on linear co-polymers, or delivered on conventional particle delivery systems based on liposomes.

Advantageously, star polymers of the present disclosure can be modulated to optimize the pharmacokinetics and pharmacodynamics of a range of ligands (L).

The star polymers of the present disclosure can be used to display ligands and modulate the pharmacokinetics of the ligands. Alternatively, or in addition, the star polymers of the present disclosure can be used for the delivery of ligands (L).

The ligand (L) may be a peptide and the linker precursor (Z2) may be attached to the N-terminal amino acid of the peptide, the C-terminal amino acid of the peptide, or to a side chain of any one or more amino acid residues present in the peptide.

In certain embodiments, the ligand (L) has a molecular weight of from about 250 to about 10,000 Da. Ligands with relatively low molecular weight, e.g., less than about 10,000 Da can typically be accessed synthetically and are often suitable for use in organic solvents.

In certain embodiments, the ligand (L) is a peptide that binds to checkpoint molecules, such as PD1, PD-L1 and CTLA-4, such as antagonists of checkpoint molecules. In some embodiments the peptide binds to VEGF receptors, such as peptide-based antagonists of VEGF receptors.

In certain embodiments, the ligand (L) is a peptide that binds to B cell receptors and encompasses an epitope(s) derived from an immunogen(s) isolated from infectious organisms or cancer cells. In other embodiments, the ligand is a peptide that binds to T cell receptors and encompasses an epitope(s) derived from immunogen(s) isolated from infectious organisms or cancer cells. In still other embodiments, the ligand is a peptide that binds to T cell receptors and encompasses an epitope(s) derived from a self-protein. The peptide-based ligand (L) comprising an epitope(s) from infectious organisms may be from any infectious organism, such as a protein or glycoprotein derived from a fungus, bacterium, protozoan or virus. Alternatively, the peptide-based ligand (L) comprises an epitope from a tumor-associated antigen including self-antigens or tumor-specific neoantigens; the peptide-based ligand (L) may also comprise epitopes from self-proteins that are not tumor-associated.

The peptide antigen used as a ligand (L) may be any antigen that is useful for inducing an immune response in a subject. The peptide antigen may be used to induce either a pro-inflammatory or tolerogenic immune response depending on the nature of the immune response required for the application. In some embodiments, the peptide antigen is a tumor-associated antigen, such as a self-antigen, neoantigen or tumor-associated viral antigen (e.g., HPV E6/E7). In other embodiments, the peptide antigen is an infectious disease antigen, such as a peptide derived from a protein isolated from a virus, bacteria, fungi or protozoan microbial pathogen. In still other embodiments, the peptide antigen is a peptide derived from an allergen or an autoantigen, which is known or suspected to cause allergies or autoimmunity.

The peptide antigen is comprised of a sequence of amino acids or a peptide mimetic that can induce an immune response, such as a T cell or B cell response in a subject. In some embodiments, the peptide antigen comprises an amino acid or amino acids with a post-translational modification, non-natural amino acids or peptide-mimetics. The peptide antigen may be any sequence of natural, non-natural or post-translationally modified amino acids, peptide-mimetics, or any combination thereof, that have an antigen or predicted antigen, i.e. an antigen with a T cell or B cell epitope.

Immunogenic compositions of star polymers displaying peptide-based immunogens may comprise a single antigen, or the star polymer may comprise two or more different peptide antigens each having a unique antigen composition. In some embodiments, the star polymer includes only a single antigen. In some embodiments, the single peptide antigen comprises both B cell and T cell epitopes. In other embodiments, the star polymer comprises two different antigens. In some embodiments, wherein the star polymer comprises two different antigens, one of the antigens comprises a B cell epitope and the other antigen comprises a T cell epitope. In still other embodiments, the star polymer comprises up to 50 different peptide antigens each having a unique antigen composition. In some embodiments, the immunogenic compositions comprise star polymers that each comprise 20 different peptide antigens. In other embodiments, the immunogenic compositions comprise star polymers that comprise 5 different peptide antigens. In some embodiments, the immunogenic compositions comprise a mixture of up to 50 different star polymers each containing a unique peptide antigen. In other embodiments, the immunogenic compositions comprise up to 20 different star polymers each containing a unique peptide antigen. In still other embodiments, the immunogenic compositions comprise a single star polymer containing a single peptide antigen.

The length of the peptide antigen depends on the specific application and the route for producing the peptide antigen (A). The peptide antigen should minimally comprise at least a single T cell or B cell epitope. Therefore, wherein the T cell and/or B cell epitopes of an immunogen are known or can be predicted, a peptide antigen that comprises only the minimal epitopes of the immunogen (sometimes referred to as a minimal immunogen) can be produced by synthetic means and used to induce or modulate immune responses against those specific B cell and/or T cell epitopes that are known or predicted. Such synthetic peptide antigens comprising T cell and/or B cell epitopes typically comprise between about 5 to about 50 amino acids. In preferred embodiments, the peptide antigen produced by synthetic means is between about 7 to 35 amino acids, e.g., 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids.

For peptide antigens produced by synthetic means, it was observed that improved immune responses, e.g., antibody responses, to B cell epitopes was observed when the peptide antigen was linked to the polymer arms (A) through a spacer that increases distance between the peptide antigen ligand (L) and the polymer arm (A). In some embodiments of star polymers delivering ligands (L) comprising peptide-based antigens, the peptide is linked to polymer arms through a hydrophilic poly(ethylene oxide) (PEG) spacer, with between 2 to 36 ethylene oxide units, which is incorporated into the peptide either at the N- or C-terminus during solid-phase peptide synthesis.

In other embodiments, the peptide antigen is a fragment of a polypeptide. In still other cases, the peptide antigen is a full-length polypeptide, such as a protein antigen that may be recombinantly expressed.

In some embodiments, the peptide antigen is a minimal CD8 or CD4 T cell epitope that comprises the portions of a tumor-associated antigen, infectious disease antigen, allergen or auto-antigen that are known or predicted in silico (or measured empirically) to bind MHC-I or MHC-II molecules. For tumor-associated antigens, the peptide antigen that is a minimal CD8 or CD4 T cell epitope that is predicted in silico (or measured empirically) to bind MHC-I or MHC-II molecules should also be a sequence of amino acids that is unique to the tumor cell. Algorithms for predicting MHC-I or MHC-II binding are widely available (see Lundegaard et al., Nucleic Acids Res., 36:W509-W512, 2008 and http://www.cbs.dtu.dk/services/NetMHC/). In some embodiments of a personalized therapy for a particular subject (e.g., patient), the peptide antigen comprising a star polymer may comprise a minimal CD8 T cell epitope from a tumor-associated antigen, infectious disease antigen, allergen or auto-antigen that is typically a 7-13 amino acid peptide that is predicted to have <1,000 nM binding affinity for a particular MHC-I allele that is expressed by that subject. In some embodiments of a personalized therapy for a particular subject (e.g., patient), the peptide antigen may comprise a minimal CD4 T cell epitope from a tumor-associated antigen, infectious disease antigen, allergen or auto-antigen that is a 10-16 amino acid peptide that is predicted to have <1,000 nM binding affinity for a particular MHC-II allele that is expressed by that subject. In a preferred embodiment, when a minimal CD8 or CD4 T cell epitope cannot be identified for a tumor-associated antigen, infectious disease antigen, allergen or auto-antigen, or when the tumor-associated antigen, infectious disease antigen, allergen or auto-antigen contains multiple CD8 and CD4 T cell epitopes, the peptide antigen may be between 16-35 amino acids may be up to 50 amino acids, e.g., up to 35 amino acids, up to 25 amino acids, or up to 20 amino acids, or up to 16 amino acids such that it may contain all possible CD8 or CD4 T cell epitopes.

In some embodiments, the peptide antigen is a minimal B cell immunogen (or minimal epitope) that comprises the portions of a tumor-associated antigen, infectious disease antigen, allergen or auto-antigen that are known or predicted in silico (or measured empirically) to bind to specific antibodies. In some embodiments, the peptide antigen is a minimal immunogen that binds to B cells that give rise to neutralizing antibodies.

In some embodiments of the present disclosure, the peptide antigen is derived from tumor-associated antigens. Tumor-associated antigens can either be self-antigens that are present on healthy cells but are preferentially expressed by tumor cells, or neoantigens, which are aberrant proteins that are specific to tumor cells and are unique to individual patients. Suitable self-antigens include antigens that are preferentially expressed by tumor cells, such as CLPP, Cyclin-A1, MAGE-A1, MAGE-C1, MAGE-C2, SSX2, XAgElb/GAGED2a, Melan-A/MART-1, TRP-1, Tyrosinase, CD45, glypican-3, IGF2B3, Kallikrein 4, KIF20A, Lengsin, Meloe, MUC5AC, surviving, prostatic acid phosphatase, NY-ESO-1 and MAGE-A3. Neoantigens arise from the inherent genetic instability of cancers, which can lead to mutations in DNA, RNA splice variants and changes in post-translational modification, all potentially leading to de novo protein products that are referred to collectively as neoantigens or sometimes predicted neoantigens. DNA mutations include changes to the DNA including nonsynonymous missense mutations, nonsense mutations, insertions, deletions, chromosomal inversions and chromosomal translocations, all potentially resulting in novel gene products and therefore neoantigens. RNA splice site changes can result in novel protein products and missense mutations can introduce amino acids permissive to post-translational modifications (e.g. phosphorylation) that may be antigenic. The instability of tumor cells can furthermore result in epigenetic changes and the activation of certain transcription factors that may result in selective expression of certain antigens by tumor cells that are not expressed by healthy, non-cancerous cells.

Star polymers used in personalized cancer vaccines should include peptide antigens that comprise the portions of tumor-associated antigens that are unique to tumor cells. Peptides antigens comprising neoantigens arising from a missense mutation should encompass the amino acid change encoded by 1 or more nucleotide polymorphisms. Peptide antigens comprising neoantigens that arise from frameshift mutations, splice site variants, insertions, inversions and deletions should encompass the novel peptide sequences and junctions of novel peptide sequences. Peptide antigens comprising neoantigens with novel post-translational modifications should encompass the amino acids bearing the post-translational modification(s), such as a phosphate or glycan. In preferred embodiments, the peptide antigen comprises the 0-25 amino acids on either side flanking the amino acid change or novel junction that arises due to a mutation. In one embodiment, the peptide antigen is a neoantigen sequence that comprises the 12 amino acids on either side flanking the amino acid change that arises from a single nucleotide polymorphism, for example, a 25 amino acid peptide, wherein the 13^{th} amino acid is the amino acid residue resulting from the single nucleotide polymorphism. In some embodiments, the peptide antigen is a neoantigen sequence that comprises the 12 amino acids on either side flanking an amino acid with a novel post-translational modification, for example, a 25 amino acid peptide, wherein the 13^{th} amino acid is the amino acid residue resulting from the novel post-translational modification site. In other embodiments, the peptide antigen is a neoantigen sequence that comprises 0-12 amino acids on either side flanking a novel junction created by an insertion, deletion or inversion. In some cases, the peptide antigen comprising neoantigens resulting from novel sequences can encompass the entire novel sequence, including 0-25 amino acids on either side of novel junctions that may also arise.

Tumor-associated antigens suitable as peptide antigens for immunogenic compositions of the present disclosure can be identified through various techniques that are familiar to one skilled in the art. Tumor-associated antigens can be identified by assessing protein expression of tumor cells as compared with healthy cells, i.e., non-cancerous cells from a subject. Suitable methods for assessing protein expression include but are not limited to immunohistochemistry, immunofluorescence, western blot, chromatography (i.e., size-exclusion chromatography), ELISA, flow cytometry and mass spectrometry. Proteins preferentially expressed by tumor cells but not healthy cells or by a limited number of healthy cells (e.g., CD20) are suitable tumor-associated antigens. DNA and RNA sequencing of patient tumor biopsies followed by bio-informatics to identify mutations in protein-coding DNA that are expressed as RNA and produce peptides predicted to bind to MHC-I or MHC-II alleles on patient antigen presenting cells (APCs), may also be used to identify tumor-associated antigens that are suitable as peptide antigens for immunogenic compositions of the present disclosure.

In certain embodiments, tumor-associated antigens suitable as peptide antigens for immunogenic compositions are identified using mass spectrometry. Suitable peptide antigens are peptides identified by mass spectrometry following elution from the MHC molecules from patient tumor biopsies but not from healthy tissues from the same subject (i.e., the peptide antigens are only present on tumor cells but not healthy cells from the same subject). Mass spectrometry may be used alone or in combination with other techniques to identify tumor-associated antigens. Those skilled in the art recognize that there are many methods for identifying tumor-associated antigens, such as neoantigens (see Yadav et al., Nature, 515:572-576, 2014) that are suitable as peptide antigens for the practice of the disclosed invention.

In certain embodiments, the tumor-associated antigens used as peptide antigens are clonal or nearly clonal within the population of neoplastic cells, which may be considered heterogeneous in other respects.

Tumor-associated antigens selected for use as peptide antigens in personalized cancer vaccination schemes may be selected based on mass spectrometry confirmation of peptide-MHC binding and/or in silico predicted MHC binding affinity and RNA expression levels within tumors. These data provide information on whether or not a tumor-associated antigen is expressed and presented by tumor cells and would therefore be a suitable target for T cells. Such criteria may be used to select the peptide antigens used in a personalized cancer vaccine.

Cancer vaccines may include peptide antigens that comprise tumor-associated antigens that are patient-specific and/or tumor-associated antigens that are shared between patients. For example, the tumor-associated antigen can be a conserved self-antigen, such as NY-ESO-1 (testicular cancer) or gp100 (melanoma), or the antigen may be a cryptic epitope, such as Na17 (melanoma) that is not typically expressed by healthy cells but is conserved between patients. Immunogenic compositions of the present disclosure may include peptide antigens that arise from so-called hot-spot mutations that are frequent mutations in certain genes or gene regions that occur more frequently than would be predicted by chance.

Non-limiting examples of hot spot mutations include the V600E mutation in BRAF protein, which is common to melanoma, papillary thyroid and colorectal carcinomas, or KRAS G12 mutations, which are among the most common mutations, such as KRAS G12C. A number of suitable self-antigens as well as neoantigens that arise from hotspot mutations are known and are incorporated herein by reference: see Chang et al., Nature Biotechnology, 34:155-163, 2016; Vigneron, N., et al, Cancer Immunology, 13:15-20, 2013.

In some embodiments, the peptide antigen can be from a hematological tumor. Non-limiting examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

In some embodiments, the peptide antigen can be from a solid tumor. Non-limiting examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma). In several examples, a tumor is melanoma, lung cancer, lymphoma breast cancer or colon cancer.

In some embodiments, the peptide antigen is a tumor-associated antigen from a breast cancer, such as a ductal carcinoma or a lobular carcinoma. In some embodiments, the peptide antigen is a tumor-associated antigen from a prostate cancer. In some embodiments, peptide antigen is a tumor-associated antigen from a skin cancer, such as a basal cell carcinoma, a squamous cell carcinoma, a Kaposi's sarcoma, or a melanoma. In some embodiments, the peptide antigen is a tumor-associated antigen from a lung cancer, such as an adenocarcinoma, a bronchiolaveolar carcinoma, a large cell carcinoma, or a small cell carcinoma. In some embodiments, the peptide antigen is a tumor-associated antigen from a brain cancer, such as a glioblastoma or a meningioma. In some embodiments, the peptide antigen is a tumor-associated antigen from a colon cancer. In some embodiments, the peptide antigen is a tumor-associated antigen from a liver cancer, such as a hepatocellular carcinoma. In some embodiments, the peptide antigen is a tumor-associated antigen from a pancreatic cancer. In some embodiments, peptide antigen is a tumor-associated antigen from a kidney cancer, such as a renal cell carcinoma. In some embodiments, the peptide antigen is a tumor-associated antigen from a testicular cancer.

In some embodiments, the peptide antigen is a tumor-associated antigen derived from premalignant conditions, such as variants of carcinoma in situ, or vulvar intraepithelial neoplasia, cervical intraepithelial neoplasia, or vaginal intraepithelial neoplasia.

In some embodiments, the peptide antigen is an antigen from an infectious agent, such as a virus, a bacterium, or a fungus. In several embodiments, the peptide antigen is a peptide or glycopeptide derived from an infectious agent; for example, the HIV Envelope fusion peptide or a V3 or V1/V2 glycopeptide from HIV. In other embodiments, the peptide antigen is a minimal immunogen from influenza virus. In some embodiments the antigen is a hepatitis antigen. In still other embodiments, the peptide antigen is a minimal immunogen from HPV. In still other embodiments, the peptide antigen is a minimal immunogen from an emerging infectious disease, such as a peptide antigen from SARS, SARS-CoV-2 or MERS. Suitable minimal immunogens derived from coronaviruses include those derived from the receptor binding domain of the spike glycoprotein.

In some embodiments, the peptide antigen represents an auto-antigen. The auto-antigen may be identified and selected on the basis of screening a subject's own T cells for auto-reactivity against self-antigens presented in the context a patient's own MHC-I molecules. Alternatively, the peptide antigens may be selected using in silico methods to predict potential auto-antigens that (i) have a predicted high affinity for binding a subjects' own MHC-I molecules and (ii) are expressed and/or known to be associated with pathology accounting for a subject's auto-immune syndrome. In other embodiments, the peptide antigen represents a CD4 epitope derived from an allergen and is selected on the basis of the peptide antigen having a high binding affinity for a patient's own MHC-II molecules.

Those skilled in the art recognize that any peptide, protein or post-translationally modified protein (e.g., glycoprotein) that leads to an immune response and is useful in the prevention or treatment of a disease can be selected for use as a peptide antigen for use in the immunogenic compositions of the present invention.

In certain embodiments, the ligand (L) is a saccharide that binds to lectin receptors, such as CD22. In other embodiments, the ligand is a synthetic or naturally occurring agonist of extracellular pattern recognition receptors (PRRs) and has immunostimulatory properties. Suitable PRR agonists (PRRa) include agonists of Toll-like receptor-1 (TLR-1), TLR-2, TLR-4, TLR-5 and TLR-6; agonists of NOD-like receptors (NLRS) and agonists of C-type lectin receptors.

In some embodiments, the ligand (L) binds to C-type lectin receptors (CLRs) and is used to promote uptake by certain antigen presenting cells (APCs). In several embodiments, the ligand that binds to CLRs is a modified mannose and has the structure:

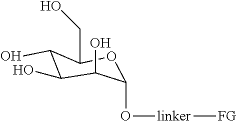

wherein the “linker” is any suitable linker molecule and FG is any suitable functional group that can be used to attach the linker modified mannose to a polymer arm (A). In some embodiments, the linker is PEG and FG is an azide.

In other embodiments, the ligand that binds to CLRs is a tetrasaccharide that binds to DC-SIGN and has the structure:

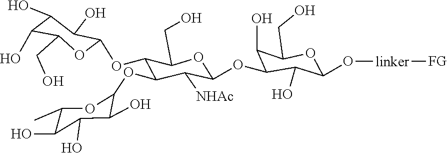

wherein the “linker” is any suitable linker molecule and FG is any suitable functional group that can be used to attach the linker modified mannose to a polymer arm (A). In some embodiments, the linker is PEG and FG is an azide.

In certain embodiments, the ligand (L) has a molecular weight of greater than about 10,000 Da. Ligands with relatively high molecular weight, e.g., greater than about 10,000 Da are typically accessed by producing the ligand recombinantly using an expression system and are often not suitable for use in organic solvents during the manufacturing of the star polymer.

Other suitable ligands (L) include therapeutic antibodies or antibody fragments useful for the treatment of a disease. Therapeutic antibody molecules include antibodies directed against pathogens, cancer cells, soluble host proteins, toxins, as well as extracellular receptors and ion channels that may be blocked or stimulated to modulate signalling within the cell.

Suitable antibodies for use as ligands (L) include antibodies directed against tumor antigens. Non-limiting examples of antibodies directed against tumor antigens include antibodies directed against CD19, CD20, CD22, CD30, CD33, CD38, CD51, EGFR, PDGF-R, VEGFR, SLAMF7, integrin αvβ3, carbonic anhydrase 9, HER2, GD2 ganglioside, mesothelin, TAG-72. Suitable antibodies include antibodies against immune checkpoint molecules that can be used to reverse or modulate immune suppression. Non-limiting examples include PD1, PD-L1, OX-40, CTLA-4, 41BB. Suitable antibodies include agonists of the immune response, including but not limited to antibodies directed against CD40.

Suitable antibodies include those that can modify disease, including the prevention, mitigation or reversal of disease, such as antibodies directed against beta-amyloid, sclerostin, IL-6, TNF-alpha, VEGF, VEGFR, IL-5, IL-12, IL-23, Kallikrein, PCSK9, BAFF, CD125 or similar such targets of antibodies.

In some embodiments, the ligand molecule is a peptide-MHC complex, e.g., a complex of a CD8 or CD4 T cell epitope with an MHC-I or MHC-II epitope, which may be used for inducing tolerance, when not provided with an additional immune stimulus, or may be used for activating and/or expanding T cells when used in combination with an immunostimulatory molecule.

Density of L

The present inventors have unexpectedly found that the density of the ligand (L) has a profound impact on biological activity for certain applications described herein. For example, the present inventors have identified that starting polymer displaying >5 ligands (L) are optimal for inducing downstream cellular signalling cascades across applications. Specifically, when the ligand (L) is a peptide-based B cell immunogen, greater than 5, typically 15 or more ligands were required to induce B cell activation and the induction of antibodies in vivo. For larger ligands (L), including antibodies, 5 or more ligand molecules per star polymer were found to be suitable for activity.

For example, increasing the density of peptide-based B cell immunogens, as ligands (L), arrayed on star polymers of the present disclosure results in increased antibody responses. In certain embodiments, star polymers of the present disclosure used as vaccines for inducing antibody responses include more than 5 immunogens per star polymer, preferably between 5 and 60. In some embodiments, vaccines based on star polymers of the present disclosure have an average of between 5 and 15 immunogens arrayed on the surface. In other embodiments, vaccines based on star polymers of the present disclosure have an average of between 15 and 25 immunogens arrayed on the surface; between 20 and 30; or, between 25 and 35. In preferred embodiments, vaccines based on star polymers of the present disclosure have an average of between 15 to 40, such as between 25 and 35, immunogens arrayed on the surface. In still other embodiments, vaccines based on star polymers of the present disclosure have an average of up to 60 immunogens arrayed on the surface.

Compositions of Star Polymers for Inducing an Antibody Response

Protein or peptide-based B cell immunogens can be displayed on star polymers of the present disclosure to induce an antibody response against one or more epitopes present on the immunogen. The immunogen may be derived from an infectious organism, tumor cells or allergens. The immunogen may be a full-length protein that contains multiple B cell epitopes, or a short peptide, e.g., a peptide or modified peptide, such as a glycopeptide, that includes only a single epitope.

As discussed herein, various parameters of star polymers of the present disclosure can be optimized to maximize antibody responses induced against B cell immunogens.

Preferred embodiments of star polymers displaying B cell immunogens can be represented by the schematic:

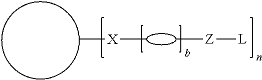

In some embodiments, the ligand (L) is a B cell immunogen between about 5 to 50 amino acids in length; b is an integer number of repeating units of hydrophilic monomer (B), which is typically between about 50 to 450, X is a linker that typically comprises an amide and Z is a linker that typically comprises a triazole, and the core is preferably a PAMAM dendrimer of generation G3, G4 or G5, preferably G5. A non-limiting example is provided here, wherein the hydrophilic monomers are HPMA:

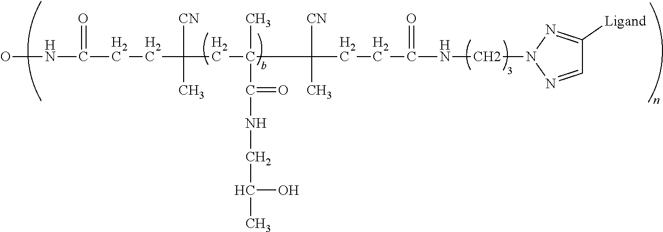

In preferred embodiments, the hydrophilic monomer is HPMA; the linkers X and Z are derived from excess Initiator and CTA during polymerization, respectively,

Additional Components of Star Polymers Used as Vaccines

Vaccines based on star polymers of the present disclosure minimally comprise a core (O), arms (A), ligands (L) and an immunogen, e.g., a peptide-based B cell epitope. Additional components may be included to enhance the immune response induced. For example, in some embodiments a peptide-based CD4 helper epitope is attached to between 5 to 50% of the polymer arms (A) of the star polymer. In other embodiments, immunostimulatory small molecule drugs (D) are linked to the surface of the core (O) or in a multivalent array along the polymer arms (A), represented as. In still other embodiments, vaccines based on star polymers of the present disclosure minimally comprising a core, arms and peptide antigens as ligands (L) may include both CD4 helper epitopes and immunostimulatory small molecule drugs (D).

Compositions for Avoiding Antibody Responses

When the star polymers of the present disclosure are used for applications other than for inducing an antibody response, it may be beneficial to prevent anti-ligand or anti-drug antibodies that can be induced to ligands (L) or drugs (D) arrayed on the star polymers. Unexpectedly, we disclose herein that certain polymer arm (A) compositions and certain drugs (D) can be incorporated into the structure of star polymers of the present disclosure to prevent antibody responses directed against the star polymers.

Unexpectedly, poly(anionic) polymers and/or those with saccharides that bind CD22 were found to prevent the induction of antibody responses against arrayed ligands (L) displayed on the surface of the star polymers.

In some embodiments, the ligand that binds to CD22 is a trisaccharide that has the structure:

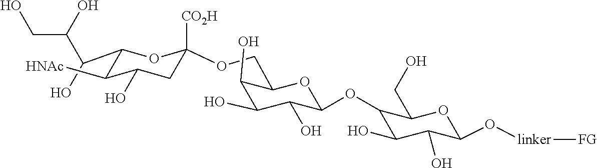

wherein the “linker” is any suitable linker molecule and FG is any suitable functional group that can be used to attach the linker modified mannose to a polymer arm (A). In some embodiments, the linker is PEG and FG is an azide.

Compositions for Inducing Tolerance and Immune Suppression

One application of the star polymers of the present disclosure is for inducing tolerance. In some embodiments, star polymers of the present disclosure with five or more peptide-MHC complexes as ligands (L) were arrayed on star polymers of the present disclosure and used to induce tolerance. In other embodiments of star polymers of the present disclosure for inducing tolerance, five or more peptide-MHC complexes were arrayed on star polymers of the present disclosure and the composition included an mTOR inhibitor as a means to dampen the immune response induced against the peptide presented in the context of MHC.

Compositions of Star Polymers for In Situ Vaccination

In addition to the array of ligands (L), star polymers of the present disclosure may also be used for the delivery of drugs (D) for cancer treatment. Accordingly, small molecule immunostimulant and/or chemotherapeutic drugs (D) may be conjugated to the core (O), at the ends of the polymer arms (A) or, preferably, multivalently on polymer arms (A) of the star polymers.

Optimization of Drug Uptake into Tumors

Herein, we report unexpected findings related to how various parameters of star polymers of the present disclosure can be used to optimize uptake of ligands (L) and/or drugs (D) into tumors and induce durable tumor regression in relevant animal models.

While any class of PRR agonist molecule could potentially be used as an immunostimulant for inducing anticancer immunity, it was found, unexpectedly, that certain classes of immunostimulants lead to unexpectedly enhanced tumor clearance as compared with other classes of immunostimulants. Herein, it is disclosed that preferred immunostimulants are those that induce the production of specific cytokines, i.e. interferons (IFNs) and/or IL-12. Thus, in preferred embodiments, star polymers of the present disclosure for cancer treatment include immunostimulants selected from agonists of Stimulator of Interferon Genes (STING), TLR-3, TLR-4, TLR-7, TLR-8, TLR-7/8 and TLR-9. For clarity, since TLR-4 is surface expressed (i.e. extracellular), agonists of TLR-4 are referred to herein as ligands (L).

Non-limiting examples of TLR-3 agonists include dsRNA, such as PolyI:C, and nucleotide base analogs; TLR-4 agonists include lipopolysaccharide (LPS) derivatives, for example, monophosphoryl lipid A (MPL) small molecules such as pyrimidoindole; TLR-7 & -8 agonists include ssRNA and nucleotide base analogs, including derivatives of imidazoquinolines, hydroxy-adenine, benzonapthyridine and loxoribine; TLR-9 agonists include unmethylated CpG and small molecules that bind to TLR-9; STING agonists include cyclic dinucleotides, and synthetic small molecules, such as alpha-mangostin and its derivatives as well as linked amidobenzimidazole (“diABZI”) and related molecules (see: Ramanjulu et al., Nature, 20:439-443, 2018).

In several embodiments, the star polymer for cancer treatment comprises small molecule drugs (D) with immunostimulant properties selected from imidazoquinoline-based agonists of TLR-7, TLR-8 and/or TLR-7 & -8. Numerous such agonists are known, including many different imidazoquinoline compounds.

Imidazoquinolines are of use as small molecule immunostimulatory drugs (D) used in star polymers found in immunogenic composition used for vaccination and/or for treating cancer or infectious diseases in the absence of a co-administered antigen. Imidazoquinolines are synthetic immunomodulatory compounds that act by binding Toll-like receptors 7 and 8 (TLR-7/TLR-8) on antigen presenting cells (e.g., dendritic cells), structurally mimicking these receptors' natural ligand, viral single-stranded RNA. Imidazoquinolines are heterocyclic compounds comprising a fused quinoline-imidazole skeleton. Derivatives, salts (including hydrates, solvates, and N-oxides), and prodrugs thereof also are contemplated by the present disclosure. Particular imidazoquinoline compounds are known in the art, see for example, U.S. Pat. Nos. 6,518,265, 4,689,338. In some non-limiting embodiments, the imidazoquinoline compound is not imiquimod and/or is not resiquimod.

In some embodiments, the drugs (D) with immunostimulatory properties can be a small molecule having a 2-aminopyridine fused to a five membered nitrogen-containing heterocyclic ring, including but not limited to imidazoquinoline amines and substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

In some embodiments, the drug (D) with immunostimulatory properties is an imidazoquinoline with the formula:

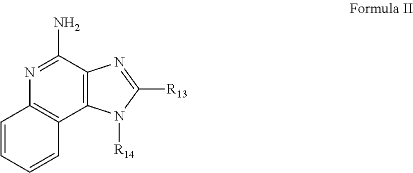

In Formula II, R₁₃ is selected from one of hydrogen, optionally-substituted lower alkyl, or optionally-substituted lower ether; and R₁₄ is selected from one of optionally substituted arylamine, or optionally substituted lower alkylamine. R₁₃ may be optionally substituted to a linker that links to a polymer.

In some embodiments, the R₁₃ included in Formula II can be selected from hydrogen,

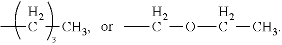

In some embodiments, R₁₄ can be selected from,

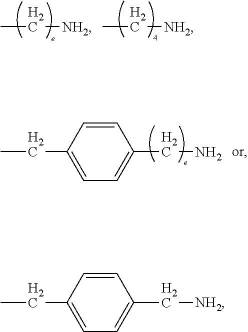

wherein e denotes the number of methylene unites is an integer from 1 to 4.

In some embodiments, R₁₄ can be

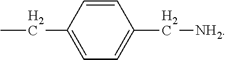

In some embodiments, R₁₄ can be

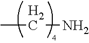

In some embodiments, R₁₃ can be

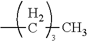

### and R₁₄ can be

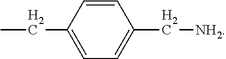

In certain embodiments, drugs (D) based on chemotherapeutic molecules are incorporated onto the star polymer. Chemotherapeutic agents include, without limitation alkylating agents (cisplatin, cyclophosphamide & temozolomide as an example), topoisomerase inhibitors (Topoisomerase I inhibitors and Topoisomerase II inhibitors), mitotic inhibitors (taxanes and Vinca alkaloids as an example), antimetabolites (5-fluorouracil, capecitabine & methotrexate as an example), and anti-tumor antibiotics (anthracycline family, actinomycin-D and bleomycin as an example). Also, included in this definition are receptor tyrosine kinase inhibitors, differentiating agents, angiogenesis inhibitors, steroids and anti-hormonal agents among others.

In a non-limiting example, the anthracycline is doxorubicin and has the structure:

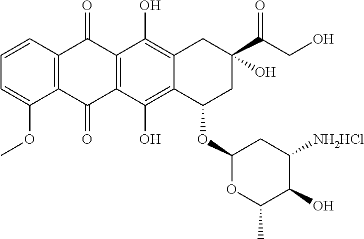

wherein the doxorubicin molecule may be linked to the star polymer arms (A) through the amine or ketone position via an amide or hydrazone bond, respectively.

While any class of chemotherapeutic could be used, it was found, unexpectedly, that certain classes of chemotherapeutics used in combination with immunostimulants lead to unexpectedly enhanced tumor clearance. Herein, it is disclosed that preferred chemotherapeutics are those that induce either or both reversal of immune-suppression and/or the induction of immunogenic cell death. Thus, in certain embodiments, star polymers of the present disclosure for cancer treatment include immunostimulants and/or chemotherapeutics, wherein the chemotherapeutics are selected from anthracyclines, taxanes, platinum compounds, 5-fluorouracil, cytaribine and other such molecules that are useful for eliminating or altering the phenotype of suppressor cells in the tumor microenvironment.

Immunostimulatory and/or chemotherapeutic drugs (D) may be attached to any suitable functional group on the star polymers of the present disclosure through any suitable means. Functional groups that can be used for attachment of drugs (D) may be located on the core (O), at the ends of the polymer arms (A) and/or in a pendant array along the backbones of the polymer arms (A). The inventors' results show that high loading of small molecule immunostimulatory and/or chemotherapeutic drugs (D) is fundamental to achieving high levels of efficacy and that maximal drug (D) loading is achieved when the small molecule drug (D) is arrayed along the backbone of the polymer arms (A).

An unexpected finding disclosed herein is that increasing loading of immunostimulatory and/or chemotherapeutic small molecule drugs (D) on star polymers of the present disclosure results in improved efficacy for cancer treatment. Therefore, in certain embodiments, star polymers of the present disclosure for cancer treatment include greater than 10 mass percent of chemotherapeutic and/or immunostimulatory small molecule drugs, such as between 10 to 80 mass percent. To achieve a high density of chemotherapeutic and/or immunostimulatory small molecule drugs (D), such drug molecules may be attached in a pendant array along the backbones of the polymer arms (A) of the star polymer.

Since the molecular weight of the star polymer without ligands (L) or small molecule drugs (D) is principally driven by the mass of each polymer arm, the mol % density of drugs (D) attached to the star polymer (i.e. the percentage of monomers of the polymer arms linked to drug molecules) can be modulated to achieve a given mass percent of immunostimulatory and/or chemotherapeutic drug, independent of the molecular weight or number of arms.

The mass percent of drug can be approximated using the following equation:

Mass percent drug=((MW D/(MWavg+(MW D*mol % D)))*mol % D)*100;

wherein MW D is the molecular weight of the small molecule drug (D); MWavg is the average MW of the monomers comprising the polymer arm (A), excluding the mass of the drug molecule linked to monomer E, and mol % D is the percentage of monomer units (E) that are linked to drug. Note: 1 mol % drug (D) means that 1 out of 100 monomer units comprising the polymer arms (A) of the star polymer are linked to drug (D). 10 mol % drug (D) means that 10 out of 100 monomer units comprising the polymer arms of the star polymer are linked to drug (D).

In a non-limiting example of a star polymer comprising small molecule drugs (D) with a molecular weight of 300 Da that are attached in a pendant array along the backbone of linear HPMA-based co-polymer arms, comprised of 143 Da HPMA monomers, at a density of about 5 mol %, the mass percent of the small molecule drug is about 9.5 mol %. In certain embodiments of star polymers used for cancer treatment, small molecule drugs between about 200-1,000 Da are arrayed along the polymer arms (A) at a density of between about 4.0 to about 50 mol % to achieve a mass percent of about 10 to about 80 mass %. In other embodiments of star polymers used for cancer treatment, small molecule drugs (D) with about 250-350 Da molecular weight are arrayed along the polymer arms at a density of between about 6 to about 40 mol % to achieve a mass percent of about 10 to about 50 mass %. In still other embodiments of star polymers used for cancer treatment, small molecule drugs (D) with about 350-450 Da molecular weight are arrayed along the polymer arms at a density of between about 5.0 to about 30 mol % to achieve a mass percent of about 10 to about 50 mass %.

Unexpectedly, however, it was observed that increasing the density of immunostimulatory and/or chemotherapeutic small molecule drugs (D) with amphiphilic or hydrophobic properties attached to statistical random copolymer arms (A) comprised entirely of hydrophilic monomers (B) and reactive monomers (E) linked to drug, leads to an increased propensity of the star polymers to form aggregates in aqueous conditions. Accordingly, attachment of amphiphilic small molecule drugs, such as aromatic heterocycles selected from imidazoquinoline-based agonists of TLR-7, TLR-8 or both TLR-7/8 or linked amidobenzimidazole-based (e.g., diABZI) agonists of STING, at high densities, e.g., greater than 5 mol %, to single block (i.e. not di-block) polymer arms (A) comprising hydrophilic monomers, e.g., HPMA, but not charged monomers, attached to a PAMAM core, led to such star polymers forming aggregates in aqueous conditions. Similarly, attachment of hydrophobic small molecule drugs, such as anthracyclines at high densities, e.g., greater than 5 mol %, to single block (i.e. not di-block) polymer arms (A) comprised of hydrophilic monomers, e.g., HPMA, but not charged monomers, attached to a PAMAM core, led to such star polymers forming aggregates in aqueous conditions. Aggregate formation of the star polymers in aqueous conditions, e.g., aqueous buffer at physiologic pH, i.e. pH 7.4, and physiologic osmolality, i.e., ˜290 mOs/Kg, presents major challenges to manufacturing and would render such formulations unsuitable for GMP manufacturing and use as a drug product for administration to humans. Note: high mol % is meant to describe a mol % that has been historically difficult to achieve using conventional compositions and methods of manufacturing. For instance, the mol % of amphiphilic or hydrophobic drugs linked to star polymers has been conventionally less than 5 mol % due to the limitations described throughout (such as low coupling efficiency and formation of aggregates). Thus 5 mol % represents a high density relative to conventional technologies.

To address the challenge of attaching high densities of amphiphilic or hydrophobic small molecule drugs to star polymers, two structural designs were introduced that unexpectedly reduced the propensity of star polymers carrying high densities of the amphiphilic or hydrophobic small molecule drugs, e.g., amphiphilic or hydrophobic immunostimulatory and/or chemotherapeutic small molecule drugs (D), to aggregate.

In some embodiments, the polymer arms of star polymers for cancer treatment comprise a di-block copolymer architecture, wherein the immunostimulatory and/or chemotherapeutic small molecule drugs (D) are attached to the block that is proximal to the core, and the other block is solvent exposed and is not attached to any small molecule drugs (D). A non-limiting example is:

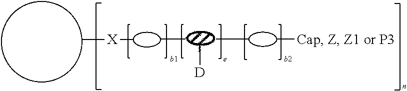

Wherein, an integer number, n, of polymer arms with di-block architecture, i.e. -(B)b1-co-(E(D))e-b-(B)b2-, comprising an integer number, b1, of hydrophilic monomers (B) and an integer number, e, of reactive monomers (E) linked to drug molecules (D) on one block of the polymer arm (A) that is proximal to the core of the star polymer, and an integer number, b2, of hydrophilic monomers on the other block of the polymer arm (A) that is distal to the core of the star polymer, are linked to a core, O, through a linker, X; additionally wherein the distal ends of each of the polymer arms are either capped with a capping group, linked to a linker precursor, Z1, or linked directly or indirectly through a linker, Z, to a pharmaceutically active compound, P3.

In some embodiments of star polymers for cancer treatment that comprise di-block copolymer arms (A) with both hydrophilic monomers (B) and reactive monomers (E) linked to drug molecules (D) on one block of the polymer arm (A) that is proximal to the core and hydrophilic monomers on the other block of the polymer arm (A) that is distal to the core, the hydrophilic monomers are selected from hydrophilic acrylamides or acrylates, as shown here in this non-limiting example:

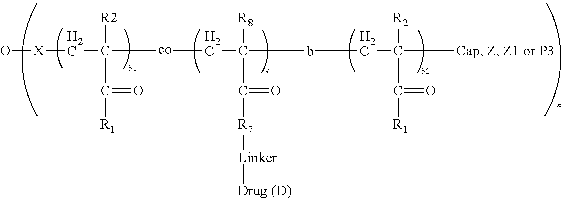

Wherein, in preferred embodiments, the hydrophilic monomers are selected from HPMA; the linker, X, comprises an amide bond; the end of each polymer arm distal to the core is capped, preferably with isobutyronitrile; the core is a PAMAM dendrimer, such as a generation 1, 2, 3, 4, 5 or 6 PAMAM dendrimer, preferably generation 3, 4 or 5; the molecular weight of the di-block polymer arms are between 5,000 and 50,000 Daltons, preferably between 20,000 and 40,000 Daltons, and the ratio of the molecular weights of each of the blocks is between 1:3 and 3:1, such as 1:3, 1:2, 1:1, 2:1 and 3:1, preferably between 1:2 and 2:1, such as 1:1 (i.e. each block is approximately the same molecular weight): n is an integer between 3 and 30, preferably greater than 5, such as 5, 6, 7, 8, 9, 10, 11, 12, 13, 14, 15, 16, 17, 18, 19, 20, 21, 22, 23, 24, 25, 26, 27, 28, 29 and 30, preferably between 10 and 30, such as between 15 and 25; the drug molecules (D) are selected from small molecule immunostimulant and/or chemotherapeutic drugs, such as imidazoquinoline-based agonists of TLR-7, TLR-8 and TLR-7/8, agonists of STING, such as linked amidobenzimidazole-based (diABZI) agonists of STING, or anthracyclines; and, the drugs (D) are linked to the reactive co-monomer through an amide, ester or hydrazone on the block proximal to the core at a density greater than 5 mol % (i.e. 5 out of 100 monomers of one block comprise reactive co-monomers linked to drug, D), preferably between 10 and 50 mol %, such as 10 mol %, 15 mol %, 20 mol %, 25 mol %, 30 mol %, 35 mol %, 40 mol %, 45 mol % or 50 mol %; and, wherein the hydrodynamic radius of the star polymer is between 5 and 25 nm, preferably between 7.5 and 15 nm.

A non-limiting example wherein the hydrophilic monomers of the above example are selected from HPMA is shown here:

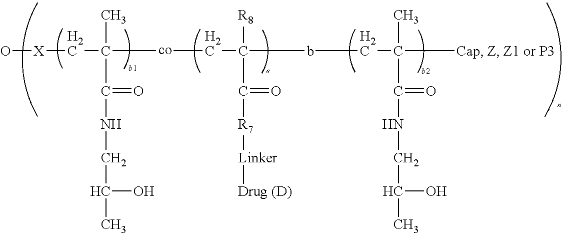

A non-limiting example of star polymers for cancer treatment that comprise di-block copolymer arms (A) with both hydrophilic HPMA monomers (B) and reactive monomers (E) linked to drug molecules (D) on one block of the polymer arm (A) that is proximal to the core and hydrophilic HPMA monomers on the other block of the polymer arm (A) that is distal to the core of the star polymer, wherein the drug molecule is an imidazoquinoline of Formula II linked to the reactive monomer E through an amide bond is shown here:

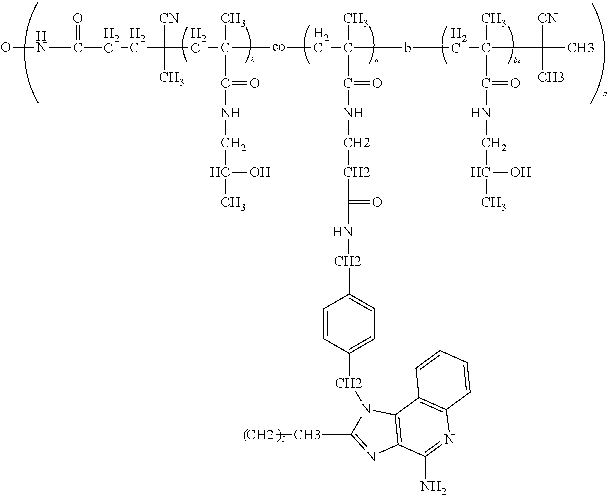

A non-limiting example of star polymers for cancer treatment that comprise di-block copolymer arms (A) with both hydrophilic HPMA monomers (B) and reactive monomers (E) linked to drug molecules (D) on one block of the polymer arm (A) that is proximal to the core and hydrophilic HPMA monomers on the other block of the polymer arm (A) that is distal to the core of the star polymer, wherein the drug molecule is a linked amidobenzimidazole-based agonist of STING linked to the reactive monomer E through an amide bond is shown here:

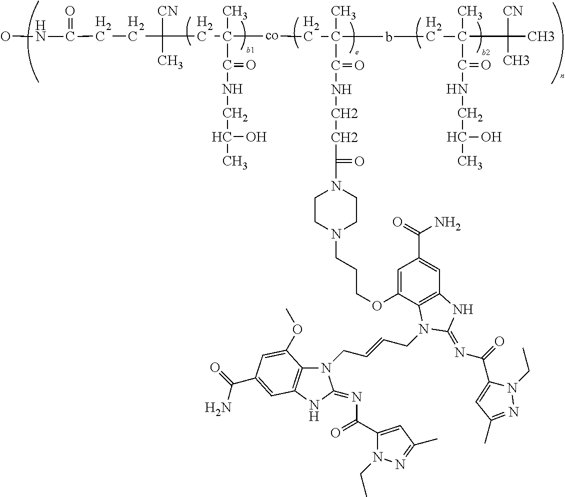

A non-limiting example of star polymers for cancer treatment that comprise di-block copolymer arms (A) with both hydrophilic HPMA monomers (B) and reactive monomers (E) linked to drug molecules (D) on one block of the polymer arm (A) that is proximal to the core and hydrophilic HPMA monomers on the other block of the polymer arm (A) that is distal to the core of the star polymer, wherein the drug molecule is an anthracycline chemotherapeutic molecule linked to the reactive monomer E through an amide bond is shown here:

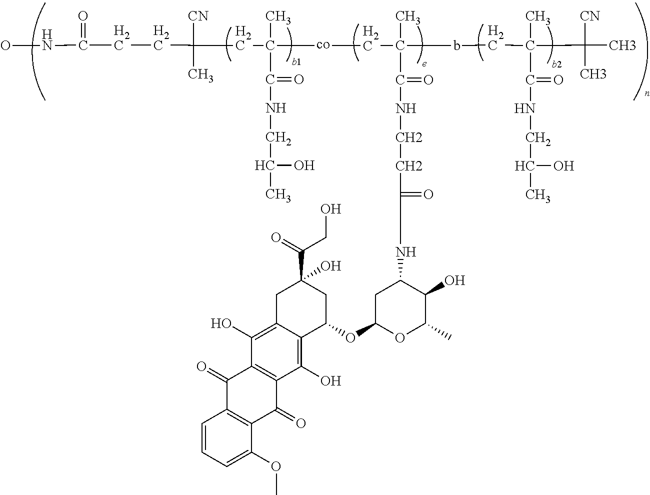

An unexpected finding was that, for star polymers for cancer treatment that comprise di-block copolymer arms (A) with both hydrophilic HPMA monomers (B) and reactive monomers (E) linked to drug molecules (D) on one block of the polymer arm (A) that is proximal to the core and hydrophilic HPMA monomers on the other block of the polymer arm (A) that is distal to the core of the star polymer, high densities, e.g., greater than 5 mol %, of amphiphilic or hydrophobic drug molecules, such as imidazoquinolines, amidobenzimidazole-based STING agonists and anthracyclines, could be attached to the block of the polymer arm proximal to the core without inducing the star polymers to aggregate.

In certain embodiments, the polymer arms (A) of star polymers for cancer treatment comprise hydrophilic monomers, immunostimulatory and/or chemotherapeutic small molecule drugs (D) linked to reactive co-monomers and charged co-monomers. A non-limiting example is:

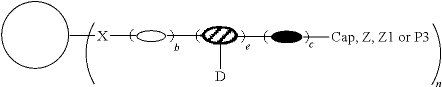

Wherein, an integer number, n, of terpolymers comprising an integer number, b, of hydrophilic monomers (B), an integer number, e, of reactive monomers (E) linked to drug molecules (D) and an integer number, c, of charged monomers, i.e. -(B)b-co-(E(D))e-co-(Cc-, are linked to a core, O, through a linker, X; additionally wherein the distal end of each of the polymer arms is either capped with a capping group, linked to a linker precursor, Z1, or linked directly or indirectly through a linker, Z, to a pharmaceutically active compound, P3.

In some embodiments of star polymers for cancer treatment that comprise terpolymer arms (A) with hydrophilic monomers (B), reactive monomers (E) linked to drug molecules (D) and charged monomers (C), the hydrophilic monomers are selected from hydrophilic acrylamides or acrylates, as shown here in this non-limiting example:

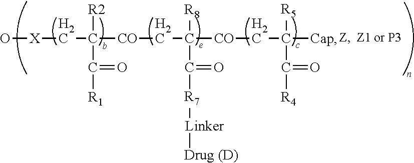

Wherein, in preferred embodiments, the hydrophilic monomer (B) is selected from HPMA and the charged monomer (C) is negatively charged, such as methacrylic acid or methacrylic acid substituted with an amino acid, e.g., beta-alanine; the linker, X, comprises an amide bond; the end of each polymer arm distal to the core is capped, preferably with isobutyronitrile; the core is a PAMAM dendrimer, such as a generation 1, 2, 3, 4, 5 or 6 PAMAM dendrimer, preferably generation 3, 4 or 5; the molecular weight of the terpolymer arms are between 5,000 and 50,000 Daltons, preferably between 20,000 and 40,000 Daltons; n is an integer between 3 and 30, preferably greater than 5, such as 5, 6, 7, 8, 9, 10, 11, 12, 13, 14, 15, 16, 17, 18, 19, 20, 21, 22, 23, 24, 25, 26, 27, 28, 29 and 30, preferably between 10 and 30 or between 15 and 25; the drug molecules (D) are selected from small molecule immunostimulant and/or chemotherapeutic drugs, such as imidazoquinoline-based agonists of TLR-7, TLR-8 and TLR-7/8, agonists of STING, such as linked amidobenzimidazole-based agonists of STING, or anthracyclines; and, the drugs (D) are linked to the reactive co-monomer through an amide, ester or hydrazone on the polymer arm at a density greater than 5 mol % (i.e. 5 out of 100 monomers comprise reactive co-monomers linked to drug, D), preferably between 10 and 50 mol %, such as 10 mol %, 15 mol %, 20 mol %, 25 mol %, 30 mol %, 35 mol %, 40 mol %, 45 mol % or 50 mol %; the density of the charged monomers are greater than 10 mol %, preferably greater than 20 mol %; and, the hydrodynamic radius of the star polymer is between 5 and 25 nm, preferably between 7.5 and 15 nm.

An unexpected finding was that a charged monomer density greater than 10 mol % was needed to prevent aggregation of star polymers with greater than 10 mol % amphiphilic or hydrophobic drug molecules (D) attached to terpolymer arms of star polymers. Thus, in preferred embodiments of star polymers with terpolymer arms, amphiphilic or hydrophobic drug molecules are attached at a density greater than 10 mol % and the density of charged monomers is selected to be 10 mol % or higher, preferably between 10 mol % and 30 mol %, such as between 10 mol % and 20 mol %, e.g., 10 mol %, 11 mol %, 12, mol %, 13 mol %, 14 mol %, 15 mol %, 16 mol %, 17 mol %, 18 mol %, 19 mol % and 20 mol %.

A non-limiting example wherein the hydrophilic monomers and charged monomers of the above example are selected from HPMA and methacrylic acid substituted with beta-alanine, respectively, is shown here:

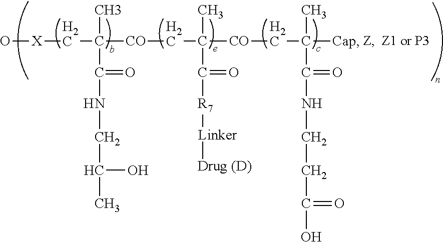

A non-limiting example of star polymers for cancer treatment that comprise terpolymer arms (A) with hydrophilic HPMA monomers (B), reactive monomers (E) linked to drug molecules (D) and charged monomers based on methacrylic acid substituted with beta-alanine, wherein the drug molecule is an imidazoquinoline of Formula II linked to the reactive monomer E through an amide bond is shown here:

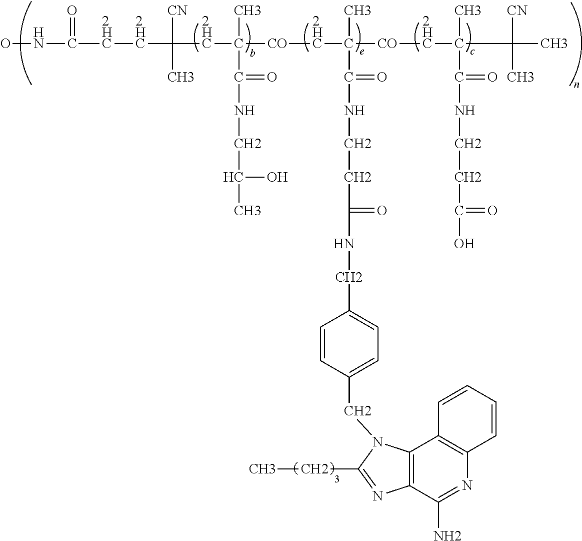

In still other embodiments, the polymer arms (A) of star polymers for cancer treatment comprise a di-block copolymer architecture, wherein hydrophilic monomers and immuno-stimulatory and/or chemotherapeutic small molecule drugs (D) linked to reactive comonomers (E) are on one block that is proximal to the core, and the second block, which is solvent exposed, includes hydrophilic monomers and charged co-monomers. A non-limiting example is:

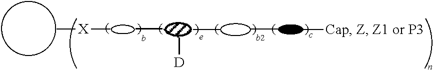

Wherein, an integer number, n, of polymer arms with di-block architecture, i.e. -(B)b1-co-(E(D))e-b-(B)b2-co-(C)c-, comprising an integer number, b1, of hydrophilic monomers (B) and an integer number, e, of reactive monomers (E) linked to drug molecules (D) on one block of the polymer arm (A) that is proximal to the core of the star polymer, and an integer number, b2, of hydrophilic monomers (B) and an integer number, c, of charged monomers (C) on the other block of the polymer arm (A) that is distal to the core of the star polymer, are linked to a core, O, through a linker, X; additionally wherein the distal ends of each polymer arm is capped with a capping group, linked to a linker precursor, Z1 or linked directly or indirectly through a linker, Z, to a pharmaceutically active compound, P3.

In some embodiments of star polymers for cancer treatment that comprise di-block copolymer arms (A) with both hydrophilic monomers (B) and reactive monomers (E) linked to drug molecules (D) on one block of the polymer arm (A) that is proximal to the core and hydrophilic monomers (B) and charged monomers (C) on the other block of the polymer arm (A) that is distal to the core of the star polymer, the hydrophilic monomers are selected from hydrophilic acrylamides or acrylates and the charged monomers are selected from acrylamides and acrylates as shown here in this non-limiting example:

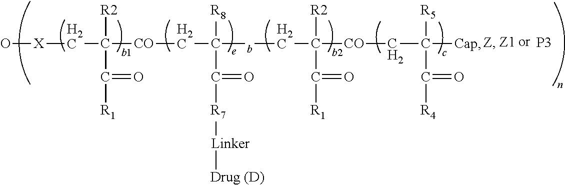

Wherein, in preferred embodiments, the hydrophilic monomer (B) is HPMA, the charged monomer (C) is negatively charged at physiologic pH, such as methacrylic acid or, in some embodiments, methacrylic acid substituted with an amino acid, e.g., beta-alanine; the linker, X, comprises an amide bond; the distal end of each polymer arm is capped, preferably with isobutyronitrile; the core is a PAMAM dendrimer, such as a generation 1, 2, 3, 4, 5 or 6 PAMAM dendrimer, preferably generation 3, 4 or 5; the molecular weight of the di-block polymer arms are between 5,000 and 50,000 Daltons, preferably between 20,000 and 40,000 Daltons, and the ratio of the molecular weights of each of the blocks is between 1:3 and 3:1, such as 1:3, 1:2, 1:1, 2:1 and 3:1, preferably between 1:2 and 2:1, such as 1:1, i.e. each block is approximately the same molecular weight; n is an integer between 3 and 30, preferably greater than 5, such as 5, 6, 7, 8, 9, 10, 11, 12, 13, 14, 15, 16, 17, 18, 19, 20, 21, 22, 23, 24, 25, 26, 27, 28, 29 and 30, preferably between 10 and 30, such as between 15 and 25; the drug molecules (D) are selected from small molecule immunostimulant and/or chemotherapeutic drugs, such as imidazoquinoline-based agonists of TLR-7, TLR-8 and TLR-7/8, agonists of STING, such as linked amidobenzimidazole-based (diABZI) agonists of STING, or anthracyclines; and, the drugs (D) are linked to the reactive co-monomer through an amide, ester or hydrazone on the block proximal to the core at a density greater than 5 mol % (i.e. 5 out of 100 monomers of one block comprise reactive co-monomers linked to drug, D), preferably between 10 and 50 mol %, such as 10 mol %, 15 mol %, 20 mol %, 25 mol %, 30 mol %, 35 mol %, 40 mol %, 45 mol % or 50 mol %; and, wherein the hydrodynamic radius of the star polymer is between 5 and 25 nm, preferably between 7.5 and 15 nm.

A non-limiting example wherein the hydrophilic monomers and charged monomers of the above example are selected from HPMA and methacrylic acid substituted with beta-alanine, respectively, is shown here:

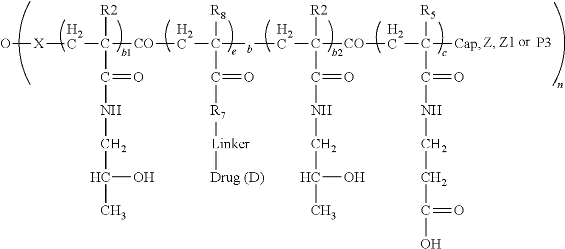

A non-limiting example of star polymers for cancer treatment that comprise di-block polymer arms (A) with both hydrophilic HPMA monomers (B) and reactive monomers (E) linked to drug molecules (D) on one block of the polymer arm (A) that is proximal to the core and both hydrophilic monomers (B) and charged monomers (C) on the other block that is distal to the core, wherein the drug molecule is an imidazoquinoline of Formula II linked to the reactive monomer E through an amide bond is shown here:

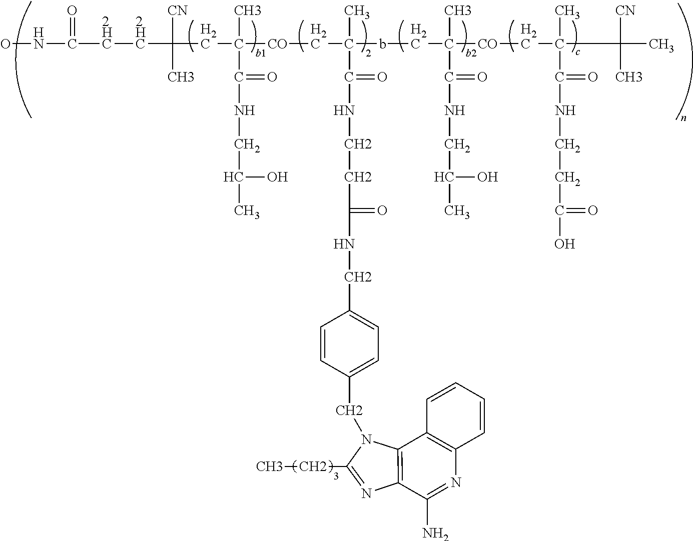

Anti-drug antibodies can have a deleterious impact on the activity of star polymers used for cancer treatment. Therefore, in certain embodiments of star polymers of the present disclosure used for cancer treatment, poly(anionic) polymers and/or those with saccharides that bind CD22 are included to prevent antibody responses generated against the star polymer or arrayed drugs (D) or any ligands (L). Unexpectedly it was found that star polymers for cancer treatment comprising poly(anionic) polymers and/or those with saccharides that bind CD22 were able to be administered repeatedly without induction of antibodies.

Star polymers of the present disclosure for cancer treatment may be actively or passively targeted to tumor tissue. Passive targeting may involve stimuli-responsiveness or the ability of the star polymer to be retained in the tumor due to properties of the microenvironment (e.g., pH, temperature, expression of certain antibodies). Alternatively, star polymers of the present disclosure for cancer treatment can also be actively targeted to tumor tissue through the use of a ligand (L) that binds extracellular receptors in the tumor microenvironment, such as tumor-specific antibodies.

### EXAMPLES

### Example 1—Synthesis of Immunostimulatory Drugs (D) for Attachment to Star Polymers

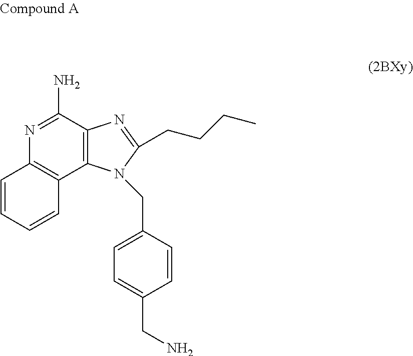 [SMILES: CCCCc1nc2c(N)nc3ccccc3c2n1Cc1ccc(CN)cc1]

Compound A, 1-(4-(aminomethyl)benzyl)-2-butyl-1H-imidazo[4,5-c]quinolin-4-amine, referred to as 2BXy, is a TLR-7/8a agonist that was synthesized as previously described (see: Lynn GM, et al., In vivo characterization of the physicochemical properties of polymer-linked TLR agonists that enhance vaccine immunogenicity. Nat Biotechnol 33(11):1201-1210, 2015, and Shukla NM, et al. Syntheses of fluorescent imidazoquinoline conjugates as probes of Toll-like receptor 7. Bioorg Med Chem Lett 20(22):6384-6386, 2010). Note: the primary amine on the benzyl group provided a reactive handle for attachment to star polymers either directly or through a linker. 1H NMR (400 MHz, DMSO-d6) δ 7.77 (dd, J=8.4, 1.4 Hz, 1H), 7.55 (dd, J=8.4, 1.2 Hz, 1H), 7.35-7.28 (m, 1H), 7.25 (d, J=7.9 Hz, 2H), 7.06-6.98 (m, 1H), 6.94 (d, J=7.9 Hz, 2H), 6.50 (s, 2H), 5.81 (s, 2H), 3.64 (s, 2H), 2.92-2.84 (m, 2H), 2.15 (s, 2H), 1.71 (q, J=7.5 Hz, 2H), 1.36 (q, J=7.4 Hz, 2H), 0.85 (t, J=7.4 Hz, 3H). MS (APCI) calculated for C₂₂H₂₅N₅ m/z 359.2, found 360.3

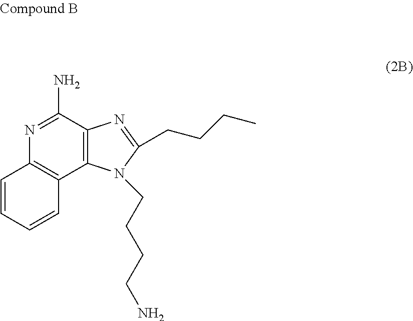 [SMILES: CCCCc1nc2c(N)nc3ccccc3c2n1CCCCN]

Compound B, sometimes referred to as “2B,” 1-(4-aminobutyl)-2-butyl-1H-imidazo[4,5-c]quinolin-4-amine, is a TLR-7/8 agonist that was synthesized as previously described (Lynn GM, et al., Nat Biotechnol 38(3):320-332, 2020). Note: the butyl amine group provided a reactive handle for attachment to star polymers either directly or through a linker. ¹H NMR (400 MHZ, DMSO-d6) δ 8.03 (d, J=8.1 HZ, 1H), 7.59 (d, J=8.1 Hz, 1H), 7.41 (t, J=7.41 Hz, 1H), 7.25 (t, J=7.4 Hz, 1H), 6.47 (s, 2H), 4.49 (t, J=7.4 Hz, 2H), 2.91 (t, J=7.78 Hz, 2H), 2.57 (t, J=6.64 Hz, 1H), 1.80 (m, 4H), 1.46 (sep, J=7.75 Hz, 4H), 0.96 (t, J=7.4 Hz, 3H). MS (ESI) calculated for C₁₈H₂₅N₅, m/z 311.21, found 312.3.

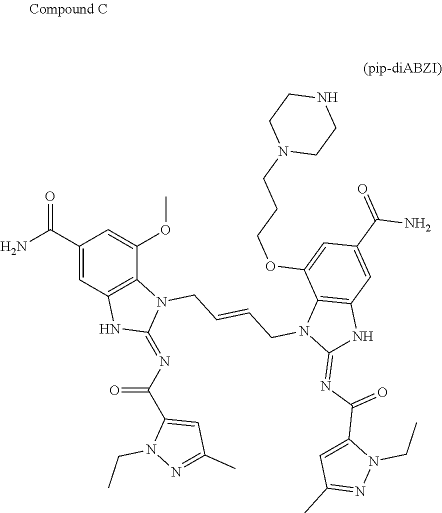 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCNCC3)c21]

Compound C, sometimes referred to as “pip-diABZI,” is a piperarzine modified linked amidobenzimidazole-based STING agonist that was synthesized in a similar manner as was described for a morpholine derivative (“Compound 3” in the reference Ramanjulu JM, et al., Nature 564:439-443, 2018). Note: the piperazine was introduced to provide a reactive-handle for attachment to star polymers either directly or through a linker. Sometimes pip-diABZI is referred to generically as “diABZI.” ¹H NMR (400 MHZ, DMSO-d6) conforms to structure. HPLC purity at 220 nm, 99.8% AUC. MS (ESI) calculated for C₄₂H₅₂N₁₄O₆, m/z 848.42, found 849.5.

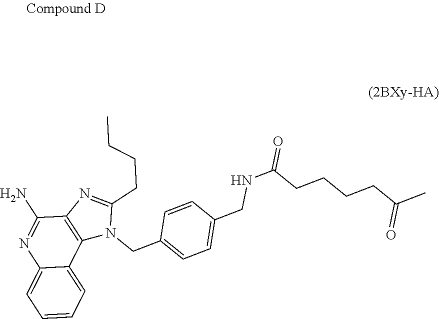 [SMILES: CCCCc1nc2c(N)nc3ccccc3c2n1Cc1ccc(CNC(=O)CCCCC(C)=O)cc1]

Compound D, N-(4-((4-amino-2-butyl-1H-imidazo[4,5-c]quinolin-1-yl)methyl)benzyl)-6-oxoheptanamide, referred to as 2BXy-HA is a TLR-7/8a agonist that was modified with a ketone, 6-oxohepantanoic acid (HA), to enable linkage to star polymers through a pH-sensitive hydrazone bond.

To a solution of 6-oxoheptanoic acid (36 mg, 0.25 mmol) in DCM (5.0 mL) was added EDC (48 mg, 0.25 mmol). Sequentially, 1-(4-(aminomethyl)benzyl)-2-butyl-1H-imidazo[4,5-c]quinolin-4-amine (50 mg, 0.14 mmol), Et₃N (21 mg, 0.15 mmol) and DMAP (3.0 mg, 0.025 mmol) were added and stirred for 16 h at room temperature. The solution was partitioned between DCM (30 mL) and water (15 mL). The organic layer was washed with sat'd NH₄Cl (15 mL), sat'd NaHCO₃ (2×15 mL), dried over Na₂SO₄, filtered and concentrated. Upon drying, the product was isolated as a light yellow/brown foamy solid. HPLC purity at 220 nm, >95.0% AUC. MS (ESI) calculated for C₂₉H₃₅N₅O₂, m/z 485.3, found 486.2.

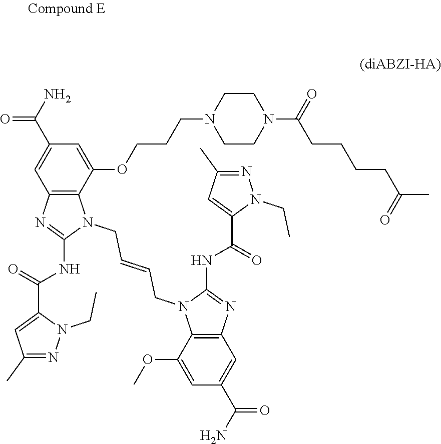 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)CCCCC(C)=O)CC3)c21]

Compound E, (E)-1-(4-(5-carbamoyl-2-(1-ethyl-3-methyl-1H-pyrazole-5-carboxamido)-7-(3-(4-(6-oxoheptanoyl)piperazin-1-yl)propoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide, referred to as pip-diABZI-HA (or sometimes herein as “diABZI”). Note: a ketone, 6-oxohepantanoic acid (HA), was introduced to enable linkage to star polymers through a pH-sensitive hydrazone bond

To 6-oxoheptanoic acid (0.80 mg, 0.056 mmol) in DMF (0.5 mL) was added (E)-1-(4-(5-carbamoyl-2-(1-ethyl-3-methyl-1H-pyrazole-5-carboxamido)-7-(3-(piperazin-1-yl)propoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide (5 mg, 0.0059 mmol). DIEA (3.0 mg, 0.023 mmol) was added followed by HATU (2.0 mg, 0.0056 mmol). The solution was stirred for 2 hours. The DMF was removed, the sample was dried under vacuum, and used in the subsequent step without further purification or characterization. HPLC purity at 220 nm, >95.0% AUC. MS (ESI) calculated for C₄₉H₆₂N₁₄O₈, m/z 974.5, found 488 (m/2).

### Example 2—Synthesis of Monomers, Initiators, CTAs and Amplifying Linkers

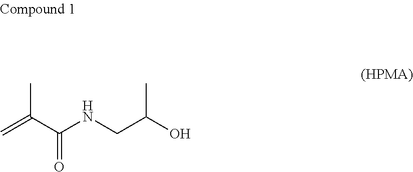

Compound 1. N-(2-Hydroxypropyl)methacrylamide (HPMA) is an example of a hydrophilic monomer (B), specifically meth(acrylamide)-based monomer. HPMA was synthesized by reacting 1-amino-2-propanol with methacryloyl chloride. To a 1 L round-bottom flask equipped with magnetic stir bar, 1-amino-2-propanol (60.0 mL, 0.777 mol), sodium bicarbonate (60.27 g, 0.717 mol), 4-methoxyphenol (1.00 g, 8.1 mmol), and 200 mL of dichloromethane (DCM) were added. The flask was immersed in an acetone-dry ice bath for 15 min with vigorous stirring. Methacryloyl chloride (70.0 mL, 0.723 mol) dissolved in 80 mL of DCM was added dropwise under Ar (g) over 3 h. The reaction was allowed to proceed at r.t. for another 30 min. After removing the salt, crude product was purified via flash chromatography using a silica gel column (Biotage SNAP ultra 100 g) and gradient eluent DCM/MeOH with MeOH increased from 0 to 10% (v/v). The solid thus obtained after solvent removal was then recrystallized from acetone to yield HPMA as white crystal (22.4 g, 21.6%). ESI-MS: m/z=144.1 (M-H)⁺.

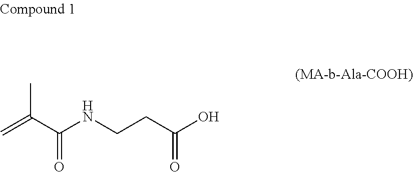

Compound 2. N-methacryloyl-3-aminopropanoic acid (MA-b-Ala-COOH) was synthesized by reacting beta-alanine (15.07 g, 169.1 mmol) to methacrylic anhydride (28.6 g, 185.5 mmol) in the presence of 4-methoxyphenol (0.218 g, 1.76 mmol) in a 100 mL round bottom flask at r.t. over weekend. The mixture was purified by flash chromatography using a silica gel column (Biotage SNAP ultra 100 g) and gradient eluent DCM/MeOH with MeOH increased from 0 to 10% (v/v). After combining fractions and removing solvent, product was recrystallized from EtOAc/Et2O (1/1 v/v) at -20° C., yielding a white crystal (15.22 g, 57.3% yield). ¹H NMR (DMSO-d₆, ppm): δ12.25 (s, 1H), 7.96 (s, 1H), 5.63 (s, 1H), 5.32 (s, 1H), 3.30 (q, 2H), 2.43 (t, 3H), 1.81 (s, 3H).

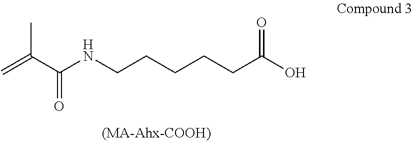

Compound 3. N-methacryloyl-6-aminohexanoic acid (MA-Ahx-COOH) was synthesized by reacting 6-aminohexonic acid (0.252 g, 1.92 mmol) to methacrylic anhydride (0.582 g, 3.78 mmol) in the presence of 4-methoxyphenol (4 mg, 0.03 mmol) in a 20 mL scintillation vial at r.t. overnight. The product was purified by recrystallizing from EtOAc/Et2O (1/1 v/v) at −20° C., yielding a white crystal. ¹H NMR (D₂O, ppm): δ1.32 (—CH₂CH₂CH₂COOH), δ1.52 (—CH₂CH₂COOH), δ1.58 (—NHCH₂CH₂—), δ1.88 (—CH₃), δ2.35 (—CH₂COOH), δ3.22 (—NHCH₂—), δ5.35 and 5.61 (CH₂═CH).

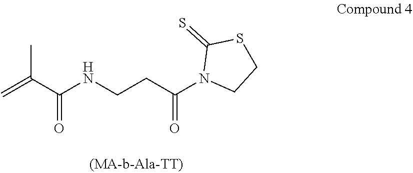 [SMILES: C=C(C)C(=O)NCCC(=O)N1CCSC1=S]

Compound 4. N-Methacryloyl-3-aminopropanoic acid-thiazolidine-2-thione (MA-b-Ala-TT) is an example of a reactive monomer (E). MA-b-Ala-TT was prepared by reacting Compound 2, MA-b-Ala-COOH (5.05 g, 32 mmol), 1,3-thiazolidine-2-thione (4.39 g, 37 mmol), EDC (8.09 g, 42 mmol), DMAP (0.45 g, 4 mmol), and 100 mL DCM were mixed in a 250 mL round bottom flask. It was allowed to react 1 h before the product was washed by 1M HCl (2×) and DI water (1×). Upon solvent removal, yellow solid product was collected (7.15 g, 86.1% yield). ¹H NMR (DMSO-d₆, ppm): δ7.96 (s, 1H), 5.63 (s, 1H), 5.32 (s, 1H), 4.91 (t, 2H), 3.32 (m, 6H), 1.78 (s, 3H). ESI-MS: m/z=281.0 (M-Na)⁺.

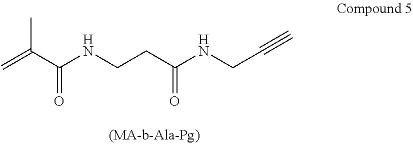

Compound 5. MA-b-Ala-Pg is an example of a reactive monomer (E). MA-b-Ala-Pg was prepared by reacting Compound 4, MA-b-Ala-TT (2.067 g, 8.01 mmol) to propargylamine (0.473 g, 8.588 mmol) in the presence of triethylamine (0.799 g, 7.892 mmol) in a 22 mL DCM for 1.5 h at r.t. The product was purified by recrystallizing from acetone at −20° C. for two times, yielding a white crystal (1.08 g, 69.5% yield). ¹H NMR (DMSO-d₆, ppm): 68.35 (t, 1H), 7.96 (t, 3H), 5.62 (s, 1H), 5.31 (s, 1H), 3.83 (d, 2H), 3.28 (q, 2H), 3.12 (s, 1H), 2.27 (t, 2H), 1.78 (s, 3H).

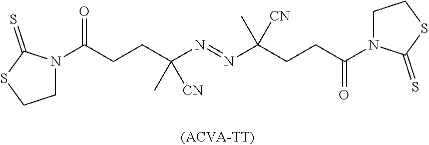

Compound 6. 2-[1-Cyano-1-methyl-4-oxo-4-(2-thioxo-thiazolidin-3-yl)-butylazo]-2-methyl-5-oxo-5-(2-thioxothiazolidin-3-yl)-pentanenitrile, “ACVA-TT,” is a TT-functionalized initiator, which can be used to incorporate TT, activated carbonyl groups, to the ends of the polymer arms (A) during polymerization or capping (i.e. by replacing the CTA of a living polymer). ACVA-TT was synthesized by activating the carboxylic acids in 4,4′-azobis(4-cyanovaleric acid) (ACVA-COOH) with 2-thiazoline-2-thiol via N,N′-diisopropylcarbodiimide (DIC) coupling reaction. To a 20 mL scintillation vial, ACVA-COOH (501.5 mg, 1.79 mmol), 2-thiazoline-2-thiol (411.8 mg, 3.46 mmol), 4-(dimethylamino)pyridine (DMAP, 10.6 mg, 0.087 mmol), and 15 mL of DCM were added. The mixture was stirred vigorously in an ice-bath for 15 min before DIC (497.1 mg, 3.94 mmol) was added. The mixture was allowed to slowly warm up to r.t. and react for another 15 min before it was washed with saturated solution of NaHCO₃ (20 mL×2), DI water (20 mL×1). The organic phase was then dried over MgSO₄ and evaporated to yield dry product, which was purified by recrystallizing from DCM/Et₂O at −20° C. After decanting the solvent, bright yellow powder was obtained (658.3 mg, 76.2%). ESI-MS: m/z=483.1 (M-H)⁺.

Compound 7

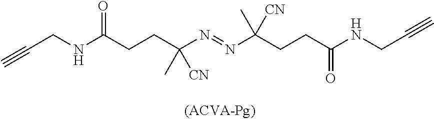 [SMILES: C#CCNC(=O)CCC(C)(C#N)/N=N/C(C)(C#N)CCC(=O)NCC#C]

Compound 7. 4—Cyano-4-(1-cyano-3-ethynylcarbamoyl-1-methylpropylazo)-N-ethynyl-4-methylbutyramide, “ACVA-Pg,” is a propargyl functionalized initiator, which can be used to incorporate Pg groups to the ends of polymer arms (A) during polymerization or capping (i.e. by replacing the CTA of a living polymer). ACVA-Pg was synthesized by reacting ACVA-TT with 3-amino-1-propyne. To a 20 mL scintillation vial, ACVA-TT (329.7 mg, 0.684 mmol), 3-amino-1-propyne (99.76 mg, 1.81 mmol), and 10 mL of DCM were added. Triethylamine (253 μL, 1.82 mmol) was then added to the mixture. The reaction was allowed to proceed for another 1 h at r.t. before solvent was removed. The crude product was purified via flash chromatography using a C-18 column (Biotage SNAP Ultra C-18) and a gradient of 0-95% acetonitrile in H₂O (0.05% TFA) over 20 CVs (product eluted at 30-40% acetonitrile). Fractions containing pure product were pool and dried to yield white solid (190.3 mg, 78.5%). ESI-MS: m/z=355.2 (M-H)⁺.

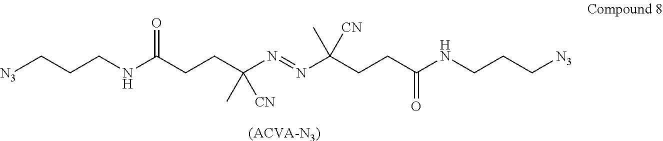 [SMILES: CC(C#N)(CCC(=O)NCCCN=[N+]=[N-])/N=N/C(C)(C#N)CCC(=O)NCCCN=[N+]=[N-] ; "(ACVA-N3)"]

Compound 8. ACVA-N₃ is an Azide-functionalized initiator, which can be used to incorporate Azide groups to the ends of polymer arms (A) during polymerization or capping (i.e. by replacing the CTA of a living polymer). ACVA-N₃ was synthesized by reacting ACVA with 1-azido-3-propanamine. To a 20 mL scintillation vial, ACVA (250.0 mg, 0.893 mmol), 1-azido-3-propanamine (187.7 mg, 1.87 mmol), and 5 mL of DCM were added. EDC (375.2, 1.96 mmol) was then added to the mixture over 20 min. The reaction was allowed to proceed for another 1 h at r.t. before solvent was removed. The crude product was recrystallized from EtOAc/Et₂O to yield white solid (130.0 mg, 32.8%). ESI-MS: m/z=445.2 (M-H)⁺.

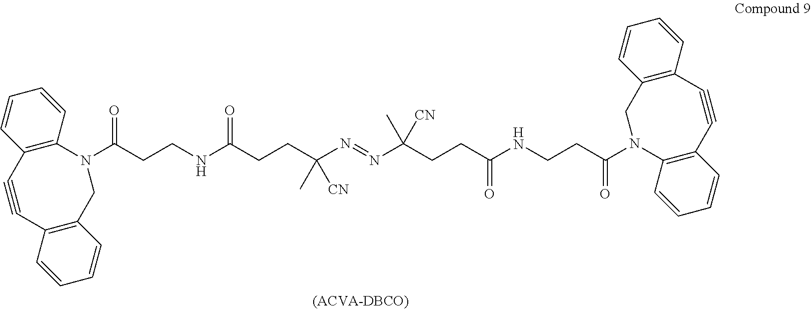 [SMILES: CC(C#N)(CCC(=O)NCCC(=O)N1Cc2ccccc2C#Cc2ccccc21)/N=N/C(C)(C#N)CCC(=O)NCCC(=O)N1Cc2ccccc2C#Cc2ccccc21 ; "(ACVA-DBCO)"]

Compound 9. ACVA-DBCO is a DBCO functionalized initiator, which is an example of a strained-alkyne functionalized initiator that can be used to incorporate strained-alkynes to the ends of polymer arms (A) during polymerization or capping (i.e. by replacing the CTA of a living polymer). ACVA-DBCO was synthesized by reacting ACVA-TT with DBCO-amine. To a 20 mL scintillation vial, ACVA-TT (201.4 mg, 0.417 mmol), DBCO-amine (229.2 mg, 0.829 mmol), and 1 mL of DCM were added. The reaction was allowed to proceed for 1 h at r.t. before solvent was removed. The crude product was purified by flash chromatography using a silica gel column and a gradient of 0-5% MeOH in DCM to yield white solid (314.4 mg, 95.1%). ESI-MS: m/z=797.3 (M-H)⁺.

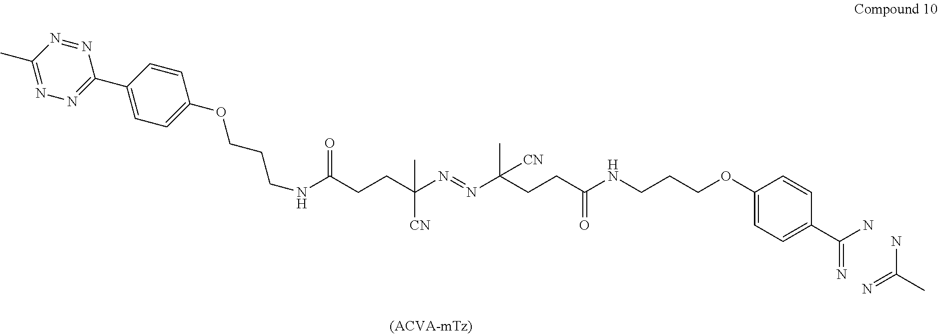

Compound 10. ACVA-mTz is a methyletrazinme functionalized initiator, which is an example of a tetrazine functionalized initiator that can be used to incorporate tetrazines to the ends of polymer arms (A) during polymerization or capping (i.e. by replacing the CTA of a living polymer). ACVA-mTz was synthesized by reacting ACVA-TT with methyltetrazine propylamine (mTz-amine) using triethylamine as the catalyst. To a 20 mL scintillation vial, ACVA-TT (162.2 mg, 0.427 mmol), mTz-amine (120.8 mg, 0.492 mmol), trimethylamine (124.9 μL, 0.896 mmol), and 4 mL of DCM were added. The reaction was allowed to proceed for 1 h at r.t. before solvent was removed. The crude product was purified by flash chromatography using a C-18 column to yield white solid (166.8 mg, 53.2%). ESI-MS: m/z=735.3 (M-H)⁺.

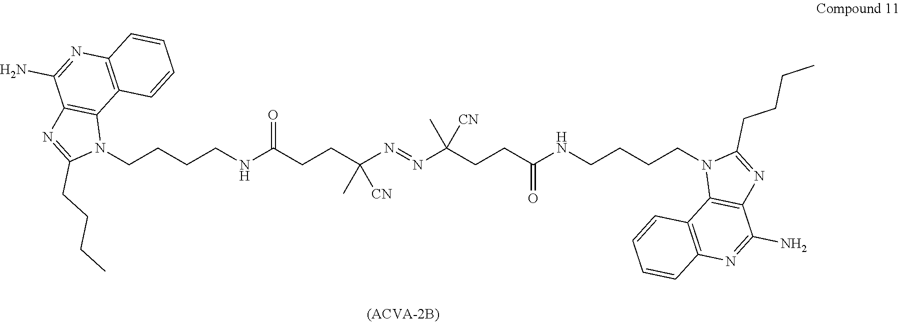 [SMILES: CCCCc1nc2c(N)nc3ccccc3c2n1CCCCNC(=O)CCC(C)(C#N)/N=N/C(C)(C#N)CCC(=O)NCCCCn1c(CCCC)nc2c(N)nc3ccccc3c21]

Compound 11. ACVA-2B is a 2B functionalized initiator, which is an example of a TLR-7/8a (and more broadly drug, (D)) functionalized initiator that can be used to incorporate TLR-7/8a to the ends of polymer arms (A) during polymerization or capping (i.e. by replacing the CTA of a living polymer). ACVA-2B was synthesized by reacting ACVA-TT with 2B. To a 20 mL scintillation vial, ACVA-TT (200.5 mg, 0.415 mmol), 2B, Compound B, (258.7 mg, 0.831 mmol), and 1 mL of DCM were added. The reaction was allowed to proceed for 1 h at r.t. before solvent was removed. The crude product was purified on a preparatory HPLC system using a gradient of 27-47% acetonitrile/H₂O (0.05% TFA) over 12 minutes on an Agilent Prep C-18 column, 50×100 mm, 5 μm. The product fractions were pooled and lyophilized yielding white solid (214.7 mg, 59.5%). ESI-MS: m/z=868.2 (M-H)⁺.

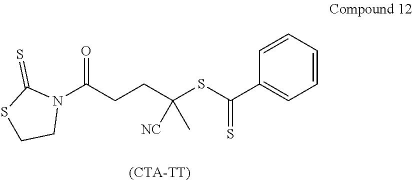

Compound 12. Dithiobenzoic acid 1-cyano-1-methyl-4-oxo-4-(2-thioxothiazolidin-3-yl)butyl ester, “CTA-TT,” is a TT-functionalized chain transfer agent (CTA), which can be used to introduce TT functional groups onto polymer arms (A) during polymerization. CTA-TT was synthesized by activating the carboxylic acid in 4-cyano-4-(phenylcarbonothioylthio)pentanoic acid (CTA-COOH) with 2-thiazoline-2-thiol. To a 20 mL scintillation vial, CTA-COOH (499.8 mg, 1.79 mmol), 2-thiazoline-2-thiol (196.5 mg, 1.65 mmol), DMAP (8 mg, 0.065 mmol), and 10 mL of DCM were added. The mixture was stirred vigorously in an ice-bath for 15 min before EDC (446.2 mg, 2.33 mmol) was added. The mixture was allowed to slowly warm up to r.t. and react for another 15 min before it was washed with saturated solution of NaHCO₃ (10 mL×2) and DI water (10 mL×2). The organic phase was then dried over MgSO₄ and evaporated to yield dry product, which was purified on a preparatory HPLC system using a gradient of 58-78% acetonitrile/H₂O (0.05% TFA) over 12 minutes on an Agilent Prep C-18 column, 30×100 mm, 5 μm. The product eluted at 6.5 minutes and the product fractions were pooled and lyophilized yielding red viscous liquid (400.0 mg, 63.8%). ESI-MS: m/z=381.0 (M-H)⁺.

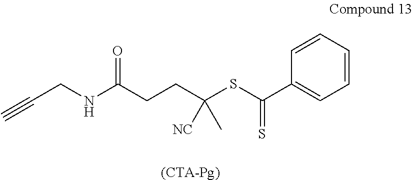 [SMILES: C#CCNC(=O)CCC(C)(C#N)SC(=S)c1ccccc1 ; "(CTA-Pg)"]

Compound 13. Dithiobenzoic acid 1-cyano-1-methyl-3-prop-2-ynylcarbamoylpropyl ester “CTA-Pg,” is a Pg-functionalized CTA, which can be used to introduce Pg functional groups onto polymer arms (A) during polymerization. CTA-Pg was synthesized by reacting CTA-COOH with 3-amino-1-propyne. To a 20 mL scintillation vial, CTA-COOH (100.0 mg, 0.358 mmol), 3-amino-1-propyne (21.69 mg, 0.394 mmol), HATU (272.2 mg, 0.716 mmol), DIEA (185.0 mg, 1.432 mmol), and 4 mL of DMF were added. The mixture was stirred at r.t. for 2 h before it was washed with saturated solution of NaHCO₃ (10 mL×2) and brine (10 mL×1). The organic phase was then dried over MgSO₄ and evaporated to yield dry product, which was purified on a preparatory HPLC system using a gradient of 40-70% acetonitrile/H₂O (0.05% TFA) over 12 minutes on an Agilent Prep C-18 column, 50×100 mm, 5 μm. The product eluted at 8.5 minutes and the product fractions were pooled and lyophilized yielding red viscous liquid (54.0 mg, 47.7%). ESI-MS: m/z=317.1 (M-H)⁺.

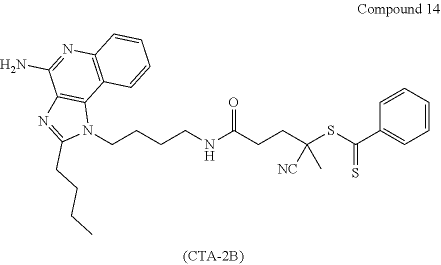 [SMILES: CCCCc1nc2c(N)nc3ccccc3c2n1CCCCNC(=O)CCC(C)(C#N)SC(=S)c1ccccc1]

Compound 14. CTA-2B, is a 2B-functionalized CTA, which is an example of a TLR-7/8a or more broadly (drug) functionalized CTA that can be used to introduce TLR-7/8a functional groups onto polymer arms (A) during polymerization. CTA-2B was synthesized by reacting CTA-NHS with 2B, Compound B. To a 20 mL scintillation vial, CTA-NHS (200.6 mg, 0.533 mmol), 2B (165.6 mg, 0.532 mmol), and 3 mL of DCM were added. The reaction was allowed to proceed for 40 min at r.t. before it was washed with DI water (10 mL×2). The organic phase was then dried over MgSO₄ and evaporated to yield dry product as dark red solid (250 mg, 82.1%). ESI-MS: m/z=573.7 (M-H)⁺.

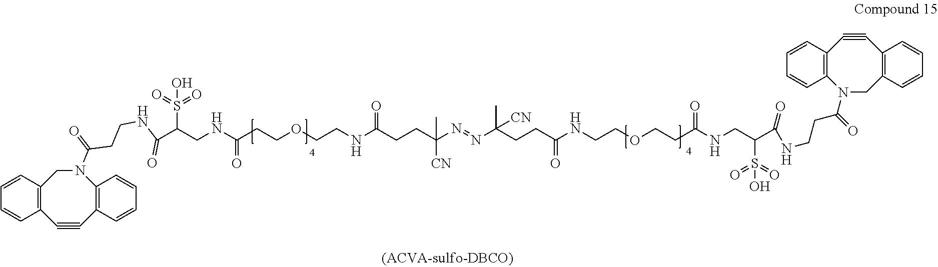

Compound 15. ACVA-sulfo-DBCO, is an example of a water-soluble strained-alkyne functionalized initiator, which can be used to introduce water-soluble strained alkynes onto the ends of polymer arms (A) during polymerization or capping. ACVA-sulfo-DBCO was synthesized by reacting ACVA-TT with sulfo-DBCO-PEG4-amine. ACVA-TT (32.2 mg, 0.067 mmol) and sulfo-DBCO-PEG4-amine (100.0 mg, 0.148 mmol) were dissolved in 2 mL of DCM before triethylamine (30.0 mg, 0.30 mol) was added. The reaction was allowed to proceed for 1 h at r.t. The crude product was purified by flash chromatography using a silica gel column (Biotage SNAP ultra 25 g), and a gradient of 5-20% MeOH in DCM over 20 CVs (product eluted at 18% MeOH). Fractions containing pure product were combined and dried to yield final product (115.2 mg, 84.1%). ESI-MS: m/z=797.4 [(M-2H)]²⁺.

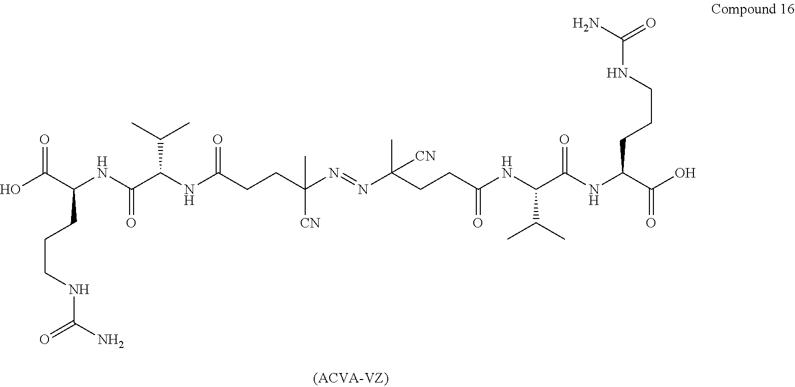 [SMILES: CC(C)[C@H](NC(=O)CCC(C)(C#N)/N=N/C(C)(C#N)CCC(=O)N[C@H](C(=O)N[C@@H](CCCNC(N)=O)C(=O)O)C(C)C)C(=O)N[C@@H](CCCNC(N)=O)C(=O)O]

Compound 16. ACVA-VZ is an example of a degradable peptide-functionalized initiator, which can be used to introduce degradable peptides onto the ends of polymer arms (A) during polymerization or capping. ACVA-VZ was synthesized by reacting ACVA-TT with valine-citrulline (VZ) peptide. ACVA-TT (62.3 mg, 0.13 mmol) and VZ (100.0 mg, 0.36 mmol) were dissolved in 1 mL of DMSO before triethylamine (44.2 mg, 0.44 mmol) was added. The reaction was allowed to proceed for 2 h at r.t. The crude product was purified on a preparatory HPLC system using a gradient of 16-31% acetonitrile/H₂O (0.05% TFA) over 12 minutes on an Agilent Prep C-18 column, 50×100 mm, 5 μm. The product fractions were pooled and lyophilized to yield final product (91.5 mg, 89.1%).

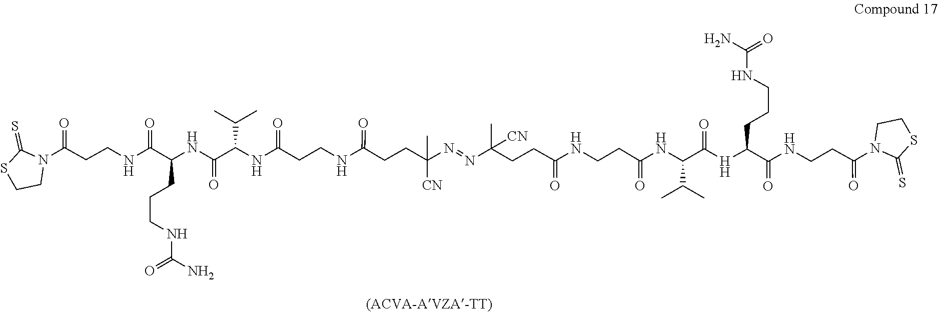

Compound 17. ACVA-A‘VZA’-TT is an example of a TT-activated degradable peptide-functionalized initiator, which can be used to introduce TT-activated degradable peptides onto the ends of polymer arms (A) during polymerization or capping. ACVA-A‘VZA’-TT was synthesized by reacting ACVA-TT with β-alanine-valine-citrulline-β-alanine (A‘VZA’) peptide to afford ACVA-A‘VZA’, followed by activating the carboxylic acids with 2-thiazoline-2-thiol. ACVA-TT (26.0 mg, 0.054 mmol) and A‘VZA’ (50.0 mg, 0.12 mmol) were dissolved in 1.5 mL of DMSO before triethylamine (48.6 mg, 0.48 mmol) was added. The reaction was allowed to proceed for 2 h at r.t. The crude product was purified on a preparatory HPLC system using a gradient of 5-40% acetonitrile/H₂O (0.05% TFA) over 12 minutes on an Agilent Prep C-18 column, 30×100 mm, 5 μm. Fractions containing targeted product were pooled and lyophilized to yield ACVA-A‘VZA’ (53.0 mg, 91.1%). ACVA-A‘VZA’ (10.0 mg, 0.0093 mmol) and 2-thiazoline-2-thiol (2.8 mg, 0.02 mmol) were dissolved in DMF before 1-[Bis(dimethylamino)methylene]-1H-1,2,3-triazolo[4,5-b]pyridinium 3-oxide hexafluorophosphate (HATU) (7.1 mg, 0.019 mmol) and triethylamine (3.8 mg, 0.037 mmol) were added. The reaction was allowed to proceed for 2 h at r.t. before the crude product was purified on a preparatory HPLC system to yield final product ACVA-A‘VZA’-TT.

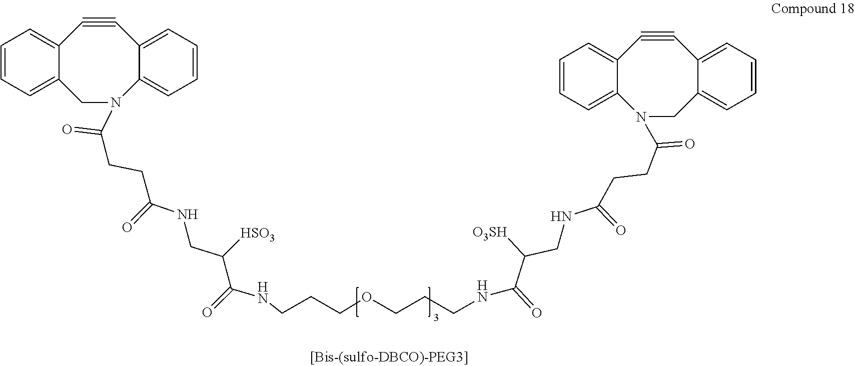

Compound 18. Bis(sulfo-DBCO)-PEG3 is a homo-bifunctional linker that was synthesized by reacting NH2-PEG3-NH2 with sulfo-DBCO-tetrafluorophenyl (TFP) ester. NH2-PEG3-NH2 (8.3 mg, 0.037 mmol) and sulfo-DBCO-TFP ester (50.0 mg, 0.083 mmol) were dissolved in 1 mL of DCM before triethylamine (16.0 mg, 0.16 mmol) was added. The reaction was allowed to proceed for 1 h at r.t. The crude product was purified by flash chromatography using a silica gel column and a gradient of 10-20% MeOH in DCM (product eluted at 10% MeOH). Fractions containing pure product were combined and dried to yield final product (45.2 mg, 109.6%). ESI-MS: m/z=1097 (M-H)⁺.

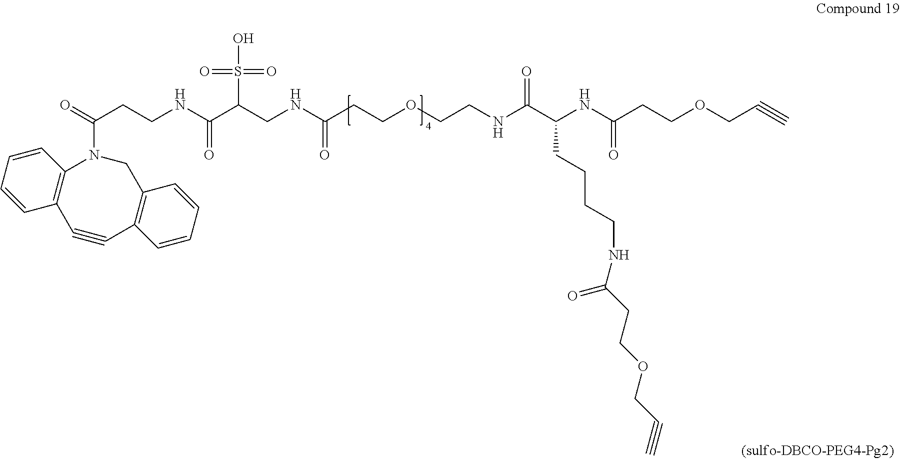

Compound 19. Amplifying linker sulfo-DBCO-PEG4-Pg2 was synthesized in three steps using propargyl NHS ester, amino-PEG4-sulfo-DBCO, and Boc-Lys(Boc)—OH as the starting materials. Boc-Lys(Boc)—OH (1.0 g, 2.89 mmol, 1 eq), TT (378.5 mg, 3.18 mmol, 1.1 eq) and EDC (719.4 mg, 3.75 mmol, 1.3 eq) were dissolved in 10 mL of DCM. DMAP (35.3 mg, 0.29 mmol, 0.1 eq) as a 100 mg/mL stock solution in DCM was added. The solution turned bright yellow and was allowed to react at room temperature for 1 h. DCM was removed under vacuum before the crude product was dissolved in 700 uL of DMSO and precipitated in 50 mL of 0.1M HCl (twice) and DI water. The intermediate, Boc-Lys(Boc)-TT was provided as a yellow solid.

Boc-Lys(Boc)-TT (238.1 mg, 0.53 mmol, 2.41 eq) and sulfo-DBCO-PEG4-NH2 (150.5 mg, 0.22 mmol, 1 eq) were dissolved in DMSO following the addition of TEA (74.2 uL, 0.53 mmol, 2.41 eq). The reaction was stirred at room temperature for 1 hr. The product was purified by flash reverse phase chromatography using a gradient of 0-95% acetonitrile/H₂O (0.05% TFA) over 20 CVs. Pure fractions were combined, frozen at −80C and lyophilized to afford the intermediate Boc-Lys(Boc)-PEG4-sulfo-DBCO as an off white solid. Boc-Lys(Boc)-PEG4-sulfo-DBCO (77.9 mg, 0.08 mmol, 1 eq) was dissolved in 700 uL of DCM. Then, 5 uL of DI water, 5 uL of triisopropylsilane (TIPS), and 300 uL of TFA was added to the reaction flask. The Boc deprotection reaction was allowed to proceed for 30 minutes at room temperature. DCM and TFA were removed by blowing air over the reaction mixture before the intermediate, NH2-Lys(NH2)-PEG4-sulfo-DBCO was dried under high vacuum to yield a dark oil.

NH2-Lys(NH2)-PEG4-sulfo-DBCO (37 mg, 0.046 mmol, 1 eq) was dissolved in 1 mL of DMSO before TEA (19.3 uL, 0.14 mmol, 3 eq) was added. After stirring for 5 minutes at room temperature, propargyl NHS ester (22.8 mg, 0.1 mmol, 2.2 eq) was added to the reaction flask. After 1 hr the reaction was complete and confirmed by LC-MS. The product, sulfo-DBCO-PEG4-Pg2 was used without further purification. ESI-MS: m/z=1023.4 (M-H)Y.

### Example 3—Synthesis of Polymer Arms (A)

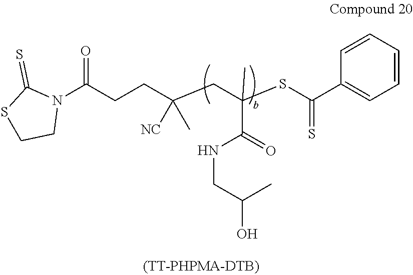

Compound 20 is a polymer arm (A) example of a homopolymer comprised of hydrophilic monomers (B). TT-functionalized poly[N-(2-hydroxypropyl)methacrylamide] (TT-PHPMA-DTB) was synthesized via the RAFT polymerization of HPMA using CTA-TT as a chain transfer agent and ACVA-TT as an initiator in tert-butanol (tBuOH) at 70° C. for 16 h. The initial monomer concentration [HPMA]₀=1 mol/L, the molar ratio [CTA-TT]₀:[ACVA-TT]₀=1:0.5, and [HPMA]₀:[CTA-TT]₀ varied to obtain polymers with different chain lengths. The following procedure was employed for a typical polymerization to produce TT-PHPMA-DTB targeting a molecular weight of 10 kDa: HPMA (572.0 mg, 4.00 mmol) was dissolved in 4 mL of tBuOH. CTA-TT (15.2 mg, 0.040 mmol) and ACVA-TT (9.65 mg, 0.020 mmol) were dissolved in anhydrous DMSO before mixing with the monomer solution. The mixture was transferred to a 5 mL ampule, which was sealed with a rubber septum and sparged with Ar (g) at r.t. for 30 min. The flask was then immersed in a water circulator preheated to 70° C. and polymerize for 16 h. The polymer was purified by precipitating against acetone for 3 times. After drying in vacuum oven overnight, light pink powder was obtained (277.3 mg, 40.1% yield). Number-average (Mₙ) and weight-average molecular weight (M_{w}) were 10.05 kDa and 10.30 kDa, respectively, and polydispersity (PDI) was 1.02 measured by GPC-MALS. The chain end functionalities measured by UV-Vis spectroscopy [ε₃₀₅ (TT)=10300 L/(mol·cm), ε₃₀₅ (DTB)=12600 L/(mol·cm)] showed that (TT+DTB) %=95.3%.

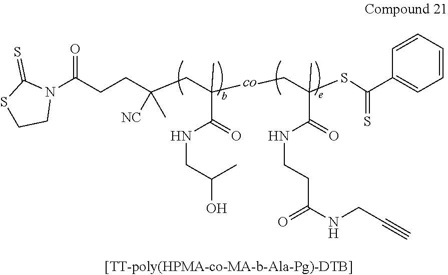

Compound 21 is a polymer arm (A) example of a co-polymer with hydrophilic monomers and reactive monomers (E) with alkyne groups. TT-poly(HPMA-co-MA-b-Ala-Pg)-DTB random copolymer was synthesized via the RAFT polymerization of HPMA and MA-b-Ala-Pg using CTA-TT as a chain transfer agent and ACVA-TT as an initiator in tert-butanol (tBuOH)/N,N-dimethylacetamide (DMAc) at 70° C. for 16 h. The initial monomer concentration [ ΣM]₀=[HPMA+MA-b-Ala-Pg]₀=1 mol/L and the molar ratio [CTA-TT]₀:[ACVA-TT]₀=1:0.5. [ ΣM]₀:[CTA-TT]₀ is varied to target polymers with different chain lengths, while the molar percentage of reactive site-containing comonomer MA-b-Ala-Pg controls the maximum number of cargo molecules (e.g., small molecule drugs, peptides) each polymer chain carries. The following procedure was employed for a typical polymerization to produce TT-poly(HPMA-co-MA-b-Ala-Pg)-DTB targeting 5 mol % of comonomer MA-b-Ala-Pg and a molecular weight of 40 kDa: HPMA (340.7 mg, 2.375 mmol) and MA-b-Ala-Pg (24.1 mg, 0.125 mmol) were dissolved in 2.13 mL of tBuOH. CTA-TT (3.2 mg, 0.008 mmol) as a 100 mg/mL stock solution in anhydrous DMAc and ACVA-TT (2.0 mg, 0.004 mmol) as a 50 mg/mL stock solution in anhydrous DMAc were then added to the monomer solution. The mixture was transferred to a 5 mL ampule, which was sealed with a rubber septum and sparged with Ar (g) at r.t. for 30 min. The flask was then immersed in a water circulator preheated to 70° C. and polymerize for 16 h. The resulted polymer was purified by precipitating against acetone for 3 times. After drying in vacuum oven overnight, light pink powder was obtained (208.9 mg, 57.7% yield). Number-average (Mₙ) and weight-average molecular weight (M_{w}) were 39.27 kDa and 42.85 kDa, respectively, and polydispersity (PDI) was 1.09 measured by GPC-MALS. The chain end functionalities measured by UV-Vis spectroscopy [ε₃₀₅ (TT)=10300 L/(mol·cm), ε₃₀₅ (DTB)=12600 L/(mol·cm)] showed that (TT+DTB) %=121.8%.

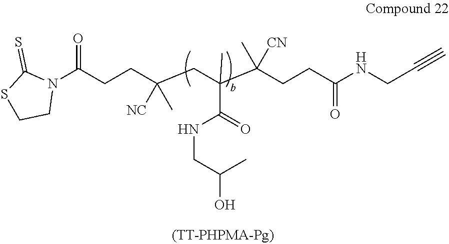

Compound 22 is a polymer arm (A) example of a homopolymer comprised of hydrophilic monomers (B) with two different end group functionalities (heterotelechelic). The propargyl functionality was introduced by reacting TT-PHPMA-DTB with 10-20 molar excess of ACVA-Pg.

Example of reaction: Dry polymer TT-PHPMA-DTB (198 mg, 19.7 μmol) and ACVA-Pg (70.3 mg, 198.9 umol) was dissolved in 3.0 mL of anhydrous DMSO. The solution was transferred to a 5 mL ampule, which was sealed with a rubber septum and sparged with Ar (g) at r.t. for 30 min. The flask was then immersed in a water circulator preheated to 70° C. and react for 3 h. The polymer was purified by precipitating against acetone for 3 times. After drying in vacuum oven overnight, off-white powder was obtained. Mn and M_{w} were 10.80 kDa and 12.10 kDa, respectively, and PDI was 1.12 measured by GPC-MALS. The chain end functionalities measured by UV-Vis spectroscopy [ε₃₀₅ (TT)=10300 L/(mol·cm)] showed that (TT) %=100%. Note: in this example, the TT group was added to the polymer during the polymerization step and the Pg functionality was added to the other end during capping.

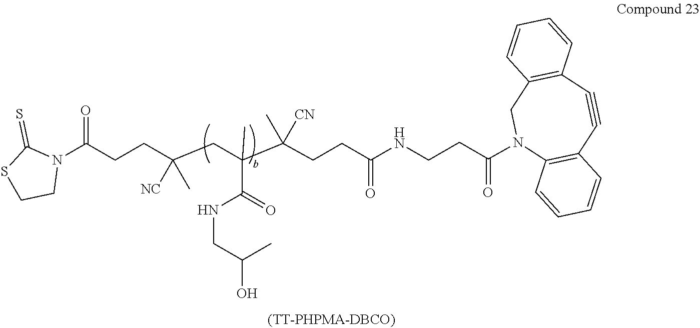

Compound 23 is a polymer arm (A) example of a homopolymer comprised of hydrophilic monomers (B) with two different end group functionalities (heterotelechelic). TT-PHPMA-DBCO was synthesized using the same method as described for as Compound 22, except that ACVA-Pg was replaced by ACVA-DBCO. Note: in this example, the TT group was added to the polymer during the polymerization step and the strained-alkyne functionality was added to the other end during capping.

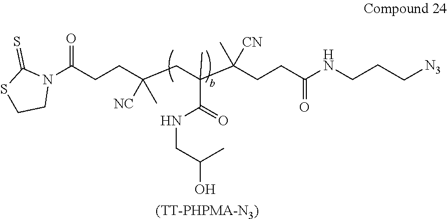

Compound 24 is a polymer arm (A) example of a homopolymer comprised of hydrophilic monomers (B) with two different end group functionalities (heterotelechelic). TT-PHPMA-N₃ was synthesized using the same method as described for as Compound 22, except that ACVA-Pg was replaced by ACVA-N₃. Note: in this example, the TT group was added to the polymer during the polymerization step and the N₃ functionality was added to the other end during capping.

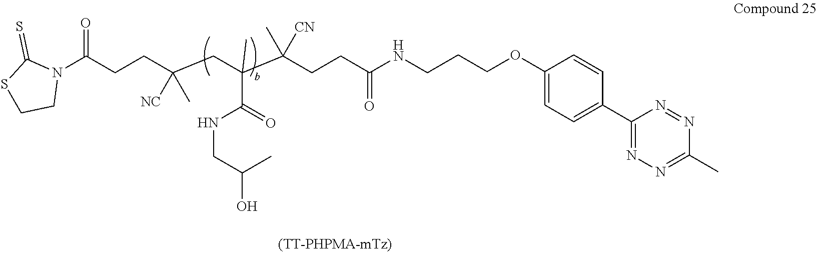

Compound 25 is a polymer arm (A) example of a homopolymer comprised of hydrophilic monomers (B) with two different end group functionalities (heterotelechelic). TT-PHPMA-mTz was synthesized using the same method as described for as Compound 22, except that ACVA-Pg was replaced by ACVA-mTz. Note: in this example, the TT group was added to the polymer during the polymerization step and the methyltetrazine functionality was added to the other end during capping.

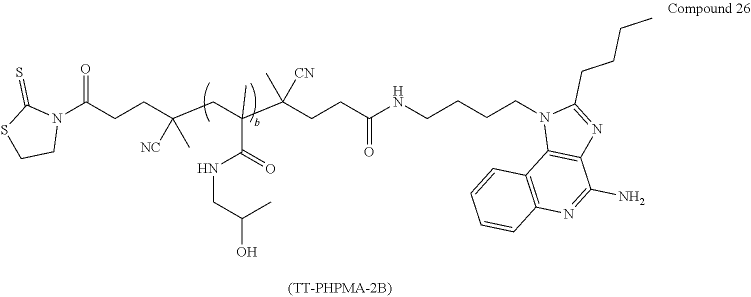

Compound 26 is a polymer arm (A) example of a homopolymer comprised of hydrophilic monomers (B) with two different end group functionalities (heterotelechelic). TT-PHPMA-2B was synthesized using the same method as described for as Compound 22, except that ACVA-Pg was replaced by ACVA-2B. Note: in this example, the TT group was added to the polymer during the polymerization step and the 2B functionality was added to the other end during capping.

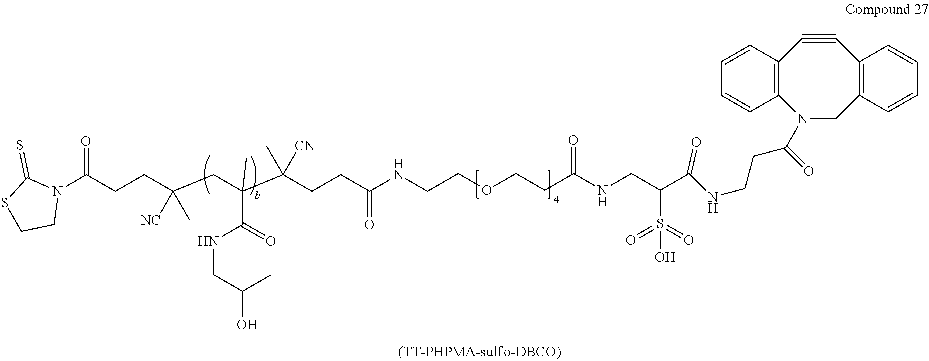

Compound 27 is a polymer arm (A) example of a homopolymer comprised of hydrophilic monomers (B) with two different end group functionalities (heterotelechelic). TT-PHPMA-sulfo-DBCO was synthesized in the same manner as Compound 22, TT-PHPMA-Pg except that ACVA-Pg was replaced with ACVA-sulfo-DBCO. Note: in this example, the TT group was added to the polymer during the polymerization step and the water-soluble strained-alkyne functionality was added to the other end during capping.

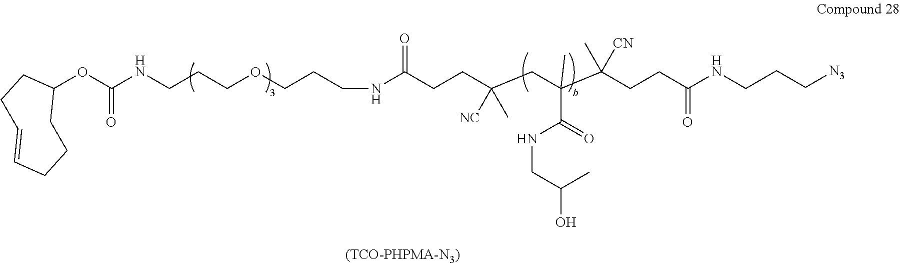

Compound 28. is a polymer arm (A) example of a homopolymer comprised of hydrophilic monomers (B) with two different end group functionalities (heterotelechelic). TCO-PHPMA-N₃ was synthesized by reacting the carbonylthiazolidine-2-thione (TT) of Compound 24, TT-PHPMA-N₃, with 5-7 molar excess of TCO-PEG3-amine using triethylamine as the catalyst. The following procedure was employed for a typical synthesis procedure for TCO-PHPMA-N₃ from TT-PHPMA-N₃: TT-PHPMA₄₀k-N₃ (62.1 mg, 1.6 μmol) and TCO-PEG3-amine (3.5 mg, 9.6 μmol) were dissolved in 800 μL of anhydrous DMSO. Triethylamine (1.3 mg, 12.7 μmol) was then added to the mixture and the reaction was allowed to proceed for 5 h at r.t. The product was purified by precipitating against acetone (6-8× volume) for three times. After drying in vacuum oven overnight, off-white solid was obtained (57.9 mg, 92.4%).

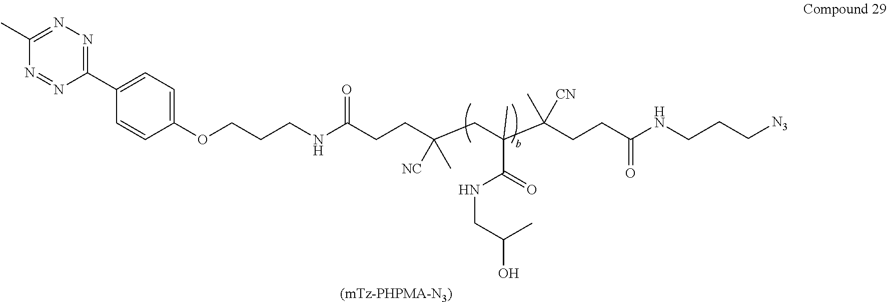

Compound 29 is a polymer arm (A) example of a homopolymer comprised of hydrophilic monomers (B) with two different end group functionalities (heterotelechelic). mTz-PHPMA-N₃ was synthesized by reacting the carbonylthiazolidine-2-thione (TT) of Compound 24 with 5-7 molar excess of mTz-amine. The following procedure was employed for a typical synthesis procedure for mTz-PHPMA-N₃ from TT-PHPMA-N₃: to a 1.5 mL centrifuge tube, TT-PHPMA₄₀ₖ-N₃ (80 mg, 2.05 μmol) and 400 μL of anhydrous DMSO were added. The polymer was fully dissolved before mTz-amine (58.0 L, 10.3 μmol) as a 50 mg/mL stock solution in DMSO was added. The mixture was allowed to proceed overnight at r.t. Then the polymer was purified by precipitating against acetone for 3 times. After drying in vacuum oven overnight, pink powder was obtained (60.6 mg, 75.8% yield). Mₙ and M_{w} were 37.9 kDa and 41.2 kDa, respectively, and PDI was 1.09 measured by GPC-MALS. The chain end functionalities measured by UV-Vis spectroscopy [ε₂₆₈ (mTz)=14629 L/(mol·cm) showed that (mTz) %=96.3%.

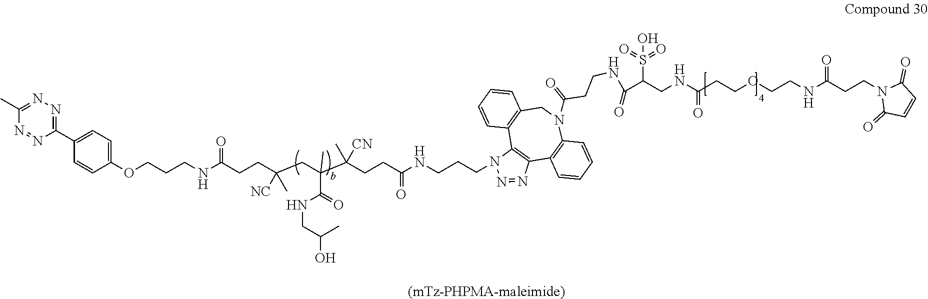

Compound 30 is a polymer arm (A) example of a homopolymer comprised of hydrophilic monomers (B) with two different end group functionalities (heterotelechelic). mTz-PHPMA-maleimide was synthesized by reacting the azide group (N₃) of Compound 29, mTz-PHPMA-N₃, with 10 molar excess of sulfo-DBCO-PEG4-maleimide. The following procedure was employed for a typical synthesis procedure for mTz-PHPMA-MI from mTz-PHPMA-N₃: mTz-PHPMA₅₆ₖ-N₃ (11.9 mg, 0.21 mol) was dissolved in 50 μL of anhydrous DMSO before sulfo-DBCO-PEG4-maleimide (1.8 mg, 100 mg/mL in anhydrous DMSO, 2.1 μmol) was added. The reaction was allowed to proceed for 16 h at r.t. before the product was purified by precipitating against acetone (6-8× volume) for three times. After drying in vacuum oven overnight, light pink solid was obtained (9.2 mg, 76.2%).

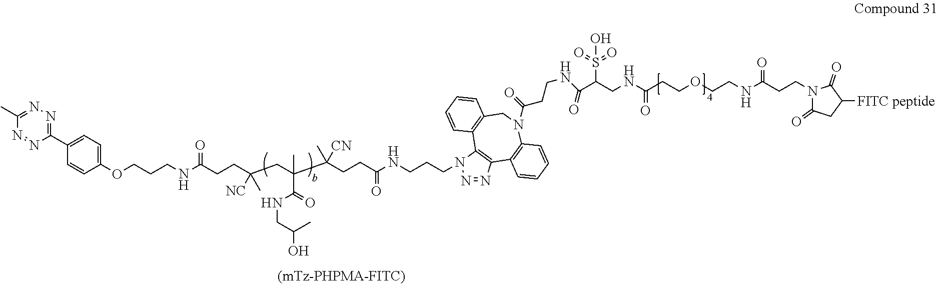

Compound 31 is a polymer arm (A) example of a homopolymer comprised of hydrophilic monomers (B) with two different end group functionalities (heterotelechelic). mTz-PHPMA-FITC peptide was synthesized by conjugating a peptide containing a FITC dye (FITC-Ahx-GSGSGSCG) to Compound 30, mTz-PHPMA-maleimide through maleimide-thiol coupling chemistry. The following procedure was employed for a typical synthesis: mTz-PHPMA₅₆ₖ-maleimide (2.0 mg, 0.036 μmol) was dissolved in 10 μL of anhydrous DMSO before FITC-peptide (2.0 mg, 20 mg/mL in anhydrous DMSO, 0.047 μmol) was added. The reaction was allowed to proceed for 16 h at r.t. before characterized using gel permeation chromatography (GPC). The resulted conjugate showed targeted UV absorbance at 488 nm (FITC absorbance wavelength) where the original polymer has no absorbance.

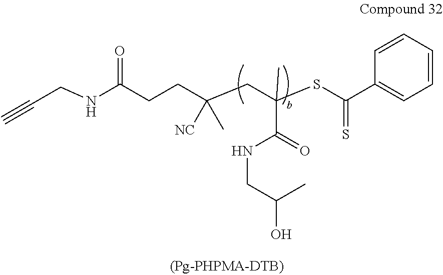

Compound 32 is a polymer arm (A) example of a homopolymer comprised of hydrophilic monomers (B) with two different end group functionalities (heterotelechelic). Note: the dithiobenzoate (DTB) present on the polymer indicates that the polymer is living and can add on additional monomers or can be capped. Pg-PHPMA-DTB was synthesized using the same method as described for as Compound 20, except that ACVA-TT and CTA-TT were replaced by ACVA-Pg and CTA-Pg.

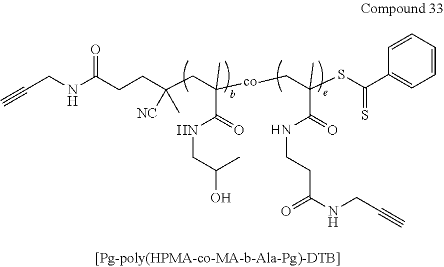

Compound 33 is a polymer arm (A) example of a copolymer comprised of hydrophilic monomers (B) and reactive monomers (E) with two different end group functionalities (heterotelechelic). Note: the dithiobenzoate (DTB) present on the polymer indicates that the polymer is living and can add on additional monomers or can be capped. Pg-poly(HPMA-co-MA-b-Ala-Pg)-DTB random copolymer was synthesized following the same synthetic procedure as described for Compound 21, TT-poly(HPMA-co-MA-b-Ala-Pg)-DTB, except using CTA-Pg and ACVA-Pg. Light pink powder was obtained with 48.2% yield. Number-average (Mₙ) and weight-average molecular weight (M_{w}) were 36.34 kDa and 40.06 kDa, respectively, and polydispersity (PDI) was 1.10 measured by GPC-MALS. The chain end functionalities measured by UV-Vis spectroscopy [ε₃₀₅ (DTB)=12600 L/(mol·cm)] showed that DTB %=112.5%.

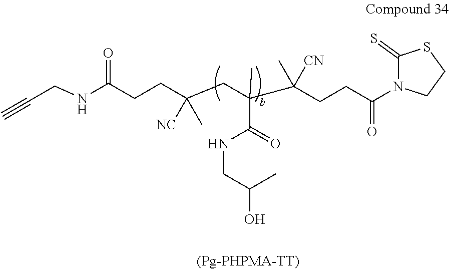

Compound 34, Pg-PHPMA-TT, was synthesized from Compound 32 using the same method as described for as Compound 22 except that ACVA-TT was used instead of ACVA-Pg. Note: in this example, the Pg group was added to the polymer during the polymerization step and the TT functionality was added to the other end during capping.

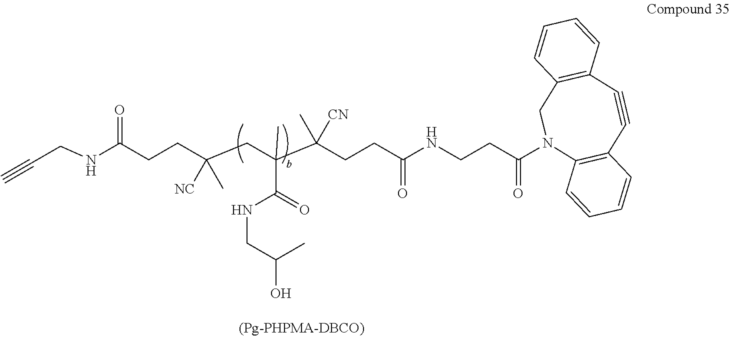

Compound 35. Pg-PHPMA-DBCO was synthesized using the same method as described for as Compound 34 except that ACVA-DBCO was used instead of with ACVA-TT. Note: in this example, the Pg group was added to the polymer during the polymerization step and the strained-alkyne functionality was added to the other end during capping.

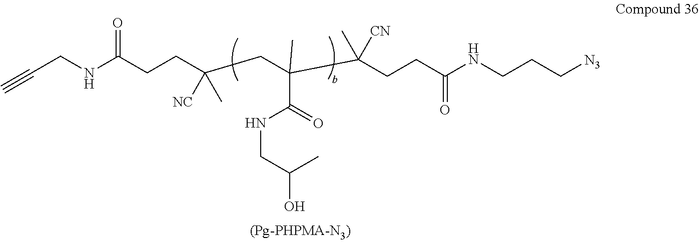

Compound 36. Pg-PHPMA-N₃ was synthesized using the same method as described for as Compound 34 but ACVA-N₃ was used instead of with ACVA-TT. Note: in this example, the Pg group was added to the polymer during the polymerization step and the azide functionality was added to the other end during capping.

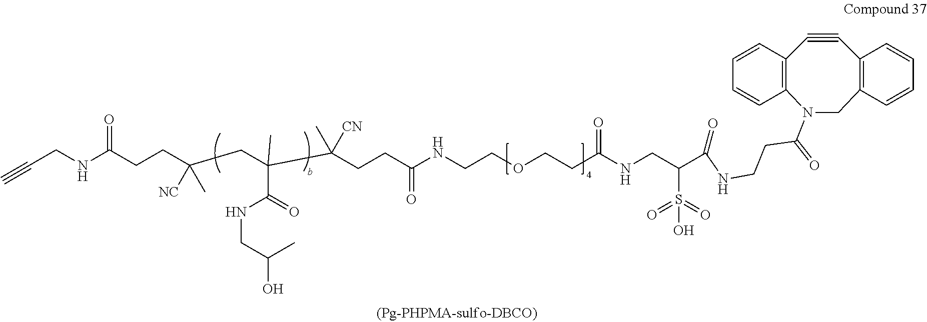

Compound 37. Pg-PHPMA-sulfo-DBCO was synthesized using the same method as described for Compound 34, Pg-PHPMA-TT, except that ACVA-TT was replaced by ACVA-sulfo-DBCO. Note: in this example, the Pg group was added to the polymer during the polymerization step and the water-soluble strained-alkyne functionality was added to the other end during capping.

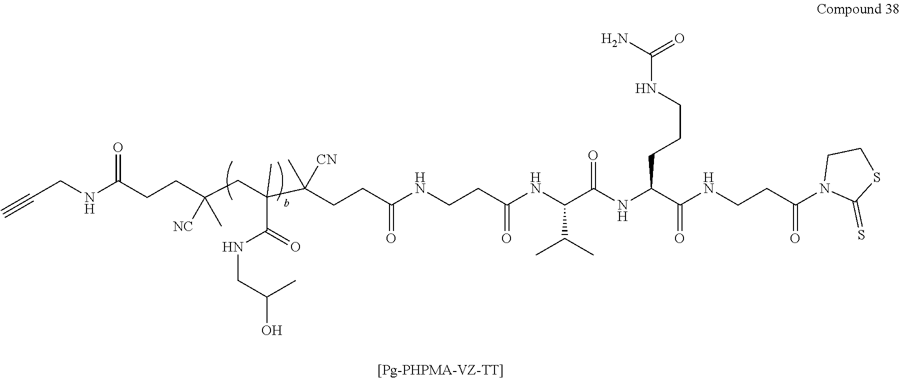

Compound 38. Pg-PHPMA-VZ-TT was synthesized using the same method as described for Compound 34, Pg-PHPMA-TT, except that ACVA-TT were replaced by ACVA-VZ-TT. Note: in this example, the Pg group was added to the polymer during the polymerization step and the TT-activated peptide was added to the other end during capping.

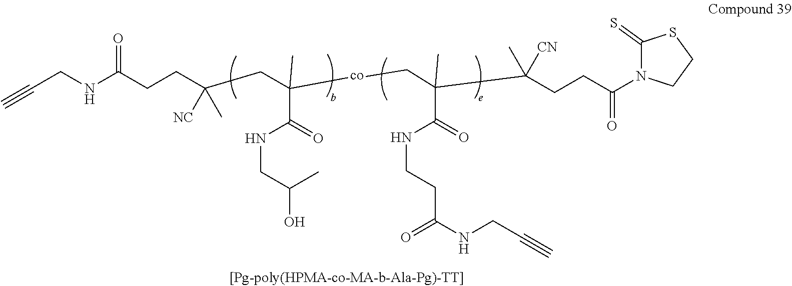

Compound 39. Pg-poly(HPMA-co-MA-b-Ala-Pg)-TT was synthesized by capping Compound 33 Pg-poly(HPMA-co-MA-b-Ala-Pg)-DTB with ACVA-TT using the same method as described for Compound 34, Pg-PHPMA-TT. Note: in this example, the Pg group was added to the polymer during the polymerization step and the TT functionality was added to the other end during capping.

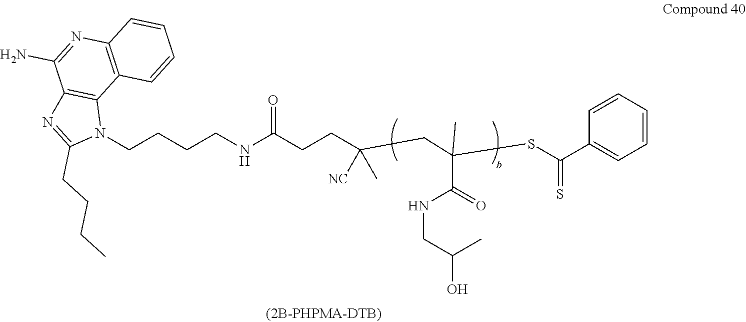

Compound 40. 2B-PHPMA-DTB was synthesized using the same method as described for Compound 20, TT-PHPMA-DTB, except that ACVA-TT and CTA-TT were replaced by ACVA-2B and CTA-2B, and [M]₀:[CTA-2B]₀ is adjusted to target Mₙ=10 kDa. Light pink powder was obtained with 48.2% yield. Number-average (Mₙ) and weight-average molecular weight (M_{w}) were 11.86 kDa and 12.82 kDa, respectively, and polydispersity (PDI) was 1.08 measured by GPC-MALS.

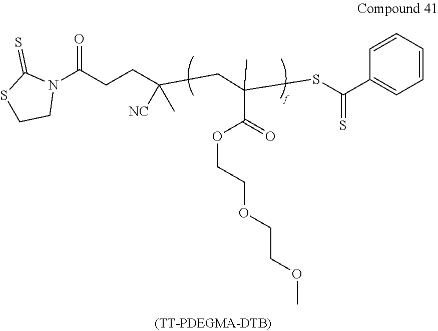

Compound 41. T T-PDEGMA-DTB was synthesized via the RAFT polymerization of DEGMA using CTA-TT as a chain transfer agent and ACVA-TT as an initiator in 1,4-dioxane/DMSO at 70° C. for 3 h. The initial monomer concentration [DEGMA]₀=4.0 mol/L, the molar ratio [CTA-TT]₀:[ACVA-TT]₀=1:0.2, and [DEGMA]₀:[CTA-TT]₀ varied to obtain polymers with different chain lengths. The following procedure was employed for a typical polymerization to produce TT-PDEGMA-DTB targeting a molecular weight of 20 kDa: DEGMA (1003.0 mg, 5.32 mmol) was dissolved in 1.3 mL of 1,4-dioxane. CTA-T T (16.87 mg, 0.044 mmol) as a 100 mg/mL stock solution in anhydrous DMSO and ACVA-TT (4.28 mg, 0.009 mmol) as a 50 mg/mL stock solution in anhydrous DMSO were added to the monomer solution. The mixture was transferred to a 5 mL ampule, which was sealed with a rubber septum and sparged with Ar (g) at r.t. for 30 min. The flask was then immersed in a water circulator preheated to 70° C. and polymerize for 3 h. The polymer was purified by precipitating against diethyl ether for 3 times. After drying in vacuum oven overnight, pink solid was obtained (460.7 mg, 45.2% yield). Number-average (Mₙ) and weight-average molecular weight (M_{w}) were 21.53 kDa and 22.09 kDa, respectively, and polydispersity (PDI) was 1.03 measured by GPC-MALS.

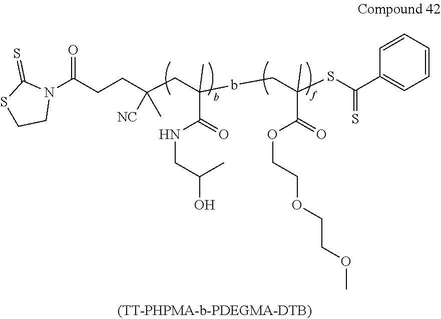

Compound 42. TT-PHPMA-b-PDEGMA-DTB was synthesized via a chain-extension polymerization through the RAFT mechanism of DEGMA using Compound 20, TT-PHPMA-DTB, as the macromolecular chain transfer agent (macro-CTA) and 2,2′-azobis(2-methylpropionitrile) (AIBN) as an initiator in tBuOH/DMAc (5/5, v/v) at 70° C. for 16 h. [DEGMA]₀=0.67 mol/L and [macro-CTA]₀:[AIBN]₀=1:0.2. For example, when TT-PHPMA12.8k-DTB was used as the macro-CTA and [DEGMA]₀:[macro-CTA]₀ is adjusted to 100 targeting Mₙ (PDEGMA)=20 kDa. TT-PHPMA-DTB (257.0 mg, 20.0 μmol) was dissolved in 1.5 mL of anhydrous DMAc. AIBN (0.66 mg, 4.0 μmol) as a 50 mg/mL stock solution in anhydrous DMAc, DEGMA (376.4 mg, 2.00 mmol) and 1.5 mL of anhydrous tBuOH was then added to the macro-CTA solution. The mixture was transferred to a 5 mL ampule, which was sealed with a rubber septum and sparged with Ar (g) at r.t. for 30 min. The flask was then immersed in a water circulator preheated to 70° C. and polymerize for 18 h. The polymer was purified by precipitating against diethyl ether for 3 times. After drying in vacuum oven overnight, light pink solid was obtained (537.1 mg, 84.8% yield). Number-average (Mₙ) and weight-average molecular weight (M_{w}) were 32.27 kDa and 34.33 kDa, respectively, and polydispersity (PDI) was 1.06 measured by GPC-MALS.

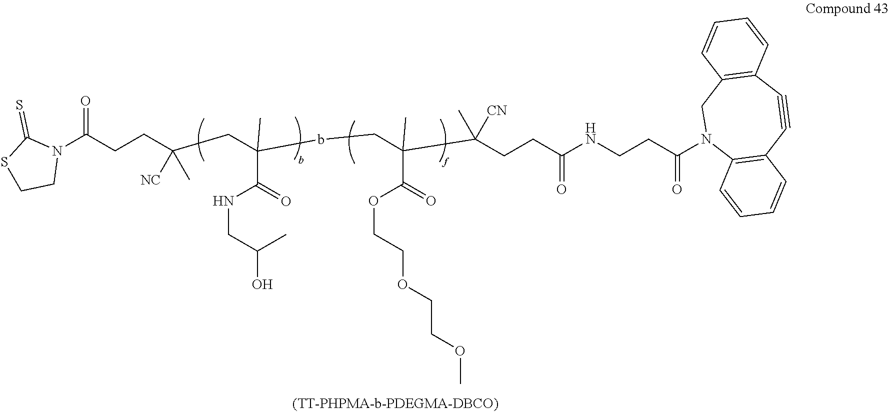

Compound 43. TT-PHPMA-b-PDEGMA-DBCO was synthesized by capping Compound 42, TT-PHPMA-b-PDEGMA-DTB, with ACVA-DBCO using the same method as described for Compound 23, TT-PHPMA-DTB.

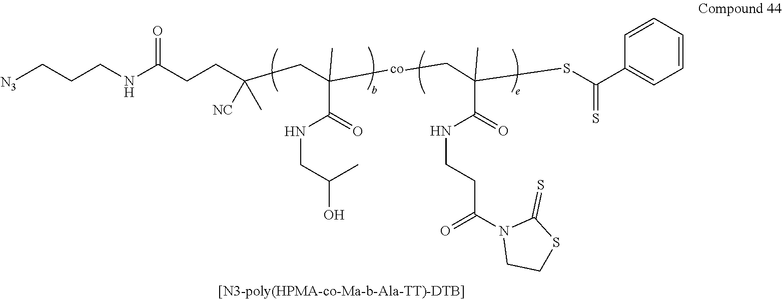

Compound 44. N₃-poly(HPMA-co-Ma-b-Ala-TT)-DTB was synthesized via the RAFT polymerization of HPMA and Ma-b-Ala-TT using CTA-N₃ as a chain transfer agent and ACVA-N₃ as an initiator in 1:1 tert-butanol (tBuOH) and dimethylacetamide (DMAc) at 70° C. for 16 h. The initial monomer concentration [HPMA/Ma-b-Ala-TT]₀=1 mol/L with [HPMA]₀:[Ma-b-Ala-TT]₀=7:3, the molar ratio [CTA-N₃]₀:[ACVA-N₃]₀=1:0.5, and [HPMA/Ma-b-Ala-TT]₀:[CTA-N₃]₀ varied to obtain polymers with different chain lengths. The following procedure was employed for a typical polymerization to produce N₃-poly(HPMA-co-Ma-b-Ala-TT)-DTB targeting molecular weight of 40 kDa: HPMA (1503.50 mg, 10.50 mmol) was dissolved in 9.5 mL tBuOH. Ma-b-Ala-TT (1162.60 mg, 4.50 mmol) was dissolved in 9.5 mL anhydrous DMAc and combined with HPMA solution. CTA-N₃ (19.70 mg, 0.055 mmol) and ACVA-N₃ (12.10 mg, 0.027 mmol) were dissolved in anhydrous DMAc before mixing with monomer solution. The mixture was transferred to a 20 mL ampule, which was sealed with a rubber septum and sparged with Ar (g) at r.t. for 45 min. The flask was then immersed in a water circulator preheated to 70° C. and polymerize for 16 h. The polymer was purified by precipitating against acetone three times. After drying in a vacuum oven overnight, an orange powder was obtained (1498 mg, 55.8% yield). Number-average (Mₙ) and weight-average molecular weight (M_{w}) were 36.63 kDa and 37.71 kDa, respectively, and polydispersity (PDI) was 1.03 measured by GPC-MALS. The arrayed functionality was measured by UV-Vis spectroscopy [ε₃₀₅ (TT)=10300 L/(mol·cm)] showed 34.2 mol % TT.

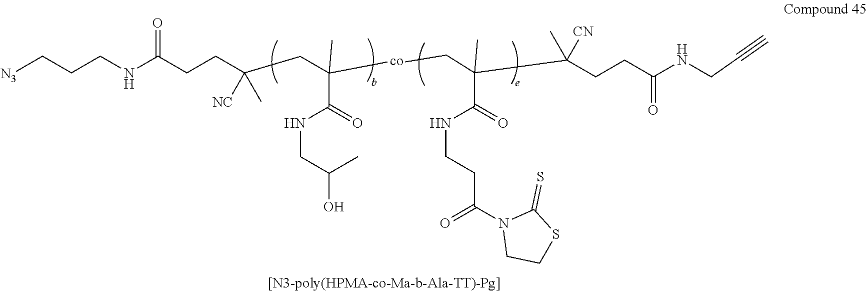

Compound 45 is an example of a polymer arm comprised of a copolymer with hydrophilic monomers (B) and reactive monomer (E). N₃-poly(HPMA-co-Ma-b-Ala-TT)-Pg was synthesized by capping Compound 44, N₃-poly(HPMA-co-Ma-b-Ala-TT)-DTB with ACVA-Pg following the same synthetic procedure as Compound 22.

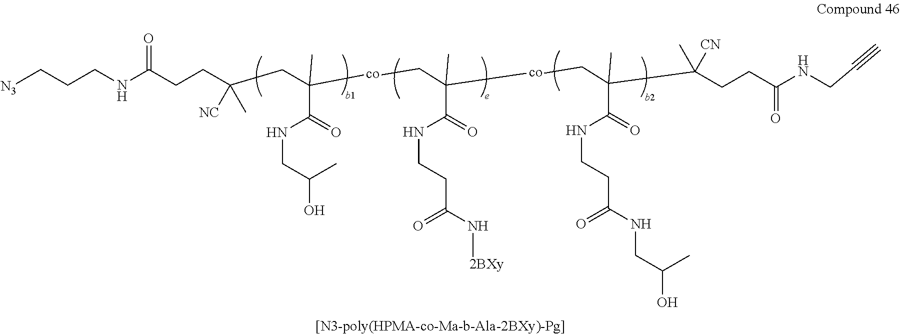

Compound 46 is an example of a polymer arm comprised of a copolymer with hydrophilic monomers (B) and reactive monomer (E), wherein the reactive monomers are linked to a drug (D), i.e. the TLR-7/8a, 2BXy, through an amide bond. N₃-poly(HPMA-co-Ma-b-Ala-2BXy)-Pg was synthesized by reacting the carbonylthiazolidine-2-thione (TT) groups of Compound 45 with 2BXy (Compound A) and amino-2-propanol in the molar ratio [2BXy]: [amino-2-propanol]=1:2. Specifically, N₃-poly(HPMA-co-Ma-b-Ala-TT)-Pg (40.00 mg, 1.05 μmol polymer, 72 μmol TT) and 2 mL of DMSO were added to a 20 mL scintillation vial. The polymer was fully dissolved before the addition of 2BXy (7.80 mg, 21.77 μmol) and triethylamine (15.10 μL, 110 μmol). The reaction was allowed to proceed at r.t. for 2 h before the addition of amino-2-propanol (4.50 mg, 60 μmol) and additional hour afterward. The polymer was then purified by dialysis against methanol for 2 h three times using reconstituted cellulose (RC) membrane with a molecular weight cutoff (MWCO) of 20 kDa. The polymer was collected by precipitating against diethyl ether and dried overnight in a vacuum oven. The product was obtained as a white powder (31.4 mg, 70.6% yield). Mₙ and M_{w} were 50.21 kDa and 54.95 kDa, respectively, and PDI was 1.09 measured by GPC-MALS. The 2BXy content measured by UV-Vis spectroscopy [ε₃₂₅ (2BXy)=5012 L/(mol·cm) showed 10.28 mol % 2BXy.

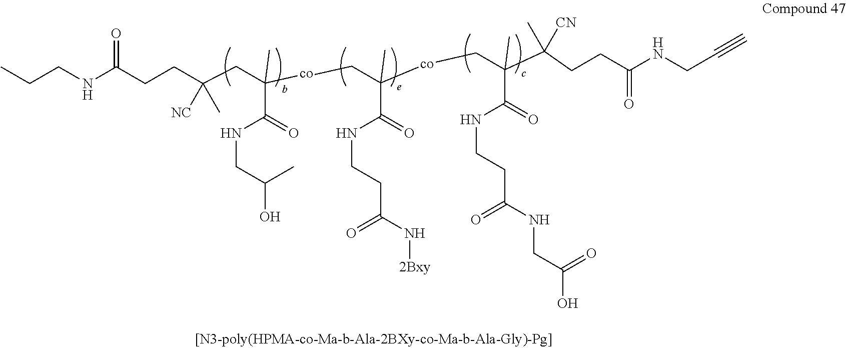

Compound 47 is an example of a polymer arm comprised of a terpolymer with hydrophilic monomers (B), reactive monomers (E) linked to a drug (D), i.e. the TLR-7/8a, 2BXy, and charged monomers (C) with a carboxylic acid group, which is negatively charged at pH 7.4. Note: drug is linked to the reactive monomer through an amide bond. N₃-poly(HPMA-co-Ma-b-Ala-2BXy-co-Ma-b-Ala-Gly)-Pg was synthesized in the same manner as Compound 46 but glycine was used instead of amino-2-propanol and the ratio of DMSO:PBS(1×)=4:1 was used as the solvent.

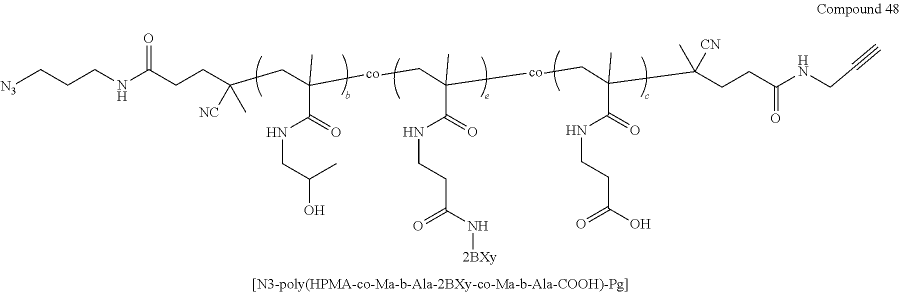

Compound 48 is an example of a polymer arm comprised of a terpolymer with hydrophilic monomers (B), reactive monomers (E) linked to a drug (D), i.e. the TLR-7/8a, 2BXy, and charged monomers (C) with a carboxylic acid group, which is negatively charged at pH 7.4. Note: the drug is linked to the reactive monomer through an amide bond. N₃-poly(HPMA-co-Ma-b-Ala-2BXy-co-Ma-b-Ala-COOH)-Pg was synthesized in the same manner as Compound 46 but amino-2-propanol was not used, instead the remaining TT groups were hydrolyzed with 0.01M NaOH after addition of 2BXy.

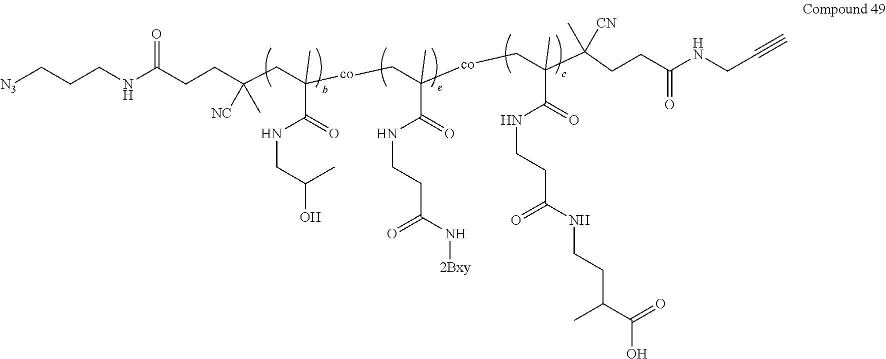

[N₃-poly(HPMA-co-Ma-b-Ala-2BXy-co-Ma-b-Ala-methylbutanoic acid)-Pg]

Compound 49 is an example of a polymer arm comprised of a terpolymer with hydrophilic monomers (B), reactive monomers (E) linked to a drug (D), i.e. the TLR-7/8a, 2BXy, and charged monomers (C) with a carboxylic acid group, which is negatively charged at pH 7.4. Note: the drug is linked to the reactive monomer through an amide bond. N₃-poly(HPMA-co-Ma-b-Ala-2BXy-co-Ma-b-Ala-methylbutanoic acid)-Pg was synthesized in the same manner as Compound 46 but 4-amino-2-methylbutanoic acid was used instead of amino-2-propanol.

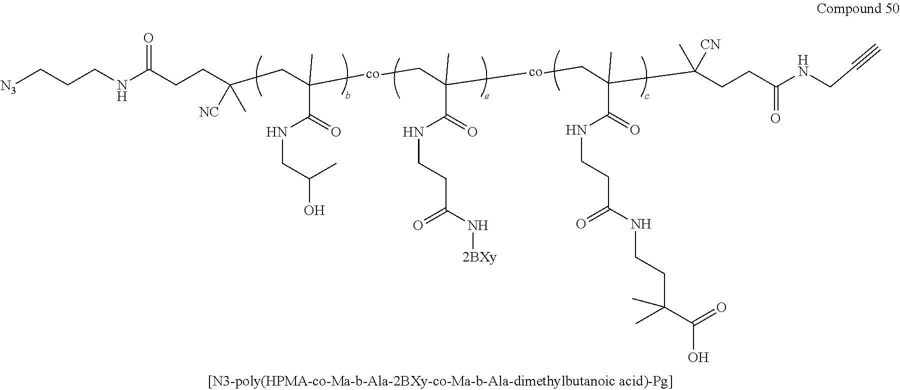

Compound 50 is an example of a polymer arm comprised of a terpolymer with hydrophilic monomers (B), reactive monomers (E) linked to a drug (D), i.e. the TLR-7/8a, 2BXy, and charged monomers (C) with a carboxylic acid group, which is negatively charged at pH 7.4. Note: the drug is linked to the reactive monomer through an amide bond. N₃-poly(HPMA-co-Ma-b-Ala-2BXy-co-Ma-b-Ala-dimethylbutanoic acid)-Pg was synthesized in the same manner as Compound 46 but 4-amino-2,2-dimethylbutanoic acid was used instead of amino-2-propanol.

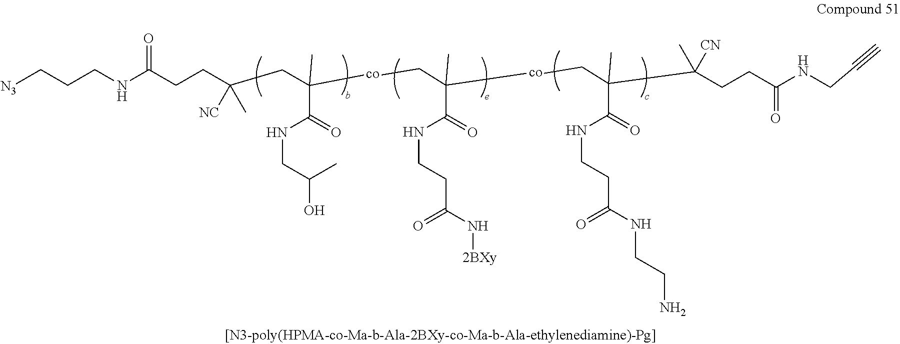

Compound 51 is an example of a polymer arm comprised of a terpolymer with hydrophilic monomers (B), reactive monomers (E) linked to a drug (D), i.e. the TLR-7/8a, 2BXy, and charged monomers (C) with an amine group, which is positively charged at pH 7.4. Note: the drug is linked to the reactive monomer through an amide bond. N₃-poly(HPMA-co-Ma-b-Ala-2BXy-co-Ma-b-Ala-ethylenediamine)-Pg was synthesized in the same manner as Compound 46 but ethylenediamine was used instead of amino-2-propanol.

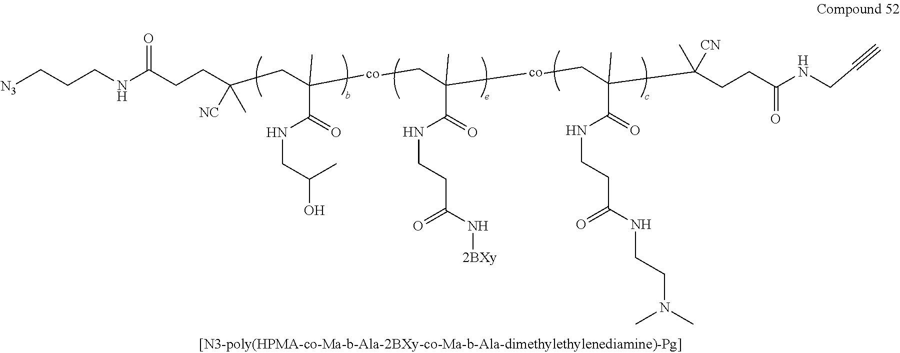

Compound 52 is an example of a polymer arm comprised of a terpolymer with hydrophilic monomers (B), reactive monomers (E) linked to a drug (D), i.e. the TLR-7/8a, 2BXy, and charged monomers (C) with a tertiary amine group, which is positively charged at pH 7.4. Note: the drug is linked to the reactive monomer through an amide bond. N₃-poly(HPMA-co-Ma-b-Ala-2BXy-co-Ma-b-Ala-dimethylethylenediamine)-Pg was synthesized in the same manner as Compound 46 but N,N′-dimethylethylenediamine was used instead of amino-2-propanol.

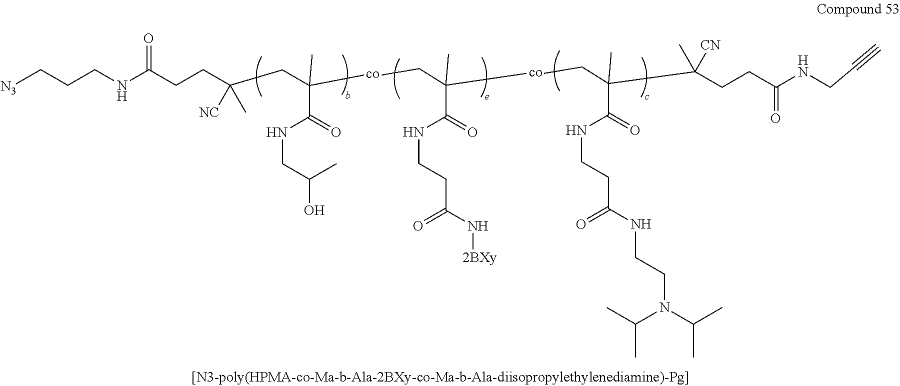

Compound 53 is an example of a polymer arm comprised of a terpolymer with hydrophilic monomers (B), reactive monomers (E) linked to a drug (D), i.e. the TLR-7/8a, 2BXy, and charged monomers (C) with a tertiary amine group, which is positively charged at pH 7.4. Note: the drug is linked to the reactive monomer through an amide bond. N₃-poly(HPMA-co-Ma-b-Ala-2BXy-co-Ma-b-Ala-diisopropylethylenediamine)-Pg was synthesized in the same manner as Compound 46 but N,N′-diisopropylethylenediamine was used instead of amino-2-propanol.

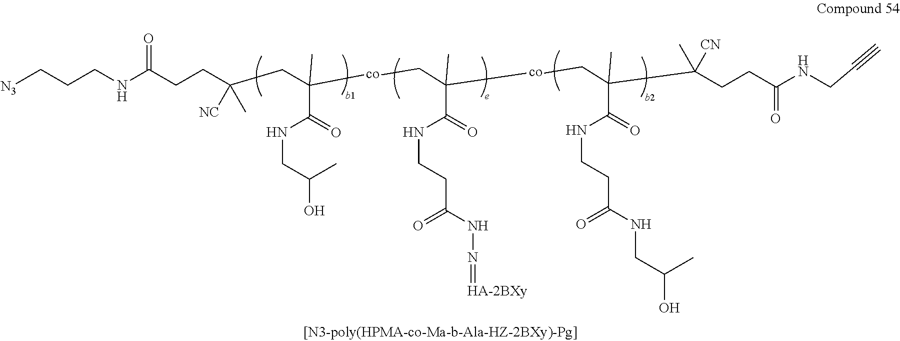

Compound 54 is an example of a polymer arm comprised of hydrophilic monomers (B) and reactive monomers (E) linked to a drug (D), i.e. the TLR-7/8a, 2BXy, through a hydrazone bond. N₃-poly(HPMA-co-Ma-b-Ala-HZ-2BXy)-Pg was synthesized by reacting the TT groups of Compound 44 with hydrazine monohydrate and amino-2-propanol in the molar ratio [hydrazine]:[amino-2-propanol]=1:2 and forming a hydrazone linkage to Compound D, 2BXy-HA, through these polymer-bound hydrazides. Specifically, N₃-poly(HPMA-co-Ma-b-Ala-TT)-Pg (10.00 mg, 0.26 μmol) and 100 μL of methanol were added to a 2 mL vial. The polymer was fully dissolved before the addition of hydrazine monohydrate (0.27 mg, 5.43 μmol). The reaction was allowed to proceed at r.t. for 30 minutes before the addition of amino-2-propanol (1.02 mg, 13.61 μmol) and additional hour afterward. The 2BXy-HA (3.17 mg, 6.53 μmol) and 32 μL DMSO were added to the vial just prior to addition of acetic acid (20.61 μL, 360 μmol). The reaction was allowed to proceed at r.t. overnight. The polymer was then purified by dialysis against methanol for 2 h three times using reconstituted cellulose (RC) membrane with a molecular weight cutoff (MWCO) of 25 kDa. The polymer was collected by precipitating against diethyl ether and dried overnight in a vacuum oven. The product was obtained as a white powder. Mₙ and M_{w} were 59.61 kDa and 61.09 kDa, respectively, and PDI was 1.02 measured by GPC-MALS. The 2Bxy content measured by UV-Vis spectroscopy [ε₃₂₅ (2Bxy)=5012 L/(mol·cm) showed 9.79 mol % 2Bxy.

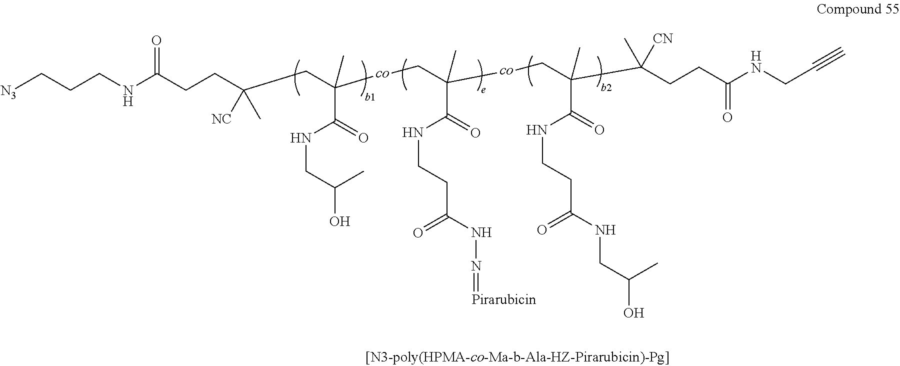

Compound 55 is an example of a polymer arm comprised of hydrophilic monomers (B) and reactive monomers (E) linked to a drug (D), i.e. the cytotoxic anthracycline, Pirarubicin, through a hydrazone bond. N₃-poly(HPMA-co-Ma-b-Ala-HZ-Pirarubicin)-Pg was synthesized in the same manner as Compound 54 but pirarubicin, which contains a ketone, was used instead of 2BXy-HA.

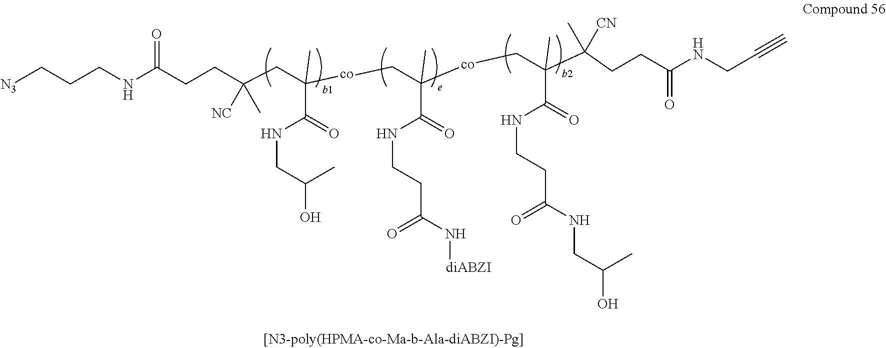

Compound 56 is an example of a polymer arm comprised of hydrophilic monomers (B) and reactive monomers (E) linked to a drug (D), i.e. the STING agonist pip-diABZI, through an amide bond. N₃-poly(HPMA-co-Ma-b-Ala-diABZI)-Pg was synthesized in the same manner as Compound 46 but Compound C, pip-diABZI, was used instead of 2BXy.

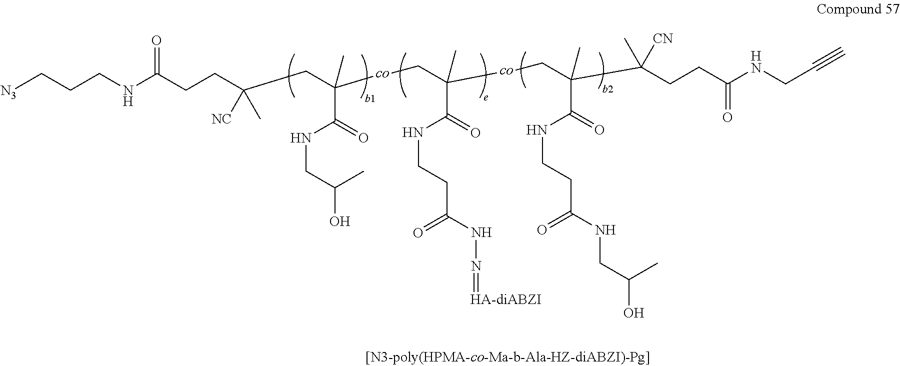

Compound 57 is an example of a polymer arm comprised of hydrophilic monomers (B) and reactive monomers (E) linked to a drug (D), i.e. the STING agonist pip-diABZI-HA, through a hydrazone bond. N₃-poly(HPMA-co-Ma-b-Ala-HZ-diABZI)-Pg was synthesized in the same manner as Compound 54 but Compound E, diABZI-HA, was used instead of 2BXy-HA and DMSO was used as the solvent.

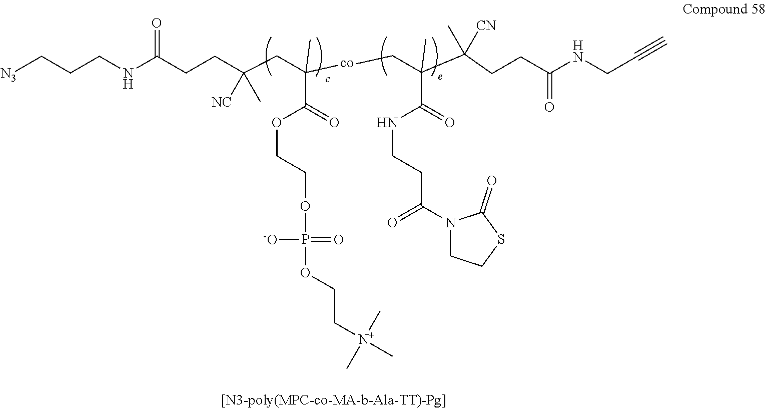

Compound 58. N₃-poly(MPC-co-MA-b-Ala-TT)-Pg random copolymer was synthesized by polymerizing zwitterionic monomer MPC and amine-reactive monomer MA-b-Ala-TT in anhydrous MeOH following the same synthetic procedure as described for Compound 44, N₃-poly(HPMA-co-MA-b-Ala-TT)-DTB. Take one synthesis as an example, [MPC]₀:[MA-b-Ala-TT]₀=7/3 and [MPC+MA-b-Ala-TT]₀:[CTA-N₃]₀ was adjusted to target a 60 kDa copolymer containing 30 mol % of reactive monomer. The polymerization was allowed to proceed at 70° C. for 16 h followed with purification. The resulted polymer was then used to react with 20 eq. of ACVA-Pg, following the same synthetic procedure as Compound 45, TT-PHPMA-Pg, yielding light yellow powder. Number-average (Mₙ) molecular weight was 47.02 kDa and polydispersity (PDI) was 1.04 measured by GPC-MALS. The TT functionalities measured by UV-Vis spectroscopy [ε₃₀₅ (TT)=11300 L/(mol·cm)] showed that TT %=12.41%.

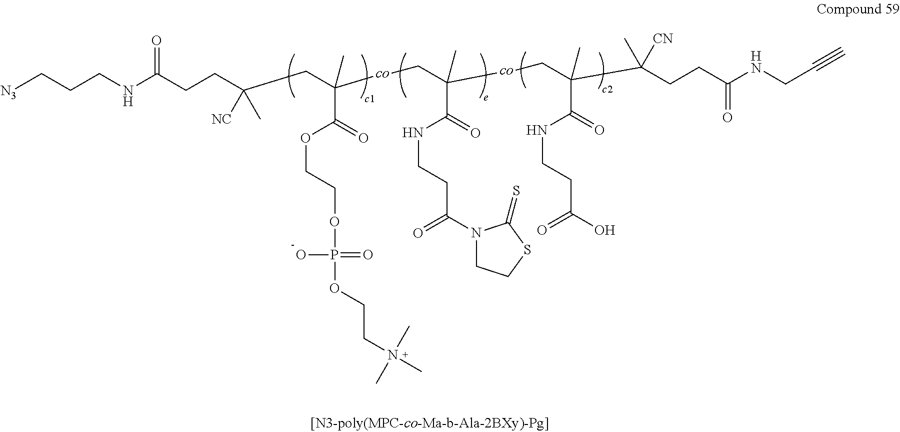

Compound 59. N₃-poly(MPC-co-Ma-b-Ala-2Bxy)-Pg was synthesized and purified in the same manner as Compound 46 except 2BXy was reacted with Compound 58 in excess without amino-2-propanol.

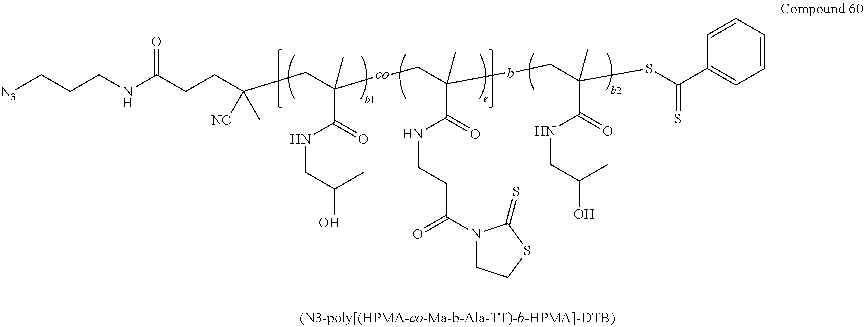

Compound 60. N₃-poly[(HPMA-co-Ma-b-Ala-TT)-b-HPMA]-DTB was synthesized via a chain-extension polymerization through the RAFT mechanism of HPMA using Compound 44, N₃-poly(HPMA-co-Ma-b-Ala-TT)-DTB, as a macromolecular chain transfer agent (macro-CTA) and 2,2′-azobis(2-methylpropionitrile) (AIBN) as an initiator in tBuOH/DMAc (6/4, v/v) at 70° C. for 18 h. [HPMA]₀:[macro-CTA]₀ was varied to obtain block copolymers with different chain lengths. The initial monomer concentration [HPMA]₀=0.9 mol/L and the molar ratio [macro-CTA]₀:[AIBN]₀=1:0.2. For example, HPMA (258.3 mg, 1.80 mmol) was dissolved in 1.2 mL of anhydrous tBuOH. N₃-poly(HPMA-co-Ma-b-Ala-TT)-DTB (208.5 mg, 9.0 μmol) was dissolved in 0.8 mL of anhydrous DMAc before mixing with the monomer solution. AIBN (0.26 mg, 1.67 μmol) as a 50 mg/mL stock solution in anhydrous DMAc was then added to the mixture. The mixture was transferred to a 5 mL ampule, which was sealed with a rubber septum and sparged with Ar (g) at r.t. for 20 min. The flask was then immersed in a water circulator preheated to 70° C. and polymerize for 18 h. The polymer was purified by precipitating against acetone/diethyl ether (3/1, v/v) for 3 times. After drying in vacuum oven overnight, light orange powder was obtained (277.0 mg, 59.3% yield). Number-average (Mₙ) and weight-average molecular weight (M_{w}) were 33.07 kDa and 37.06 kDa, respectively, and polydispersity (PDI) was 1.12 measured by GPC-MALS. The TT functionalities measured by UV-Vis spectroscopy [ε₃₀₅ (TT)=10300 L/(mol·cm), ε₃₀₅ (DTB)=12600 L/(mol·cm)] showed that the number of TT and DTB functionalities per polymer chain is 26 (12.6 mol % TT).

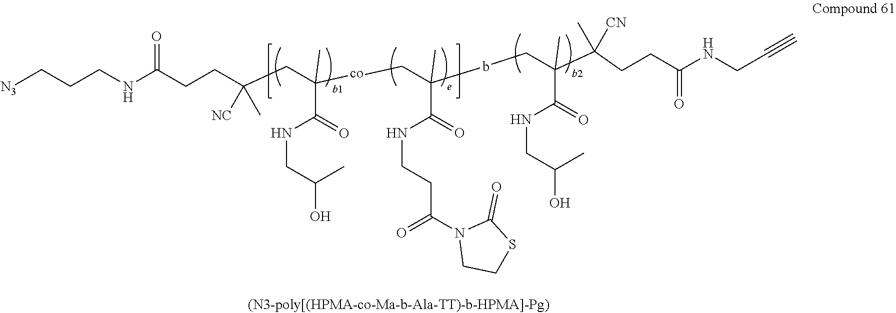

Compound 61 is an example of a polymer arm with di-block architecture comprised of hydrophilic monomers (B) and reactive monomers (E) on one block and only hydrophilic monomers on the other block. Note: in this example the di-block polymer is heterotelechelic with different functionalities on each end of the polymer arm. N₃-poly[(HPMA-co-Ma-b-Ala-TT)-b-HPMA]-Pg was synthesized by capping Compound 60 using ACVA-Pg in the same manner as Compound 22.

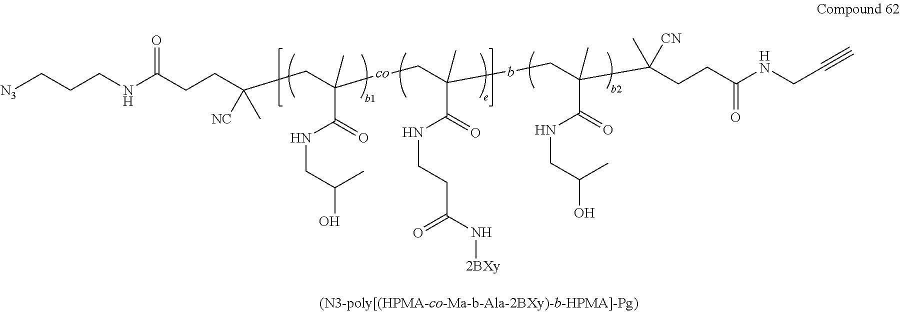

Compound 62 is an example of a polymer arm with di-block architecture comprised of hydrophilic monomers (B) and reactive monomers (E) linked to drug (i.e. the TLR-7/8a, 2BXy) through an amide bond on one block and only hydrophilic monomers on the other block. Note: in this example the di-block polymer is heterotelechelic with different functionalities on each end of the polymer arm. N₃-poly[(HPMA-co-Ma-b-Ala-2BXy)-b-HPMA]-Pg was synthesized by reacting the carbonylthiazolidine-2-thione (TT) groups of Compound 61 with excess 2BXy (Compound A). Specifically, N₃-poly[(HPMA-co-Ma-b-Ala-TT)-b-HPMA]-Pg (30.0 mg, 0.91 μmol, 22.5 μmol TT groups) and 0.6 mL of anhydrous DMSO were added to a 20 mL scintillation vial. The polymer was fully dissolved before the addition of 2BXy (8.3 mg, 23.1 μmol, dissolved in 900 μL anhydrous DMSO) and triethylamine (3.5 μL, 82.0 μmol). The reaction was allowed to proceed at r.t. for overnight. The product was then purified precipitating against diethyl ether and dried overnight in a vacuum oven. The product was obtained as a white powder (26.8 mg, 70.0% yield). Mₙ and M_{w} were 35.8 kDa and 45.8 kDa, respectively, and PDI was 1.28 measured by GPC-MALS. The 2BXy content measured by UV-Vis spectroscopy [ε₃₂₅ (2BXy)=5012 L/(mol·cm) showed 11.62 mol % 2BXy.

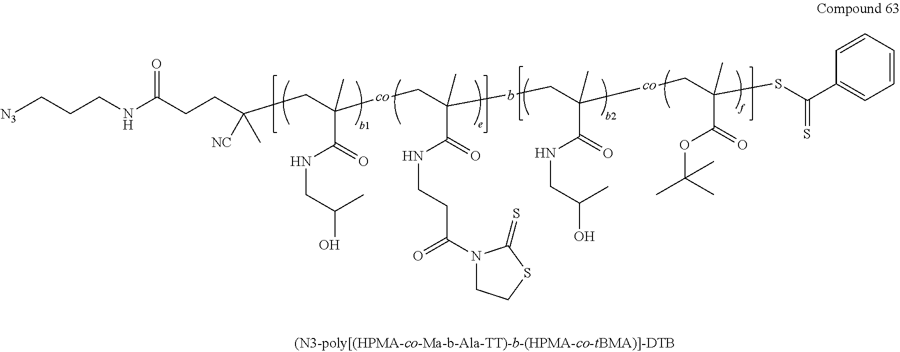

Compound 63. N₃-poly[(HPMA-co-Ma-b-Ala-TT)-b-(HPMA-co-tBMA)]-DTB was synthesized in the same manner as Compound 60 by polymerizing tert-butyl methacrylate (tBMA) and HPMA at ratio [HPMA]₀:[tBMA]₀=9:1.

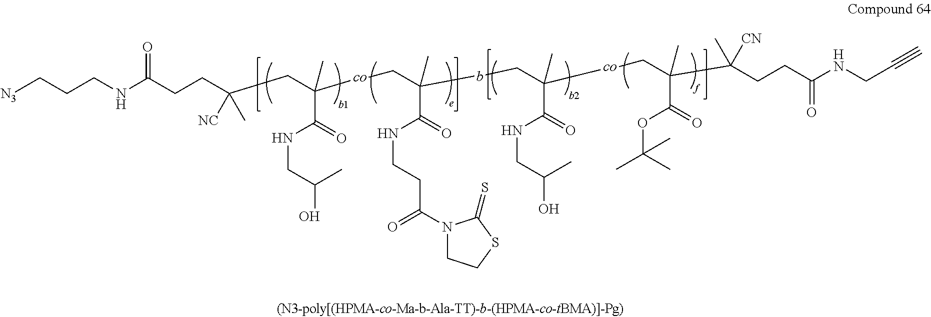

Compound 64. N₃-poly[(HPMA-co-Ma-b-Ala-TT)-b-(HPMA-co-tBMA)]-Pg was synthesized in the same manner as Compound 61.

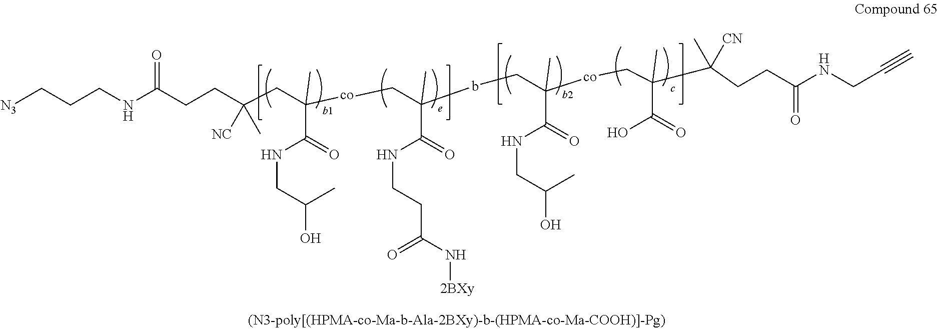

Compound 65 is an example of a polymer arm with di-block architecture comprised of hydrophilic monomers (B) and reactive monomers (E) linked to drug (i.e. the TLR-7/8a 2BXy) through an amide bond on one block and both hydrophilic monomers (B) and charged monomers (C) with a carboxylic acid functional group on the other block. Note: in this example the di-block polymer is heterotelechelic with different functionalities on each end of the polymer arm. N₃-poly[(HPMA-co-Ma-b-Ala-2BXy)-b-(HPMA-co-Ma-COOH]-Pg was synthesized by reacting Compound 64 with 2BXy following the same protocol as Compound 62. Then tBMA was deprotected by dissolving the polymer in 95/2.5/2.5 TFA/TIPS/H₂O at 10 mM and sonicating for 5 minutes. The following procedure was employed for a typical deprotection: N₃-poly[(HPMA-co-Ma-b-Ala-2BXy)-b-(HPMA-co-tBMA)]-Pg (45.4 mg, 1.15 μmol) was dissolved in 100 μL 95/2.5/2.5 TFA/TIPS/H₂O and sonicated for 5 minutes. The polymer was then purified by precipitating against diethyl ether three times. After drying in a vacuum oven overnight, a white powder was obtained. Number-average (Mₙ) and weight-average molecular weight (M_{w}) were 39.5 kDa and 50.1 kDa, respectively, and polydispersity (PDI) was 1.27 measured by GPC-MALS. The 2BXy content measured by UV-Vis spectroscopy [ε₃₂₅ (2BXy)=5012 L/(mol·cm) showed 10.8 mol % 2BXy.

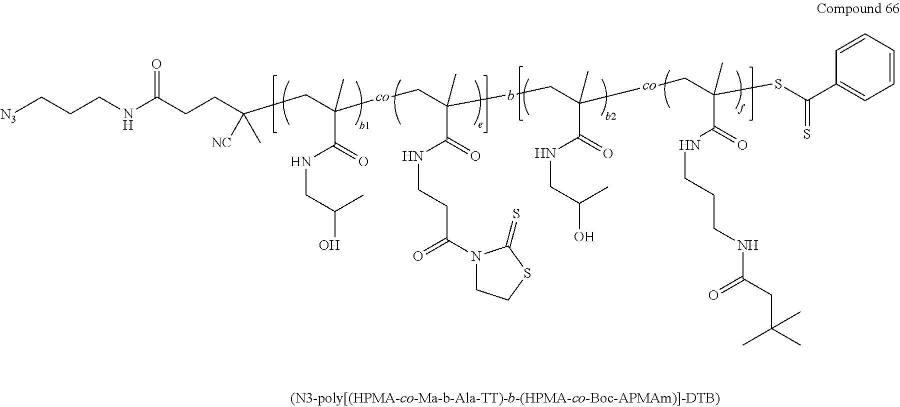

Compound 66. N₃-poly[(HPMA-co-Ma-b-Ala-TT)-b-(HPMA-co-Boc-APMAm)]-DTB was synthesized in the same manner as Compound 63 but tBMA was replaced with N-(t-Boc-aminopropyl)methacrylamide (Boc-APMAm).

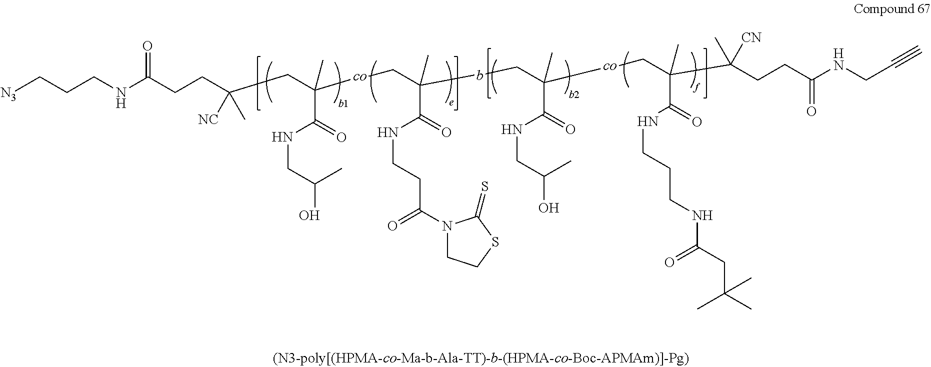

Compound 67. N₃-poly[(HPMA-co-Ma-b-Ala-TT)-b-(HPMA-co-Boc-APMAm)]-Pg was synthesized in the same manner as Compound 61.

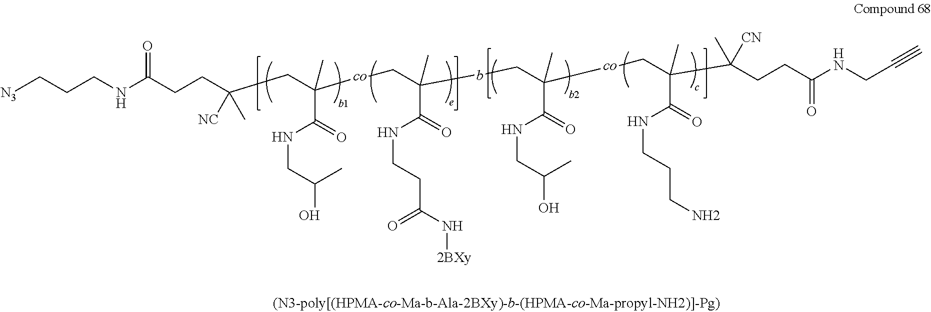

Compound 68 is an example of a polymer arm with di-block architecture comprised of hydrophilic monomers (B) and reactive monomers (E) linked to drug (i.e. the TLR-7/8a 2BXy) through an amide bond on one block and both hydrophilic monomers (B) and charged monomers (C) with an amide functional group on the other block. Note: in this example the di-block polymer is heterotelechelic with different functionalities on each end of the polymer arm. N₃-poly[(HPMA-co-Ma-b-Ala-2BXy)-b-(HPMA-co-Ma-propyl-NH2)]-Pg was synthesized in the same manner as Compound 65.

### Example 4—Synthesis of Dendrimer Cores

Compound 69 is an example of an X1 linker precursor linked to a core through a PEG linker. Trans-Cyclooctene (TCO)-functionalized G3 PAMAM dendrimer, PAMAM(G3)-g-(PEG₄-TCO)n, was synthesized by reacting TCO-PEG₄-NHS ester with G3 PAMAM dendrimer cores. The following procedure was employed to produce PAMAM Gen 3.0 dendrimers with 16 TCO functional groups (PAMAM Gen3-16TCO): Into a 20 mL scintillation vial, TCO-PEG₄-NHS ester solution (30.9 L, 100 mg/mL in methanol, 5.79 μmol), PAMAM Gen 3.0 dendrimer solution (14.48 μL, 20 wt % in methanol, 0.36 μmol), and 250 μL of anhydrous DMSO were added. Methanol solvent was then removed by applying vacuum before the addition of triethylamine (1.6 μL, 11.6 μmol). The mixture was allowed to stir overnight at r.t. Triethylamine was removed by applying vacuum and the solution was stored at -20° C. for future use (assuming 100% yield).

Compound 70 is an example of an X1 linker precursor linked to a core through a PEG linker. Azide-functionalized G5 PAMAM dendrimer, PAMAM(G5)-g-(PEG4-N₃)n, was synthesized by reacting N₃-PEG₄-NHS ester with PAMAM cores. The following procedure was employed to produce PAMAM Gen 5.0 dendrimers with 64 azide functional groups (PAMAM Gen5-64N₃): Into a 20 mL scintillation vial, N₃—PEG₄-NHS ester solution (21.6 μL, 100 mg/mL in methanol, 5.55 μmol), PAMAM Gen 5.0 dendrimer solution (62.7 μL, 5 wt % in methanol, 86.7 nmol), and 125 μL of anhydrous DMSO were added. Methanol solvent was then removed by applying vacuum before the addition of triethylamine (1.54 μL, 11.1 μmol). The mixture was allowed to stir overnight at r.t. Triethylamine was removed by applying vacuum and the solution was stored at −20° C. for future use (assuming 100% yield).

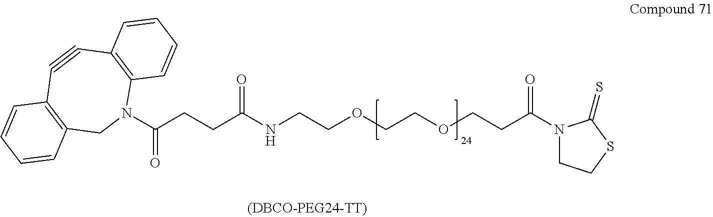

Compound 71. DBCO-PEG24-TT was synthesized via a two-step reaction from the starting compound Amino-PEG24-Acid. Amino-PEG24-acid (400 mg, 1 eq) was dissolved in THF to a concentration of 100 mg/mL. DBCO-NHS (154 mg, 1.1 eq) was dissolved in THF to a concentration of 50 mg/mL and added to the solution of Amino-PEG24-acid. Triethylamine (71 mg, 2 eq) was then added to the reaction mixture, which was incubated overnight with stirring at room temperature. reacted overnight at room temperature. The crude product was purified on a preparatory HPLC using a gradient of 25-55% acetonitrile/H₂O (0.05% TFA) over 12 minutes on an Agilent Prep C-18 column, 50×100 mm, 5 μm. The product fractions were pooled and lyophilized yielding light yellow oily solid DBCO-PEG24-acid (271.9 mg, 54.4%). DBCO-PEG24-acid (265.8 mg, 1 eq) was then dissolved in DCM to a concentration of 50 mg/mL. Thiazolidine-2-thione (24.3 mg, 1.1 eq) was likewise dissolved in DCM to a concentration of 100 mg/mL and added to the solution of DBCO-PEG24-acid. 1-Ethyl-3-(3-dimethylaminopropyl)carbodiimide (EDC) (86 mg, 2.4 eq) was dissolved in DCM to a concentration of 100 mg/mL and added to the reaction mixture. The reaction mixture was then cooled on wet ice and 4-Dimethylaminopyridine (DMAP) (1.1 mg, 0.05 eq) was added as a catalyst. The reaction was allowed to warm to room temperature while reacting for two hours, after which the product DBCO-PEG24-TT was purified on a preparatory HPLC using a gradient of 37-67% acetonitrile/H₂O (0.05% TFA) over 12 minutes on an Agilent Prep C-18 column, 50×100 mm, 5 μm. The product fractions were pooled and lyophilized yielding yellow oily solid DBCO-PEG24-TT (206.9 mg, 72.5%).

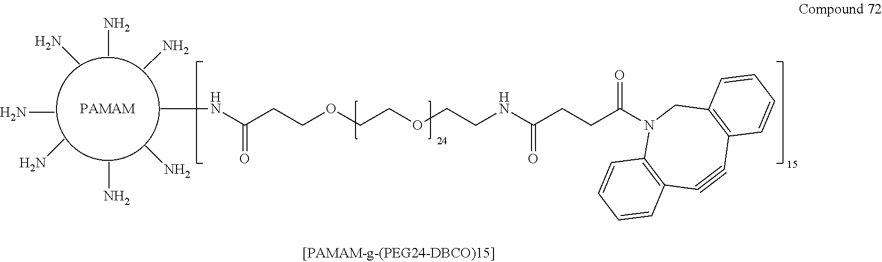

Compound 72 is an example of an X1 linker precursor linked to a core through a PEG linker, wherein the PEG in this example has 24 units of ethylene oxide. PAMAM(G5)-g-(PEG24-DBCO)₁₅ was synthesized by reacting DBCO-PEG24-TT with PAMAM dendrimer to yield a PAMAM dendrimer functionalized with 15 DBCO moieties with an extended 24-PEG linker. DBCO-PEG24-TT (20 mg, 15 eq) was dissolved in 0.6 mL of THF and added to PAMAM generation 5 (G5) (25 mg, 1 eq, 5 wt % in MeOH). The reaction was allowed to proceed for two hours at room temperature and monitored via analytical HPLC. Unreacted DBCO-PEG24-TT or hydrolyzed DBCO-PEG24-acid was then removed via dialysis against 200 mL pure THF using a 25 kDa MWCO RC membrane. Dialyzed product was diluted with 2 mL DMSO, after which THF was removed by vacuum. Product concentration in DMSO was then determined by DBCO UV absorbance from the extinction coefficient. Yield 65.3%.

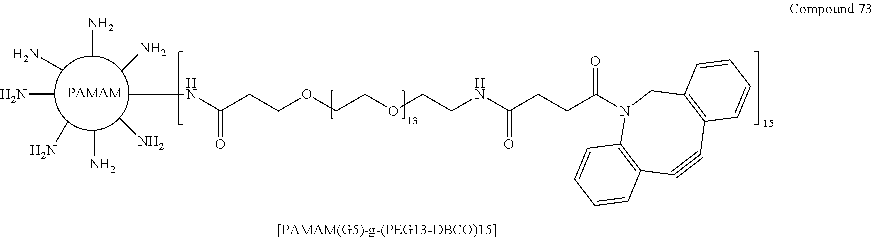

Compound 73 is an example of an X1 linker precursor linked to a core through a PEG linker, wherein the PEG in this example has 13 units of ethylene oxide. PAMAM(G5)-g-(PEG13-DBCO)₁₅ was synthesized by reacting DBCO-PEG13-NHS with PAMAM dendrimer in the same manner as Compound 72.

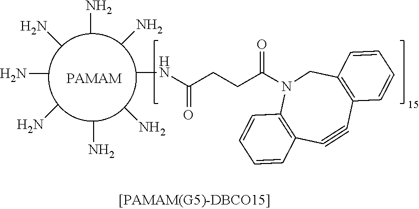

Compound 74 is an example of an X1 linker precursor linked to a core through a short linker. PAMAM(G5)-g-DBCO15 was synthesized by reacting DBCO-amine with PAMAM dendrimer in the same manner as Compound 72.

### Example 5—Synthesis of Star Polymers for Ligand Array by Route 1

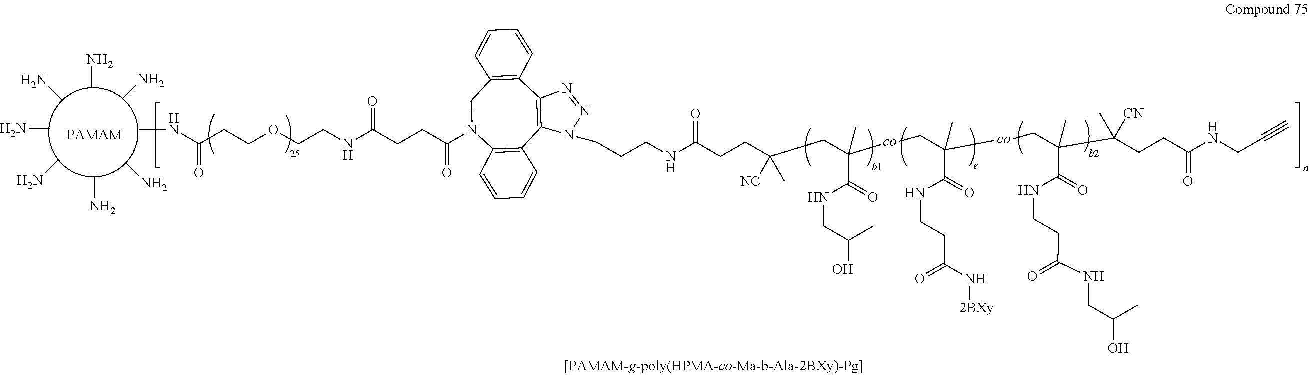

Compound 75 is an example of a star polymer with polymer arms comprised of hydrophilic monomers (B) and reactive monomers (E) linked to drug (D), i.e. the TLR-7/8a, 2BXy, through an amide bond. PAMAM-g-poly(HPMA-co-Ma-b-Ala-2BXy)-Pg was synthesized by reacting Compound 72 PAMAM(G5)-g-(PEG24-DBCO)₁₅ with Compound 46 to yield a star nanoparticle (star NP). Example synthesis: N₃-poly(HPMA-co-Ma-b-Ala-2Bxy)-Pg (3.55 mg, 75.0 nmol) and PAMAM(G5)-g-(PEG24-DBCO)₁₅ (0.501 mg, 150 nmol) were dissolved in 200 μL DMSO. The reaction was allowed to proceed at r.t. overnight. Precipitate reaction solution into diethyl ether and dry overnight in vacuum oven to yield white powder. Number-average (M) and weight-average molecular weight (M_{w}) were 818.3 kDa and 998.4 kDa, respectively, and polydispersity (PDI) was 1.22 measured by GPC-MALS. Using Mₙ it was determined that the star NP was composed of 15.3 arms.

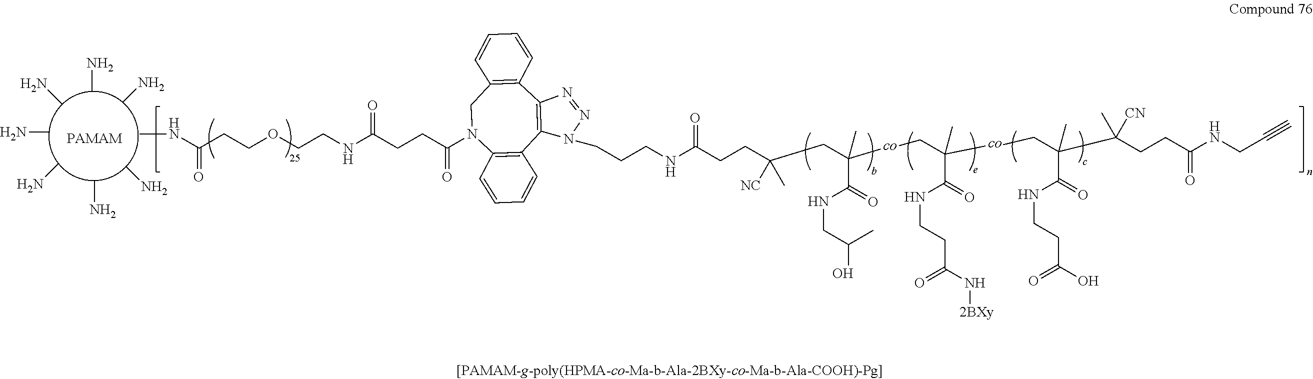

Compound 76 is an example of a star polymer with polymer arms comprised of hydrophilic monomers (B), reactive monomers (E) linked to drug (D), i.e. the TLR-7/8a 2BXy, through an amide bond, and charged monomers with a carboxylic acid functional group. PAMAM-g-poly(HPMA-co-Ma-b-Ala-2BXy-co-Ma-b-Ala-COOH)-Pg was synthesized using Compound 72 and Compound 48 in the same manner as Compound 75.

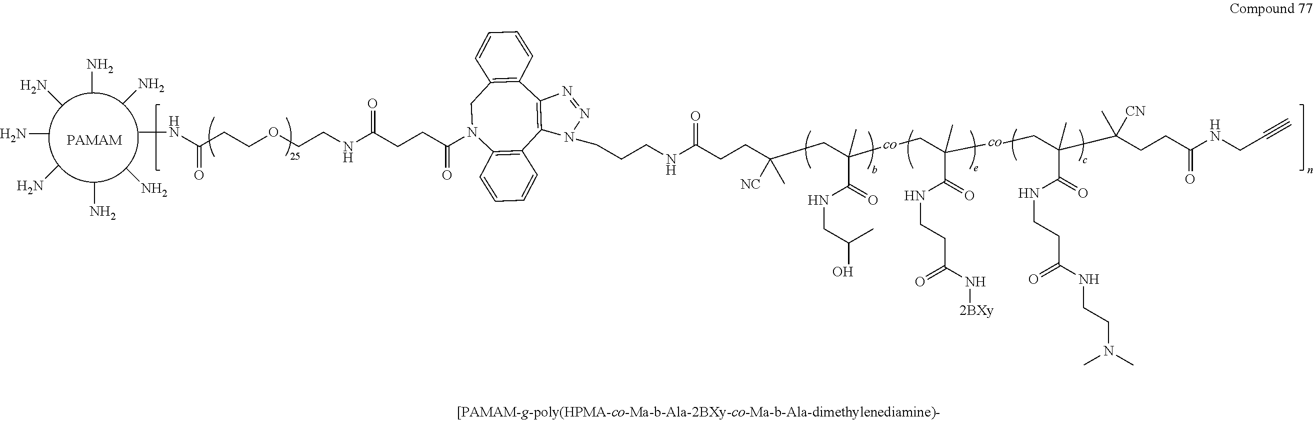

Compound 77 is an example of a star polymer with polymer arms comprised of hydrophilic monomers (B), reactive monomers (E) linked to drug (D), i.e. the TLR-7/8a, 2BXy, through an amide bond, and charged monomers with a tertiary amine functional group. PAMAM-g-poly(HPMA-co-Ma-b-Ala-2BXy-co-Ma-b-Ala-dimethylethylenediamine)-Pg was synthesized using Compound 72 and Compound 52 in the same manner as Compound 75.

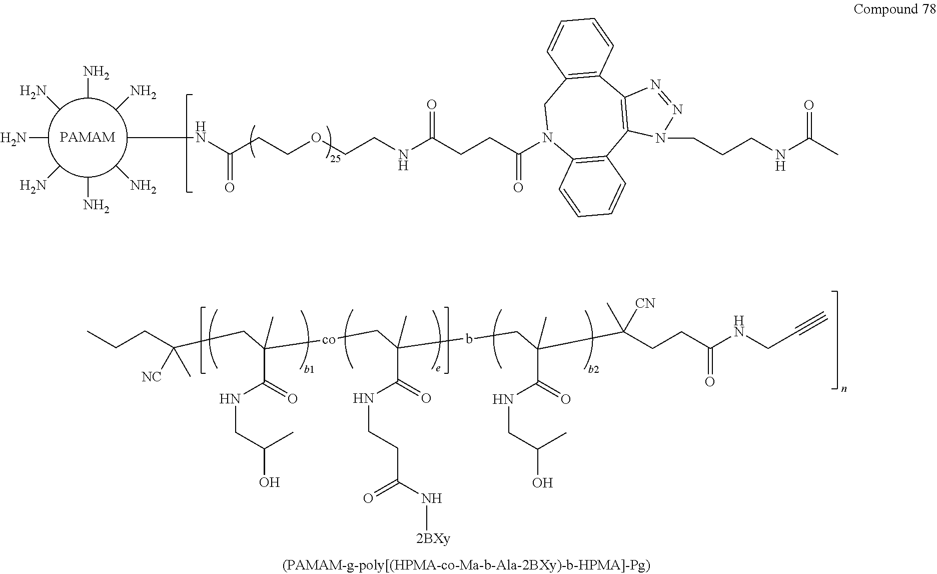

Compound 78 is an example of a star polymer with polymer arms with di-block architecture comprised of hydrophilic monomers (B) and reactive monomers (E) linked to drug (D), i.e. the TLR-7/8a, 2BXy, through an amide bond on one block proximal to the star polymer core and only hydrophilic monomers (B) on the other block distal to the core. PAMAM-g-poly[(HPMA-co-Ma-b-Ala-2BXy)-b-HPMA]-Pg was synthesized using Compound 72 and Compound 62 in the same manner as Compound 75.

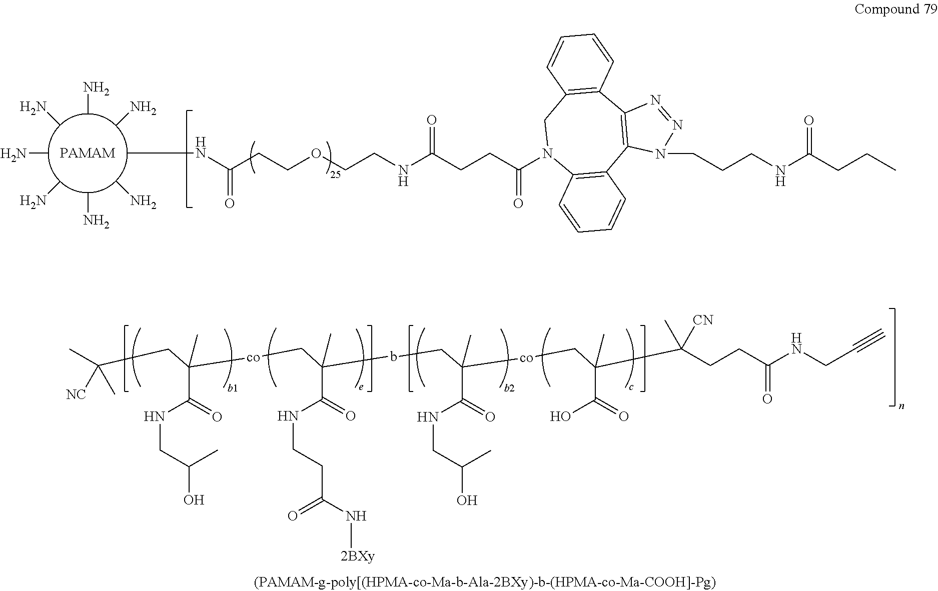

Compound 79 is an example of a star polymer with polymer arms with di-block architecture comprised of hydrophilic monomers (B) and reactive monomers (E) linked to drug (D), i.e. the TLR-7/8a, 2BXy, through an amide bond on one block proximal to the star polymer core, and both hydrophilic monomers (B) and charged monomers (C) with a carboxylic acid functional group on the other block distal to the core. PAMAM-g-poly[(HPMA-co-Ma-b-Ala-2BXy)-b-(HPMA-co-Ma-COOH]-Pg was synthesized using Compound 72 and Compound 66 in the same manner as Compound 75.

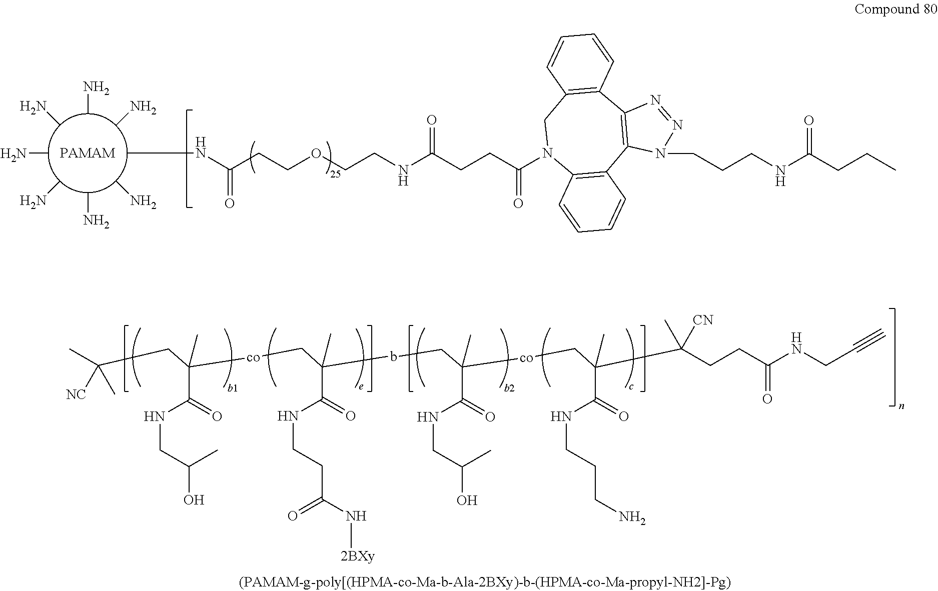

Compound 80 is an example of a star polymer with polymer arms with di-block architecture comprised of hydrophilic monomers (B) and reactive monomers (E) linked to drug (D), i.e. the TLR-7/8a, 2BXy, through an amide bond on one block proximal to the star polymer core, and both hydrophilic monomers (B) and charged monomers (C) with an amine functional group on the other block distal to the core. PAMAM-g-poly[(HPMA-co-Ma-b-Ala-2BXy)-b-(HPMA-co-Ma-propyl-NH2]-Pg was synthesized using Compound 72 and Compound 68 in the same manner as Compound 75.

### Example 6—Synthesis of Star Polymers for Ligand Display by Route 2

For route 2 synthesis, star polymer carriers of ligands (L), drugs (D) or both L and D, were prepared by reacting linear polymer arms, which contain L and/or drug reactive linker(s), with dendrimer cores to generate star polymers that are reactive towards L and/or D, i.e. L and/or D are added after attachment of polymers arms (A) to the core.

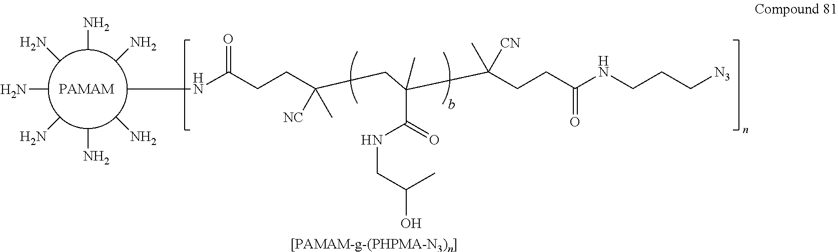

Compound 81 is an example of a star polymer, wherein the polymers arms (A) are linked to the core through a linker X that comprises an amide and are terminated with a Z1 linker precursor that comprises an azide. The following procedure was employed to produce azide-functionalized star NP with TT/NH2 linkages [PAMAM-g-(PHPMA-N₃)ₙ] by acylation between TT on PHPMA arm and primary amine on PAMAM core: TT-PHPMA-N₃ (376.3 mg, 7.68 μmol) was dissolved in 1.5 mL of anhydrous DMSO in a 15 mL falcon tube. PAMAM dendrimer generation 3.0 solution (19.2 μL of 20 wt % in MeOH solution, 15.36 μmol of -NH₂ groups) was added to the tube. The reaction was allowed to proceed at r.t. overnight. The star polymer was purified using spin column (Amicon, 70 mL, MWCO 50 kDa) and lyophilized to yield white solid (300.0 mg, 78.9% yield). Number-average (Mₙ) and weight-average molecular weight (M_{w}) were 848.9 kDa and 914.4 kDa, respectively, and polydispersity (PDI) was 1.08 measured by GPC-MALS.

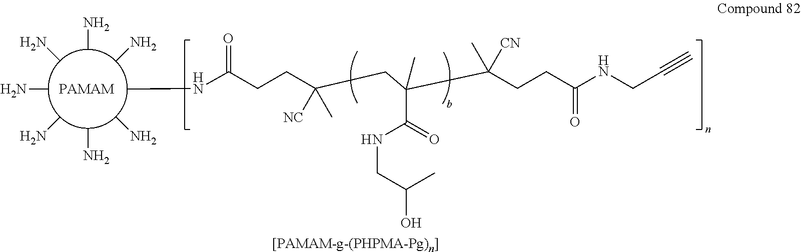

Compound 82 is an example of a star polymer, wherein the polymers arms (A) are linked to the core through a linker X that comprises an amide and are terminated with a Z1 linker precursor that comprises a propargyl. Propargyl-functionalized star polymers with TT/NH2 linkages [PAMAM-g-(PHPMA-Pg)˜] were prepared by acylation between TT-PHPMA-Pg and primary amine on PAMAM dendrimer using the same method as described for Compound 81.

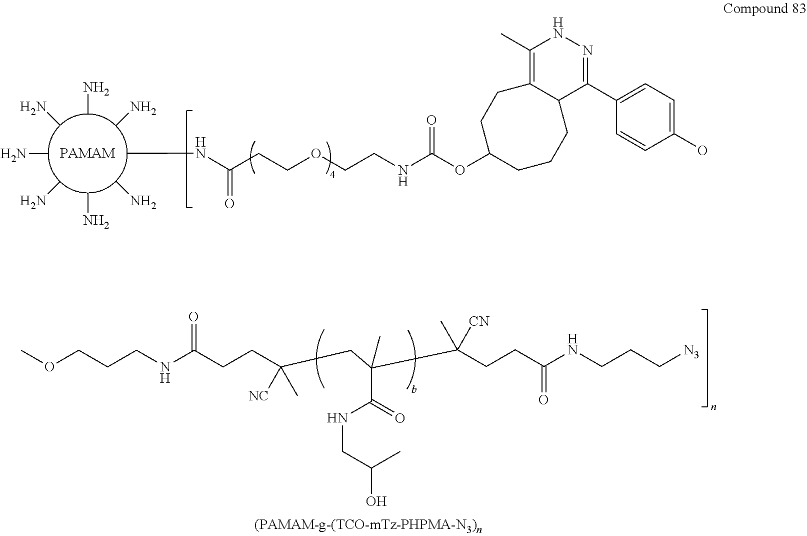

Compound 83 is an example of a star polymer, wherein the polymers arms (A) are linked to the core through a linker X that comprises the product of methyltetrazine and TCO and are terminated with a Z1 linker precursor that comprises an azide. Azide-functionalized star polymers with mTz/TCO linkages [PAMAM-g-(TCO-mTz-PHPMA-N₃)ₙ] were prepared using “click” chemistry between the mTz group on Compound 29, mTz-PHPMA-N₃ and TCO groups on Compound 69, PAMAM-TCO dendrimer in the same manner as described for as described for Compound 81.

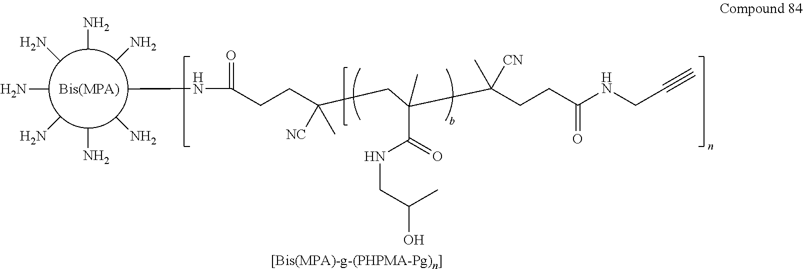

Compound 84 is an example of a star polymer, wherein the polymers arms (A) are linked to the core through a linker X that comprises an amide and are terminated with a Z1 linker precursor that comprises a propargyl. Bis(MPA)-g-(PHPMA-Pg)n was synthesized using the same method as described for Compound 82, PAMAM-g-(PHPMA-Pg)n, except that PAMAM dendrimer was replaced by bis(MPA) and triethylamine (TEA) was added to deprotonate amine groups on bis(MPA) core, with TT/NH2/TEA=0.8/1/1. White solid was obtained with 22.4% yield. Number-average (Mₙ) and weight-average molecular weight (M_{w}) were 327.2 kDa and 388.5 kDa, respectively, and polydispersity (PDI) was 1.19 measured by GPC-MALS.

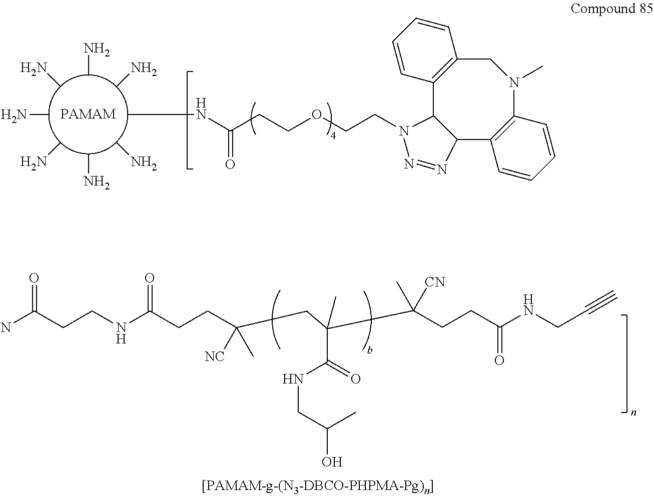

Compound 85 is an example of a star polymer, wherein the polymers arms (A) are linked to the core through a linker X that comprises a triazole and are terminated with a Z1 linker precursor that comprises a propargyl. Propargyl-functionalized star polymers with DBCO/N₃ linkages [PAMAM-g-(N₃-DBCO-PHPMA-Pg)˜] were prepared using “click” chemistry between the DBCO group on Compound 35, Pg-PHPMA-DBCO and azide groups on Compound 70, PAMAM-N₃ dendrimer in the same manner as described for as described for Compound 81.

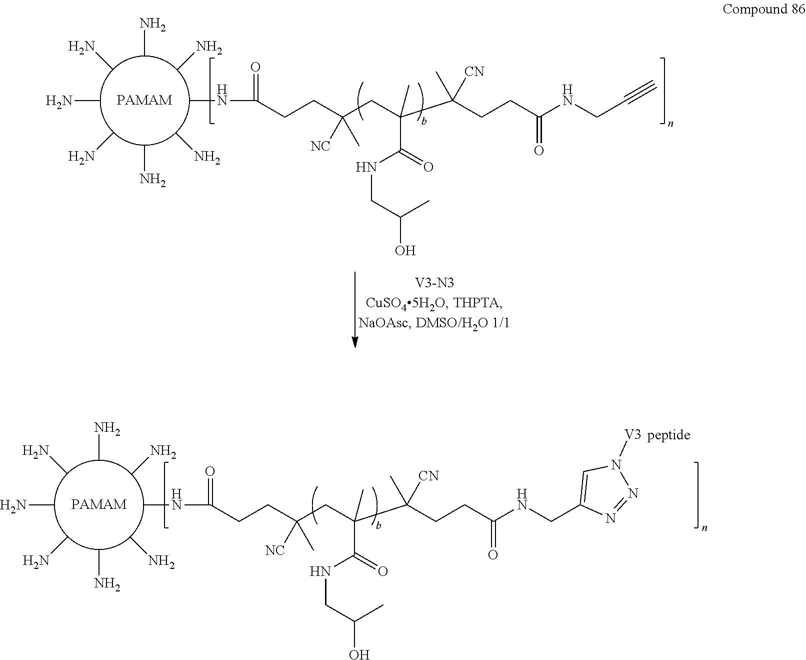

Compound 86. Star polymers displaying multiple B cell immunogens (peptide-N₃ or “V3-N₃”) on the surface was synthesized via copper-catalyzed alkyne-azide “click” chemistry. [peptide-N₃]₀:[Pg]₀ molar ratio is adjusted to vary V3 loading per each star molecule and HPLC was used to ensure quantitative conversion. For example, star polymer PAMAM-g-(PHPMA15k-Pg)₃₀] (1.5 mg, 100 nmol Pg), V3-N₃ (0.27 mg, 78 nmol), CuSO₄.5H₂O (0.40 mg, 1.6 μmol), sodium ascorbate (NaOAsc, 0.32 mg, 1.6 μmol), and THPTA (0.69 μg, 1.6 μmol) were mixed in 87 μL of DMSO/H₂O cosolvent (1/1 v/v). The reaction was allowed to proceed at r.t. overnight. HPLC characterization was performed to confirm quantitative conversion of V3-N₃ peptide. The reaction mixture was diluted to 3× the original volume with MeOH/H₂O cosolvent (1/1, v/v). The product was then purified by dialyzing against 2 rounds of MeOH/H₂O (1/1, v/v) with 0.01% ethylenediaminetetraacetic acid (EDTA), MeOH/H₂O cosolvent (1/1, v/v) and 2 rounds of H₂O. The resulting solution was lyophilized to yield off-white solid product (1.2 mg, 67.8% yield).

### Example 7—TLR-7/8a Attachment to the Core of a Star Polymer Displaying Peptide-Based B Cell Immunogens

The TLR7/8 agonist adjuvant (2Bxy) was attached to the PAMAM core of the star polymer in two steps using short heterobifunctional PEG linkers. First, the star polymer (7.21e⁻⁵ mol —NH₂ groups, 0.352 g) was dissolved in DMSO (10 wt % solution), mixed with NHS-PEG₄-DBCO (2.25e-⁻⁵ mol, 0.015 g) in 0.146 mL of DMSO and allowed to react 3 h at r.t. Second, 2Bxy-N₃ (2.25e⁻⁵ mol, 0.011 g) was added to the reaction mixture and reacted for 3 h at r.t. After that, the product was purified by the gravity SEC using Sephadex LH-20 in methanol and precipitated into diethyl ether yielding 0.342 g of white solid. M_{w} and M_{w}/Mₙ of the star polymer bearing multiple TLR7/8 agonists were 476.1 kDa and 1.12, respectively. Azide group-containing peptide immunogens were attached to the terminal propargyl groups on the PHPMA grafts of the star polymers via Cu^{I} catalyzed cycloaddition reaction in the presence of TBTA in DMSO/H₂O (2:1) mixture. For example, equimolar amounts of star polymer (6.10e⁻⁷ mol ˜Pg groups, 10.0 mg), V3 peptide (6.10e⁻⁷ mol, 2.1 mg) and TBTA (6.10e⁻⁷ mol, 0.32 mg) were dissolved in DMSO (5 wt. % solution) and bubbled with argon. Then, the equimolar amount of CuBr (6.10e⁻⁷ mol, 0.09 mg) was added to the reaction mixture; the solution was diluted with distilled water and allowed to react overnight at r.t. The resulting star-shaped co-polymer/V3 peptide conjugate was mixed with 1 ml of 8-hydroxyquinoline (1 wt. % solution in methanol) and consecutively purified by the gravity SEC using Toyopearl HW-40F and Sephadex LH-20 in methanol. The methanol was evaporated to dryness and the residue was dissolved with a defined volume of DMSO.

### Example 8—Use of a Star Polymer Displaying Peptide-Based Antigens as Ligands (L) that Bind to B Cell Receptors as a Vaccine for Inducing Antibody Responses

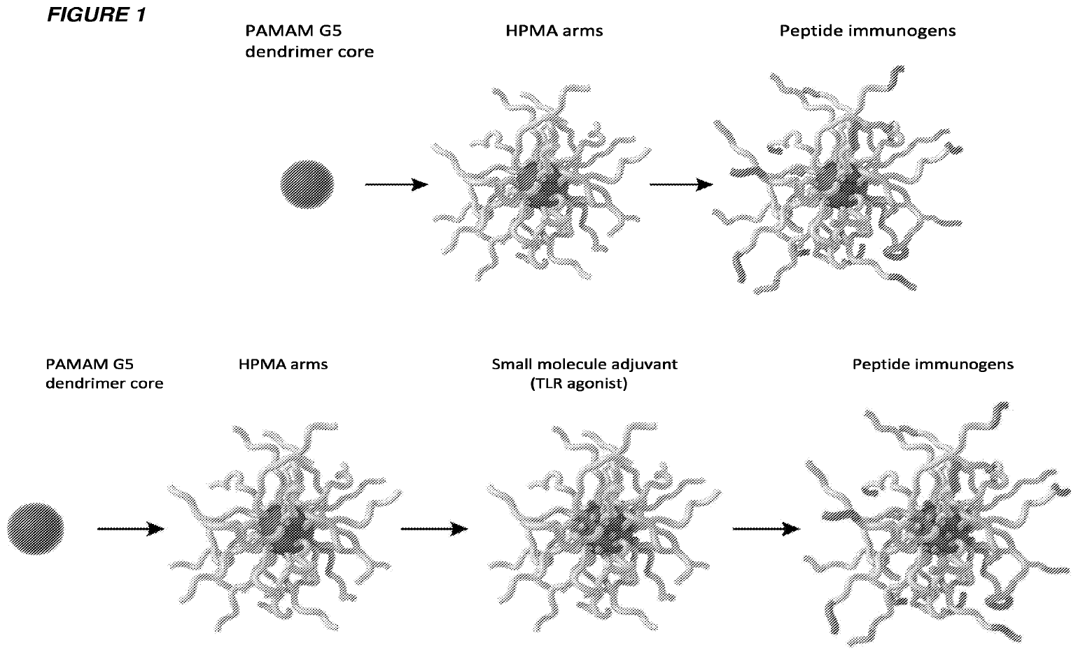

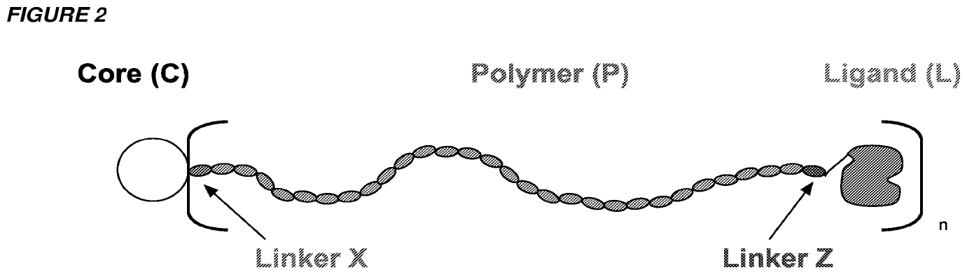

Peptide minimal immunogens, i.e. peptide-based antigens comprising minimal epitopes, can be used to elicit antibodies against specific epitopes of infectious organisms or cancer cells. Herein, we designed peptide-based antigens as minimal HIV immunogens that mimic multiple epitopes from the HIV envelope (Env) glycoprotein and attached these to the ends of polymer arms (A) radiating from the core of dendrimer-based star polymers to produce star polymer vaccines. In some embodiments of the star polymer vaccines comprising HIV minimal immunogens, a CD4 helper epitope, i.e. “PADRE,” and/or TLR-7/8a, were attached to the ends of additional polymer arms and the core, respectively, as represented schematically in FIG. 1, which may be represented more generically as shown in FIG. 2.

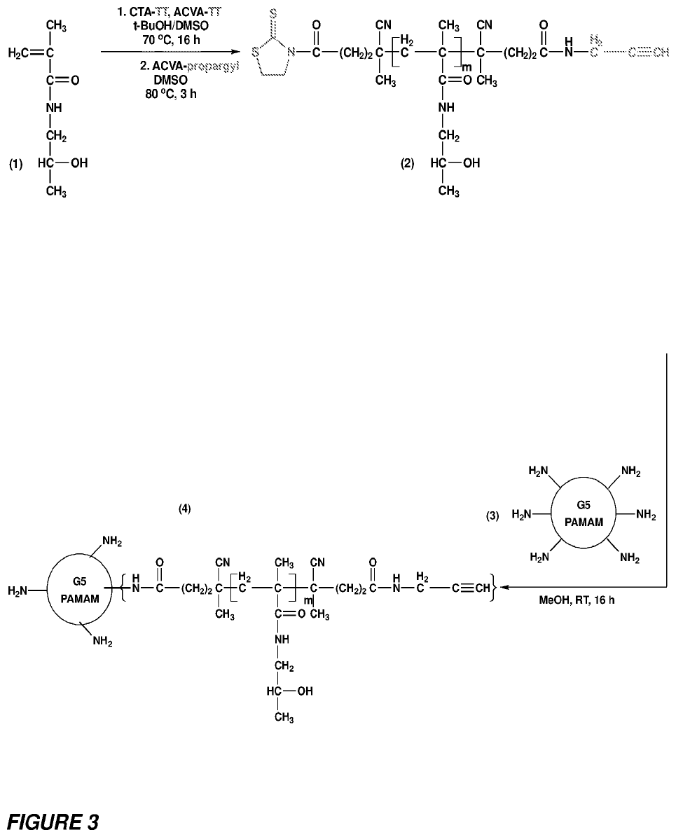

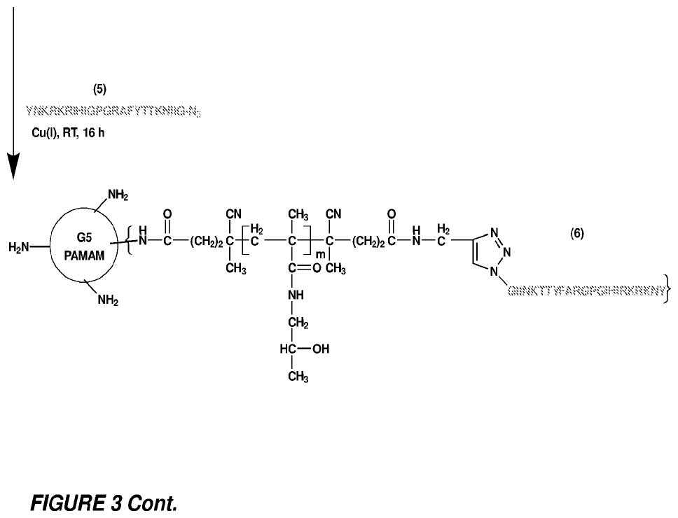

After attaching the peptide-based antigen, YNKRKRIHIGPGRAFYTTKNIIG (SEQ ID NO: 3), referred to as the “V3” minimal immunogen or ligand (L) on ˜30 10 kDa HPMA-based polymer arms linked to a G5 PAMAM dendrimer core using the synthetic route show in in FIG. 3, the hydrodynamic radius of the resulting star polymer vaccine was found to be 13 nm by dynamic light scattering (FIG. 4). Similar measurements were obtained when a mixture of V3 and PADRE T-helper peptides, i.e. peptide antigens with the sequence AKFVAAWTLKAAA (SEQ ID NO: 4), were attached at 1:1 ratio to 10 kDa HPMA-based polymer arms radiating from a G5 PAMAM dendrimer core; though, the radius increased slightly when a small molecule TLR7/8 agonist was attached to the core (24 nm) of the star polymer vaccine, possibly suggesting a conformational difference in the flexible HPMA arms when an amphiphilic agonist is attached to the core (FIG. 4).

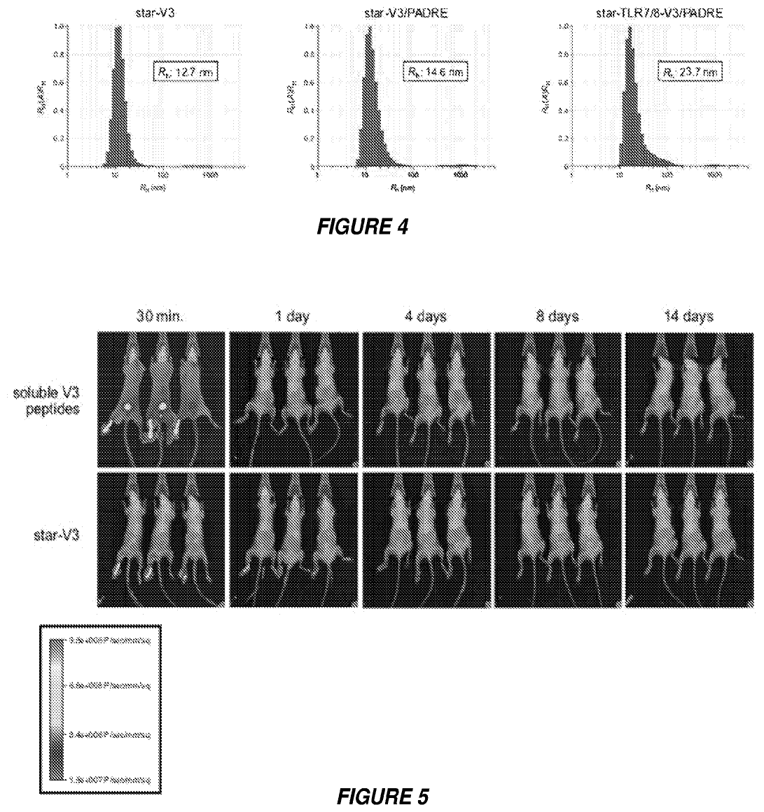

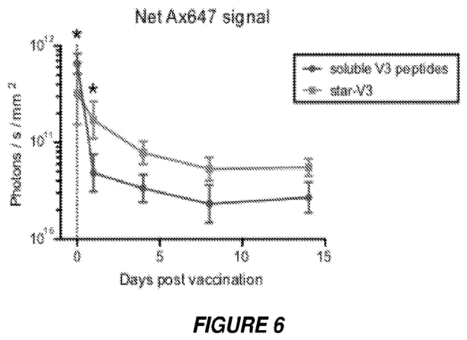

To evaluate the how attachment of peptide-based antigens to star polymers impacts pharmacokinetics, we conjugated fluorescence dye molecules to a star polymer vaccine comprised of the HIV Env minimal immunogen, “V3,” linked to HPMA-based polymer arms radiating from a PAMAM dendrimer core and tracked the material in vivo following vaccination. As a control, unconjugated, soluble V3 peptides were found to rapidly disseminate throughout the body by 30 minutes post injection (FIGS. 5 and 6). For the remainder of the 2-week observation period, the soluble peptides had a biodistribution localized mostly to the liver and spleen and, to a lesser extent, the site of injection. Strikingly, when arrayed on star polymers the V3 peptides did not show a disseminated biodistribution at any time point, but could only be visualized at the injection site and at the liver and spleen region. Because soluble V3 peptides could rapidly diffuse from the site of injection, we quantified the signal in the footpad region over time. Indeed, there was consistently more V3 immunogen remaining at the site of injection over time in mice vaccinated with the star polymer as compared to the mice that were injected with free peptide (FIG. 6). These data demonstrate how star polymers can be used to limit distribution and slow clearance of peptide-based antigens, as well as ligands (L), more generally, following injection into tissues that require localized and prolonged activity.

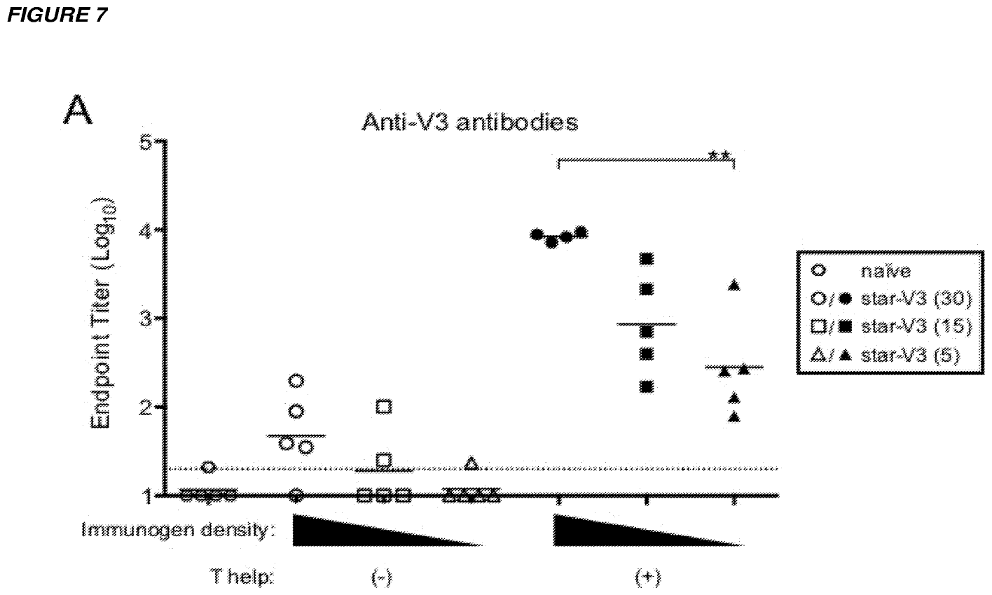

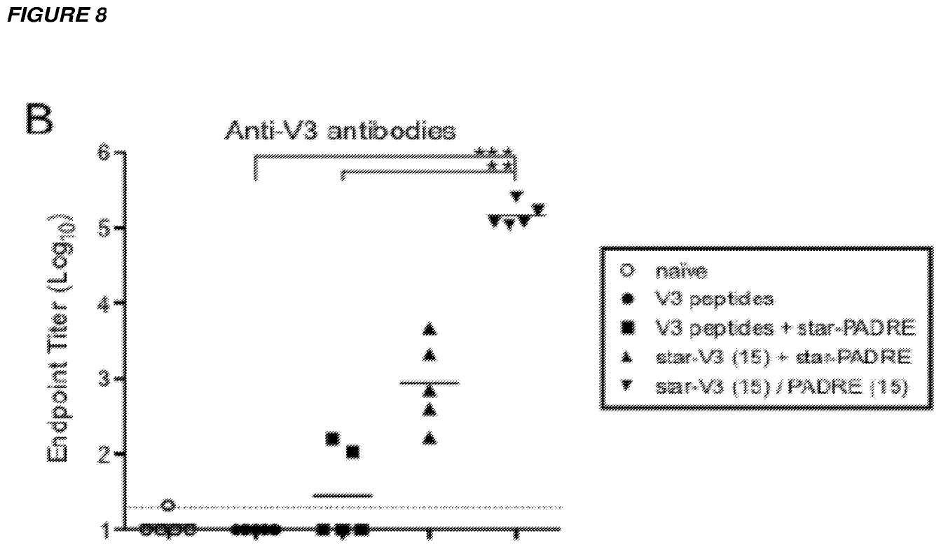

We next evaluated how the density of ligands (L) arrayed on the star polymer impacts in vivo activity. Keeping a constant immunogen dose across all groups, mice were immunized with star polymers bearing 5, 15, or 30 V3 peptides as minimal immunogens arrayed on star polymers (FIG. 7). Binding antibody titers were directly correlated with antigen density, with increasing magnitude of antibodies generated with increasing densities of ligand (L), i.e. V3, arrayed on the star polymers. Furthermore, antibody titers to all groups increased by ˜2 logs when the star polymers were co-administered with star polymers displaying T cell helper (PADRE) peptides (FIG. 8). While soluble V3 peptides were non-immunogenic and could not be significantly improved by the addition of star polymers containing PADRE, a mixture of star polymers displaying either V3 and PADRE peptides was highly immunogenic for inducing antibody responses (FIG. 8). Interestingly, star polymers displaying 15 V3 peptides and 15 PADRE peptides as ligands (L) on the same star polymer elicited V3 titers that were 2 logs higher than when two separate star polymers either bearing V3 or PADRE were mixed together (FIG. 8).

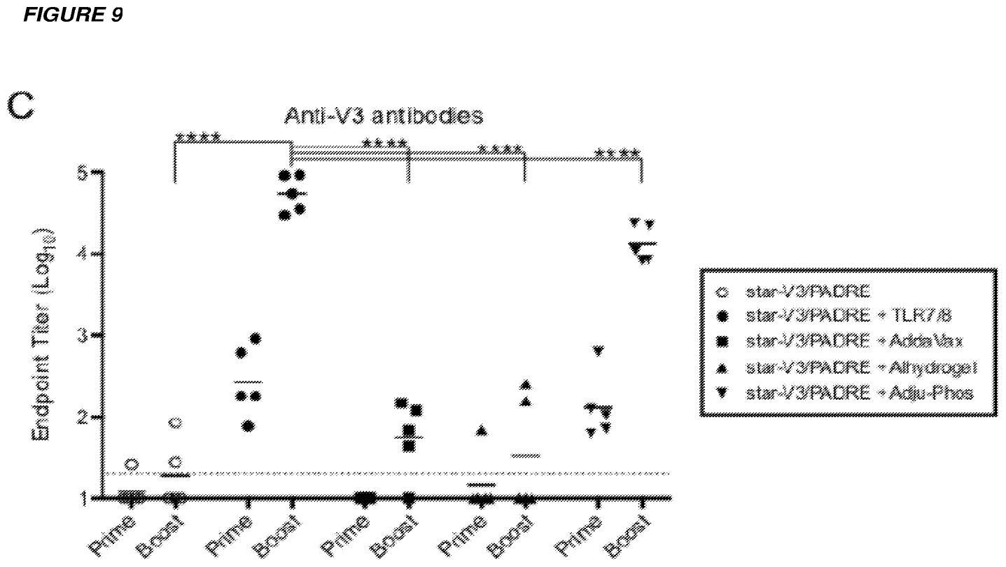

We next examined how several different adjuvants that have been used clinically altered the antibody response elicited by star polymers. Consistent with prior studies by us and others [2, 3], the TLR7/8 agonist was found to be the most potent of all adjuvants tested, followed by Adju-Phos (FIG. 9).

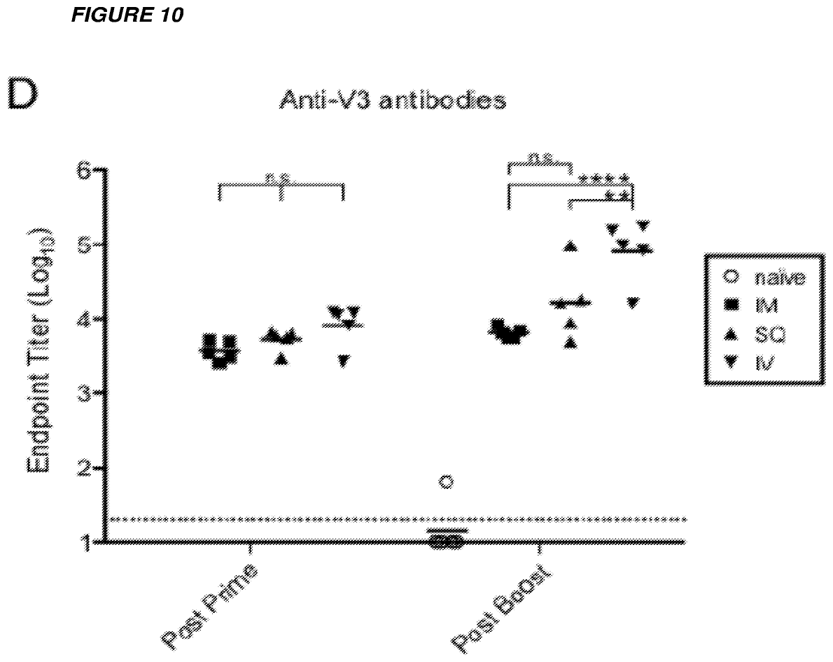

We also evaluated the immunogenicity of star polymer vaccines using different routes of administration. Mice were immunized intramuscularly (IM), subcutaneously (SC) and intravenously (IV). While no difference was observed after 1 immunization, mice immunized by the IV route had ˜1 log higher antibody titers than the IM and SC groups after a boost (FIG. 10).

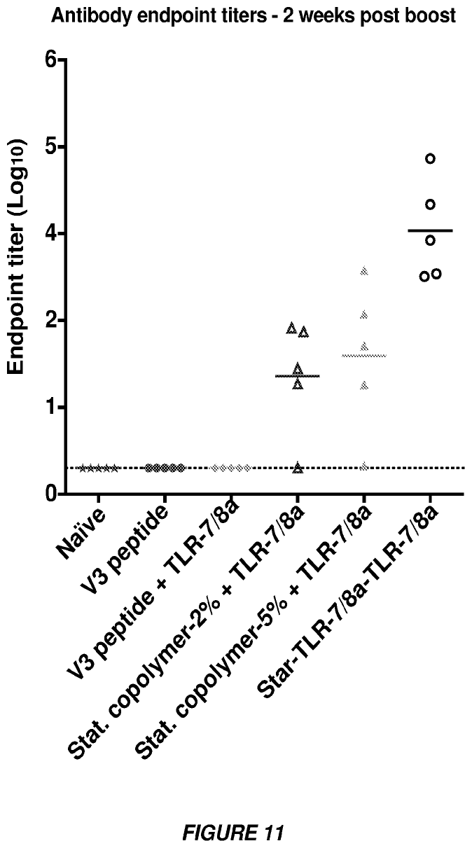

Notably, star polymer vaccines displaying the V3 minimal immunogen as the ligand (L) led to higher magnitude antibody responses as compared with statistical co-polymers displaying similar densities of the same ligand (L), suggesting that the size and/or architecture may be important to the activity of vaccines comprising peptide antigens as minimal immunogens (FIG. 11).

In conclusion, the data show that star polymers densely arraying peptide-based antigens effectively engage B cells and elicit high titer antibody responses in mammals.

To extend these findings, additional star polymers were prepared using minimal immunogens derived from flu (i.e. LNDKHSNGTIKDRSPYR (SEQ ID NO:6), DPNGWTGTDNNFS (SEQ ID NO:7) and RNNILRTQESE (SEQ ID NO:8)), hepatitis B (i.e. PRVRGLYFL (SEQ ID NO:9), HPV (i.e. QLYQTCKAAGTCPSDVIPKI (SEQ ID NO:10)) and Malaria (i.e. EDNEKLRKPKHKKLKQPADGNPDPNANPNVDPNAN (SEQ ID NO:11), ILRNQYNNIIELEKTKHIIHNKKDTYKYDIKLKESDILMFYMKEETIVESGN (SEQ ID NO:12) and VLNKKEKKPRGIDFTETDELEQTDIVQNGNDKLVKVKENETIHFKFNSNQKLEIKE (SEQ ID NO:13)) that were linked via a triazole to the ends of HPMA-based polymer arms radiating from a PAMAM dendrimer core at high densities (i.e. n>15) to produce star polymer vaccines for inducing antibody responses. Notably, all of the different compositions of star polymer vaccines were effective for inducing antibody responses in mice, which demonstrates the broad potential of the star polymer compositions described herein as platforms for displaying B cell immunogens for use as vaccines.

### Example 9—Impact of Polymer Arm (A) Molecular Weight on Star Polymer Rh

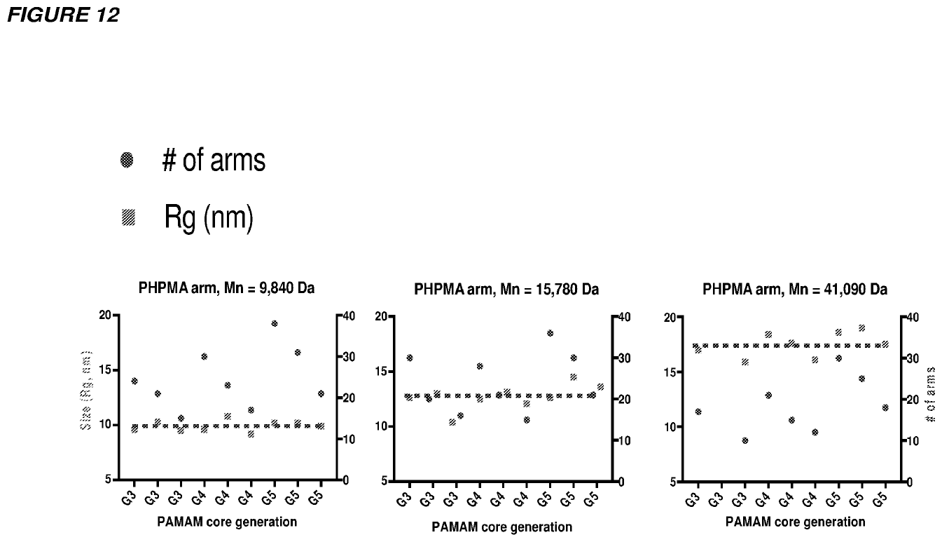

The impact that polymer arm density, polymer arm molecular weight and dendrimer core generation have on the size (Rg) of star polymers was investigated. Unexpectedly, the radius of star polymers, both radius of gyration (Rg) and hydrodynamic radius (Rh) was principally dictated by the polymer arm molecular weight but not the number of arms or the generation of the dendrimer core (FIG. 12).

To investigate how the length of polymer arms (A) and the density of ligands (L) arrayed on star polymers impacts biological activity, a library of star polymers with varying arm length and ligand (L) density were synthesized, characterized for physicochemical properties and then evaluated in vivo. Polymer arms based on Pg-PHPMA-TT were synthesized using the same synthetic procedure as for the preparation of Compound 34 except that the monomer, chain transfer agent and initiator ratio (i.e. [M]₀:[CTA]₀:[I]₀) was adjusted to produce four HPMA-based polymers arms of varying molecular weight (15.0, 26.4, 54.1 and 88.4 kDa) as summarized in Table 1, below. Each of the different molecular weight HPMA-based polymers bearing an X2 linker precursor comprising a TT-activated acid was then reacted with a PAMAM Generation 5 core with 128 amine functionalities at different ratios of TT (X2) to amine (X1) to generate star polymers with between 27-28 or 15-16 arms (n) per star polymer. Note that the polymer arms (A) were attached to the core (O) using the same procedure as described for Compound 82, except with varying molar ratio of polymer arm and amine functionalities on PAMAM (Gen 5.0).

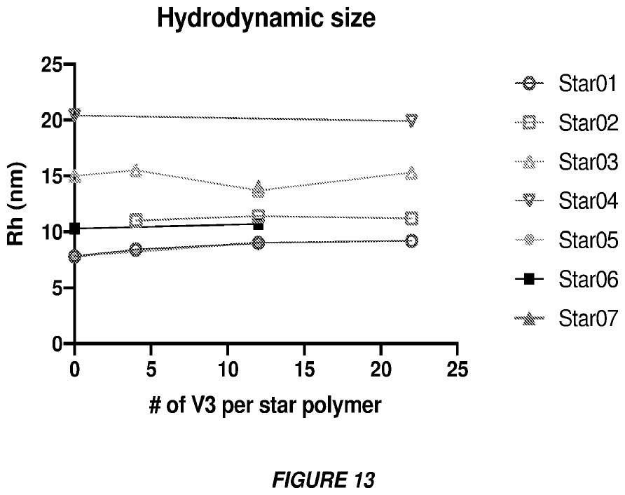

Next, the HIV Env minimal immunogen, V3, was linked at different densities (4, 12 or 22 V3 peptides per star polymer) via a linker Z comprising a triazole to the star polymers of varying molecular weight and arm density (referred to as Star01 through Star07), using the same method as described for Compound 86 to generate star polymers with varying arm length and ligand density (FIG. 13).

The hydrodynamic behavior of the different star polymers is shown in FIG. 13. In brief, the data substantiate that increasing polymers arm length, i.e. increasing polymer arm (A) molecular weight, is associated with increased Rh independent of the numbers of arms or density of ligands (L) attached. To assess how Rh and ligand density impact biological activity, each of the different star polymers delivering the V3 immunogen were administered to mice at day 0 and 14 and antibody responses generated against the V3 immunogen were assessed two weeks after the second administration. Notably, there was a correlation between increasing Rh and ligand density (i.e. V3 per star polymer) on antibody responses.

TABLE 1
Synthesis and characterization of star-V3 conjugates

| | PAMAM (G5) | Pg-PHPMA-TT arm | | TT/NH2 | Star polymer | | |
|---|---|---|---|---|---|---|---|
| Sample | # of NH2 | [M]₀:[CTA]₀:[I]₀ | Mn (kDa) | molar ratio | Mn (kDa) | Mw/Mn | Arm # |
| Star01 | 128 | 120:1:0.25 | 15.0 | 0.5 | 435.5 | 1.06 | 27 |
| Star02 | 128 | 240:1:0.25 | 26.4 | 0.5 | 764.1 | 1.06 | 28 |
| Star03 | 128 | 600:1:0.25 | 54.1 | 0.5 | 1520.2 | 1.05 | 28 |
| Star04 | 128 | 1200:1:0.25 | 88.4 | 0.63 | 2512.6 | 1.08 | 28 |
| Star05 | 128 | 120:1:0.25 | 15.0 | 0.28 | 260.6 | 1.01 | 15 |
| Star06 | 128 | 240:1:0.25 | 26.4 | 0.28 | 463.2 | 1.05 | 16 |
| Star07 | 128 | 600:1:0.25 | 54.1 | 0.33 | 848.6 | 1.03 | 15 |

### Example 10—Star Polymers with an Ester-Based Core

A variety of branched molecules can be used as cores for generating star polymers. As an alternative to PAMAM, amide-based cores, star polymers were produced using either generation 2, 4 or 5 Bis(MPA), ester-based cores. In short, TT-activated HPMA-based polymer arms (A) were reacted with bis(MPA) cores in the presence of triethylamine to generate the star polymers summarized in Table 2.

TABLE 2
Star polymers synthesized from bis(MPA) cores.

| | bis(MPA) core | | Pg- | TT/NH2/ | Star polymer | | |
|---|---|---|---|---|---|---|---|
| | (TFA salt) | | PHPMA- | TEA | properties | | |
| Sample | | # of | TT arm | molar | Mn | Mw/ | Arm # |
| # | Generation | NH2 | Mn (kDa) | ratio | (kDa) | Mn | (n) |
| 1 | G2 | 12 | 11.02 | 1/1/1 | 92.4 | 1.03 | 8.2 |
| 2 | G4 | 48 | 11.02 | 0.5/1/1 | 178.0 | 1.04 | 15.4 |
| 3 | G5 | 96 | 10 | 0.4/1/1 | 164.3 | 1.02 | 14.6 |
| 4 | G5 | 96 | 10 | 0.8/1/1 | 303.8 | 1.05 | 28.6 |

### Example 11—Methods for Preventing Star Polymer Cross-Linking During Manufacturing

Consistent manufacturing of uniform formulations is key to ensuring the success of any drug products for human use. Accordingly, star polymer manufacturing should ensure that star polymer compositions have uniform characteristics that are not variable between different batches.

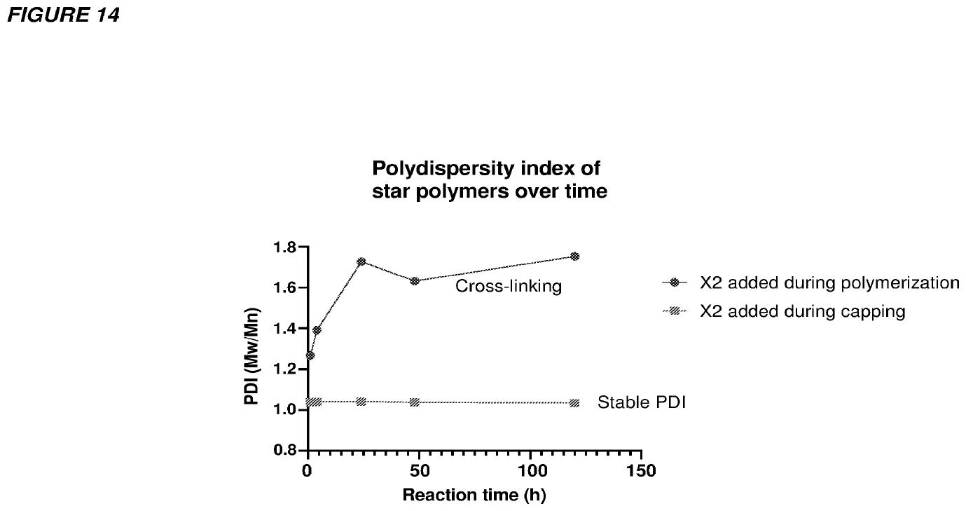

A key finding reported herein is that the process for introducing the linker precursor X2 on the star polymer can impact star polymer manufacturability. While the X2 linker precursor can be introduced on the polymer arm (A) either (i) during polymerization, i.e., by using a CTA and initiator functionalized with X2 (e.g. CTA-TT and ACVA-TT) or (ii) during the capping step, i.e., by reacting a polymer arm terminated with a CTA (e.g. pHPMA-DTB) with excess initiator functionalized with X2 (e.g., ACVA-TT), an unexpected finding reported herein is that introduction of X2 during the polymerization step results in polymers arms prone to cross-linking star polymers as indicated by the high polydispersity index of star polymers produced by this route (FIG. 14). In contrast, introduction of X2 linker precursor onto polymers arms during the capping step results in polymer arms that do not result in cross-linked star polymers. A non-limiting explanation for these results is that introduction of the X2 linker precursor on a polymer arm during polymerization, which is subsequently reacted with excess initiator during the capping step, results in a polymer arm impurity that is bifunctional for the linker precursor X2, i.e. the linker precursor X2 is linked to both ends of the polymer arm.

Based on these findings, several manufacturing innovations were introduced to reduce the potential for cross-linking to occur. As shown in FIG. 14, the risk of cross-linking can be eliminated by introducing the linker precursor X2 onto polymer arms during the capping step, rather than the polymerization step. However, for compositions of polymer arms that require the addition of the linker precursor X2 to the polymer arm during polymerization, two additional steps can be undertaken to reduce cross-linking, thereby improving manufacturability: (i) the concentration of the polymer arms in the reaction can be reduced and/or (ii) the time of the reaction can be reduced. Notably, it was observed that reducing the polymer arm concentration to 1 mM from 10 mM reduced the polydispersity index (PDI) from about 1.7 to 1.07, indicating a marked reduction in cross-linking. Additionally, keeping the reaction time to 1-hour results in a PDI of ˜1.05. Taken together, these results suggest that the linker precursor X2 should be introduced at any time after polymerization, e.g., during the capping step. Otherwise, if X2 must be added to the polymer arms during polymerization than the concentration of polymer arms during grafting to the core should be reduced to 1 mM or less and reaction time capped at 48 hours to prevent excessive cross-linking of the star polymers.

### Example 12—Methods for Improving Arm Coupling Efficiency to Star Polymers

Steric hindrance has historically prevented the efficient coupling of high densities of drug (D), e.g., greater than 10 mol %, to star polymers. Steric hindrance can also present challenges to coupling high densities of ligands with >10,000 Dalton molecular weight to star polymers. Therefore, it may be preferred to first attach drugs (D) and/or ligands (L) to polymer arms (A), and then couple these polymer arms to cores to generate star polymers linked to drugs and/or ligands, which is a manufacturing process herein referred to as Route 1. A major challenge for Route is that polymer arms bearing high densities of drug (D) and/or high molecular weight ligands (L) are relatively bulky and typically do not couple efficiently to cores to generate star polymers.

An unexpected finding reported herein is that bulky polymer arms with high densities of drugs (D) and/or ligands (L) of moderate to higher molecular weight could be more efficiently coupled to cores by introducing 4 or more ethylene oxide units onto X1 or on the linker between X1 and the core.

Accordingly, the grafting efficiency, measured as mass percent conversion of polymer arms to the dendrimer core, was improved by extending the X1 linker precursor from the core using PEG13 or PEG24 (Table 3). These results show that the grafting efficiency can be improved markedly using linker precursors X1 linked to cores (O) through a PEG linker.

TABLE 3
Polymer arm grafting efficiency.

| | | | # of | % |
|---|---|---|---|---|
| X1 | Polymer arm | M_{N} | arms | Conversion |
| DBCO | Pg-poly[(HPMA)-b-(HPMA- | 739.4 | 17.2 | 13.8 |
| | co-Ma-b-Ala-2BXy)]-N3 | | | |
| PEG13- | Pg-poly[(HPMA)-b-(HPMA- | 869.3 | 20.2 | 28.5 |
| DBCO | co-Ma-b-Ala-2BXy)]-N3 | | | |
| PEG24- | Pg-poly[(HPMA)-b-(HPMA- | 812.8 | 18.6 | 67.1 |
| DBCO | co-Ma-b-Ala-2BXy)]-N3 | | | |

### Example 13—Polymers with Block Architecture and/or Charged Monomers Enable Efficient Loading (i.e. High Densities) of Amphiphilic or Hydrophobic Drugs on Star Polymers

Increased drug (D) and ligand (L) loading per star polymer was associated with enhanced biological activity. Therefore, compositions and methods of manufacturing star polymers that enable consistent manufacturing of uniform formulations of star polymers with high drug (D) and/or or ligand (L) densities are needed. In addition to the aforementioned challenges associated with the process for manufacturing star polymers with high densities of drug (D) and/or ligand (L), the chemical composition of the drug (D) or ligand (L) itself can also pose challenges. Specifically, amphiphilic or hydrophobic drugs (D), such as small molecule drugs comprising cyclic ring structures, such as aromatic heterocycles, attached to star polymers at high densities can cause aggregation of the star polymers, which can present challenges to manufacturing drug products for human use.

To address this challenge, two design features were introduced that enable loading of high densities of drug (D) and/or ligands (L) on star polymers without the resulting star polymer carriers of drugs and/or ligands aggregating. The two innovations were to either or both (i) use star polymers comprised of polymer arms (A) with diblock architecture wherein drug and/or ligand are attached to the block of the polymer arm (A) that is proximal to the core (0) and (ii) include charged monomers on the polymer arm (A).

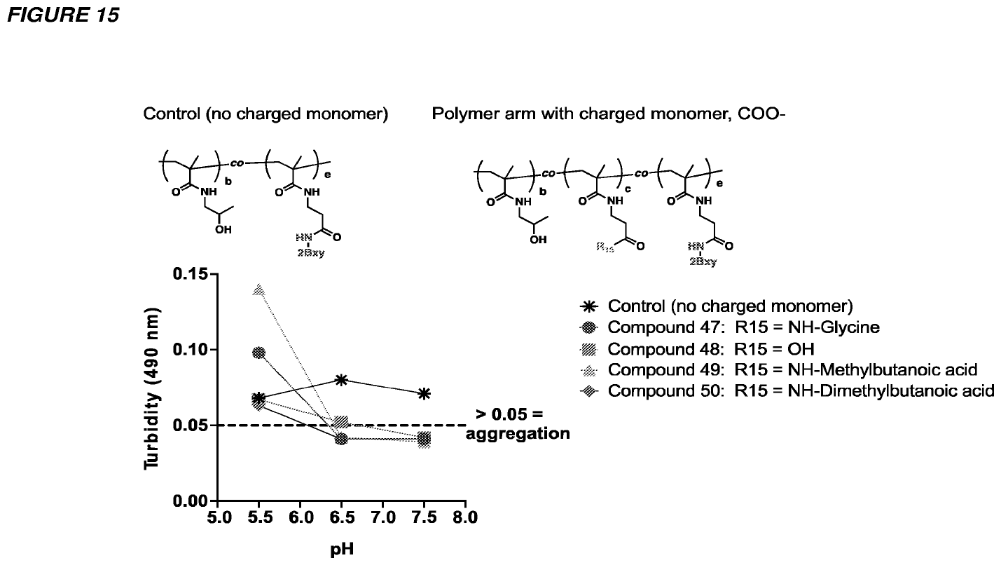

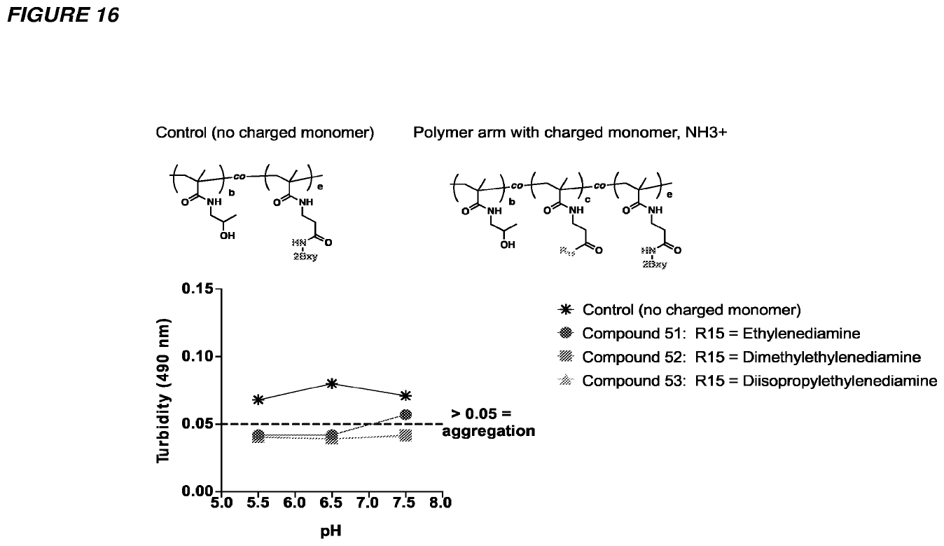

It was unknown a priori what composition and magnitude of charge would be needed to fully solubilize polymer arms with high densities of amphiphilic or hydrophobic drug (D) molecules. Therefore, we attached high densities (˜10 mol %) of the small molecule aromatic heterocycle, 2BXy, which is a TLR-7/8a, to ˜40 kDa HPMA-based polymer arms (A) through a reactive monomer (E), wherein the polymer arm (A) comprised HPMA monomers as the majority hydrophilic monomer (B) and optionally included either 10 or 20 mol % charged monomers (C) comprising either negatively or positively charged functional groups. Notably, whereas the copolymer without charged monomers formed aggregates at physiologic pH, ˜pH 7.4, as indicated by turbidity measurements (FIG. 15), polymer arms (A) with negatively charged carboxylic acid groups did not form aggregates at physiologic pH. Similarly, polymer arms (A) that also included primary or tertiary amines, which can be protonated at physiologic pH, did not aggregate at physiologic pH. Notably, polymer arms with ethylene diamine but not propylene diamine showed some tendency to form aggregates at physiologic pH (FIG. 16).

Based on these data, two different compositions of star polymers were generated with terpolymers comprised of hydrophilic monomers (HPMA), reactive monomers linked to drug (MA-b-Ala-2BXy) and charged monomers with either negative (Ma-b-Ala-COOH) or positive (Ma-b-Ala-DMEDA) functional groups (at physiologic pH). Notably, both star polymers (Compounds 76 and 77, Table 4) were stable in aqueous buffer (PBS) at physiologic pH. Importantly, preserving the small size (Rh˜10 nm) of the star polymers with high densities (˜10 mol %) of the TLR-7/8a by using high densities (˜20 mol %) of charged monomers was also associated with improved biological activity.

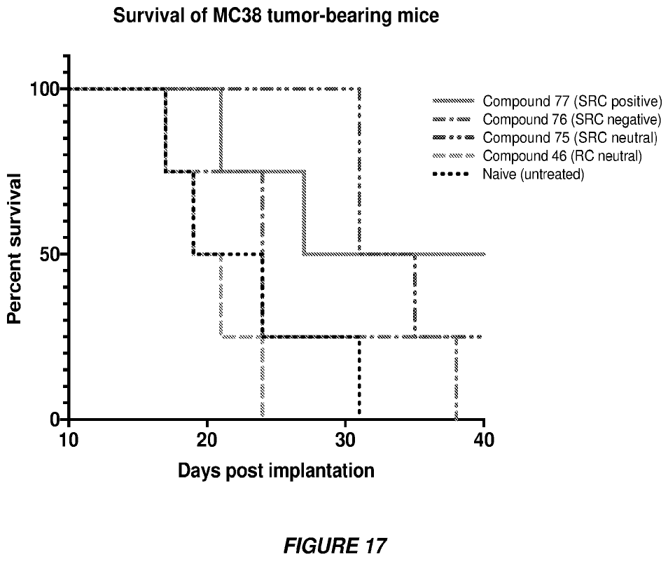

Specifically, mice with MC38 tumors treated with the star polymers comprising TLR-7/8a and charged monomers had improved survival as compared with mice that received neutral star polymers with random coil architecture that did not include charged monomers (Compound 75, FIG. 17).

TABLE 4
star polymers with polymer arms that include charged monomers and high densities of drug (D).

| | | | | mol % (#), | mol % | | |
|---|---|---|---|---|---|---|---|
| Cmpd | | Mn | | monomer | (#) TLR- | Arm | Turbidity, |
| # | Composition | (kDa) | PDI | C | 7/8a | # (n) | pH 7.4 |
| 48 | N3-poly(HPMA-co-Ma-b-Ala-2BXy- | 43.1 | 1.07 | 20 (50) | 10.3 | N.A. | 0.043 |
| | co-Ma-b-Ala)-Pg | | | | (22) | | |
| 51 | N3-poly(HPMA-co-Ma-b-Ala-2BXy- | 46.1 | 1.10 | 20 (50) | 10.3 | N.A. | 0.044 |
| | co-Ma-b-Ala-DMEDA)-Pg | | | | (22) | | |
| 76 | PAMAM-g-(poly(HPMA-co-Ma-b-Ala- | 483.4 | 1.19 | 20 (50) | 10.3 | 10.1 | 0.044 |
| | 2BXy-co-Ma-b-Ala))n-Pg | | | | (22) | | |
| 77 | PAMAM-g-(poly(HPMA-co-Ma-b-Ala- | 665.6 | 1.19 | 20 (50) | 10.3 | 13.4 | 0.052 |
| | 2BXy-co-Ma-b-Ala-DMEDA))n-Pg | | | | (22) | | |

Finally, star polymers with polymer arms (A) with di-block architecture were found to accommodate high densities (>10 mol %) of TLR-7/8a without forming aggregates (Table 5).

TABLE 5
star polymers with polymer arms that have di-block architecture and high densities of drug (D).

| | | | | mol % | | | Size, |
|---|---|---|---|---|---|---|---|
| Cmpd | | Mn | | (#) TLR- | Arm | Turbidity, | Rh |
| # | Composition | (kDa) | PDI | 7/8a | # (n) | pH 7.4 | (nm) |
| 46 | N3-poly(HPMA-co-Ma-b-Ala-2BXy)-Pg | 50.2 | 1.10 | 10.3 | N.A. | Aggregate | >1000 |
| | | | | (22) | | | |
| 62 | N3-poly((HPMA-co-Ma-b-Ala-2BXy)-b- | 35.8 | 1.28 | 11.6 | N.A. | 0.039 | 6.6 |
| | HPMA)-Pg | | | (23) | | | |
| 78 | PAMAM-g-(poly((HPMA-co-Ma-b-Ala- | 588.2 | 1.34 | 11.6 | 15.0 | 0.041 | 12.9 |
| | 2BXy)-b-HPMA))n-Pg | | | (23) | | | |

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

1. A star polymer of formula O[P1]-([X]-A[P2]-[Z]-[P3])n where O is a core; A is a polymer arm attached to the core; X is a linker molecule between the core and the polymer arm; Z is a linker molecule between the end of the polymer arm and P3; P1, P2 and P3 are each independently one or more compounds that act extracellularly or intracellularly, n is an integer number; [ ] denotes that the group is optional; and at least one of P1, P2 or P3 is present.

2. The star polymer of claim 1, wherein any one or more of P1, P2 or P3 is a ligand (L) comprising a pharmaceutically active compound that acts extracellularly.

3. (canceled)

4. The star polymer of claim 2, the star polymer having the formula O-([X]-A[(D)]-[Z]-L)n, where P2 is a drug (D) comprising a pharmaceutically active compound that acts intracellularly and [ ] denotes that the group is optional, wherein n is an integer number greater than or equal to 2, or wherein n is greater than or equal to 5.

5. (canceled)

6. The star polymer of claim 4, wherein:
a) the majority monomer units comprising the polymer arm (A) are selected from hydrophilic monomers; or
b) the polymer arms (A) comprise negatively charged functional groups; or
c) the majority monomer units comprising the polymer arm (A) are selected from hydrophilic monomers, wherein the polymer arms (A) comprise negatively charged functional groups.

7. (canceled)

8. The star polymer of claim 7, wherein the polymer arm (A) comprises 1 to 20 mol % co-monomers comprising negatively charged functional groups.

9. The star polymer of claim 8, wherein the co-monomers comprising negatively charged functional groups comprise poly(anionic) oligomers or polymers.

10. The star polymer of claim 9, wherein:
a) the polymer arms (A) comprises a di-block copolymer architecture; or
b) any co-monomers comprising negatively charged functional groups are on a first block of the di-block copolymer that is proximal to the ligand (L); or
c) the polymer arms (A) comprise a di-block copolymer architecture and any co-monomers comprising negatively charged functional groups are on a first block of the di-block copolymer that is proximal to the ligand (L.

11. (canceled)

12. The star polymer of claim 10, wherein one or more drugs (D), if present, are attached to co-monomers on a second block of the di-block copolymer that is proximal to the core (O), and the first block is solvent exposed and is not attached to any drugs (D).

13. The star polymer of claim 4, wherein the polymer arm length is selected to:
a) increase the size of the star polymer as a means to increase the persistence of activity of the star polymer in selected tissues; or
b) control the hydrodynamic radius of the star polymer; or
c) increase the size of the star polymer as a means to increase the persistence of activity of the star polymer in selected tissues and control the hydrodynamic radius of the star polymer.

14. (canceled)

15. The star polymer of claim 4, wherein:
a) the polymer arm molecular weight is greater than about 10,000 Daltons.

16. (canceled)

17. The star polymer of claim 4, comprising:
a) two or more ligands (L), which may be the same or different, and the ligands (L) are selected from compounds that bind to extracellular receptors selected from protein or peptide antigens, therapeutic antibodies or antibody fragments, peptide-MHC complexes, agonists of TLRs 1, 2, 4, 5, 6, CLRs or NLRs, or combinations thereof; and/or
b) one or more amplifying linkers that enable attachment of two or more ligands (L), which may be the same or different, on the ends of at least some of the polymer arms (A).

18. (canceled)

19. The star polymer of claim 4, wherein
a) the density of ligands (L) attached to the star polymer is greater than 5; or
b) saccharides that bind to the lectin receptor, CD22L, are placed at or near the ends of the polymer arms (A) proximal to the ligand (L); or
c) the density of ligands (L) attached to the star polymer is greater than 5 and saccharides that bind to the lectin receptor, CD22L, are placed at or near the ends of the polymer arms (A) proximal to the ligand (L).

20. (canceled)

21. The star polymer of claim 4, wherein:
a) the drugs (D), if present, are arrayed along the polymer arms (A) at a density greater than about 3 mol %; and/or
b) the drugs (D) have a molecular weight of between about 200-1,000 Da and are arrayed along the polymer arms (A) at a density of between about 4.0 to about 50 mol % to achieve a mass percent of about 10 to about 80 mass %.

22. (canceled)

23. The star polymer of claim 4, wherein the polymer arm (A) comprises hydrophilic monomers selected from acrylates, (meth)acrylates, acrylamides, (meth)acrylamides, allyl ethers, vinyl acetates, vinyl amides, substituted styrenes, amino acids, acrylonitrile, heterocyclic monomers (i.e. ethylene oxide), saccharides, phosphoesters, phosphonamides, sulfonate esters, sulfonamides, or combinations thereof.

24. The star polymer of claim 4:
a) the core (O) has greater than 5 points of attachment for polymer arms (A); or
b) the core (O) comprises a branched polymer or dendrimer; or
c) the core (O) has greater than 5 points of attachment for polymer arms (A) and comprises a branched polymer or dendrimer.

25. (canceled)

26. The star polymer of claim 24, wherein the dendrimer or branched polymer that is used to form the core (O) has surface amine groups used for the attachment of polymer arms (A), or wherein the core (O) is a dendrimer selected from PAMAM, bis(MPA) or lysine, or wherein the core (O) is a branched polymer that comprises monomers selected from poly(amino acids) or saccharides.

27. (canceled)

28. (canceled)

29. The star polymer of claim 1, wherein any one or more of P1, P2 or P3 is a drug (D) comprising a pharmaceutically active compound that acts intracellularly.

30. The star polymer of claim 1, the star polymer having the formula O-([X]-A(D)-[Z]-[L])n, where L is a ligand comprising a pharmaceutically active compound that acts extracellularly; D is a drug (D) comprising a pharmaceutically active compound that acts intracellularly.

31. The star polymer of claim 30, wherein n is greater than or equal to 5.

32. The star polymer of claim 30, wherein:
a) the majority monomer units comprising the polymer arm (A) are selected from hydrophilic monomers; or
b) the polymer arms (A) comprise negatively charged functional groups; or
c) the majority monomer units comprising the polymer arm (A) are selected from hydrophilic monomers, wherein the polymer arms (A) comprise negatively charged functional groups.

33. (canceled)

34. The star polymer of claim 32, wherein the polymer arm (A) comprises 1 to 20 mol % co-monomers comprising negatively charged functional groups.

35. The star polymer of claim 32, wherein the co-monomers comprising negatively charged functional groups comprise poly(anionic) oligomers or polymers.

36. The star polymer of claim 35, wherein the drugs (D) are arrayed along the polymer arms (A) at a density greater than about 3 mol %.

37. The star polymer of claim 36, wherein the drug (D) has a molecular weight of between about 200-1,000 Da and the drugs (D) are arrayed along the polymer arms (A) at a density of between about 4 to about 50 mol % to achieve a mass percent of about 5 to about 80 mass %.

38. The star polymer of claim 30, wherein:
a) the polymer arms (A) comprise a di-block copolymer architecture; or
b) any co-monomers comprising negatively charged functional groups are on a first block of the di-block copolymer that is distal to the core (O) and solvent exposed; or
c) the polymer arms (A) comprise a di-block copolymer architecture and any co-monomers comprising negatively charged functional groups are on a first block of the di-block copolymer that is distal to the core (O) and solvent exposed.

39. (canceled)

40. The star polymer of claim 38, wherein the one or more drugs (D) are attached to co-monomers on a second block of the di-block copolymer that is proximal to the core (O), and the first block is solvent exposed and is not attached to any drugs (D).

41. The star polymer of claim 30, wherein the polymer arm length is selected to:
a) increase the size of the star polymer as a means to increase the persistence of activity of the star polymer in selected tissues; or
b) control the hydrodynamic radius of the star polymer; or
c) increase the size of the star polymer as a means to increase the persistence of activity of the star polymer in selected tissues and control the hydrodynamic radius of the star polymer.

42. (canceled)

43. The star polymer of claim 40, wherein:
a) the polymer arm molecular weight is between about than about 5,000 to about 50,000 Daltons; or
b) the hydrodynamic radius of the star polymer is between about 5 nm and about 15 nm; or
c) the polymer arm molecular weight is between about than about 5,000 to about 50,000 Daltons and the hydrodynamic radius of the star polymer is between about 5 nm and about 15 nm.

44. (canceled)

45. The star polymer of claim 30, wherein:
a) the ligand (L), if present, is selected from compounds that bind to extracellular receptors selected from protein or peptide antigens, therapeutic antibodies or antibody fragments, peptide-MHC complexes, agonists of TLRs 1, 2, 4, 5, 6, CLRs or NLRs; or combinations thereof; or
b) the star polymer further comprises one or more amplifying linkers that enable attachment of two or more ligands (L), which may be the same or different, on the ends of at least some of the polymer arms (A); or
c) the ligand (L), if present, is selected from compounds that bind to extracellular receptors selected from protein or peptide antigens, therapeutic antibodies or antibody fragments, peptide-MHC complexes, agonists of TLRs 1, 2, 4, 5, 6, CLRs or NLRs; or combinations thereof, and the star polymer further comprises one or more amplifying linkers that enable attachment of two or more ligands (L), which may be the same or different, on the ends of at least some of the polymer arms (A).

46. (canceled)

47. The star polymer of claim 30, wherein:
a) the density of ligands (L) attached to the star polymer is greater than 5; and/or
b) the star polymer further comprises saccharides at or near the ends of the polymer arms (A) proximal to the ligand (L), wherein the saccharides bind to the lectin receptor, CD22L.

48. (canceled)

49. The star polymer of claim 30, wherein the polymer arm (A) comprises hydrophilic monomers selected from acrylates, (meth)acrylates, acrylamides, (meth)acrylamides, allyl ethers, vinyl acetates, vinyl amides, substituted styrenes, amino acids, acrylonitrile, heterocyclic monomers (i.e. ethylene oxide), saccharides, phosphoesters, phosphonamides, sulfonate esters, sulfonamides, or combinations thereof.

50. The star polymer of claim 30, wherein:
a) the core (O) has greater than 5 points of attachment for polymer arms (A); or
b) the core (O) comprises a branched polymer or dendrimer; or
c) the core (O) has greater than 5 points of attachment for polymer arms (A) and comprises a branched polymer or dendrimer.

51. (canceled)

52. The star polymer of claim 50, wherein the dendrimer or branched polymer that is used to form the core (O) has surface amine groups used for the attachment of polymer arms (A), or wherein the core (O) is a dendrimer selected from PAMAM, bis(MPA) or lysine, or wherein the core (O) is a branched polymer that comprises monomers selected from poly(amino acids) or saccharides.

53. (canceled)

54. (canceled)

55. A composition for sustaining activity of a pharmaceutically active compound that acts extracellularly comprising the star polymer of claim 1, wherein L is present in the star polymer and the star polymer has a hydrodynamic radius greater than 20 nm Rh.

56. An antitumor composition comprising the star polymer of claim 1, wherein D is present and selected from small molecule chemotherapeutic and/or immunostimulant drugs (D) and the star polymer has a hydrodynamic radius of from about 10 to about 15 nm Rh.

57. An antiviral composition comprising the star polymer of claim 1, wherein L is present in the star polymer.

58. A vaccine composition for inducing antibody responses comprising the star polymer of claim 1, wherein the polymer arm molecular weights are an average of about 10 kDa to about 60 kDa.

59. A process for preparing a star polymer according to claim 1, the process comprising:
reacting a heterotelechelic polymer arm (A) comprising a linker precursor Z1 with a ligand (L) comprising a linker precursor Z2 under conditions to form a linker molecule (Z) between the polymer arm (A) and the ligand (L):
X2-A[P2]-Z1+Z2-L→X2-A[P2]-Z-L,
and reacting the polymer arm-linker-ligand molecule comprising a linker precursor X2 with a core comprising a plurality of linker precursors X1 to form the star polymer:
O-X1+X2-A[P2]-Z-L→O-(X-A[P2]-Z-L)n; or
reacting a heterotelechelic polymer arm (A) comprising a linker precursor X2 with a core comprising a plurality of linker precursors X1 under conditions to form a core (0) attached to a plurality of polymer arms (A) via a linker molecule (X):
O-X1+X2-A[P2]-Z1→O(X-A[P2]-Z1)n, and
reacting the core-linker-polymer arm molecule comprising a linker precursor Z1 with a ligand (L) comprising a linker precursor Z2 under conditions to form a linker molecule (Z) between the polymer arm (A) and the ligand (L) to form the star polymer:
O-(X-A[P2]-Z1)n+Z2-L→O-(X-A[P2]-Z-L)n.

60. (canceled)
